(19) United States
(12) Patent Application Publication
(10) Pub. No.: US 2012/0157448 A1
(43) Pub. Date: Jun. 21, 2012

(54) COMPOUNDS FOR TREATING NEURODEGENERATIVE DISEASES

(76) Inventors: Adam Cook, Boulder, CO (US); Indrani W. Gunawardana, Boulder, CO (US); Kevin W. Hunt, Boulder, CO (US); Nicholas C. Kallan, Boulder, CO (US); Andrew T. Metcalf, Boulder, CO (US); Brad Newhouse, Boulder, CO (US); Tony P. Tang, Boulder, CO (US); Allen A. Thomas, Boulder, CO (US); Michael Siu, South San Francisco, CA (US); Malcolm Huestis, South San Francisco, CA (US); Matthew Volgraf, South San Francisco, CA (US)

(21) Appl. No.: 13/302,876

(22) Filed: Nov. 22, 2011

Related U.S. Application Data
(60) Provisional application No. 61/416,182, filed on Nov. 22, 2010.

(51) Int. Cl. A61K 31/4439; C07D 491/20; A61K 31/527; C07D 513/10; A61K 31/444; A61K 31/424; C07D 498/10; C07D 513/20; A61P 25/28; C07D 491/107; C07D 498/20; A61K 31/547

(52) U.S. Cl. 514/227.2; 546/15; 514/278; 544/231; 514/272; 544/6; 548/216; 514/374; 544/230; 514/256

(57) ABSTRACT

The invention provides novel tricyclic compounds of Formula I′ that inhibit β-secretase cleavage of APP and are useful as therapeutic agents for treating neurodegenerative diseases.

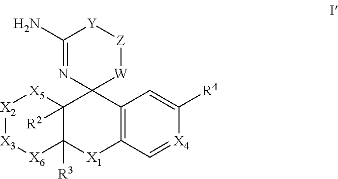

### PRIORITY OF INVENTION

This application claims priority to U.S. Provisional Application No. 61/416,182 that was filed on 22 Nov. 2010. The entire content of this provisional application is hereby incorporated herein by reference.

### BACKGROUND OF THE INVENTION

1. Field of the Invention

The present invention relates to organic compounds useful for inhibition of (—secretase enzymatic activity and the therapy and/or prophylaxis of neurodegenerative diseases associated therewith. More particularly, certain tricyclic compounds useful in the treatment and prevention of neurodegenerative diseases, such as Alzheimer's disease, are provided herein.

2. Description of the State of the Art

Alzheimer's disease (AD) is a neurological disorder thought to be primarily caused by amyloid plaques, an accumulation of abnormal protein deposits in the brain. It is believed that an increase in the production and accumulation of amyloid beta peptides (also referred to as Aβ or A-beta) in plaques leads to nerve cell death, which contributes to the development and progression of AD. Loss of nerve cells due to amyloid plaques in strategic brain areas, in turn, causes reduction in the neurotransmitters and impairment of memory. The proteins principally responsible for the plaque build up include amyloid precursor protein (APP) and presenilin I and II (PSI and PSII). Mutations in each of these three proteins have been observed to enhance proteolytic processing of APP via an intracellular pathway that produces Aβ peptides ranging from 39 to 43 amino acids. The Aβ 1-42 fragment has a particularly high propensity of forming aggregates due to two very hydrophobic amino acid residues at its C-terminus. Thus, Aβ 1-42 fragment is believed to be mainly responsible for the initiation of neuritic amyloid plaque formation in AD and is therefore actively being pursued as a therapeutic target. Anti-Aβ antibodies have been shown to reverse the histologic and cognitive impairments in mice which overexpress Aβ and are currently being tested in human clinical trials. Effective treatment requires anti-Aβ antibodies to cross the blood-brain barrier (BBB), however, antibodies typically cross the BBB very poorly and accumulate in the brain in low concentration.

Different forms of APP range in size from 695-770 amino acids, localize to the cell surface, and have a single C-terminal transmembrane domain. Aβ is derived from a region of APP adjacent to and containing a portion of the transmembrane domain. Normally, processing of APP by α-secretase cleaves the midregion of the Aβ sequence adjacent to the membrane and releases a soluble, extracellular domain fragment of APP from the cell surface referred to as APP-α. APP-α is not thought to contribute to AD. On the other hand, pathological processing of APP by the proteases β-secretase (also referred to as β-site of APP cleaving enzyme” (BACE-1), memapsin-2 and Aspartyl Protease 2 (Asp2)) followed by γ-secretase cleavage, at sites which are located N-terminal and C-terminal to the α-secretase cleavage site, respectively, produces a very different result than processing at the α site, i.e. the release of amyloidogenic Aβ peptides, in particular, Aβ 1-42. Processing at the β- and γ-secretase sites can occur in both the endoplasmic reticulum and in the endosomal/lysosomal pathway after reinternalization of cell surface APP. Dysregulation of intracellular pathways for proteolytic processing may be central to the pathophysiology of AD. In the case of amyloid plaque formation, mutations in APP, PS1 or PS2 consistently alter the proteolytic processing of APP so as to enhance Aβ 1-42 formation.

The initial processing of APP by β-secretase results in a soluble N-APP, which has recently been implicated in neuronal cell death through a pathway independent of amyloid plaque formation. N-APP is involved in normal pruning of neurons in early development in which relatively unused neurons and their nerve-fiber connections (axons) wither and degenerate. Recently, however, it has been shown that N-APP binds to and activates the apoptotic death receptor 6 (DR6) in vitro, which is expressed on axons in response to trophic factor (e.g., nerve growth factor) withdrawal resulting in axonal degeneration. The aging process can lead to a reduction in the levels of growth factors in certain areas of the brain and/or the ability to sense growth factors. This in turn would lead to the release of N-APP fragment by cleavage of APP on neuronal surfaces, activating nearby DR6 receptors to initiate the axonal shrinkage and neuronal degeneration of Alzheimer's.

See also, Rauk, Arvi. “The chemistry of Alzheimer's disease.” Chem. Soc. Rev. 38 (2009): p. 2698-2715; Vassar, Robert, Dora M. Kovacs, Riqiang Yan and Philip C. Wong. “The •-Secretase Enzyme BACE in Health and Alzheimer's disease: Regulation, Cell Biology, Function, and Therapeutic Potential. J. Neurosci. 29(41) (2009): 12787-12794; and Silvestri, Romano. “Boom in the Development of Non-Peptidic β-Secretase (BACE1) Inhibitors for the Treatment of Alzheimer's Disease.” Medicinal Research Reviews. Vol. 29, No. 2 (2009): p. 295-338.

Since β-secretase cleavage of APP is essential for both amyloid plaque formation and DR6-mediated apoptosis, it is a key target in the search for therapeutic agents for treating AD.

### SUMMARY OF THE INVENTION

In one aspect of the present invention there is provided novel compounds having the general Formula I′:

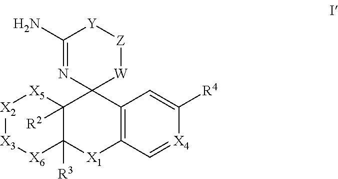

and stereoisomers, diastereomers, enantiomers, tautomers and pharmaceutically acceptable salts thereof, wherein W, X₁, X₂, X₃, X₄, X₅, X₆, Y, Z, R², R³ and R⁴ are as defined herein.

In another aspect of the invention, there are provided pharmaceutical compositions comprising compounds of Formula I′, I′a, I′b, I′c, I′d, I′e, I′f, I′g, I′h, I′i, I′j, I′k, I′l, I′m, I′n, I′o, I′p, I, Ia, Ib, Ic, Id, Ie, If, Ig, Ih, Ii, Ij, Ik, Il, Im, In, Io and Ip and a pharmaceutically acceptable carrier, diluent or excipient.

In another aspect of the invention, there is provided a method of inhibiting cleavage of APP by β-secretase in a mammal comprising administering to said mammal an effective amount of a compound of Formula I′, I′a, I′b, I′c, I′d, I′e, I′g, I′h, I′i, I′j, I′k, I′l, I′m, I′n, I′o, I′p, I, Ia, Ib, Ic, Id, Ie, If, Ig, Ih, Ii, Ij, Ik, Il, Im, In, Io and Ip.

In another aspect of the invention, there is provided a method for treating a disease or condition mediated by the cleavage of APP by β-secretase in a mammal, comprising administering to said mammal an effective amount of a compound of Formula I′, I′a, I′b, I′c, I′d, I′e, I′g, I′h, I′i, I′j, I′k, I′l, I′m, I′n, I′o, I′p, I, Ia, Ib, Ic, Id, Ie, If, Ig, Ih, Ii, Ij, Ik, Il, Im, In, Io and Ip.

In another aspect of the invention, there is provided a use of a compound of Formula I′, I′a, I′b, I′c, I′d, I′e, I′g, I′h, I′i, I′j, I′k, I′l, I′m, I′n, I′o, I′p, I, Ia, Ib, Ic, Id, Ie, If, Ig, Ih, Ii, Ij, Ik, Il, Im, In, Io and Ip in the manufacture of a medicament for the treatment of neurodegenerative diseases, such as Alzheimer's disease.

In another aspect of the invention, there is provided a use of a compound of Formula I′, I′a, I′b, I′c, I′d, I′e, I′h, I′i, I′j, I′k, I′l, I′m, I′n, I′o, I′p, I, Ia, Ib, Ic, Id, Ie, If, Ig, Ih, Ii, Ij, Ik, Il, Im, In, Io and Ip in the treatment of neurodegenerative diseases, such as Alzheimer's disease.

Another aspect provides intermediates for preparing compounds of Formula I′, I′a, I′b, I′c, I′d, I′e, I′g, I′h, I′i, I′j, I′k, I′l, I′m, I′n, I′o, I′p, I, Ia, Ib, Ic, Id, Ie, If, Ig, Ih, Ii, Ij, Ik, Il, Im, In, Io and Ip. Certain compounds of Formula I′, I′a, I′b, I′c, I′d, I′e, I′g, I′h, I′i, I′j, I′k, I′l, I′m, I′n, I′o, I′p, I, Ia, Ib, Ic, Id, Ie, If, Ig, Ih, Ij, Ik, Il, Im, In, Io and Ip may be used as intermediates for other compounds of Formula I′, I′a, I′b, I′c, I′d, I′e, I′f, I′g, I′h, I′i, I′j, I′k, I′1, I′m, I′n, I′o, I′p, I, Ia, Ib, Ic, Id, Ie, If, Ig, Ih, Ii, Ij, Ik, Il, Im, In, Io and Ip.

Another aspect includes processes for preparing, methods of separation, and methods of purification of the compounds described herein.

### DETAILED DESCRIPTION OF THE INVENTION

### Definitions

The term “acyl” means a carbonyl containing substituent represented by the formula —C(O)—R, in which R is hydrogen, alkyl, alkoxy, amino, a carbocycle, a heterocycle, carbocycle-substituted alkyl or heterocycle-substituted alkyl, wherein the alkyl, alkoxy, amino, carbocycle and heterocycle are as defined herein. Acyl groups include alkanoyl (e.g., acetyl), aroyl (e.g., benzoyl), and heteroaroyl.

The term “alkoxycarbonyl” means the group —C(═O)OR in which R is alkyl. A particular alkoxycarbonyl group is C₁-C₆ alkoxycarbonyl, wherein the R group is C₁-C₆ alkyl. Optionally substituted alkoxycarbonyl means the alkyl group is optionally substituted.

The term “alkyl” means a branched or unbranched, saturated or unsaturated (i.e., alkenyl, alkynyl) aliphatic hydrocarbon group, having up to 12 carbon atoms unless otherwise specified. When used as part of another term, for example “alkylamino”, the alkyl portion may be a saturated hydrocarbon chain, however also includes unsaturated hydrocarbon carbon chains such as “alkenylamino” and “alkynylamino. Examples of particular alkyl groups are methyl, ethyl, n-propyl, isopropyl, n-butyl, iso-butyl, sec-butyl, tert-butyl, n-pentyl, 2-methylbutyl, 2,2-dimethylpropyl, n-hexyl, 2-methylpentyl, 2,2-dimethylbutyl, n-heptyl, 3-heptyl, 2-methylhexyl, and the like. The terms “lower alkyl” “C₁-C₄ alkyl” and “alkyl of 1 to 4 carbon atoms” are synonymous and used interchangeably to mean methyl, ethyl, 1-propyl, isopropyl, cyclopropyl, 1-butyl, sec-butyl or t-butyl. In other examples, the alkyl group is C₁-C₂, C₁-C₃, C₁-C₄, C₁-C₅ or C₁-C₆. Unless specified otherwise, substituted alkyl groups contain one, two, three or four substituents which may be the same or different. Alkyl substituents are, unless otherwise specified, halogen, amino, hydroxyl, protected hydroxyl, mercapto, carboxy, alkoxy, nitro, cyano, amidino, guanidino, urea, oxo, sulfonyl, sulfinyl, aminosulfonyl, alkylsulfonylamino, arylsulfonylamino, aminocarbonyl, acylamino, alkoxy, acyl, acyloxy, an optionally substituted carbocycle and an optionally substituted heterocycle. Examples of the above substituted alkyl groups include, but are not limited to; cyanomethyl, nitromethyl, hydroxymethyl, trityloxymethyl, propionyloxymethyl, aminomethyl, carboxymethyl, carboxyethyl, carboxypropyl, alkyloxycarbonylmethyl, allyloxycarbonylaminomethyl, carbamoyloxymethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichloro(n-butyl), 2-amino(iso-propyl), 2-carbamoyloxyethyl and the like. The alkyl group may also be substituted with a carbocycle group. Examples include cyclopropylmethyl, cyclobutylmethyl, cyclopentylmethyl, and cyclohexylmethyl groups, as well as the corresponding -ethyl, -propyl, -butyl, -pentyl, -hexyl groups, etc. Substituted alkyls include substituted methyls, e.g., a methyl group substituted by the same substituents as the “substituted Cₙ-Cₘ alkyl” group. Examples of the substituted methyl group include groups such as hydroxymethyl, protected hydroxymethyl (e.g., tetrahydropyranyloxymethyl), acetoxymethyl, carbamoyloxymethyl, trifluoromethyl, chloromethyl, carboxymethyl, bromomethyl and iodomethyl.

The terms “alkenyl” and “alkynyl” also include linear or branched-chain radicals of carbon atoms.

The term “alkoxy” means the group —O(alkyl), wherein the alkyl is linear or branched-chain. The alkyl may be substituted by the same substituents as the “substituted alkyl” group. C₁-C₆ alkoxy means —O(C₁-C₆ alkyl).

The term “amidine” means the group —C(NH)—NHR in which R is hydrogen, alkyl, a carbocycle, a heterocycle, carbocycle-substituted alkyl or heterocycle-substituted alkyl, wherein the alkyl, alkoxy, carbocycle and heterocycle are as defined herein. A particular amidine is the group —NH—C(NH)—NH₂.

The term “amino” means primary (i.e., —NH₂), secondary (i.e., —NRH) and tertiary (i.e., —NRR) amines in which R is hydrogen, alkyl, alkoxy, a carbocycle, a heterocycle, carbocycle-substituted alkyl or heterocycle-substituted alkyl, wherein the alkyl, alkoxy, carbocycle and heterocycle are as defined herein. Particular secondary and tertiary amines are alkylamine, dialkylamine, arylamine, diarylamine, aralkylamine and diaralkylamine, wherein the alkyl is as herein defined and optionally substituted. Particular secondary and tertiary amines are methylamine, ethylamine, propylamine, isopropylamine, phenylamine, benzylamine dimethylamine, diethylamine, dipropylamine and disopropylamine.

The term “amino-protecting group” as used herein refers to a derivative of the groups commonly employed to block or protect an amino group while reactions are carried out on other functional groups on the compound. Examples of such protecting groups include carbamates, amides, alkyl and aryl groups, imines, as well as many N-heteroatom derivatives which can be removed to regenerate the desired amine group. Particular amino protecting groups are acetyl, trifluoroacetyl, t-butyloxycarbonyl (“Boc”), benzyloxycarbonyl (“CBz”) and 9-fluorenylmethyleneoxycarbonyl (“Fmoc”). Further examples of these groups, and other protecting groups, are found in T. W. Greene, et al. Greene's Protective Groups in Organic Synthesis. New York: Wiley Interscience, 2006.

The term “aryl” when used alone or as part of another term means a carbocyclic aromatic group whether or not fused having the number of carbon atoms designated or if no number is designated, up to 14 carbon atoms. Particular aryl groups are phenyl, naphthyl, biphenyl, phenanthrenyl, naphthacenyl, and the like (see e.g., Dean, J. A. Lange's Handbook of Chemistry. 15th ed. New York: McGraw-Hill Professional, 1998). A particular aryl is phenyl. Substituted phenyl or substituted aryl means a phenyl group or aryl group substituted with one, two, three, four or five substituents, for example 1-2, 1-3 or 1-4 substituents chosen, unless otherwise specified, from halogen (F, Cl, Br, I), hydroxy, protected hydroxy, cyano, nitro, alkyl (for example C₁-C₆ alkyl), alkoxy (for example C₁-C₆ alkoxy), benzyloxy, carboxy, protected carboxy, carboxymethyl, protected carboxymethyl, hydroxymethyl, protected hydroxymethyl, aminomethyl, protected aminomethyl, trifluoromethyl, alkylsulfonylamino, alkylsulfonylaminoalkyl, arylsulfonylamino, arylsulonylaminoalkyl, heterocyclylsulfonylamino, heterocyclylsulfonylaminoalkyl, heterocyclyl, aryl, or other groups specified. One or more methyne (CH) and/or methylene (CH₂) groups in these substituents may in turn be substituted with a similar group as those denoted above. Examples of the term “substituted phenyl” includes, but is not limited to, a mono- or di(halo)phenyl group such as 2-chlorophenyl, 2-bromophenyl, 4-chlorophenyl, 2,6-dichlorophenyl, 2,5-dichlorophenyl, 3,4-dichlorophenyl, 3-chlorophenyl, 3-bromophenyl, 4-bromophenyl, 3,4-dibromophenyl, 3-chloro-4-fluorophenyl, 2-fluorophenyl and the like; a mono- or di(hydroxy)phenyl group such as 4-hydroxyphenyl, 3-hydroxyphenyl, 2,4-dihydroxyphenyl, the protected-hydroxy derivatives thereof and the like; a nitrophenyl group such as 3- or 4-nitrophenyl; a cyanophenyl group, for example, 4-cyanophenyl; a mono- or di(lower alkyl)phenyl group such as 4-methylphenyl, 2,4-dimethylphenyl, 2-methylphenyl, 4-(isopropyl)phenyl, 4-ethylphenyl, 3-(n-propyl)phenyl and the like; a mono or di(alkoxy)phenyl group, for example, 3,4-dimethoxyphenyl, 3-methoxy-4-benzyloxyphenyl, 3-methoxy-4-(1-chloromethyl)benzyloxy-phenyl, 3-ethoxyphenyl, 4-(isopropoxy)phenyl, 4-(t-butoxy)phenyl, 3-ethoxy-4-methoxyphenyl and the like; 3- or 4-trifluoromethylphenyl; a mono- or dicarboxyphenyl or (protected carboxy)phenyl group such as 4-carboxyphenyl; a mono- or di(hydroxymethyl)phenyl or (protected hydroxymethyl)phenyl such as 3-(protected hydroxymethyl)phenyl or 3,4-di(hydroxymethyl)phenyl; a mono- or di(aminomethyl)phenyl or (protected aminomethyl)phenyl such as 2-(aminomethyl)phenyl or 2,4-(protected aminomethyl)phenyl; a mono- or di(N-(methylsulfonylamino))phenyl such as 3-(N-methylsulfonylamino))phenyl; disubstituted phenyl groups such as 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl and 2-hydroxy-4-chlorophenyl; trisubstituted phenyl groups such as 3-methoxy-4-benzyloxy-6-methylsulfonylamino and 3-methoxy-4-benzyloxy-6-phenylsulfonylamino; tetrasubstituted phenyl groups such as 3-methoxy-4-benzyloxy-5-methyl-6-phenyl sulfonylamino. Particular substituted phenyl groups include the 2-chlorophenyl, 2-aminophenyl, 2-bromophenyl, 3-methoxyphenyl, 3-ethoxy-phenyl, 4-benzyloxyphenyl, 4-methoxyphenyl, 3-ethoxy-4-benzyloxyphenyl, 3,4-diethoxyphenyl, 3-methoxy-4-benzyloxyphenyl, 3-methoxy-4-(1-chloromethyl)benzyloxy-phenyl, 3-methoxy-4-(1-chloromethyl)benzyloxy-6-methyl sulfonyl aminophenyl groups. Fused aryl rings may also be substituted with any, for example 1, 2 or 3, of the substituents specified herein in the same manner as substituted alkyl groups.

The terms “carbocyclyl”, “carbocyclic”, “carbocycle” and “carbocyclo” alone and when used as a moiety in a complex group such as a carbocycloalkyl group, refer to a mono-, bi-, or tricyclic aliphatic ring having 3 to 14 carbon atoms, for example 3 to 7 carbon atoms or 3 to 6 carbon atoms, which may be saturated or unsaturated, aromatic or non-aromatic. Particular saturated carbocyclic groups are cyclopropyl, cyclobutyl, cyclopentyl and cyclohexyl groups. A particular saturated carbocycle is cyclopropyl. Another particular saturated carbocycle is cyclohexyl. Particular unsaturated carbocycles are aromatic, e.g., aryl groups as previously defined, for example phenyl. The terms “substituted carbocyclyl”, “carbocycle” and “carbocyclo” mean these groups substituted by the same substituents as the “substituted alkyl” group.

The term “carboxy-protecting group” as used herein refers to one of the ester derivatives of the carboxylic acid group commonly employed to block or protect the carboxylic acid group while reactions are carried out on other functional groups on the compound. Examples of such carboxylic acid protecting groups include 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxybenzhydryl, 2,2′,4,4′-tetramethoxybenzhydryl, alkyl such as t-butyl or t-amyl, trityl, 4-methoxytrityl, 4,4′-dimethoxytrityl, 4,4′,4″-trimethoxytrityl, 2-phenylprop-2-yl, trimethylsilyl, t-butyldimethylsilyl, phenacyl, 2,2,2-trichloroethyl, beta-(trimethylsilyl)ethyl, beta-(di(n-butyl)methylsilyl)ethyl, p-toluenesulfonylethyl, 4-nitrobenzylsulfonylethyl, allyl, cinnamyl, 1-(trimethylsilylmethyl)prop-1-en-3-yl, and like moieties. The species of carboxy-protecting group employed is not critical so long as the derivatized carboxylic acid is stable to the condition of subsequent reaction(s) on other positions of the molecule and can be removed at the appropriate point without disrupting the remainder of the molecule. In particular, it is important not to subject a carboxy-protected molecule to strong nucleophilic bases, such as lithium hydroxide or NaOH, or reductive conditions employing highly activated metal hydrides such as LiAlH₄. Such harsh removal conditions are also to be avoided when removing amino-protecting groups and hydroxy-protecting groups, discussed below. Particular carboxylic acid protecting groups are the alkyl (e.g., methyl, ethyl, t-butyl), allyl, benzyl and p-nitrobenzyl groups. The term “protected carboxy” refers to a carboxy group substituted with one of the above carboxy-protecting groups. Further examples are found in Greene's Protective Groups in Organic Synthesis, supra.

The terms “comprise” and “comprising” when used herein are non-limiting in scope, i.e., are intended to specify the presence of the stated features, integers, components, or steps, but do not preclude the presence or addition such features, integers, components, steps, or groups thereof.

The term “guanidine” means the group —NH—C(NH)—NHR in which R is hydrogen, alkyl, alkoxy, a carbocycle, a heterocycle, carbocycle-substituted alkyl or heterocycle-substituted alkyl, wherein the alkyl, alkoxy, carbocycle and heterocycle are as defined herein. A particular guanidine is the group —NH—C(NH)—NH₂.

The term “hydroxy-protecting group” as used herein refers to a derivative of the hydroxy group commonly employed to block or protect the hydroxy group while reactions are carried out on other functional groups on the compound. Examples of such protecting groups include tetrahydropyranyloxy, benzoyl, acetoxy, carbamoyloxy, benzyl, and silylethers (e.g., tert-butyldimethylsilyl (“TBS”), tert-butyldiphenylsilyl (“TBDPS”)) groups. Further examples are found in Greene's Protective Groups in Organic Synthesis, supra. The term “protected hydroxy” refers to a hydroxy group substituted with one of the above hydroxy-protecting groups.

The term “heterocyclic group”, “heterocyclic”, “heterocycle”, “heterocyclyl”, or “heterocyclo” alone and when used as a moiety in a complex group such as a heterocycloalkyl group, are used interchangeably and refer to any mono-, bi-, or tricyclic, saturated or unsaturated, aromatic (heteroaryl) or non-aromatic ring having the number of atoms designated, generally from 5 to about 14 ring atoms, where the ring atoms are carbon and at least one heteroatom (nitrogen, sulfur or oxygen), for example 1 to 4 heteroatoms. The sulfur heteroatoms may optionally be oxidized (e.g., SO, SO₂), and any nitrogen heteroatom may optionally be quaternized. Typically, a 5-membered ring has 0 to 2 double bonds and 6- or 7-membered ring has 0 to 3 double bonds. In a particular embodiment, heterocyclic groups are four to seven membered cyclic groups containing one, two or three heteroatoms selected from the group consisting of nitrogen, oxygen and sulfur. Particular non-aromatic heterocycles are morpholinyl (morpholino), pyrrolidinyl, oxiranyl, oxetanyl, tetrahydrofuranyl, 2,3-dihydrofuranyl, 2H-pyranyl, tetrahydropyranyl, thiiranyl, thietanyl, tetrahydrothietanyl, aziridinyl, azetidinyl, 1-methyl-2-pyrrolyl, piperazinyl and piperidinyl. A “heterocycloalkyl” group is a heterocycle group as defined above covalently bonded to an alkyl group as defined above. Particular 5-membered heterocycles containing a sulfur or oxygen atom and one to three nitrogen atoms are thiazolyl, in particular thiazol-2-yl and thiazol-2-yl N-oxide; thiadiazolyl, in particular 1,3,4-thiadiazol-5-yl and 1,2,4-thiadiazol-5-yl; oxazolyl, for example oxazol-2-yl; and oxadiazolyl, such as 1,3,4-oxadiazol-5-yl and 1,2,4-oxadiazol-5-yl. Particular 5-membered ring heterocycles containing 2 to 4 nitrogen atoms include imidazolyl, such as imidazol-2-yl; triazolyl, such as 1,3,4-triazol-5-yl, 1,2,3-triazol-5-yl, and 1,2,4-triazol-5-yl; and tetrazolyl, such as 1H-tetrazol-5-yl. Particular benzo-fused 5-membered heterocycles are benzoxazol-2-yl, benzthiazol-2-yl and benzimidazol-2-yl. Particular 6-membered heterocycles contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl; and pyrazinyl. The pyridine N-oxides and pyridazine N-oxides and the pyridyl, pyrimid-2-yl, pyrimid-4-yl, pyridazinyl and the 1,3,4-triazin-2-yl groups, are a particular group. Substituents for “optionally substituted heterocycles”, and further examples of the 5- and 6-membered ring systems discussed above can be found in W. Druckheimer et al., U.S. Pat. No. 4,278,793. In a particular embodiment, such optionally substituted heterocycle groups are substituted with hydroxyl, alkyl, alkoxy, acyl, halogen, mercapto, oxo, carboxyl, acyl, halo-substituted alkyl, amino, cyano, nitro, amidino and guanidino.

The term “heteroaryl” alone and when used as a moiety in a complex group such as a heteroaralkyl group, refers to any mono-, bi-, or tricyclic aromatic ring system having the number of atoms designated where at least one ring is a 5-, 6- or 7-membered ring containing from one to four heteroatoms selected from the group nitrogen, oxygen, and sulfur, and in a particular embodiment at least one heteroatom is nitrogen (see Lange's Handbook of Chemistry, supra). In a particular embodiment, the heteroaryl is a 5-membered aromatic ring containing one, two or three heteroatoms selected from nitrogen, oxygen and sulfur. Included in the definition are any bicyclic groups where any of the above heteroaryl rings are fused to a benzene ring. Particular heteroaryls incorporate a nitrogen or oxygen heteroatom. In a particular embodiment, the heteroaryl is a 5-membered aromatic ring containing one, two or three heteroatoms selected from nitrogen, oxygen and sulfur. In a particular embodiment, the heteroaryl group is a 6-membered aromatic ring containing one, two or three heteroatoms selected from nitrogen, oxygen and sulfur. The following are examples of the heteroaryl groups (substituted and unsubstituted): thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, thiazinyl, oxazinyl, triazinyl, thiadiazinyl, oxadiazinyl, dithiazinyl, dioxazinyl, oxathiazinyl, tetrazinyl, thiatriazinyl, oxatriazinyl, dithiadiazinyl, imidazolinyl, dihydropyrimidyl, tetrahydropyrimidyl, tetrazolo[1,5-b]pyridazinyl and purinyl, as well as benzo-fused derivatives, for example benzoxazolyl, benzofuryl, benzothiazolyl, benzothiadiazolyl, benzotriazolyl, benzoimidazolyl and indolyl. In a particular embodiment the heteroaryl group may be: 1,3-thiazol-2-yl, 4-(carboxymethyl)-5-methyl-1,3-thiazol-2-yl, 4-(carboxymethyl)-5-methyl-1,3-thiazol-2-yl sodium salt, 1,2,4-thiadiazol-5-yl, 3-methyl-1,2,4-thiadiazol-5-yl, 1,3,4-triazol-5-yl, 2-methyl-1,3,4-triazol-5-yl, 2-hydroxy-1,3,4-triazol-5-yl, 2-carboxy-4-methyl-1,3,4-triazol-5-yl sodium salt, 2-carboxy-4-methyl-1,3,4-triazol-5-yl, 1,3-oxazol-2-yl, 1,3,4-oxadiazol-5-yl, 2-methyl-1,3,4-oxadiazol-5-yl, 2-(hydroxymethyl)-1,3,4-oxadiazol-5-yl, 1,2,4-oxadiazol-5-yl, 1,3,4-thiadiazol-5-yl, 2-thiol-1,3,4-thiadiazol-5-yl, 2-(methylthio)-1,3,4-thiadiazol-5-yl, 2-amino-1,3,4-thiadiazol-5-yl, 1H-tetrazol-5-yl, 1-methyl-1H-tetrazol-5-yl, 1-(1-(dimethylamino)eth-2-yl)-1H-tetrazol-5-yl, 1-(carboxymethyl)-1H-tetrazol-5-yl, 1-(carboxymethyl)-1H-tetrazol-5-yl sodium salt, 1-(methylsulfonic acid)-1H-tetrazol-5-yl, 1-(methylsulfonic acid)-1H-tetrazol-5-yl sodium salt, 2-methyl-1H-tetrazol-5-yl, 1,2,3-triazol-5-yl, 1-methyl-1,2,3-triazol-5-yl, 2-methyl-1,2,3-triazol-5-yl, 4-methyl-1,2,3-triazol-5-yl, pyrid-2-yl N-oxide, 6-methoxy-2-(n-oxide)-pyridaz-3-yl, 6-hydroxypyridaz-3-yl, 1-methylpyrid-2-yl, 1-methylpyrid-4-yl, 2-hydroxypyrimid-4-yl, 1,4,5,6-tetrahydro-5,6-dioxo-4-methyl-as-triazin-3-yl, 1,4,5,6-tetrahydro-4-(formylmethyl)-5,6-dioxo-as-triazin-3-yl, 2,5-dihydro-5-oxo-6-hydroxy-astriazin-3-yl, 2,5-dihydro-5-oxo-6-hydroxy-as-triazin-3-yl sodium salt, 2,5-dihydro-5-oxo-6-hydroxy-2-methyl-astriazin-3-yl sodium salt, 2,5-dihydro-5-oxo-6-hydroxy-2-methyl-as-triazin-3-yl, 2,5-dihydro-5-oxo-6-methoxy-2-methyl-as-triazin-3-yl, 2,5-dihydro-5-oxo-as-triazin-3-yl, 2,5-dihydro-5-oxo-2-methyl-as-triazin-3-yl, 2,5-dihydro-5-oxo-2,6-dimethyl-as-triazin-3-yl, tetrazolo[1,5-b]pyridazin-6-yl and 8-aminotetrazolo[1,5-b]-pyridazin-6-yl. An alternative group of “heteroaryl” includes; 4-(carboxymethyl)-5-methyl-1,3-thiazol-2-yl, 4-(carboxymethyl)-5-methyl-1,3-thiazol-2-yl sodium salt, 1,3,4-triazol-5-yl, 2-methyl-1,3,4-triazol-5-yl, 1H-tetrazol-5-yl, 1-methyl-1H-tetrazol-5-yl, 1-(1-(dimethylamino)eth-2-yl)-1H-tetrazol-5-yl, 1-(carboxymethyl)-1H-tetrazol-5-yl, 1-(carboxymethyl)-1H-tetrazol-5-yl sodium salt, 1-(methylsulfonic acid)-1H-tetrazol-5-yl, 1-(methylsulfonic acid)-1H-tetrazol-5-yl sodium salt, 1,2,3-triazol-5-yl, 1,4,5,6-tetrahydro-5,6-dioxo-4-methyl-as-triazin-3-yl, 1,4,5,6-tetrahydro-4-(2-formylmethyl)-5,6-dioxo-as-triazin-3-yl, 2,5-dihydro-5-oxo-6-hydroxy-2-methyl-as-triazin-3-yl sodium salt, 2,5-dihydro-5-oxo-6-hydroxy-2-methyl-as-triazin-3-yl, tetrazolo[1,5-b]pyridazin-6-yl, or 8-aminotetrazolo[1,5-b]pyridazin-6-yl. Heteroaryl groups are optionally substituted as described for heterocycles.

The term “inhibitor” means a compound which reduces or prevents the enzymatic cleavage of APP by β-secretase. Alternatively, “inhibitor” means a compound which prevents or slows the formation of beta-amyloid plaques in mammalian brain. Alternatively, “inhibitor” means a compound that prevents or slows the progression of a disease or condition associated with β-secretase enzymatic activity, e.g., cleavage of APP. Alternatively, “inhibitor” means a compound which prevents Alzheimer's disease. Alternatively, “inhibitor” means a compound which slows the progression of Alzheimer's disease or its symptoms.

The term “optionally substituted” unless otherwise specified means that a group may be unsubstituted or substituted by one or more (e.g., 0, 1, 2, 3 or 4) of the substituents listed for that group in which said substituents may be the same or different. In a particular embodiment, an optionally substituted group has 1 substituent. In another embodiment an optionally substituted group has 2 substituents. In another embodiment an optionally substituted group has 3 substituents.

The term “pharmaceutically acceptable” indicates that the substance or composition is compatible chemically and/or toxicologically, with the other ingredients comprising a formulation, and/or the mammal being treated therewith.

The term “pharmaceutically acceptable salts” include both acid and base addition salts.

The term “pharmaceutically acceptable acid addition salt” refers to those salts which retain the biological effectiveness and properties of the free bases and which are not biologically or otherwise undesirable, formed with inorganic acids such as hydrochloric acid, hydrobromic acid, sulfuric acid, nitric acid, carbonic acid, phosphoric acid and the like, and organic acids may be selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, salicyclic acid and the like.

The term “pharmaceutically acceptable base addition salts” include those derived from inorganic bases such as sodium, potassium, lithium, ammonium, calcium, magnesium, iron, zinc, copper, manganese, aluminum salts and the like. Particularly base addition salts are the ammonium, potassium, sodium, calcium and magnesium salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, polyamine resins and the like. Particularly organic non-toxic bases are isopropylamine, diethylamine, ethanolamine, trimethamine, dicyclohexylamine, choline, and caffeine.

The term “sulfanyl” means —S—R group in which R is alkyl, a carbocycle, a heterocycle, carbocycle-substituted alkyl or heterocycle-substituted alkyl, wherein the alkyl, carbocycle and heterocycle are as defined herein. Particular sulfanyl groups are alkylsulfanyl (i.e., —S-alkyl), for example methylsulfanyl; arylsulfanyl, for example phenylsulfanyl; and aralkylsulfanyl, for example benzylsulfanyl.

The term “sulfinyl” means —SO—R group in which R is hydrogen, alkyl, a carbocycle, a heterocycle, carbocycle-substituted alkyl or heterocycle-substituted alkyl, wherein the alkyl, carbocycle and heterocycle are as defined herein. Particular sulfinyl groups are alkylsulfinyl (i.e., —SO-alkyl), for example methylsulfinyl; arylsulfinyl, for example phenylsulfinyl; and aralkylsulfinyl, for example benzylsulfinyl.

The term “sulfonyl” means a —SO₂—R group in which R is hydrogen, alkyl, a carbocycle, a heterocycle, carbocycle-substituted alkyl or heterocycle-substituted alkyl wherein the alkyl, carbocycle and heterocycle are as defined herein. Particular sulfonyl groups are alkylsulfonyl (i.e., —SO₂-alkyl), for example methylsulfonyl; arylsulfonyl, for example phenylsulfonyl; and aralkylsulfonyl, for example benzylsulfonyl.

The terms “treat” or “treatment” refer to therapeutic, prophylactic, palliative or preventative measures. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder, as well as those prone to have the condition or disorder or those in which the condition or disorder is to be prevented.

The phrases “therapeutically effective amount” or “effective amount” mean an amount of a compound described herein that, when administered to a mammal in need of such treatment, sufficient to (i) treat or prevent the particular disease, condition, or disorder, (ii) attenuate, ameliorate, or eliminate one or more symptoms of the particular disease, condition, or disorder, or (iii) prevent or delay the onset of one or more symptoms of the particular disease, condition, or disorder described herein. The amount of a compound that will correspond to such an amount will vary depending upon factors such as the particular compound, disease condition and its severity, the identity (e.g., weight) of the mammal in need of treatment, but can nevertheless be routinely determined by one skilled in the art. The “effective amount” of the compound to be administered will be governed by such considerations, and is the minimum amount necessary to inhibit cleavage of APP by β-secretase, for example by 10% or greater in situ. In a particular embodiment an “effective amount” of the compound inhibits cleavage of APP by β-secretase by 25% or greater in situ. In a particular embodiment the effective amount inhibits cleavage of APP by β-secretase by 50% or greater in situ. In a particular embodiment the effective amount inhibits cleavage of APP by β-secretase by 70% or greater in situ. In a particular embodiment the effective amount inhibits cleavage of APP by β-secretase by 80% or greater in situ. In a particular embodiment the effective amount inhibits cleavage of APP by β-secretase by 90% or greater in situ. Such amount may be below the amount that is toxic to normal cells, or the mammal as a whole. Alternatively, an “effective amount” is the amount of compound necessary to reduce A-beta levels in plasma or cerebrospinal fluid of a mammal, for example, by 10% or greater. In a particular embodiment, an “effective amount” is the amount of compound necessary to reduce A-beta levels in plasma or cerebrospinal fluid of a mammal by 25% or greater. In a particular embodiment, an “effective amount” is the amount of compound necessary to reduce A-beta levels in plasma or cerebrospinal fluid of a mammal by 50% or greater. In a particular embodiment, an “effective amount” is the amount of compound necessary to reduce A-beta levels in plasma or cerebrospinal fluid of a mammal by 75% or greater. Alternatively, an “effective amount” of the compound may be the amount of compound necessary to slow the progression of AD or symptoms thereof.

Abbreviations are sometimes used in conjunction with elemental abbreviations and chemical structures, for example, methanol (“MeOH”), ethanol (“EtOH”) or ethyl acetate (“EtOAc”). Additional abbreviations used throughout the application may include, for example, benzyl (“Bn”), phenyl (“Ph”) and acetate (“Ac”).

Tricyclic Compounds

Provided herein are compounds, and pharmaceutical formulations thereof, that are potentially useful in the treatment of diseases, conditions and/or disorders modulated by BACE-1.

One embodiment provides compounds of Formula I′:

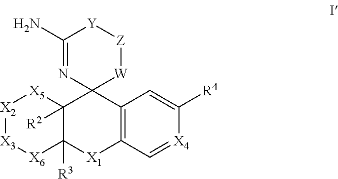

and stereoisomers, diastereomers, enantiomers, tautomers and pharmaceutically acceptable salts thereof, wherein:

W is CR¹²R¹³;

Y is O, S or NR¹;

Z is a bond, CH₂ or C(═O), provided when Z is C(═O) then Y is NR¹;

X₁ is selected from O, S, S(O), SO₂, NR¹⁰ and CHR¹⁰;

X₂ is selected from CR⁵R⁶, NR⁷ and O;

X₃ is selected from a bond, CR⁸R⁹ and O, provided when X₃ is O then X₂ is CR⁵R⁶;

X₄ is selected from CR¹¹ and N;

X₅ is selected from CR¹⁴R¹⁵ and O, provided when X₅ is O, then X₂ is CR⁵R⁶ and X₃ is CR⁸R⁹ or a bond;

X₆ is CR¹⁶R¹⁷;

R¹ is selected from hydrogen, alkyl, aralkyl, heteroaryl and heteroaralkyl;

R² and R³ are hydrogen or alkyl;

R⁴ is selected from hydrogen, hydroxy, halogen, amino, cyano, nitro, alkyl, alkoxy, acyl, acyloxy, acylamino, alkoxycarbonyl, sulfonyl, sulfinyl, sulfanyl, aryloxy, a carbocycle and a heterocycle wherein said amino, alkyl, alkoxy, acyl, acyloxy, alkoxycarbonyl, sulfonyl, sulfinyl, sulfanyl, aryloxy, carbocycle and heterocycle are optionally substituted with SF₅, hydroxy, halogen, amino, cyano, nitro, oxo, optionally substituted alkyl, optionally substituted alkoxy, sulfanyl, acyl, alkoxycarbonyl, haloalkyl, optionally substituted carbocycle or optionally substituted heterocycle;

R⁵ and R⁶ are independently selected from hydrogen, hydroxy, halogen, amino, cyano, nitro, alkyl, alkoxy, acyl, acyloxy, alkoxycarbonyl, sulfonyl, sulfinyl, sulfanyl, aryloxy, a carbocycle and a heterocycle, wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl or optionally substituted carbocycle, or

R⁵ and R⁶ taken together form an oxo group, or

R⁵ and R⁶ together with the atom to which they are attached form a carbocycle or heterocycle;

R⁷ is selected from hydrogen, alkyl, alkoxy, acyl, acyloxy, alkoxycarbonyl, sulfonyl, sulfinyl, sulfanyl, aryloxy, a carbocycle and a heterocycle, wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl or optionally substituted carbocycle;

R⁸ and R⁹ are independently selected from hydrogen, hydroxy, halogen, amino, cyano, nitro, alkyl, alkoxy, acyl, acyloxy, alkoxycarbonyl, sulfonyl, sulfinyl, sulfanyl, aryloxy, a carbocycle and a heterocycle, wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl or optionally substituted carbocycle, or

R⁸ and R⁹ taken together form an oxo, or

R⁸ and R⁹ together with the atom to which they are attached form a carbocycle or heterocycle;

R¹⁰ is selected from hydrogen, halogen and alkyl;

R¹¹ is selected from hydrogen, halogen and alkyl;

R¹² and R¹³ are independently selected from hydrogen and alkyl, or

R¹² and R¹³ together with the atom to which they are attached form a 3 to 6 membered carbocycle or heterocycle;

R¹⁴ and R¹⁵ are independently selected from hydrogen and C₁-C₃ alkyl; and

R¹⁶ and R¹⁷ are independently selected from hydrogen and halogen.

In certain embodiments of Formula I′, the compound has Formula I:

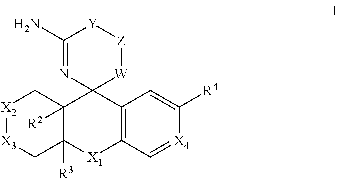

and stereoisomers, diastereomers, enantiomers, tautomers and pharmaceutically acceptable salts thereof, wherein:

W is CR¹²R¹³;

Y is O, S or NR¹;

Z is a bond, CH₂ or C(═O), provided when Z is C(═O) then Y is NR¹;

X₁ is selected from O, S, S(O), SO₂, NR¹⁰ and CHR¹⁰;

X₂ is selected from CR⁵R⁶, NR⁷ and O;

X₃ is selected from a bond, CR⁸R⁹ and O, provided when X₃ is O then X₂ is CR⁵R⁶;

X₄ is selected from CR¹¹ and N;

R¹ is selected from hydrogen, alkyl, aralkyl, heteroaryl and heteroaralkyl;

R² and R³ are hydrogen or alkyl;

R⁴ is selected from hydrogen, hydroxy, halogen, amino, cyano, nitro, alkyl, alkoxy, acyl, acyloxy, acylamino, alkoxycarbonyl, sulfonyl, sulfinyl, sulfanyl, aryloxy, a carbocycle and a heterocycle wherein said amino, alkyl, alkoxy, acyl, acyloxy, alkoxycarbonyl, sulfonyl, sulfinyl, sulfanyl, aryloxy, carbocycle and heterocycle are optionally substituted with SF₅, hydroxy, halogen, amino, cyano, nitro, oxo, optionally substituted alkyl, optionally substituted alkoxy, sulfanyl, acyl, alkoxycarbonyl, haloalkyl, optionally substituted carbocycle or optionally substituted heterocycle;

R⁵ and R⁶ are independently selected from hydrogen, hydroxy, halogen, amino, cyano, nitro, alkyl, alkoxy, acyl, acyloxy, alkoxycarbonyl, sulfonyl, sulfinyl, sulfanyl, aryloxy, a carbocycle and a heterocycle, wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl or optionally substituted carbocycle, or

R⁵ and R⁶ taken together form an oxo group, or

R⁵ and R⁶ together with the atom to which they are attached form a carbocycle or heterocycle;

R⁷ is selected from hydrogen, alkyl, alkoxy, acyl, acyloxy, alkoxycarbonyl, sulfonyl, sulfinyl, sulfanyl, aryloxy, a carbocycle and a heterocycle, wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl or optionally substituted carbocycle;

R⁸ and R⁹ are independently selected from hydrogen, hydroxy, halogen, amino, cyano, nitro, alkyl, alkoxy, acyl, acyloxy, alkoxycarbonyl, sulfonyl, sulfinyl, sulfanyl, aryloxy, a carbocycle and a heterocycle, wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl or optionally substituted carbocycle, or

R⁸ and R⁹ taken together form an oxo, or

R⁸ and R⁹ together with the atom to which they are attached form a carbocycle or heterocycle;

R¹⁰ is selected from hydrogen, halogen and alkyl;

R¹¹ is selected from hydrogen, halogen and alkyl; and

R¹² and R¹³ are independently selected from hydrogen and alkyl, or

R¹² and R¹³ together with the atom to which they are attached form a 3 to 6 membered carbocycle or heterocycle.

In certain embodiments of Formula I′:

W is CR¹²R¹³;

Y is O, S or NR¹;

Z is a bond, CH₂ or C(═O), provided when Z is C(═O) then Y is NR';

X₁ is selected from O, S, S(O), SO₂, NR¹⁰ and CHR¹⁰;

X₂ is selected from CR⁵R⁶, NR⁷ and O;

X₃ is selected from a bond, CR⁸R⁹ and O, provided when X₃ is O then X₂ is CR⁵R⁶;

X₄ is selected from CR¹¹ and N;

X₅ is selected from CR¹⁴R¹⁵ and O, provided when X₅ is O, then X₂ is CR⁵R⁶ and X₃ is CR⁸R⁹ or a bond;

X₆ is CR¹⁶R¹⁷;

R¹ is C₁-C₃ alkyl;

R² and R³ are independently selected from hydrogen and C₁-C₆ alkyl;

R⁴ is selected from hydrogen, halogen, C₁-C₆ alkyl, C₁-C₆ alkenyl, C₁-C₆ alkynyl, C₁-C₆ alkoxy, NHC(═O)R^{f}, C(═O)NHR^{f}, a 3 to 6 membered carbocycle, a 3 to 6 membered heterocycle, phenyl and a 5 to 6 membered heteroaryl, wherein the alkyl, alkenyl, alkynyl, alkoxy, carbocycle, heterocycle, phenyl and heteroaryl are optionally substituted with one or more R⁹ groups;

R⁵ and R⁶ are independently selected from hydrogen, halogen, OR^{a}, NR^{b}R^{c}, CN, C₁-C₆ alkyl, C₁-C₆ alkoxy, phenyl, a 3 to 6 membered heterocycle and a 5 to 6 membered heteroaryl, wherein the alkyl, phenyl, heterocycle and heteroaryl are optionally substituted with halogen or a 3 to 6 membered carbocycle, or

R⁵ and R⁶ taken together form an oxo group, or

R⁵ and R⁶ together with the atom to which they are attached form a 3 to 6 membered carbocycle or heterocycle;

R⁷ is selected from hydrogen, C₁-C₆ alkyl, C₁-C₆ alkoxycarbonyl, —C(═O)NR^{h}Rⁱ, —SO₂(C₁-C₆ alkyl), a 3 to 6 membered carbocycle, a 3 to 6 membered heterocycle, phenyl, and a 5 to 6 membered heteroaryl, wherein the alkyls, alkoxycarbonyl, carbocycle, heterocycle, phenyl and heteroaryl are optionally substituted with one or more R^{d} groups;

R⁸ and R⁹ are independently selected from hydrogen, halogen, CN, C₁-C₆ alkyl, C₁-C₆ alkenyl, C₁-C₆ alkynyl, phenyl, a 5 to 6 membered heteroaryl and OR^{e}, wherein the alkyl, alkenyl, alkynyl, alkoxy, phenyl and heteroaryl are optionally substituted with halogen, or

R⁸ and R⁹ taken together form an oxo group, or

R⁸ and R⁹ together with the atom to which they are attached form a 3 to 6 membered carbocycle or heterocycle;

R¹⁰ is selected from hydrogen, halogen and C₁-C₆ alkyl;

R¹¹ is selected from hydrogen, halogen and C₁-C₆ alkyl;

R¹² and R¹³ are independently selected from hydrogen and C₁-C₃ alkyl, or

R¹² and R¹³ together with the atom to which they are attached form a 3 to 6 membered carbocycle or heterocycle;

R¹⁴ and R¹⁵ are independently selected from hydrogen and C₁-C₃ alkyl;

R¹⁶ and R¹⁷ are independently selected from hydrogen and halogen;

R^{a} is selected from hydrogen, C₁-C₆ alkyl and (CH₂)₀₋₃(3 to 6 membered carbocyclic);

R^{b} and R^{c} are independently selected from hydrogen and C₁-C₆ alkyl, or

R^{b} and R^{c} together with the nitrogen atom to which they are attached form a 3 to 6 membered heterocyclic;

each R^{d} is selected from halogen, hydroxy, oxo, C₃-C₆ cycloalkyl and phenyl, wherein the phenyl is optionally substituted with halogen, C₁-C₃ alkyl or C₁-C₃ alkoxy;

R^{e} is selected from hydrogen and C₁-C₆ alkyl; R^{f} is C₁-C₆ alkyl, phenyl, a 5 to 6 membered heteroaryl, wherein the alkyl, phenyl and heteroaryl are optionally substituted with halogen or C₁-C₃ alkyl;

each R^{g} is independently selected from halogen, CN, SF₅, C₁-C₆ alkyl, C₁-C₆ alkoxy, a 3 to 6 membered carbocycle, a 3 to 6 membered heterocycle, phenyl, and a 5 to 6 membered heteroaryl, wherein the alkyl, alkoxy, carbocycle, heterocycle, phenyl and heteroaryl are optionally substituted with halogen; and

R^{h} and Rⁱ are independently selected from hydrogen and C₁-C₆ alkyl, wherein the alkyl is optionally substituted with halogen, CN or C₁-C₆ alkoxy.

In certain embodiments of Formula I:

W is CR¹²R¹³;

Y is O, S or NR¹;

Z is a bond, CH₂ or C(═O), provided when Z is C(═O) then Y is NR¹;

X₁ is selected from O, S, S(O), SO₂, NR¹⁰ and CHR¹⁰;

X₂ is selected from CR⁵R⁶, NR⁷ and O;

X₃ is selected from a bond, CR⁸R⁹ and O, provided when X₃ is O then X₂ is CR⁵R⁶;

X₄ is selected from CR¹¹ and N;

R¹ is C₁-C₃ alkyl;

R² and R³ are independently selected from hydrogen and C₁-C₆ alkyl;

R⁴ is selected from hydrogen, halogen, C₁-C₆ alkyl, C₁-C₆ alkenyl, C₁-C₆ alkynyl, NHC(═O)R^{f}, C(═O)NHR^{f}, a 3 to 6 membered carbocycle, a 3 to 6 membered heterocycle, phenyl and a 5 to 6 membered heteroaryl, wherein the alkyl, alkenyl, alkynyl, carbocycle, heterocycle, phenyl and heteroaryl are optionally substituted with one or more R⁹ groups;

R⁵ and R⁶ are independently selected from hydrogen, halogen, OR^{a}, NR^{b}R^{c}, CN, C₁-C₆ alkyl, phenyl, and a 5 to 6 membered heteroaryl, wherein the alkyl, phenyl and heteroaryl are optionally substituted with halogen or a 3 to 6 membered carbocycle, or

R⁵ and R⁶ taken together form an oxo group, or

R⁵ and R⁶ together with the atom to which they are attached form a 3 to 6 membered carbocycle or heterocycle;

R⁷ is selected from hydrogen, C₁-C₆ alkyl, C₁-C₆ alkoxycarbonyl, —C(═O)NR^{h}Rⁱ, —SO₂(C₁-C₆ alkyl), a 3 to 6 membered carbocycle, a 3 to 6 membered heterocycle, phenyl, and a 5 to 6 membered heteroaryl, wherein the alkyls, alkoxycarbonyl, carbocycle, heterocycle, phenyl and heteroaryl are optionally substituted with one or more R^{d} groups;

R⁸ and R⁹ are independently selected from hydrogen, halogen, CN, C₁-C₆ alkyl, C₁-C₆ alkenyl, C₁-C₆ alkynyl, phenyl, a 5 to 6 membered heteroaryl and OR^{e}, wherein the alkyl, alkenyl, alkynyl, alkoxy, phenyl and heteroaryl are optionally substituted with halogen, or

R⁸ and R⁹ taken together form an oxo group, or

R⁸ and R⁹ together with the atom to which they are attached form a 3 to 6 membered carbocycle or heterocycle;

R¹⁰ is selected from hydrogen, halogen and C₁-C₆ alkyl;

R¹¹ is selected from hydrogen, halogen and C₁-C₆ alkyl;

R¹² and R¹³ are independently selected from hydrogen and C₁-C₃ alkyl, or

R¹² and R¹³ together with the atom to which they are attached form a 3 to 6 membered carbocycle or heterocycle;

R^{a} is selected from hydrogen, C₁-C₆ alkyl and (CH₂)₀₋₃(3 to 6 membered carbocyclic);

R^{b} and R^{c} are independently selected from hydrogen and C₁-C₆ alkyl, or

R^{b} and R^{c} together with the nitrogen atom to which they are attached form a 3 to 6 membered heterocyclic;

each R^{d} is selected from halogen and phenyl, wherein the phenyl is optionally substituted with halogen, C₁-C₃ alkyl or C₁-C₃ alkoxy;

R^{e} is selected from hydrogen and C₁-C₆ alkyl;

R^{f} is C₁-C₆ alkyl, phenyl, a 5 to 6 membered heteroaryl, wherein the alkyl, phenyl and heteroaryl are optionally substituted with halogen or C₁-C₃ alkyl;

each R^{g} is independently selected from halogen, CN, SF₅, C₁-C₆ alkyl, C₁-C₆ alkoxy, a 3 to 6 membered carbocycle, a 3 to 6 membered heterocycle, phenyl, and a 5 to 6 membered heteroaryl, wherein the alkyl, alkoxy, carbocycle, heterocycle, phenyl and heteroaryl are optionally substituted with halogen; and

R^{h} and Rⁱ are independently selected from hydrogen and C₁-C₆ alkyl, wherein the alkyl is optionally substituted with halogen, CN or C₁-C₆ alkoxy.

In certain embodiments of Formula I′:

W is CR¹²R¹³;

Y is O, S or NR¹;

Z is a bond, CH₂ or C(═O), provided when Z is C(═O) then Y is NR¹;

X₁ is O;

X₂ is selected from CR⁵R⁶, NR⁷ and O;

X₃ is selected from CR⁸R⁹ and O, provided when X₃ is O then X₂ is CR⁵R⁶;

X₄ is CR¹¹;

X₅ is selected from CR¹⁴R¹⁵ and O, provided when X₅ is O, then X₂ is CR⁵R⁶ and X₃ is CR⁸R⁹ or a bond;

X₆ is CR¹⁶R¹⁷;

R¹ is CH₃;

R² and R³ are independently selected from hydrogen and C₁-C₆ alkyl;

R⁴ is selected from halogen, C₁-C₆ alkoxy, NHC(═O)R^{f}, phenyl and a 5 to 6 membered heteroaryl, wherein the alkoxy, phenyl and heteroaryl are optionally substituted with one or more R^{g} groups;

R⁵ and R⁶ are independently selected from hydrogen, halogen, OR^{a}, NR^{b}R^{c}, C₁-C₆ alkyl, C₁-C₆ alkoxy, phenyl, a 3 to 6 membered heterocycle and a 5 to 6 membered heteroaryl, wherein the phenyl, heterocycle and heteroaryl are optionally substituted with halogen, or

R⁵ and R⁶ together with the atom to which they are attached form a 3 to 6 membered heterocycle;

R⁷ is selected from hydrogen, C₁-C₆ alkyl, C₁-C₆ alkoxycarbonyl, —SO₂(C₁-C₆ alkyl), and a 3 to 6 membered heterocyclic, wherein the alkyls and alkoxycarbonyl are optionally substituted with one or more R^{d} groups;

R⁸ and R⁹ are independently selected from hydrogen and OR^{e}, or

R⁸ and R⁹ taken together form an oxo group, or

R⁸ and R⁹ together with the atom to which they are attached form a 3 to 6 membered heterocycle;

R¹¹ is hydrogen or halogen;

R¹² and R¹³ are hydrogen;

R¹⁴ and R¹⁵ are independently selected from hydrogen and C₁-C₃ alkyl;

R¹⁶ and R¹⁷ are independently selected from hydrogen and halogen;

R^{a} is selected from hydrogen, C₁-C₆ alkyl and (CH₂)₀₋₃(3 to 6 membered carbocyclic);

R^{b} and R^{c} are independently selected from hydrogen and C₁-C₆ alkyl, or

R^{b} and R^{c} together with the nitrogen atom to which they are attached form a 3 to 6 membered heterocyclic;

each R^{d} is selected from halogen, hydroxy, oxo, C₃-C₆ cycloalkyl and phenyl, wherein the phenyl is optionally substituted with C₁-C₃ alkoxy;

R^{e} is hydrogen;

R^{f} is a 5 to 6 membered heteroaryl optionally substituted with halogen or C₁-C₃ alkyl; and

each R^{g} is independently selected from halogen, CN, C₁-C₆ alkyl and C₁-C₆ alkoxy,

wherein the alkyl and alkoxy are optionally substituted with halogen.

In certain embodiments of Formula I:

W is CR¹²R¹³;

Y is O, S or NR¹;

Z is a bond, CH₂ or C(═O), provided when Z is C(═O) then Y is NR¹;

X₁ is O;

X₂ is selected from CR⁵R⁶, NR⁷ and O;

X₃ is selected from CR⁸R⁹ and O, provided when X₃ is O then X₂ is CR⁵R⁶;

X₄ is CR¹¹;

R¹ is CH₃;

R² and R³ are hydrogen;

R⁴ is selected from halogen, NHC(═O)R^{f}, phenyl and a 5 to 6 membered heteroaryl,

wherein the phenyl and heteroaryl are optionally substituted with one or more R^{g} groups;

R⁵ and R⁶ are independently selected from hydrogen, halogen, OR^{a} and NR^{b}R^{c};

R⁷ is selected from hydrogen, C₁-C₆ alkyl, C₁-C₆ alkoxycarbonyl and a 3 to 6 membered heterocyclic, wherein the alkyl and alkoxycarbonyl are optionally substituted with one or more R^{d} groups;

R⁸ and R⁹ are independently selected from hydrogen and OR^{e}, or

R⁸ and R⁹ taken together form an oxo group, or

R⁸ and R⁹ together with the atom to which they are attached form a 3 to 6 membered heterocycle;

R¹¹ is hydrogen;

R¹² and R¹³ are hydrogen;

R^{a} is selected from hydrogen, C₁-C₆ alkyl and (CH₂)₀₋₃(3 to 6 membered carbocyclic);

R^{b} and R^{c} are independently selected from hydrogen and C₁-C₆ alkyl, or

R^{b} and R^{c} together with the nitrogen atom to which they are attached form a 3 to 6 membered heterocyclic;

each R^{d} is selected from halogen and phenyl, wherein the phenyl is optionally substituted with C₁-C₃ alkoxy;

R^{e} is hydrogen;

R^{f} is a 5 to 6 membered heteroaryl optionally substituted with halogen or C₁-C₃ alkyl; and

R^{g} is halogen.

In a particular embodiment, compounds of the invention have the stereochemical orientation represented by Formula I′a:

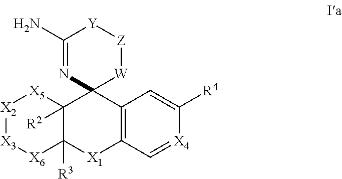

wherein W, X₁, X₂, X₃, X₄, X₅, X₆, Y, Z, R², R³ and R⁴ are as defined herein.

In a particular embodiment, compounds of the invention have the stereochemical orientation represented by Formula Ia:

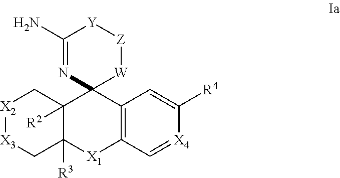

wherein W, X₁, X₂, X₃, X₄, Y, Z, R², R³ and R⁴ are as defined herein.

In a particular embodiment, compounds of the invention have the stereochemical orientation represented by Formula I′b:

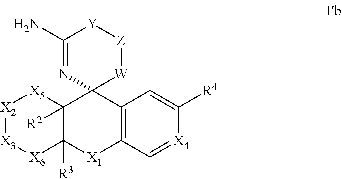

wherein W, X₁, X₂, X₃, X₄, X₅, X₆, Y, Z, R², R³ and R⁴ are as defined herein.

In a particular embodiment, compounds of the invention have the stereochemical orientation represented by Formula Ib:

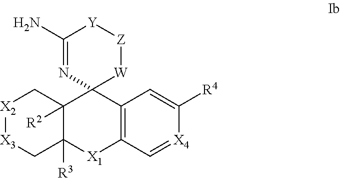

wherein W, X₁, X₂, X₃, X₄, Y, Z, R², R³ and R⁴ are as defined herein.

In a particular embodiment, compounds of the invention have the stereochemical orientation represented by Formula I′c:

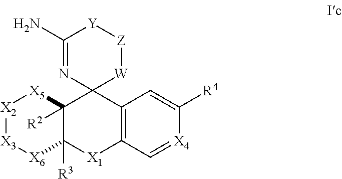

wherein W, X₁, X₂, X₃, X₄, X₅, X₆, Y, Z, R², R³ and R⁴ are as defined herein.

In a particular embodiment, compounds of the invention have the stereochemical orientation represented by Formula Ic:

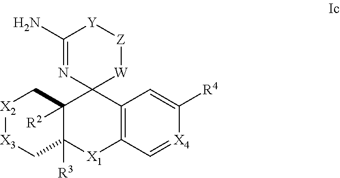

wherein W, X₁, X₂, X₃, X₄, Y, Z, R², R³ and R⁴ are as defined herein.

In a particular embodiment, compounds of the invention have the stereochemical orientation represented by Formula I′d:

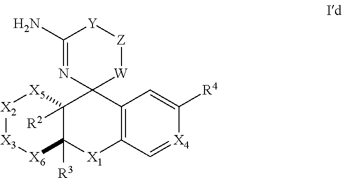

wherein W, X₁, X₂, X₃, X₄, X₅, X₆, Y, Z, R², R³ and R⁴ are as defined herein.

In a particular embodiment, compounds of the invention have the stereochemical orientation represented by Formula Id:

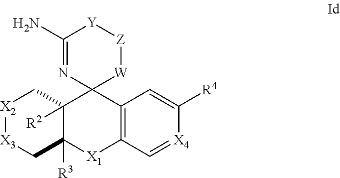

wherein W, X₁, X₂, X₃, X₄, Y, Z, R², R³ and R⁴ are as defined herein.

In a particular embodiment, compounds of the invention have the stereochemical orientation represented by Formula I′e:

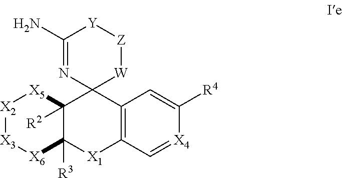

wherein W, X₁, X₂, X₃, X₄, X₅, X₆, Y, Z, R², R³ and R⁴ are as defined herein.

In a particular embodiment, compounds of the invention have the stereochemical orientation represented by Formula Ie:

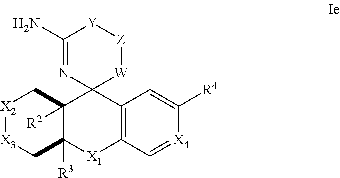

wherein W, X₁, X₂, X₃, X₄, Y, Z, R², R³ and R⁴ are as defined herein.

In a particular embodiment, compounds of the invention have the stereochemical orientation represented by Formula I′f:

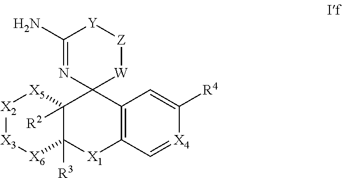

wherein W, X₁, X₂, X₃, X₄, X₅, X₆, Y, Z, R², R³ and R⁴ are as defined herein.

In a particular embodiment, compounds of the invention have the stereochemical orientation represented by Formula If:

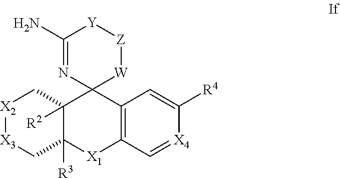

wherein W, X₁, X₂, X₃, X₄, Y, Z, R², R³ and R⁴ are as defined herein.

In a particular embodiment, compounds of the invention have the stereochemical orientation represented by Formula I′g:

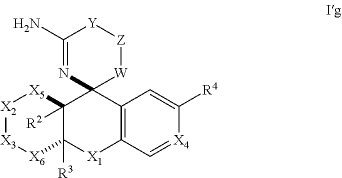

wherein W, X₁, X₂, X₃, X₄, X₅, X₆, Y, Z, R², R³ and R⁴ are as defined herein.

In a particular embodiment, compounds of the invention have the stereochemical orientation represented by Formula Ig:

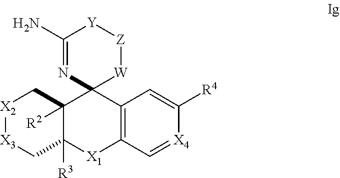

wherein W, X₁, X₂, X₃, X₄, Y, Z, R², R³ and R⁴ are as defined herein.

In a particular embodiment, compounds of the invention have the stereochemical orientation represented by Formula I′h:

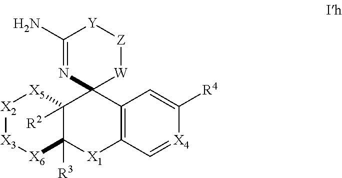

wherein W, X₁, X₂, X₃, X₄, X₅, X₆, Y, Z, R², R³ and R⁴ are as defined herein.

In a particular embodiment, compounds of the invention have the stereochemical orientation represented by Formula Ih:

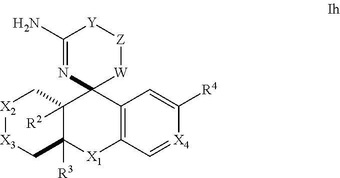

wherein W, X₁, X₂, X₃, X₄, Y, Z, R², R³ and R⁴ are as defined herein.

In a particular embodiment, compounds of the invention have the stereochemical orientation represented by Formula I′i:

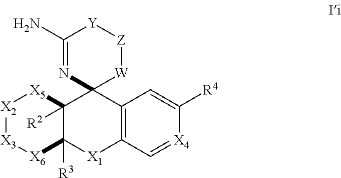

wherein W, X₁, X₂, X₃, X₄, X₅, X₆, Y, Z, R², R³ and R⁴ are as defined herein.

In a particular embodiment, compounds of the invention have the stereochemical orientation represented by Formula Ii:

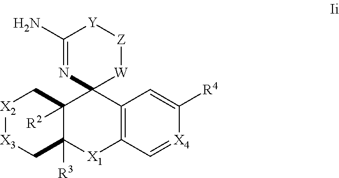

wherein W, X₁, X₂, X₃, X₄, Y, Z, R², R³ and R⁴ are as defined herein.

In a particular embodiment, compounds of the invention have the stereochemical orientation represented by Formula I′j:

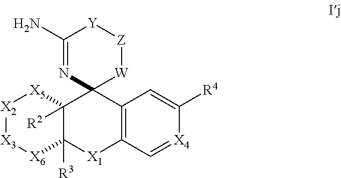

wherein W, X₁, X₂, X₃, X₄, X₅, X₆, Y, Z, R², R³ and R⁴ are as defined herein.

In a particular embodiment, compounds of the invention have the stereochemical orientation represented by Formula Ij:

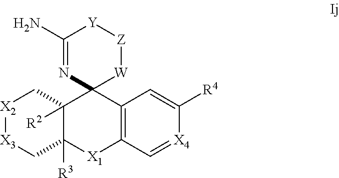

wherein W, X₁, X₂, X₃, X₄, Y, Z, R², R³ and R⁴ are as defined herein.

In a particular embodiment, compounds of the invention have the stereochemical orientation represented by Formula I′k:

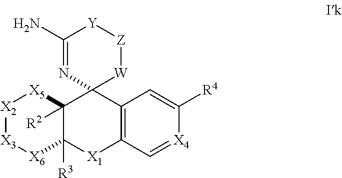

wherein W, X₁, X₂, X₃, X₄, X₅, X₆, Y, Z, R², R³ and R⁴ are as defined herein.

In a particular embodiment, compounds of the invention have the stereochemical orientation represented by Formula Ik:

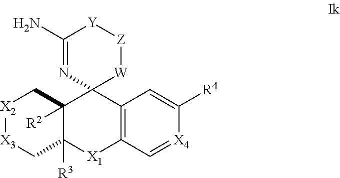

wherein W, X₁, X₂, X₃, X₄, Y, Z, R², R³ and R⁴ are as defined herein.

In a particular embodiment, compounds of the invention have the stereochemical orientation represented by Formula I′l:

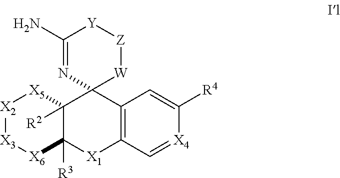

wherein W, X₁, X₂, X₃, X₄, X₅, X₆, Y, Z, R², R³ and R⁴ are as defined herein.

In a particular embodiment, compounds of the invention have the stereochemical orientation represented by Formula Il:

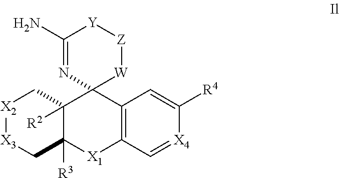

wherein W, X₁, X₂, X₃, X₄, Y, Z, R², R³ and R⁴ are as defined herein.

In a particular embodiment, compounds of the invention have the stereochemical orientation represented by Formula I′m:

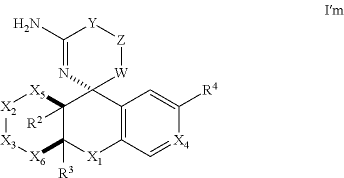

wherein W, X₁, X₂, X₃, X₄, X₅, X₆, Y, Z, R², R³ and R⁴ are as defined herein.

In a particular embodiment, compounds of the invention have the stereochemical orientation represented by Formula Im:

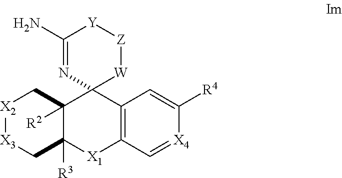

wherein W, X₁, X₂, X₃, X₄, Y, Z, R², R³ and R⁴ are as defined herein.

In a particular embodiment, compounds of the invention have the stereochemical orientation represented by Formula I′n:

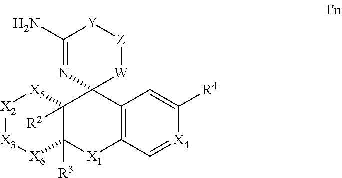

wherein W, X₁, X₂, X₃, X₄, X₅, X₆, Y, Z, R², R³ and R⁴ are as defined herein.

In a particular embodiment, compounds of the invention have the stereochemical orientation represented by Formula In:

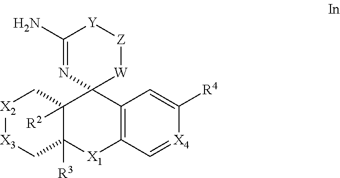

wherein W, X₁, X₂, X₃, X₄, Y, Z, R², R³ and R⁴ are as defined herein.

In certain embodiments, Y is O, S or NR¹. In certain embodiments, Y is O. In certain embodiments, Y is S. In certain embodiments, Y is NR¹. In certain embodiments, R¹ is C₁-C₃ alkyl. In certain embodiments, R¹ is methyl.

In certain embodiments, Z is a bond, CH₂ or C(═O), provided when Z is C(═O) then Y is NR¹.

In certain embodiments, Z is a bond. When Z is a bond, the compounds of Formula I have the structure of Formula I′o:

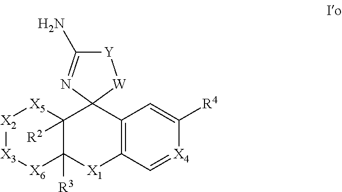

wherein W, X₁, X₂, X₃, X₄, X₅, X₆, Y, R², R³ and R⁴ are as defined herein.

In certain embodiments, Z is a bond. When Z is a bond, the compounds of Formula I have the structure of Formula Io:

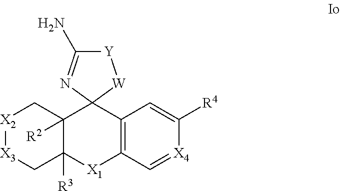

wherein W, X₁, X₂, X₃, X₄, Y, R², R³ and R⁴ are as defined herein.

In certain embodiments, Z is CH₂ or C(═O), provided when Z is C(═O) then Y is NR¹. In certain embodiments, Z is CH₂. In certain embodiments, Z is C(═O) and Y is NR¹.

In certain embodiments, (i) Y is O and Z is a bond, (ii) Y is S and Z is a bond, (iii) Y is NR¹ and Z is C(═O), or (iv) Y is S and Z is CH₂. In certain embodiments, Y is O and Z is a bond. In certain embodiments, Y is S and Z is a bond. In certain embodiments, Y is NR¹ and Z is C(═O). In certain embodiments, Y is S and Z is CH₂.

In certain embodiments, W is CR¹²R¹³. In certain embodiments, R¹² and R¹³ are independently selected from hydrogen and C₁-C₃ alkyl, or R¹² and R¹³ together with the atom to which they are attached form a 3 to 6 membered carbocycle or heterocycle. In certain embodiments, R¹² and R¹³ are hydrogen. In certain embodiments, R¹² and R¹³ together with the atom to which they are attached form a 3 to 6 membered carbocycle or heterocycle. In certain embodiments, R¹² and R¹³ together with the atom to which they are attached form a C₃-C₆ carbocycle. In certain embodiments, R¹² and R¹³ together with the atom to which they are attached form a 3 to 6 membered heterocycle, wherein the heterocycle contains one or two heteroatoms selected from nitrogen, oxygen and sulfur.

In certain embodiments, X₁ is selected from O, S, S(O), SO₂, NR¹⁰ and CHR¹⁰. In certain embodiments, X₁ is O.

In certain embodiments, X₂ is selected from CR⁵R⁶, NR⁷ and O. In certain embodiments, X₂ is CR⁵R⁶. In certain embodiments, X₂ is NR⁷. In certain embodiments, X₂ is O.

In certain embodiments, X₂ is CR⁵R⁶. In certain embodiments, R⁵ and R⁶ are independently selected from hydrogen, halogen, OR^{a}, NR^{b}R^{c}, C₁-C₆ alkyl, C₁-C₆ alkoxy, phenyl, a 3 to 6 membered heterocycle and a 5 to 6 membered heteroaryl, wherein the heteroaryl is optionally substituted with halogen. In certain embodiments, X₂ is CR⁵R⁶. In certain embodiments, R⁵ and R⁶ are independently selected from hydrogen, halogen, OR^{a}, NR^{b}R^{c}, C₁-C₆ alkyl, C₁-C₆ alkoxy and a 3 to 6 membered heterocycle. In certain embodiments, R⁵ and R⁶ are independently selected from hydrogen, halogen, OR^{a}, NR^{b}R^{c} and a 3 to 6 membered heterocycle. In certain embodiments, R^{a} is selected from hydrogen, C₁-C₆ alkyl and (CH₂)₀₋₃(3 to 6 membered carbocyclic). In certain embodiments, R^{a} is selected from hydrogen, methyl, ethyl, isopropyl, tert-butyl, cyclopropylmethyl and cyclobutyl. In certain embodiments, R^{b} and R^{c} are independently selected from hydrogen and C₁-C₆ alkyl, or R^{b} and R^{c} together with the nitrogen atom to which they are attached form a 3 to 6 membered heterocyclic. In certain embodiments, R^{b} and R^{c} are methyl. In certain embodiments, R^{b} and R^{c} together with the nitrogen atom to which they are attached form a 3 to 6 membered heterocyclic, wherein the heterocyclic is pyrrolidin-1-yl. In certain embodiments, R⁵ and R⁶ are independently selected from hydrogen, F, hydroxy, methyl, ethyl, methoxy, neopentyloxy, cyclopropylmethoxy, cyclobutoxy, isopropoxy, methoxymethyl, ethoxymethyl, dimethylamine, phenyl, pyrrolidin-1-yl, pyridin-2-yl and 5-chloropyridin-3-yl. In certain embodiments, R⁵ is selected from hydrogen, F, methyl, ethyl, hydroxy, methoxy, ethoxy, neopentyloxy, cyclopropylmethoxy, cyclobutoxy, isopropoxy, methoxymethyl, ethoxymethyl, dimethylamine, phenyl, pyrrolidin-1-yl, pyridin-2-yl and 5-chloropyridin-3-yl. In certain embodiments, R⁶ is selected from hydrogen, F, methyl, ethyl and hydroxy. In certain embodiments, R⁵ is selected from hydrogen, F, hydroxy, methyl, ethyl, neopentyloxy, cyclopropylmethoxy, methoxy, ethoxy, cyclobutoxy, isopropoxy, methoxymethyl, ethoxymethyl, dimethylamine, phenyl, pyrrolidin-1-yl, pyridin-2-yl and 5-chloropyridin-3-yl, and R⁶ is selected from hydrogen, F, methyl and ethyl.

In certain embodiments, X₂ is CR⁵R⁶. In certain embodiments, R⁵ and R⁶ are independently selected from hydrogen, halogen, OR^{a} and NR^{b}R^{c}. In certain embodiments, R^{a} is selected from hydrogen, C₁-C₆ alkyl and (CH₂)₀₋₃(3 to 6 membered carbocyclic). In certain embodiments, R^{a} is selected from hydrogen, methyl, tert-butyl and cyclopropylmethyl. In certain embodiments, R^{b} and R^{c} are independently selected from hydrogen and C₁-C₆ alkyl, or R^{b} and R^{c} together with the nitrogen atom to which they are attached form a 3 to 6 membered heterocyclic. In certain embodiments, R^{b} and R^{c} are methyl. In certain embodiments, R^{b} and R^{c} together with the nitrogen atom to which they are attached form a 3 to 6 membered heterocyclic, wherein the heterocyclic is pyrrolidin-1-yl. In certain embodiments, R⁵ and R⁶ are independently selected from hydrogen, F, hydroxy, methoxy, neopentyloxy, cyclopropylmethoxy, dimethylamine and pyrrolidin-1-yl. In certain embodiments, R⁵ is selected from hydrogen, F, hydroxy, methoxy, neopentyloxy, cyclopropylmethoxy, dimethylamine and pyrrolidin-1-yl. In certain embodiments, R⁶ is selected from hydrogen and F. In certain embodiments, R⁵ is selected from hydrogen, F, hydroxy, methoxy, neopentyloxy, cyclopropylmethoxy, dimethylamine and pyrrolidin-1-yl, and R⁶ is selected from hydrogen and F.

In certain embodiments, R⁵ and R⁶ together with the atom to which they are attached form a 3 to 6 membered carbocycle or heterocycle. In certain embodiments, R⁵ and R⁶ together with the atom to which they are attached form a 3 to 6 membered heterocycle. In certain embodiments, R⁵ and R⁶ together with the atom to which they are attached form a 3 to 6 membered heterocycle, wherein the heterocycle contains one, two or three heteroatoms selected from O, N and S. In certain embodiments, R⁵ and R⁶ together with the atom to which they are attached form a 3 to 6 membered heterocycle, wherein the heterocycle contains one or two O heteroatoms. In certain embodiments, R⁵ and R⁶ together with the atom to which they are attached form a 3 to 6 membered heterocycle, wherein the heterocycle is 1,3-dioxolane or oxetane.

In certain embodiments, X₂ is NR⁷. In certain embodiments, R⁷ is selected from hydrogen, C₁-C₆ alkyl, C₁-C₆ alkoxycarbonyl, —SO₂(C₁-C₆ alkyl) and a 4 to 6 membered heterocyclic, wherein the alkyls and alkoxycarbonyl are optionally substituted with one or more R^{d} groups. In certain embodiments, R^{d} is selected from halogen, hydroxy, oxo, C₃-C₆ cycloalkyl and phenyl, wherein the phenyl is optionally substituted with halogen, C₁-C₃ alkyl or C₁-C₃ alkoxy. In certain embodiments, R^{d} is selected from halogen, hydroxy, oxo, C₃-C₆ cycloalkyl and phenyl, wherein the phenyl is optionally substituted with C₁-C₃ alkoxy. In certain embodiments, R^{d} is selected from F, hydroxy, oxo, cyclopropyl, phenyl and 4-methoxyphenyl. In certain embodiments, R⁷ is selected from hydrogen, C₁-C₆ alkyl, C₁-C₆ alkoxycarbonyl, —SO₂(C₁-C₆ alkyl) and a 4 to 6 membered heterocyclic, wherein the alkyls and alkoxycarbonyl are optionally substituted with one or more R^{d} groups, and wherein the heterocycle contains one or two heteroatoms selected from nitrogen, oxygen and sulfur. In certain embodiments, R⁷ is selected from hydrogen, C₁-C₆ alkyl, C₁-C₆ alkoxycarbonyl, —SO₂(C₁-C₆ alkyl) and a 4 to 6 membered heterocyclic, wherein the alkyls and alkoxycarbonyl are optionally substituted with one or more R^{d} groups, and wherein the heterocycle is tetrahydropyranyl. In certain embodiments, R⁷ is selected from hydrogen, isopropyl, isobutyl, 2,2,2-trifluoroethyl, cyclopropylethyl, (4-methoxyphenyl)methyl, 2,2-difluoroethyl, CH₂CH(CH₃)₂OH, C(═O)CH₂CH(CH₃)₂, C(═O)CH₂(cyclopropyl), C(═O)O(benzyl), C(═O)OCH(CH₃)₂, S(O₂)CH₂CH(CH₃)₂, S(O₂)CH₂(cyclopropyl) and tetrahydropyran-4-yl.

In certain embodiments, X₂ is NR⁷. In certain embodiments, R⁷ is selected from hydrogen, C₁-C₆ alkyl, C₁-C₆ alkoxycarbonyl and a 4 to 6 membered heterocyclic, wherein the alkyl and alkoxycarbonyl are optionally substituted with one or more R^{d} groups. In certain embodiments, R^{d} is selected from halogen and phenyl, wherein the phenyl is optionally substituted with halogen, C₁-C₃ alkyl or C₁-C₃ alkoxy. In certain embodiments, R^{d} is selected from halogen and phenyl, wherein the phenyl is optionally substituted with C₁-C₃ alkoxy. In certain embodiments, R^{d} is selected from F, phenyl and 4-methoxyphenyl. In certain embodiments, R⁷ is selected from hydrogen, C₁-C₆ alkyl, C₁-C₆ alkoxycarbonyl and a 4 to 6 membered heterocyclic, wherein the alkyl and alkoxycarbonyl are optionally substituted with one or more R^{d} groups, and wherein the heterocycle contains one or two heteroatoms selected from nitrogen, oxygen and sulfur. In certain embodiments, R⁷ is selected from hydrogen, C₁-C₆ alkyl, C₁-C₆ alkoxycarbonyl and a 4 to 6 membered heterocyclic, wherein the alkyl and alkoxycarbonyl are optionally substituted with one or more R^{d} groups, and wherein the heterocycle is tetrahydropyranyl. In certain embodiments, R⁷ is selected from hydrogen, isopropyl, isobutyl, 2,2,2-trifluoroethyl, (4-methoxyphenyl)methyl, C(═O)O(benzyl) and tetrahydropyran-4-yl.

In certain embodiments, X₃ is selected from a bond, CR⁸R⁹ and O. In certain embodiments, X₃ is selected from CR⁸R⁹ and O. In certain embodiments, X₃ is CR⁸R⁹. In certain embodiments, X₃ is O, X₂ is CR⁵, R⁶ and X₅ is CR¹⁴R¹⁵.

In certain embodiments, X₃ is selected from a bond, CR⁸R⁹ and O. In certain embodiments, X₃ is selected from CR⁸R⁹ and O. In certain embodiments, X₃ is CR⁸R⁹. In certain embodiments, X₃ is O and X₂ is CR⁵R⁶. In certain embodiments, R⁸ and R⁹ are independently selected from hydrogen and OR^{e}, or R⁸ and R⁹ taken together form an oxo group, or R⁸ and R⁹ together with the atom to which they are attached form a 3 to 6 membered heterocycle. In certain embodiments, R⁸ and R⁹ are independently selected from hydrogen and OR^{e}. In certain embodiments, R^{e} is selected from hydrogen and C₁-C₆ alkyl. In certain embodiments, R^{e} is hydrogen. In certain embodiments, R⁸ and R⁹ are independently selected from hydrogen and OH. In certain embodiments, R⁸ is independently selected from hydrogen and OH and R⁹ is hydrogen. In certain embodiments, R⁸ and R⁹ taken together form an oxo group. In certain embodiments, R⁸ and R⁹ together with the atom to which they are attached form a 3 to 6 membered heterocycle. In certain embodiments, R⁸ and R⁹ together with the atom to which they are attached form a 3 to 6 membered heterocycle, wherein the heterocycle contains one or two heteroatoms selected from nitrogen, oxygen and sulfur. In certain embodiments, R⁸ and R⁹ together with the atom to which they are attached form a 3 to 6 membered heterocycle, wherein the heterocycle is dioxolanyl. In certain embodiments, R⁸ and R⁹ together with the atom to which they are attached form a 3 to 6 membered heterocycle, wherein the heterocycle is 1,3-dioxolan-2-yl.

In certain embodiments, when X₃ is O then X₂ is CR⁵R⁶.

In certain embodiments, X₃ is a bond. When X₃ is a bond, the compounds of Formula I′ have the structure of Formula I′p:

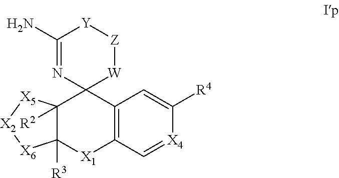

wherein W, X₁, X₂, X₄, X₅, X₆, Y, Z, R², R³ and R⁴ are as defined herein.

In certain embodiments, X₃ is a bond. When X₃ is a bond, the compounds of Formula I have the structure of Formula Ip:

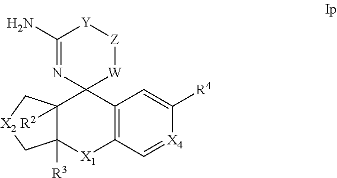

wherein W, X₁, X₂, X₄, Y, Z, R², R³ and R⁴ are as defined herein.

In certain embodiments, X₄ is CR¹¹. In certain embodiments, R¹¹ is hydrogen or halogen. In certain embodiments, X₄ is CH, CF or CCl.

In certain embodiments, X₄ is CR¹¹. In certain embodiments, R¹¹ is hydrogen. In certain embodiments, X₄ is CH.

In certain embodiments, X₅ is selected from CR¹⁴R¹⁵ and O, provided when X₅ is O, then X₂ is CR⁵R⁶ and X₃ is CR⁸R⁹ or a bond. In certain embodiments, X₅ is CR¹⁴R¹⁵. In certain embodiments, X₅ is O, X₂ is CR⁵R⁶ and X₃ is CR⁸R⁹ or a bond. In certain embodiments, R¹⁴ and R¹⁵ are independently selected from hydrogen and C₁-C₃ alkyl. In certain embodiments, R¹⁴ and R¹⁵ are independently selected from hydrogen and methyl. In certain embodiments, R¹⁴ and R¹⁵ are hydrogen. In certain embodiments, R¹⁴ is hydrogen and R¹⁵ is methyl. In certain embodiments, R¹⁴ and R¹⁵ are hydrogen, or R¹⁴ is hydrogen and R¹⁵ is methyl.

In certain embodiments, X₆ is CR¹⁶R¹⁷. In certain embodiments, R¹⁶ and R¹⁷ are independently selected from hydrogen and halogen. In certain embodiments, R¹⁶ and R¹⁷ are independently selected from hydrogen and F. In certain embodiments, R¹⁶ and R¹⁷ are hydrogen. In certain embodiments, R¹⁶ and R¹⁷ are F.

In certain embodiments, R¹ is C₁-C₃ alkyl. In certain embodiments, R¹ is methyl.

In certain embodiments, R² is hydrogen or C₁-C₆ alkyl. In certain embodiments, R² is hydrogen or C₁-C₃ alkyl. In certain embodiments, R² is hydrogen or methyl. In certain embodiments, R² is in the (S) configuration. In certain embodiments, R² is in the (R) configuration.

In certain embodiments, R² is hydrogen or C₁-C₆ alkyl. In certain embodiments, R² is hydrogen or C₁-C₃ alkyl. In certain embodiments, R² is hydrogen. In certain embodiments, R² is in the (S) configuration. In certain embodiments, R² is in the (R) configuration.

In certain embodiments, R³ is hydrogen or C₁-C₆ alkyl. In certain embodiments, R³ is hydrogen or C₁-C₃ alkyl. In certain embodiments, R³ is hydrogen or methyl. In certain embodiments, R³ is in the (S) configuration. In certain embodiments, R³ is in the (R) configuration.

In certain embodiments, R³ is hydrogen or C₁-C₆ alkyl. In certain embodiments, R³ is hydrogen or C₁-C₃ alkyl. In certain embodiments, R³ is hydrogen. In certain embodiments, R³ is in the (S) configuration. In certain embodiments, R³ is in the (R) configuration.

In certain embodiments, R² and R³ are independently selected from hydrogen and C₁-C₆ alkyl. In certain embodiments, R² and R³ are independently selected from hydrogen and C₁-C₃ alkyl. In certain embodiments, R² and R³ are independently selected from hydrogen and methyl. In certain embodiments, R² and R³ are both in the (S) configuration. In certain embodiments, R² and R³ are both in the (R) configuration. In certain embodiments, R² is in the (S) configuration and R³ is in the (R) configuration. In certain embodiments, R² is in the (R) configuration and R³ is in the (S) configuration.

In certain embodiments, R² and R³ are independently selected from hydrogen and C₁-C₆ alkyl. In certain embodiments, R² and R³ are hydrogen or C₁-C₃ alkyl. In certain embodiments, R² and R³ are hydrogen. In certain embodiments, R² and R³ are both in the (S) configuration. In certain embodiments, R² and R³ are both in the (R) configuration. In certain embodiments, R² is in the (S) configuration and R³ is in the (R) configuration. In certain embodiments, R² is in the (R) configuration and R³ is in the (S) configuration.

In certain embodiments, R⁴ is selected from halogen, C₁-C₆ alkoxy, NHC(═O)R^{f}, phenyl and a 5 to 6 membered heteroaryl, wherein the alkoxy, phenyl and heteroaryl are optionally substituted with one or more R^{g} groups. In certain embodiments, R⁴ is selected from halogen, C₁-C₆ alkoxy, NHC(═O)R^{f}, phenyl and a 5 to 6 membered heteroaryl, wherein the alkoxy, phenyl and heteroaryl are optionally substituted with one or two R^{g} groups. In certain embodiments, R⁴ is selected from halogen, C₁-C₆ alkoxy, NHC(═O)R^{f}, phenyl and a 5 to 6 membered heteroaryl, wherein the phenyl and heteroaryl are optionally substituted with one or two R^{g} groups. In certain embodiments, R^{f} is a 5 to 6 membered heteroaryl optionally substituted with halogen or C₁-C₃ alkyl. In certain embodiments, R^{f} is a 5 to 6 membered heteroaryl optionally substituted with halogen or C₁-C₃ alkyl, wherein the heteroaryl contains one, two, three or four heteroatoms selected from nitrogen, oxygen and sulfur. In certain embodiments, R^{f} is a 5 to 6 membered heteroaryl optionally substituted with halogen or C₁-C₃ alkyl, wherein the heteroaryl contains one or two heteroatoms selected from nitrogen and oxygen. In certain embodiments, R^{f} is a 5 to 6 membered heteroaryl optionally substituted with halogen or C₁-C₃ alkyl, wherein the heteroaryl is selected from oxazolyl and pyridinyl. In certain embodiments, each R^{g} is independently selected from halogen, CN, C₁-C₆ alkyl and C₁-C₆ alkoxy, wherein the alkyl and alkoxy are optionally substituted with halogen. In certain embodiments, R⁴ is selected from Br, methoxy, 5-chloropyridin-3-yl, 2-fluoropyridin-3-yl, pyrimidin-5-yl, 5-cyanopyridin-3-yl, 5-fluoropyridin-3-yl, 5-(trifluoromethyl)pyridin-3-yl, 4-(trifluoromethyl)pyridin-2-yl, 5-chloro-2-fluoropyridin-3-yl, 4-cyanopyridin-2-yl, 5-methoxypyridin3-yl, 3-chloro-5-fluorophenyl, 3-chlorophenyl, 3-cyanophenyl, 3-(difluoromethoxy)phenyl, 3,5-difluorophenyl, 3-chloro-2-fluorophenyl, 3-cyano-6-fluorophenyl, 5-chloro-2-fluorophenyl, 3-chloro-4-fluorophenyl, 3-cyano-5-fluorophenyl, 3-cyano-5-bromophenyl, 3-cyano-2-fluorophenyl, 3,5-dichlorophenyl, 3-chloro-5-(trifluoromethyl)phenyl, 3-cyano-5-chlorophenyl, NHC(═O)(2-methyloxazol-4-yl), NHC(═O)(5-chloropyridin-2-yl) and NHC(═O)(5-bromochloropyridin-2-yl).

In certain embodiments, R⁴ is selected from halogen, NHC(═O)R^{f}, phenyl and a 5 to 6 membered heteroaryl, wherein the phenyl and heteroaryl are optionally substituted with one or more R^{g} groups. In certain embodiments, R^{f} is a 5 to 6 membered heteroaryl optionally substituted with halogen or C₁-C₃ alkyl. In certain embodiments, R^{f} is a 5 to 6 membered heteroaryl optionally substituted with halogen or C₁-C₃ alkyl, wherein the heteroaryl contains one, two, three or four heteroatoms selected from nitrogen, oxygen and sulfur. In certain embodiments, R^{f} is a 5 to 6 membered heteroaryl optionally substituted with halogen or C₁-C₃ alkyl, wherein the heteroaryl contains one or two heteroatoms selected from nitrogen and oxygen. In certain embodiments, R^{f} is a 5 to 6 membered heteroaryl optionally substituted with halogen or C₁-C₃ alkyl, wherein the heteroaryl is selected from oxazolyl and pyridinyl. In certain embodiments, R^{g} is halogen. In certain embodiments, R⁴ is selected from Br, 5-chloropyridin-3-yl, 2-fluoropyridin-3-yl, pyrimidin-5-yl, 3-chloro-5-fluorophenyl, NHC(═O)(2-methyloxazol-4-yl), NHC(═O)(5-chloropyridin-2-yl) and NHC(═O)(5-bromochloropyridin-2-yl).

In certain embodiments, a compound selected from Examples 1 to 310 is provided. In certain embodiments, a compound selected from Examples 1 to 309 is provided. In certain embodiments, a compound selected from Examples 1 to 62 is provided. In certain embodiments, a compound of Formula I′ is provided, wherein the compound is not Example 310.

It will be appreciated that certain compounds described herein may contain asymmetric or chiral centers, and therefore exist in different stereoisomeric forms. It is intended that all stereoisomeric forms of the compounds described herein, including but not limited to, diastereomers, enantiomers and atropisomers, as well as mixtures thereof such as racemic mixtures, form part of the present compounds.

In the structures shown herein, where the stereochemistry of any particular chiral atom is not specified, then all stereoisomers are contemplated and included as the compounds described herein. Where stereochemistry is specified by a solid wedge or dashed line representing a particular configuration, then that stereoisomer is so specified and defined.

It will also be appreciated that certain compounds of Formula I′, I′a, I′b, I′c, I′d, I′e, I′g, I′h, I′i, I′j, I′k, I′l, I′m, I′n, I′o, I′p, I, Ia, Ib, Ic, Id, Ie, If, Ig, Ih, Ii, Ij, Ik, Il, Im, In, Io and Ip may be used as intermediates for further compounds of Formula I′, I′a, I′b, I′c, I′d, I′e, I′g, I′h, I′i, I′j, I′k, I′l, I′m, I′n, I′o, I′p, I, Ia, Ib, Ic, Id, Ie, If, Ig, Ih, Ii, Ij, Ik, Il, Im, In, Io and Ip.

It will be further appreciated that the compounds described herein may exist in unsolvated as well as solvated forms with pharmaceutically acceptable solvents such as water, ethanol, and the like, and it is intended that the compounds embrace both solvated and unsolvated forms.

Synthesis of Compounds

Compounds described herein may be synthesized by synthetic routes that include processes analogous to those well-known in the chemical arts, particularly in light of the description contained herein. The starting materials are generally available from commercial sources such as Sigma-Aldrich (St. Louis, Mo.), Alfa Aesar (Ward Hill, Mass.), or TCI (Portland, Oreg.), or are readily prepared using methods well known to those skilled in the art (e.g., prepared by methods generally described in Louis F. Fieser and Mary Fieser, Reagents for Organic Synthesis. v. 1-23, New York: Wiley 1967-2006 ed. (also available via the Wiley InterScience® website), or Beilsteins Handbuch der organischen Chemie, 4, Aufl. ed. Springer-Verlag, Berlin, including supplements (also available via the Beilstein online database)).

It will be appreciated that synthetic procedures employed in the preparation of compounds of the invention will depend on the particular substituents present in a compound. In preparing compounds of the invention, protection of remote functionalities (e.g., primary or secondary amines, etc.) of intermediates may be necessary but may not be illustrated in the following general Schemes. The need for such protection will vary depending on the nature of the remote functionality and the conditions of the preparation methods. The need for such protection is readily determined by one skilled in the art. For a general description of protecting groups and their use, see Greene's Protective Groups in Organic Synthesis, supra.

For illustrative purposes, Schemes 1-12 show general methods for preparing the compounds described herein, as well as key intermediates. For a more detailed description of the individual reaction steps, see the Examples section below. Those skilled in the art will appreciate that other synthetic routes may be used to synthesize the compounds. Although specific starting materials and reagents are depicted in the Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

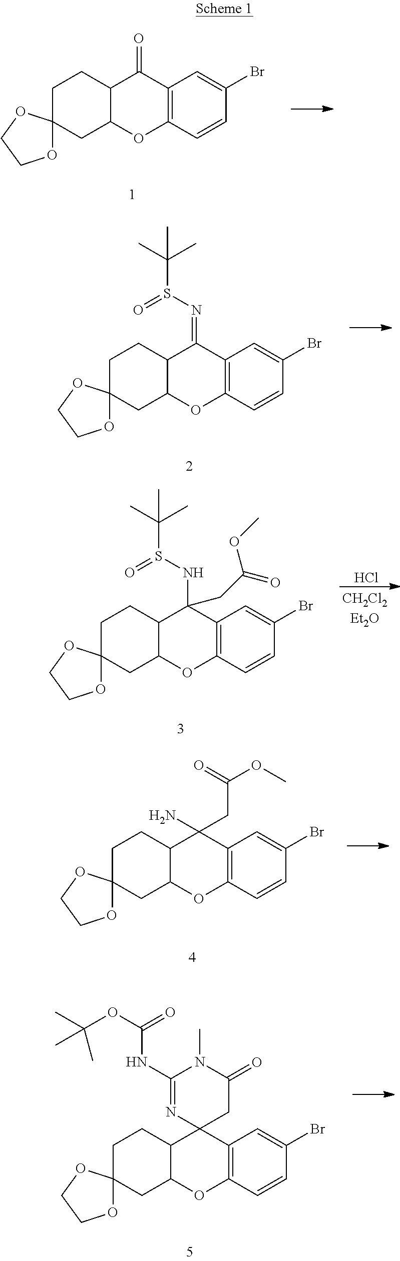

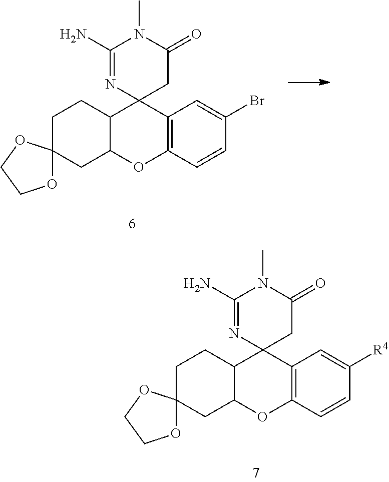

Scheme 1 shows a general scheme for the synthesis of compound 6 and 7, wherein R⁴ is defined herein. Compound 1 may be reacted with 2-methylpropane-2-sulfinamide and tetraethoxytitanium to provide compound 2. Compound 2 may be reacted with diisopropylamine, butyl lithium and methyl acetate to provide compound 3. Compound 3 may be reacted with HCl to provide compound 4. Compound 4 may be reacted with methylcarbamothioylcarbamate, N1-((ethylimino)methylene)-N3,N3-dimethylpropane-1,3-diamine hydrochloride and N-ethyl-N-isopropylpropan-2-amine to provide compound 5. Compound 5 may be deprotected to provide compound 6. When R⁴ is not bromine, a Suzuki, Negishi or Stille coupling installs the R⁴ group and provides compound 7.

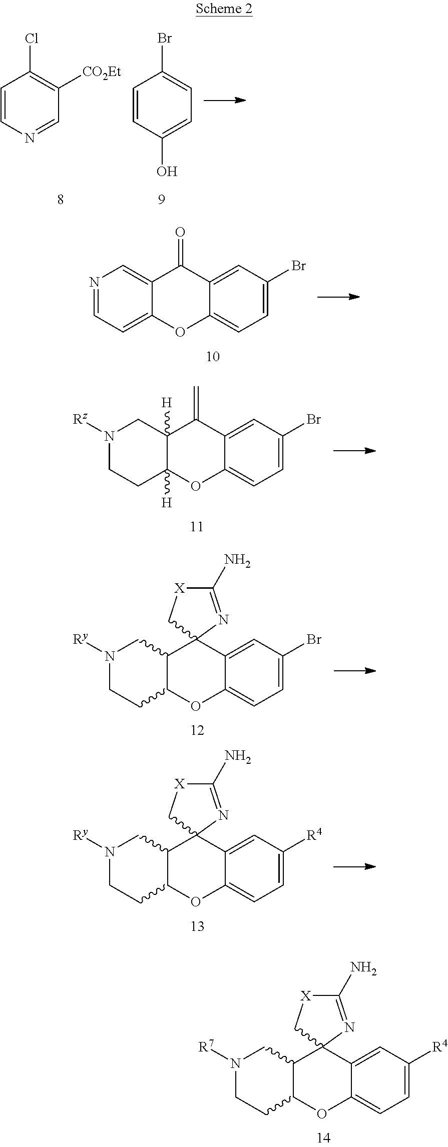

Scheme 2 shows a general scheme for the synthesis of compound 14, wherein Y, R⁴ and R⁷ are defined herein. Compound 8 may be reacted with compound 9, and the subsequent ester product is hydrolyzed with base, followed by cyclization under acidic conditions (such as H₂SO₄) to give compound 10. The R^{z} group may be installed, followed by reduction with NaBH₄, oxidation, and conversion of the resulting ketone to the olefin to give compound 11, wherein R^{z} is alkyl, benzyl or substituted benzyl. If desired, the R^{z} group may be removed, and an R^{y} may be installed at this stage, wherein R^{y} is an amino-protecting group, such as CBZ. The olefin 11 may then be converted to an amino-oxazoline or amino-thiazoline by treatment first with I₂/AgOCN or I₂/AgSCN, respectively, followed by treatment with NH₄OH to give compound 12. The R⁴ group may then be installed by a Pd-catalyzed coupling reaction to give compound 13. If desired, the R^{y} group may be removed, and R⁷ installed either via amide bond formation by coupling with an acid using an appropriate coupling reagent, sulfonylation using a sulfonyl chloride in the presence of a base, or reductive amination with a carbonyl-containing R⁷ group in the presence of a reducing agent to give compound 14.

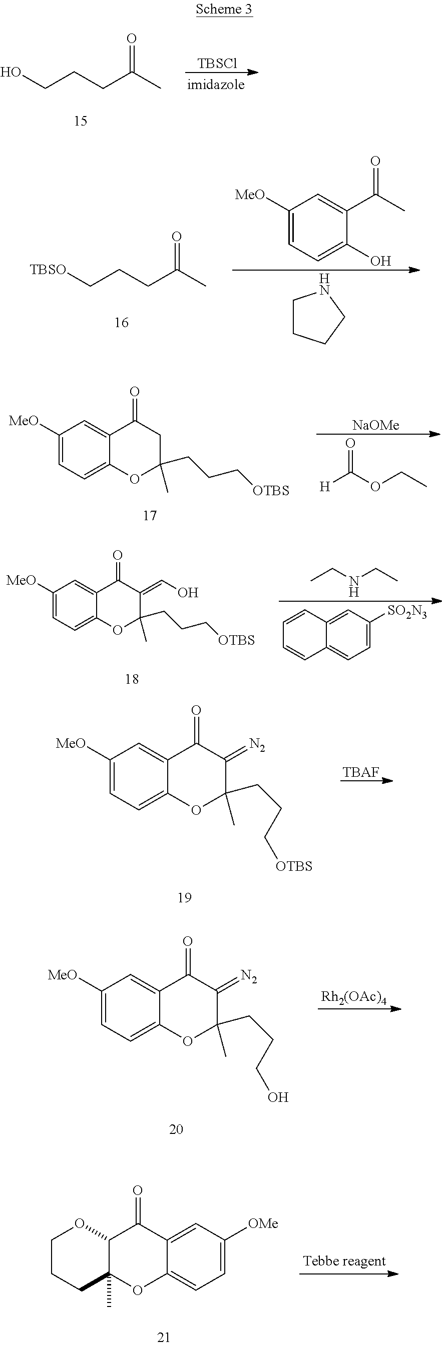

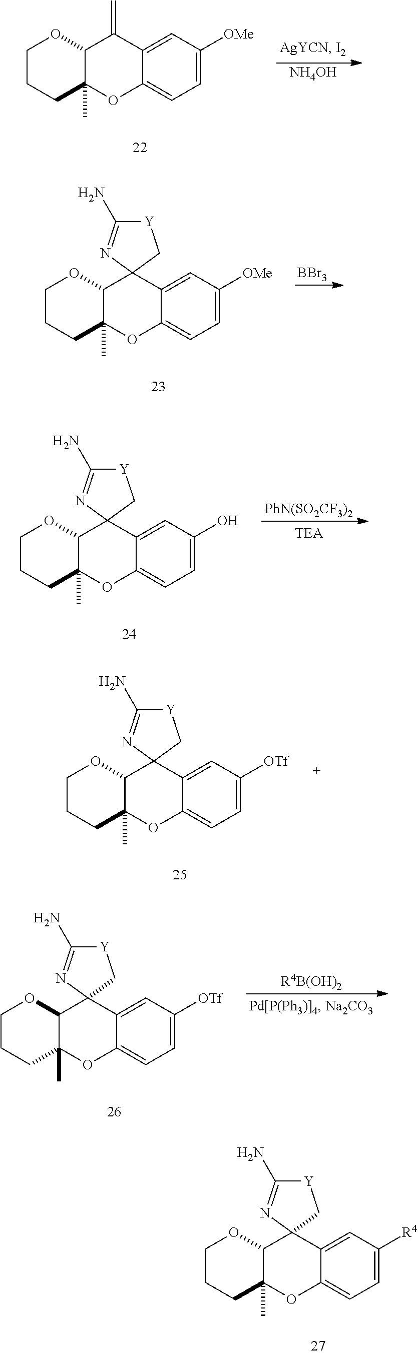

Scheme 3 depicts a general route toward compound 27, wherein Y is O or S and R⁴ is as defined herein (preferably aryl or heteroaryl). Alcohol 15 may be protected as a TBS ether 16, and then reacted with 1-(2-hydroxy-5-methoxyphenyl)ethanone in the presence of pyrrolidine to provide compound 17. Formulation of compound 17 provides compound 18, which may then be converted to the diazo compound 19 by a diazo transfer reaction using naphthalene-2-sulfonyl azide in the presence of diethylamine. After TBAF-mediated deprotection of the TBS ether to give compound 20, cyclization to afford ketone 21 may be effected by heating with catalytic Rh₂(OAc)₄. Treatment of ketone 21 with Tebbe reagent gives the alkene 22. The alkene 22 may then be converted to oxazoline or thiazoline (mixture of diastereomers) 23 with AgYCN/I₂/NH₄OH. After deprotection of the aryl methoxy group in 23 with BBr₃, the phenol group may then be converted to a triflate group giving diastereomers 25 and 26, which may be separated chromatographically. Diastereomer 25 may then be converted to compounds 27 via the Suzuki reaction. Alternatively, the diastereomers may be separated after the Suzuki reaction.

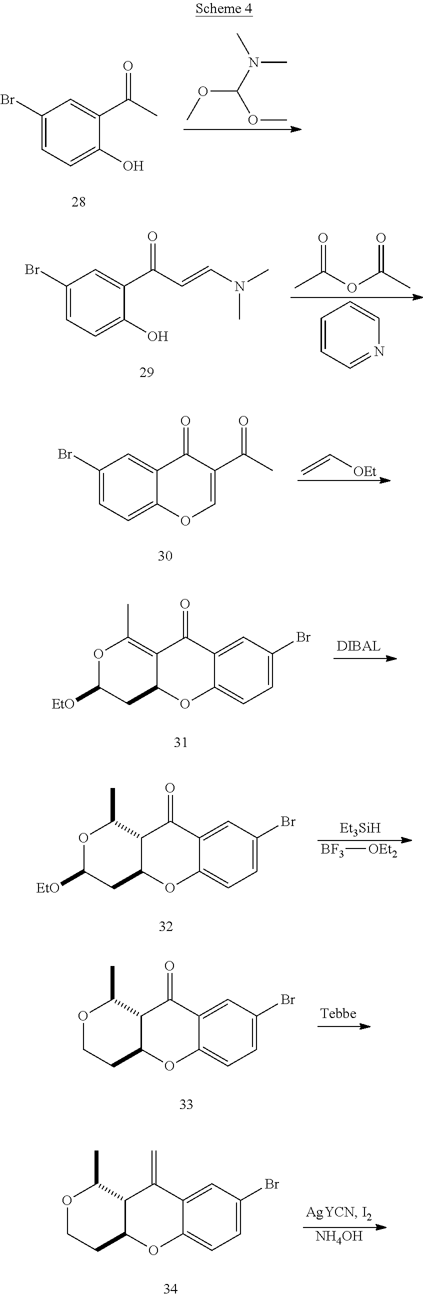

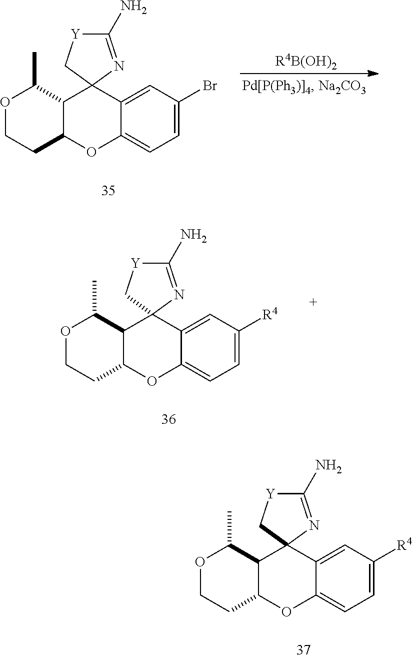

Scheme 4 depicts a general route toward compounds 36 and 37, wherein Y is O or S and R⁴ is as defined herein (preferably aryl or heteroaryl). Reaction of phenol 28 with DMF-dimethyl acetal provides enamine 29, which may then be converted to chromone 30 with acetic anhydride and pyridine according to methodology described in Phosphorus, Sulfur, and Silicon 2009, 184, 179-196. Hetero Diels-Alder reaction of 30 with ethyl vinyl ether gives compound 31, which may then be reduced at the double bond with DIBAL to provide compound 32. Removal of the ethoxy group of 32 may be accomplished with triethylsilane and boron trifluoride etherate to yield 33. Conversion of ketone 33 to alkene 34 utilizes Tebbe reagent. Alkene 34 may then be converted to a mixture of diastereomeric oxazolines or thiazolines 35 with AgYCN/I₂/NH₄OH. Suzuki reaction on the aromatic bromide provides diastereomers 36 and 37, which may be separated by chromatography.

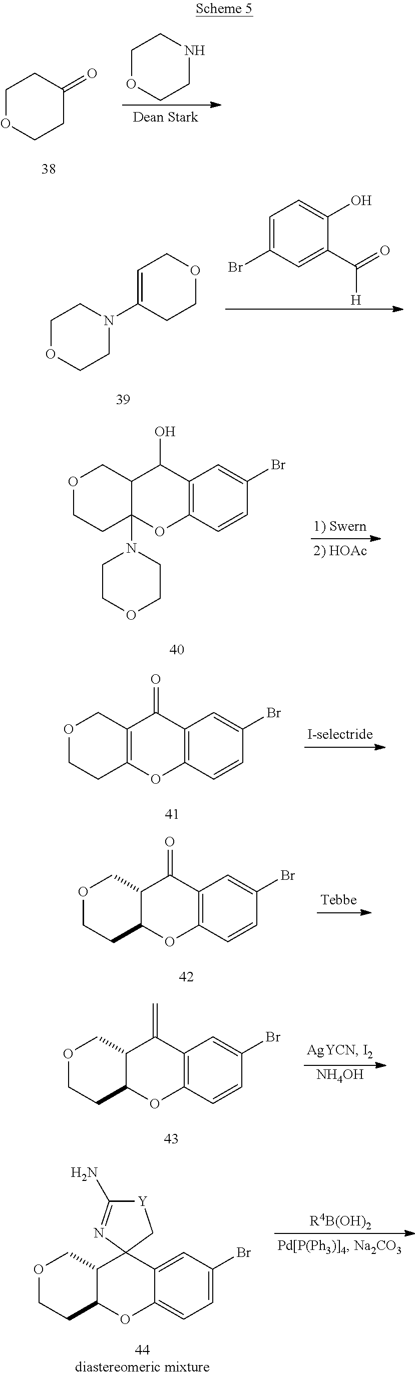

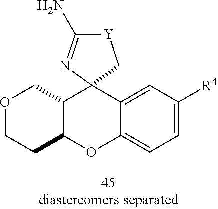

Scheme 5 depicts a general route toward compound 45, wherein Y is O or S and R⁴ is as defined herein (preferably aryl or heteroaryl). Pyran-one 38 may be condensed with morpholine to provide enamine 39, which may then be reacted with 5-bromo-2-hydroxybenzaldehyde to give compound 40. Compound 40 may be subjected to a Swern oxidation, and the morpholino group may be subsequently eliminated to give chromone 41. Reduction of the enone double bond in compound 41 with 1-selectride provides chromanone 42, which upon treatment with Tebbe reagent gives alkene 43. Reaction of alkene 43 with AgYCN/I₂/NH₄OH yields either oxazoline or thiazoline 44 as a mixture of diastereomers. Suzuki reaction of aryl bromide 44 provides compound 45, in which diastereomers may be separated.

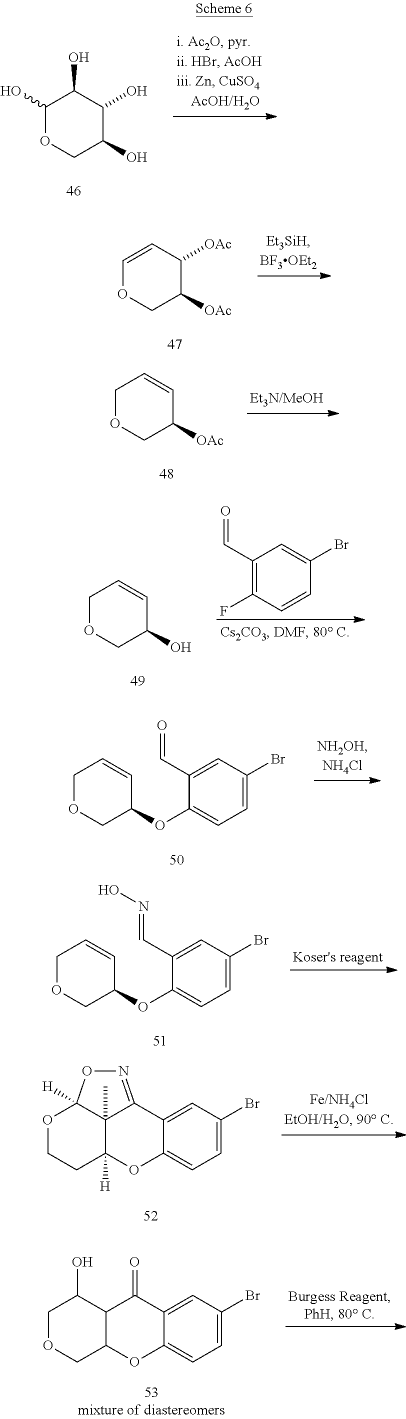

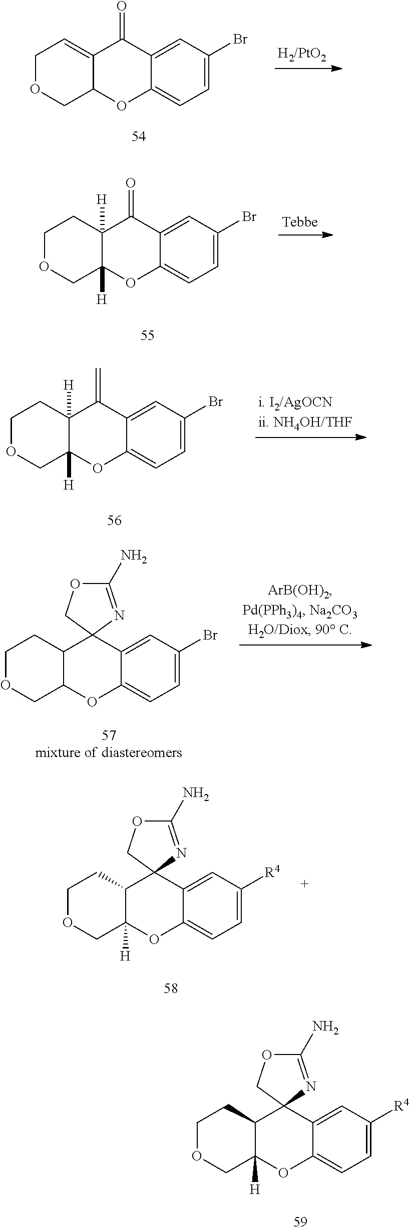

Scheme 6 depicts the general route towards compounds 58 and 59, wherein R4 is as defined herein (preferably aryl or heteroaryl). Xylose 46 is acetylated, brominated and reduced to provide diacetate 47. Reduction of compound 47 provides allylic acetate 48, which may be deacetylated to provide allylic alcohol 49. Compound 49 may be reacted with 5-bromo-2-fluoro-benzaldehyde to provide aldehyde 50, which may then be converted to the oxime 51. Oxidation/cycloaddition of compound 51 provides dihydroisoxazole 52, which may then be reduced to beta-hydroxy ketone 53 as a mixture of diastereomers. Dehydration of compound 53 provides enone 54, which may then be reduced to provide ketone 55 with a trans ring junction. Olefination of compound 55 provide olefin 56, which may then be converted to amino-oxazoline 57 as a mixture of diastereomers. The mixture of amino-oxazoline diastereomers 58 may then be converted to compounds 58 and 59 via the Suzuki reaction. Enantiopure 58 and 59 may be isolated after chiral SFC purification.

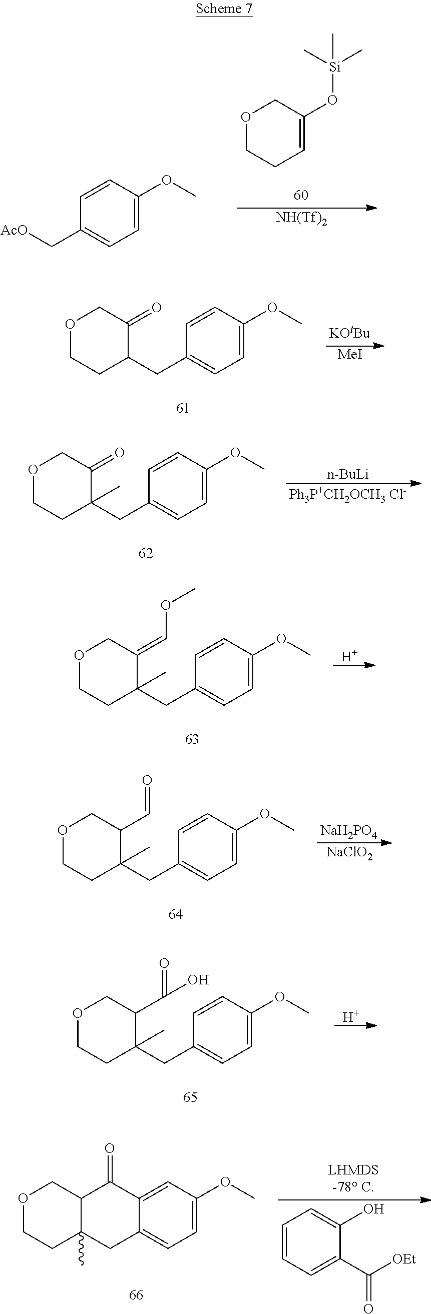

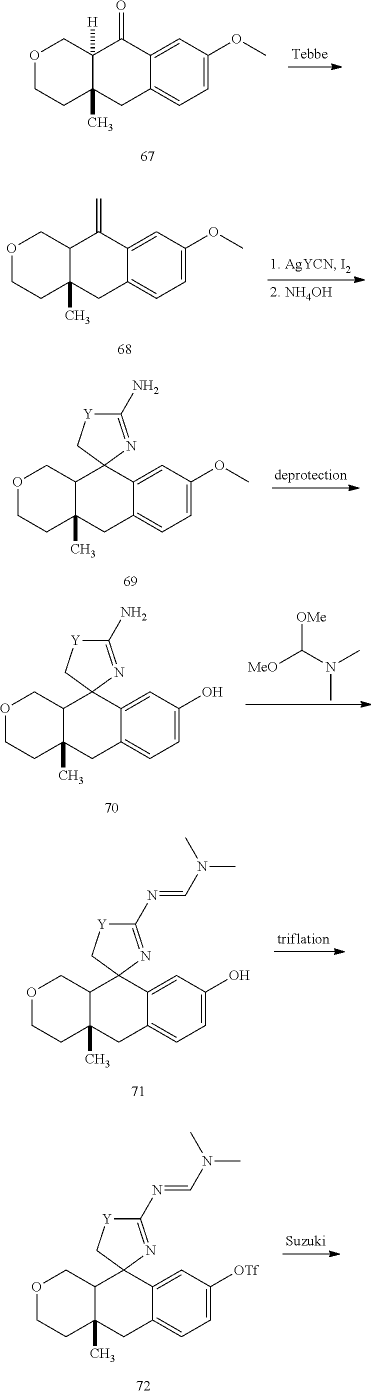

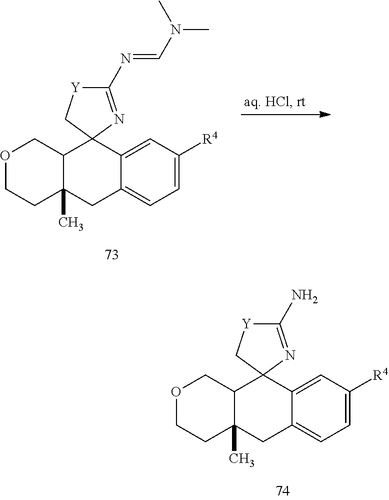

Scheme 7 describes the general synthetic route for the preparation of compound 74, wherein Y is as defined herein and R⁴ is aryl or heteroaryl. Silyl enole ether 60 was prepared according to the method described in WO 2009/43883. It may be reacted with a suitably substituted benzyl acetate in the presence of a catalyst such as NH(Tf)₂ to provide compound 61. Methylation of compound 61 can be effected with an alkylating agent, such as MeI in the presence of KOtBu to prepare compound 62. Aldehyde 64 can be prepared from Wittig reaction of ketone 62 followed by the hydrolysis of resulting vinyl ether 63. Oxidation of aldehyde 64 with an oxidizing agent will furnish the carboxylic acid 65, which in turn can be cyclized into a mixture of cis and trans ketone 66 in the presence of a acid (PPA, H₂SO₄, MSA, TFA etc). Epimerization of 66 with a base, such as LiHMDS, and quenching the resulting anion with ethyl salicylate may enrich the ratio of trans ketone 67. Ketone 67 can be subjected to olefination with Tebbe reagent or Wittig reaction to provide the alkene 68. The alkene 68 may then be converted to oxazoline or thiazoline diastereomers 69 with AgYCN/I₂/NH₄OH. After deprotection of the aryl methoxy group in 70 with HBr, the NH₂ group can be selectively protected with dimethylformimidamide group using 1,1-dimethoxy-N,N-dimethylmethanamine to give 71. The phenol group on 71 can be converted to a triflate group using triflic anhydride or 1,1,1-trifluoro-N-phenyl-N-(trifluoromethylsulfonyl)methanesulfonamide) to provide 72. Suzuki reaction of 72 followed by deprotection of dimethylformimidamide group with an acid (HCl, etc.) will furnish the diastereomers of 74, which may be separated by chromatography.

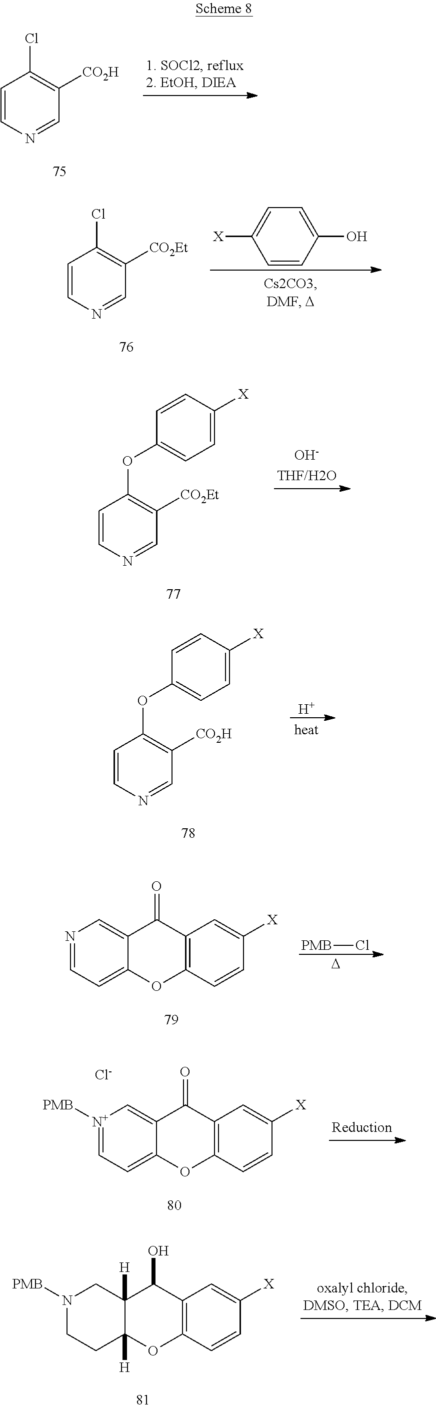

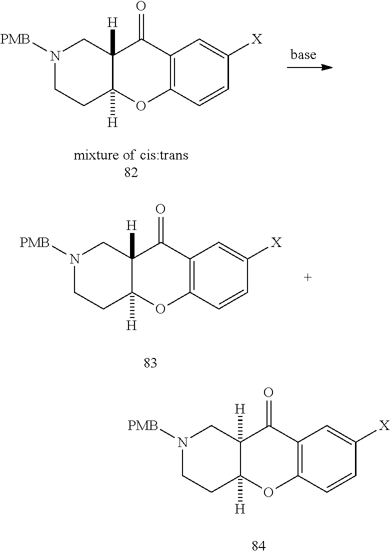

Scheme 8 describes the general route for the synthesis of 2,3,4,4a,10,10a-hexahydro-1H-chromeno[3,2-c]pyridin-10-ol core compounds 83 and 84. Ethyl 4-chloronicotinate (prepared as described in WO 2008/02472) can be subjected to SnAr reaction with appropriately substituted phenols in the presence of a suitable base, such as Cs₂CO₃, Na₂CO₃ (but not limited to) to prepare phenyl esters such as compound 77. Treatment of compound 74 with an appropriate base (such as aqueous NaOH, LiOH, KOH, etc.) will furnish the corresponding aromatic carboxylic acid 78, which in turn can be cyclized to give compound 79. The PMB-chloride salt 80, which can be prepared by the reaction of the compound 80 with PMB-Cl can be treated with a reducing agent, such as NaBH₄ to provide compound 81. The hydroxyl group then can be oxidized with oxalyl chloride in the presence of DMSO to give a mixture of cis and trans ketone 82. Treatment of the racemic ketone with a base, such as K₂CO₃, Na₂CO₃ or LiHMDS, may result in the epimerization to give a higher ratio of trans ketone 84 at this stage.

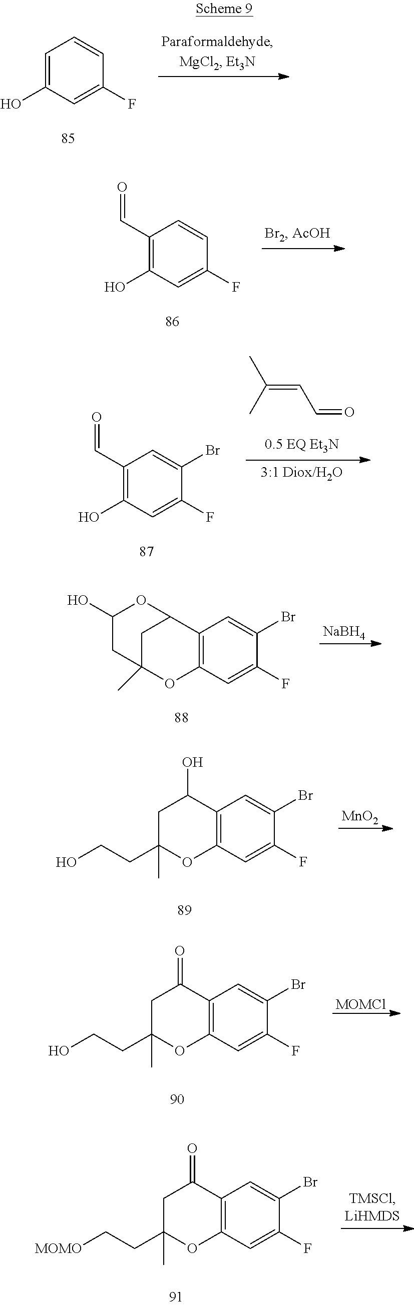

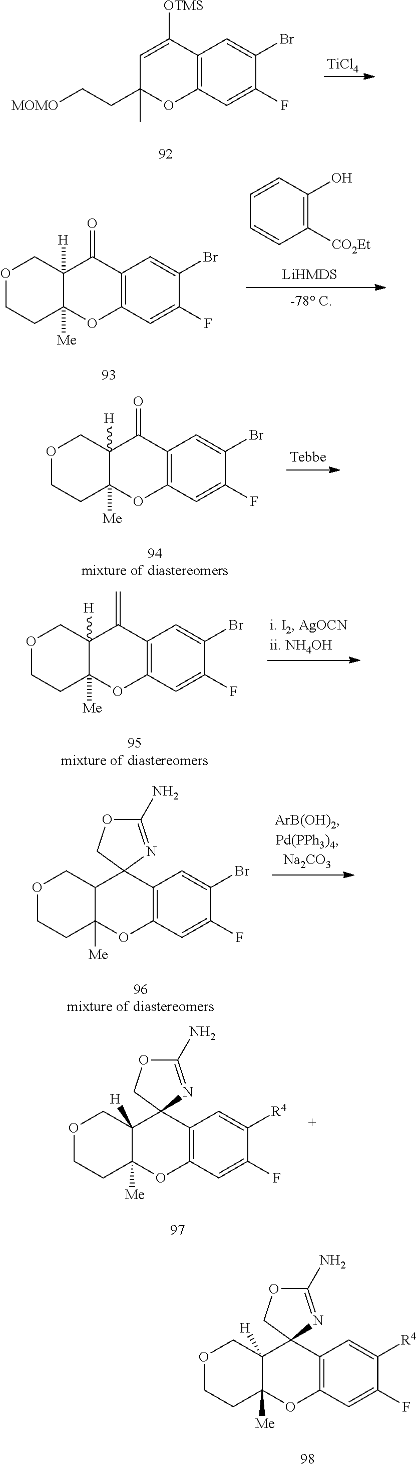

Scheme 9 depicts the general route towards compounds 97 and 98, wherein R4 is as defined herein (preferably aryl or heteroaryl). Phenol 85 is treated with paraformaldehyde and magnesium chloride to provide salicylaldehyde 86, which was subsequently treated with bromine and acetic acid to provide bromide 87. This compound was then cyclized to hemiacetal 88 in the presence of triethylamine and 3-methylbut-2-enal. Diol 89 was formed upon treatment with sodium borohydride and could then be converted to ketone 90 in the presence of magnesium dioxide. Treatment with methoxymethyl chloride provided MOM-protected ether 91. Silyl enol ether 92 was synthesized upon deprotonation with LiHMDS and trapping with TMSCl. The crude material was then treated with TiCl₄ to provide cyclic ether 93 as the cis ring junction material. The cis ketone could be epimerized after deprotonation with LiHMDS and kinetic reprotonation with ethyl salicylate to provide ketone 94 as a mixture of cis and trans material. Olefin 95 was formed upon treatment with Tebbe reagent and the spirocycle 96 could then be formed with treatment of iodine and silver cyanate followed by the addition of ammonium hydroxide. Suzuki coupling then provided the target compounds 97 and 98 which could be isolated as the single enantiomers after chiral SFC purification.

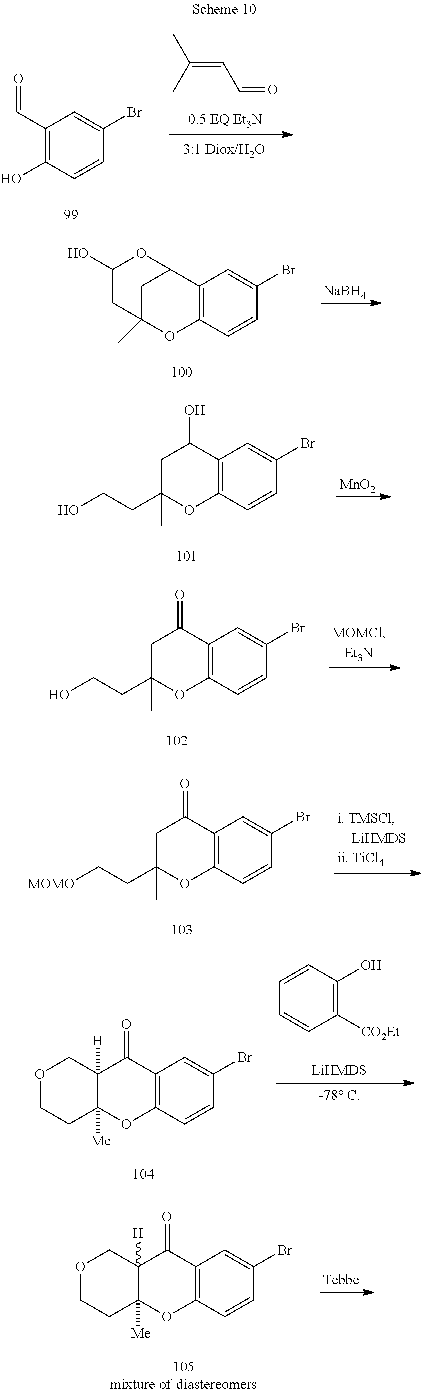

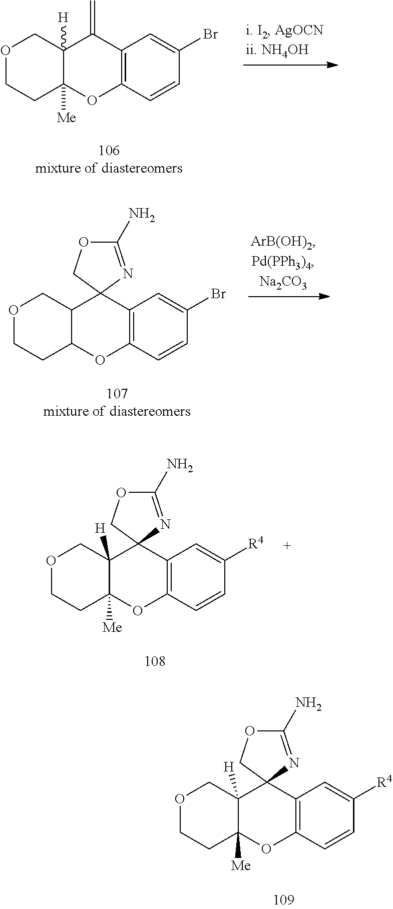

Scheme 10 depicts the general route towards compounds 99 and 100, wherein R4 is as defined herein (preferably aryl or heteroaryl). Salicylaldehyde 99 was cyclized to hemiacetal 100 in the presence of triethylamine and 3-methylbut-2-enal. Diol 101 was formed upon treatment with sodium borohydride and could then be converted to ketone 102 in the presence of magnesium dioxide. Treatment with methoxymethyl chloride provided MOM-protected ether 103. Cyclic ether 104 was synthesized upon deprotonation with LiHMDS and trapping with TMSCl. The crude material was then treated with TiCl₄ to provide cyclic ether 104 as the cis ring junction material. The cis ketone could be epimerized after deprotonation with LiHMDS and kinetic reprotonation with ethyl salicylate to provide ketone 105 as a mixture of cis and trans material. Olefin 106 was formed upon treatment with Tebbe reagent and the spirocycle 107 could then be formed with treatment of iodine and silver cyanate followed by the addition of ammonium hydroxide. Suzuki coupling then provided the target compounds 108 and 109, which could be isolated as the single enantiomers after chiral SFC purification.

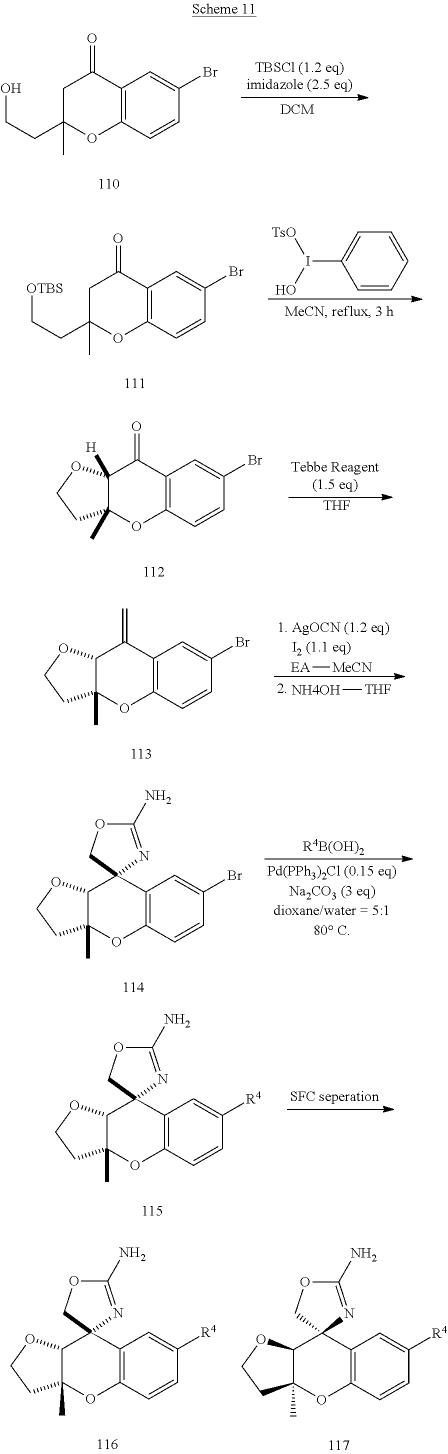

Scheme 11 depicts the general route toward compounds 116 and 117, wherein R⁴ is as defined herein (preferably aryl or heteroaryl). Treatment of alcohol 110 with tert-butyldimethylsilyl chloride and imidazole in dichloromethane provided silyl ether 111. Cyclization to tetrahydrofuran 112 was achieved by treatment of silyl ether 111 with Koser's reagent in refluxing acetonitrile. Olefination of ketone 112 using Tebbe reagent afforded alkene 113, which was treated with silver cyanate, iodine followed by ammonium hydroxide to afford aminooxazoline 114 as a single diastereomer (racemic). Suzuki coupling of bromide 114 with aryl/heteroaryl boronic acids or boronate esters yielded compound 115, which was purified by chiral supercritical fluid chromatography to provide enantiomers 116 and 117.

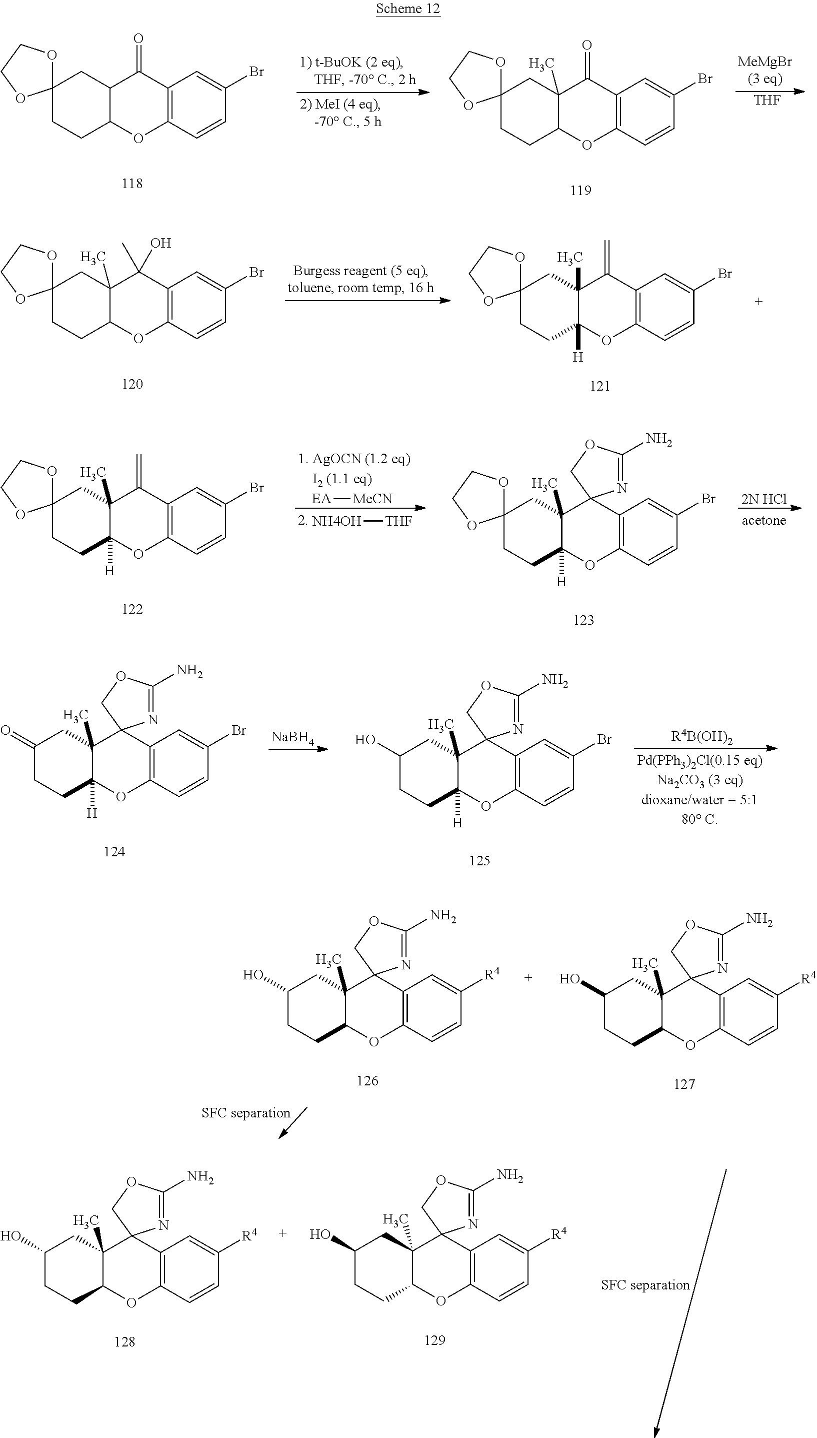

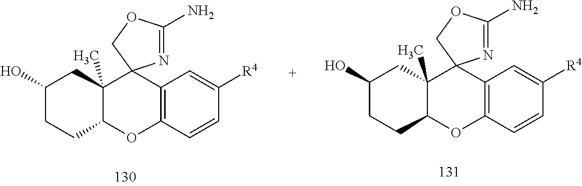

Scheme 12 depicts the general route toward compounds 128, 129, 130, and 131, wherein R⁴ is as defined herein (preferably aryl or heteroaryl. Methylation of ketone 118 using potassium tert-butoxide and iodomethane provided compound 119. Methyl magnesium bromide addition to ketone 119 afforded alcohol 120, which was subsequently treated with Burgess reagent to provide olefins 121 and 122. Olefin 122 was treated iodoisocyanate followed by ammonium hydroxide yielded aminooxazoline 123. Acidic removal of ketal 123 gave ketone 124, which was reduced with sodium borohydride to alcohol 125. Suzuki coupling of bromide 125 with aryl/heteroaryl boronic acids or boronate esters yielded compounds 126 and 127, which were further purified by chiral supercritical fluid chromatography to provide stereoisomers 128, 129, 130, and 131 of unassigned stereochemical configuration.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary, such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., an enantiomer, substantially free of its stereoisomer may be obtained by resolution of the racemic mixture using a method such as formation of diastereomers using optically active resolving agents (Eliel, E. and S. Wilen. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302). Racemic mixtures of chiral compounds described herein may be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., ed. Drug Stereochemistry: Analytical Methods and Pharmacology. New York: Marcel Dekker, Inc., 1993.

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid, can result in formation of the diastereomeric salts.

Alternatively, by method (2), the substrate to be resolved is reacted with one enantiomer of a chiral compound to form a diastereomeric pair (Eliel, E., and S. Wilen. Stereochemistry of Organic Compounds. New York: John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III, Peyton. “Resolution of (±)-5-Bromonornicotine. Synthesis of (R)- and (S)-Nornicotine of High Enantiomeric Purity.” J. Org. Chem. Vol. 47, No. 21 (1982): pp. 4165-4167), of the racemic mixture, and analyzing the ¹H NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111).

By method (3), a racemic mixture of two enantiomers can be separated by chromatography using a chiral stationary phase (Lough, W. J., ed. Chiral Liquid Chromatography. New York: Chapman and Hall, 1989; Okamoto, Yoshio, et al. “Optical resolution of dihydropyridine enantiomers by high-performance liquid chromatography using phenylcarbamates of polysaccharides as a chiral stationary phase.” J. of Chromatogr. Vol. 513 (1990): pp. 375-378). Enriched or purified enantiomers can be distinguished by methods used to distinguish other chiral molecules with asymmetric carbon atoms, such as optical rotation and circular dichroism.

Indications

The compounds of the invention inhibit the cleavage of amyloid precursor protein by β-secretase which is implicated in diseases, in particular, neurodegenerative diseases such as Alzheimer's disease. In AD, processing of APP by β-secretase produces soluble N-APP, which activates extrinsic apoptotic pathways by binding to death receptor 6. Furthermore, APP that is processed by β-secretase is subsequently cleaved by γ-secretase, thereby producing amyloid beta peptides, such as Aβ 1-42 that form amyloid plaques, which contribute to nerve cell death. Compounds of the invention inhibit enzymatic cleavage of APP by β-secretase.

Accordingly, in an aspect of the invention, there is provided a method of inhibiting cleavage of APP by β-secretase in a mammal comprising administering to said mammal an effective amount of a compound of Formula I′, I′a, I′b, I′c, I′d, I′e, I′g, I′h, I′i, I′j, I′k, I′l, I′m, I′n, I′o, I′p, I, Ia, Ib, Ic, Id, Ie, If, Ig, Ih, Ij, Ik, Il, Im, In, Io and Ip.

In another aspect of the invention, there is provided a method for treating a disease or condition mediated by the cleavage of APP by β-secretase in a mammal, comprising administering to said mammal an effective amount of a compound of Formula I′, I′a, I′b, I′c, I′d, I′e, I′f, I′g, I′h, I′i, I′j, I′k, I′l, I′m, I′n, I′o, I′p, I, Ia, Ib, Ic, Id, Ie, If, Ig, Ih, Ii, Ij, Ik, Il, Im, In, Io and Ip.

In another aspect, there is provided the use of a compound of Formula I′, I′a, I′b, I′c, I′d, I′e, I′g, I′h, I′i, I′j, I′k, I′l, I′m, I′n, I′o, I′p, I, Ia, Ib, Ic, Id, Ie, If, Ig, Ih, Ii, Ij, Ik, Il, Im, In, Io and Ip in the manufacture of a medicament for the treatment of a neurodegenerative disease. In one embodiment, the neurodegenerative disease is Alzheimer's disease.

In another aspect of the invention, there is provided a use of a compound of Formula I′, I′a, I′b, I′c, I′d, I′e, I′g, I′h, I′i, I′j, I′k, I′l, I′m, I′n, I′o, I′p, I, Ia, Ib, Ic, Id, Ie, If, Ig, Ih, Ii, Ij, Ik, Il, Im, In, Io and Ip in the treatment of neurodegenerative diseases. In one embodiment, the neurodegenerative disease is Alzheimer's disease.

Compounds of the invention may be administered prior to, concomitantly with, or following administration of other therapeutic compounds. Sequential administration of each agent may be close in time or remote in time. The other therapeutic agents may be anti-neurodegenerative with a mechanism of action that is the same as compounds of the invention, i.e., inhibit beta-secretase cleavage of APP, or a different mechanism of action, e.g., anti-Aβ antibodies. The compounds may be administered together in a unitary pharmaceutical composition or separately and, when administered separately this may occur simultaneously or sequentially in any order. Such sequential administration may be close in time or remote in time.

The invention also includes compositions containing the compounds of the invention and a carrier, diluent or excipient, as well as methods of using the compounds of the invention to prepare such compositions. In a particular embodiment, there is provided a pharmaceutical composition comprising a compound of Formula I′, I′a, I′b, I′e, I′d, I′e, I′f, I′g, I′h, I′i, I′j, I′k, I′l, I′m, I′n, I′o, I′p, I, Ia, Ib, Ie, Id, Ie, If, Ig, Ih, Ii, Ij, Ik, Il, Im, In, Io and Ip and a pharmaceutically acceptable carrier, diluent or excipient.

Typically, the compounds of the invention used in the methods of the invention are formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but may range anywhere from about 3 to about 8. Formulation in an acetate buffer at pH 5 is a suitable embodiment. In one embodiment, formulations comprising compounds of the invention are sterile. The compounds ordinarily will be stored as a solid composition, although lyophilized formulations or aqueous solutions are acceptable.

Compositions comprising compounds of the invention will be formulated, dosed, and administered in a fashion consistent with good medical practice. Factors for consideration in this context include the particular disorder being treated, the particular mammal being treated, the clinical condition of the individual patient, the cause of the disorder, the site of administration, the method of administration, the scheduling of administration, and other factors known to medical practitioners.

The compounds may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents. If parenteral administration is desired, the compositions will be sterile and in a solution or suspension form suitable for injection or infusion.

Generally, the initial pharmaceutically effective amount of the compound of the invention administered parenterally per dose will be in the range of about 0.01-100 mg/kg/day, for example about 0.1 to 20 mg/kg of patient body weight per day, with the typical initial range of compound used being 0.3 to 15 mg/kg/day. Oral unit dosage forms, such as tablets and capsules, may contain from about 25 to about 1000 mg of the compound of the invention.

The compound of the invention may be administered by any suitable means, including oral, sublingual, buccal, topical, transdermal, parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. An example of a suitable oral dosage form is a tablet containing about 25 mg, 50 mg, 100 mg, 250 mg, or 500 mg of the compound of the invention compounded with about 90-30 mg anhydrous lactose, about 5-40 mg sodium croscarmellose, about 5-30 mg polyvinylpyrrolidone (“PVP”) K30, and about 1-10 mg magnesium stearate. The powdered ingredients are first mixed together and then mixed with a solution of the PVP. The resulting composition can be dried, granulated, mixed with the magnesium stearate and compressed to tablet form using conventional equipment. An aerosol formulation can be prepared by dissolving the compound, for example 5-400 mg, of the invention in a suitable buffer solution, e.g. a phosphate buffer, adding a tonicifier, e.g., a salt such sodium chloride, if desired. The solution is typically filtered, e.g., using a 0.2 micron filter, to remove impurities and contaminants.

Another formulation may be prepared by mixing a compound described herein and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound described herein or pharmaceutical composition thereof) or aid in the manufacturing of the pharmaceutical product (i.e., medicament).

### EXAMPLES

The invention will be more fully understood by reference to the following examples. They should not, however, be construed as limiting the scope of the invention. For example, the synthesis of non-exemplified compounds may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds described herein. The identity and purity of compounds were checked by LCMS and ¹H NMR analysis.

Column chromatography was done on a Biotage system (Manufacturer: Dyax Corporation) having a silica gel column or on a silica SepPak cartridge (Waters) (unless otherwise stated). ¹H NMR spectra were recorded on a Varian instrument operating at 400 MHz. ¹H-NMR spectra were obtained as CDCl₃, CD₃OD, D₂O, (CD₃)₂SO, (CD₃)₂CO, C₆D₆, CD₃CN solutions (reported in ppm), using tetramethylsilane (0.00 ppm) or residual solvent (CDCl₃: 7.26 ppm; CD₃OD: 3.31 ppm; D₂O: 4.79 ppm; (CD₃)₂SO: 2.50 ppm; (CD₃)₂CO: 2.05 ppm; C₆D₆: 7.16 ppm; CD₃CN: 1.94 ppm) as the reference standard. When peak multiplicities are reported, the following abbreviations are used: s (singlet), d (doublet), t (triplet), q (quartet), m (multiplet), br (broadened), dd (doublet of doublets), dt (doublet of triplets). Coupling constants, when given, are reported in Hertz (Hz).

In the Examples described below, unless otherwise indicated all temperatures are set forth in degrees Celsius. Reagents were purchased from commercial suppliers such as Sigma-Aldrich, Alfa Aesar, or TCI, and were used without further purification unless otherwise indicated.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

### Biological Evaluation

### Cellular BACE-1 Inhibition Assay

The BACE inhibition properties of the compounds of the invention may be determined by the following in vitro cellular Amyloidβ 1-40 production assay.

Inhibition of Amyloidβ 1-40 production was determined by incubating cells with compound for 48 hours and quantifying the level of Amyloidβ 1-40 using an homogeneous time-resolved fluorescence (“HTRF”) immunoassay.

Materials and Methods: HEK-293 cells stably transfected with a DNA construct containing the coding sequence for the wild type APP695 sequence were grown in Dulbecco's Modified Eagle Medium (“DMEM”) supplemented with 10% fetal bovine serum, penicillin/streptomycin and 150 μg/mL G418. Cells were plated in 96-well plates at 35,000 cells/well and allowed to attach for 8-12 hours. Media was changed to DMEM supplemented with 10% fetal bovine serum, penicillin/streptomycin 15 minutes prior to compound addition. Diluted compounds were then added at a final concentration of 0.5% DMSO. After 48 hours, 4 μL of media from each well was added to a corresponding well of a 384 well plate (Perkin Elmer Cat#6008280) containing the HTRF reagents. HTRF reagents were obtained from the CisBio Amyloid β 1-40 peptide assay kit (Cat#62B40PEC) and were prepared as follows anti-peptide β (1-40)-Cryptate and anti-peptide p (1-40)-XL655 were stored in 2 plate aliquots at −80° C. Diluent and Reconstitution buffer were stored at 4° C. Aliquots of the two antibodies were diluted 1:100 with Reconstitution buffer, and this mixture was diluted 1:2 with Diluent. 12 μL of the reagent mixture was added to the required wells of the 384 well assay plate. The assay plate was incubated at 4° C. for 17 hours and then analyzed for fluorescence at 665 and 620 nm.

The following compounds were tested in the above assay. Some compounds were tested more than once, and the average is reported below.

| Example # | IC₅₀ (nM) |
|---|---|
| Example 1 | 98.45 |
| Example 2 | 116.85 |
| Example 3 | 3083.85 |
| Example 4 | 1187 |
| Example 5 | 132.8 |
| Example 9 | 43.4 |
| Example 10 | 58.45 |
| Example 11 | 69.55 |
| Example 12 | 60.7 |
| Example 14 | 73.45 |
| Example 15 | 58.65 |
| Example 16 | 171.2 |
| Example 17 | 146.7 |
| Example 25 | 24.1 |
| Example 26 | 86.55 |
| Example 27 | 64.2 |
| Example 28 | 7.8 |
| Example 29 | 8.9 |
| Example 31 | 47.3 |
| Example 32 | 118.45 |
| Example 34 | 90.2 |
| Example 37 | 852.9 |
| Example 38 | 545.85 |
| Example 41 | 1331 |
| Example 44 | 303.95 |
| Example 48 | 2549.45 |
| Example 54 | 33.45 |
| Example 88 | 153.85 |
| Example 90 | 1342.15 |
| Example 91 | 383.95 |
| Example 98 | 420.6 |
| Example 113 | 181.4 |
| Example 115 | 173.7 |
| Example 118 | 219.05 |
| Example 120 | 40.95 |
| Example 140 | 48.35 |
| Example 146 | 192.33 |
| Example 150 | 100 |
| Example 155 | 71.4 |
| Example 156 | 92.85 |
| Example 159 | 76.15 |
| Example 160 | 28.37 |
| Example 199 | 629.95 |
| Example 218 | 456.55 |
| Example 227 | 247.4 |
| Example 266 | 5289.7 |

### Example 1

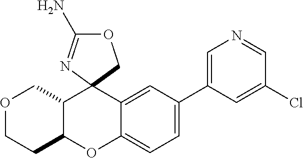

### (4S*,4a′S*,10a′S*)-8′-(5-chloropyridin-3-yl)-3′,4′,4a′,10a′-tetrahydro-1′H,5H-spiro[oxazole-4,10′-pyrano[4,3-b]chromen]-2-amine

Step A: A stainless steel bomb containing teflon-coated insert was charged with ethoxyethene (47 mL, 494 mmol) and 6-bromo-4-oxo-4H-chromene-3-carbaldehyde (25 g, 99 mmol). The mixture was heated to 100° C. with stirring for 18 hours. After cooling to room temperature, the reaction mixture was concentrated in vacuo to yield (3R,4aR)-8-bromo-3-ethoxy-4,4a-dihydropyrano[4,3-b]chromen-10(3H)-one (32 g, 98%). A 3:1 mixture of endo/exo isomers was obtained based on ¹H NMR analysis. The product did not require purification.

Step B: A Parr shaker flask was charged with (3R*,4aR*)-8-bromo-3-ethoxy-4,4a-dihydropyrano[4,3-b]chromen-10(3H)-one (20 g, 62 mmol), dioxane (200 mL), and PtO₂—H₂O (1.5 g, 6.2 mmol; “Adams' catalyst”). The reaction mixture was shaken under H₂ at 30 psi for 18 hours. The mixture was filtered through Celite®, rinsing with DCM. The mixture was concentrated, and the crude (21 g) was purified by Biotage 65 silica gel chromatography, eluting with a gradient of 10% EtOAc/hexanes to neat EtOAc. The product-containing fractions (5.6 g) and mixed fractions (2.2 g) were pooled separately. The mixed fractions were rechromatographed on a Biotage Flash 40 silica gel chromatography system, eluting with a gradient of 10%-30% EtOAc/hexanes. The product-containing fractions were combined with material from the first column to obtain (3R*,4aR*,10aS*)-8-bromo-3-ethoxy-1,4,4a,10a-tetrahydropyrano[4,3-b]chromen-10(3H)-one, 6.6 g (30%).

Step C: A 250 mL round bottomed flask plus stir bar was charged with (3R*,4aR*,10aS*)-8-bromo-3-ethoxy-1,4,4a,10a-tetrahydropyrano[4,3-b]chromen-10(3H)-one (6.6 g, 20 mmol), DCM (50 mL), and triethylsilane (26 mL, 161 mmol). The reaction mixture was cooled to 0° C. under N₂, and BF₃ etherate (10 mL, 81 mmol) was added. The reaction mixture was stirred for 30 minutes and then warmed to room temperature while stirring for 3 hours. The mixture was quenched with saturated aqueous NaHCO₃ (20 mL). The mixture was stirred for 30 minutes and then diluted with EtOAc (100 mL). The phases were separated, and the aqueous phase was re-extracted with EtOAc (50 mL). The combined organic phases were washed with saturated aqueous NaHCO₃ (100 mL), brine (100 mL), dried (MgSO₄), filtered, and concentrate to yield (4aR*,10aS*)-8-bromo-1,4,4a,10a-tetrahydropyrano[4,3-b]chromen-10(3H)-one (5.9 g, 67%). No purification was performed.

Step D: A round bottomed flask plus stir bar was charged with (4aR*,10aS*)-8-bromo-1,4,4a,10a-tetrahydropyrano[4,3-b]chromen-10(3H)-one (780 mg, 2.8 mmol), MeOH (5 mL), THF (2 mL) and K₂CO₃ (76 mg, 0.55 mmol). The mixture was stirred at room temperature for 18 hours and concentrated in vacuo. The mixture was suspended in DCM and filtered through Celite® to remove salts. The mixture was concentrated in vacuo to obtain a 2:1 ratio of trans to cis isomers of (4aR*,10aR*)-8-bromo-1,4,4a,10a-tetrahydropyrano[4,3-b]chromen-10(3H)-one (745 mg, 96%). No purification was performed.

Step E: A round bottomed flask plus stir bar was charged with (4aR*,10aR*)-8-bromo-1,4,4a,10a-tetrahydropyrano[4,3-b]chromen-10(3H)-one (1.0 g, 3.5 mmol) and anhydrous THF (5 mL). The mixture was cooled to 0° C. under N₂, and μ-chlorobis(cyclopentadienyl)(dimethylaluminium)-μ-methylenetitanium (10.6 mL, 5.30 mmol; “Tebbe's reagent”) was added. The mixture was stirred for 1 hour. The reaction mixture was very carefully poured (vigorous exotherm and bubbling) into MeOH (10 mL), and then aqueous 2N NaOH (5 mL) was added dropwise. The biphasic suspension was stirred for 15 minutes at room temperature. The biphase was filtered to remove solids through Celite®, rinsing with diethyl ether. The phases were separated, and the aqueous phase was re-extracting with diethyl ether (5 mL). The combined organics were washed with brine (20 mL), dried (MgSO₄), filtered, and concentrated. The crude was purified by Biotage Flash 40 silica gel chromatography, eluting with neat hexanes (500 mL), 5% EtOAc/hexanes, then 10% EtOAc/hexanes to fully elute product to obtain (4aS*,10aS*)-8-bromo-10-methylene-1,3,4,4a,10,10a-hexahydropyrano[4,3-b]chromene (235 mg, 21%). A 60:40 ratio of trans/cis isomers was obtained.

Step F: A stirred solution of (4aS*,10aS*)-8-bromo-10-methylene-1,3,4,4a,10,10a-hexahydropyrano[4,3-b]chromene (235 mg, 0.836 mmol) in diethyl ether (2 mL) was cooled to 0° C. under N₂. In a separate flask, silver cyanate (501 mg, 3.34 mmol) was suspended in CH₃CN (1 mL), and iodine (424 mg, 1.67 mmol) in THF (1 mL) was added to this suspension. The resulting mixture was shaken for 30 seconds. The suspension was then poured into the alkene solution at 0° C. The reaction mixture was allowed to warm to room temperature, and stirring was continued for 1 hour. The reaction mixture was filtered through Celite®, rinsing with diethyl ether, and the filtrate was concentrated. The residue was dissolved in THF (1 mL) and aqueous NH₄OH (0.5 mL) was added. The resulting mixture was stirred at room temperature for 2 hours. The reaction mixture was partitioned between ethyl acetate (50 mL) and saturated Na₂S₂O₃ (30 mL). After separating the phases, the aqueous layer was re-extracted with ethyl acetate (30 mL). The combined organic layers were washed with brine (30 mL), dried (MgSO4), filtered, and concentrated. The crude was partially purified by preparative TLC (2 mm thickness, Rf=0.48) eluting with 10% MeOH/DCM. The two trans isomers were then separated by Biotage Flash 40 silica gel chromatography eluting with 1:1 EtOAc/hexanes, neat EtOAc, 2.5% MeOH/EtOAc, then 5% MeOH/EtOAc to completely elute products and yield (4S*,4a′S*,10a′S*)-8′-bromo-3′,4′,4a′,10a′-tetrahydro-1′H,5H-spiro[oxazole-4,10′-pyrano[4,3-b]chromen]-2-amine (62 mg, 16%). There was also a yield of (4R*,4a′S*,10a′S*)-8′-bromo-3′,4′,4a′,10a′-tetrahydro-1′H,5H-spiro[oxazole-4,10′-pyrano[4,3-b]chromen]-2-amine (49 mg, 15%).

Step G: A 2 dram vial was charged with (4S*,4a′S*,10a′S*)-8′-bromo-3′,4′,4a′,10a′-tetrahydro-1′H,5H-spiro[oxazole-4,10′-pyrano[4,3-b]chromen]-2-amine (25 mg, 0.074 mmol), dioxane (0.5 mL), 5-chloropyridin-3-ylboronic acid (13 mg, 0.081 mmol), Pd(PPh₃)₄ (8.5 mg, 0.0074 mmol), and 2N aqueous Na₂CO₃ (92 μL, 0.18 mmol). The mixture was sparged with N₂ for 2 minutes and then heated to 90° C. for 3 hours. After cooling to room temperature, the reaction mixture was loaded directly on to preparative TLC plate (1 mm thickness, Rf=0.57) eluting with 10% MeOH (containing 7N NH₃) in DCM to yield (4S*,4a′S*,10a′S*)-8′-(5-chloropyridin-3-yl)-3′,4′,4a′,10a′-tetrahydro-1′H,5H-spiro[oxazole-4,10′-pyrano[4,3-b]chromen]-2-amine (12 mg, 41%). ¹H NMR (400 MHz, CDCl₃+MeOD) δ 8.47 (d, J=2 Hz, 1H), 8.30 (d, J=2 Hz, 1H), 7.75 (m, 1H), 7.38 (d, J=2 Hz, 1H), 7.25 (m, 1H), 6.77 (d, J=8 Hz, 1H), 4.36 (d, J=9 Hz, 1H), 3.95 (m, 3H), 3.90 (d, J=9.0 Hz, 1H), 3.38 (m, 1H), 3.16 (d, J=11 Hz, 1H), 2.04 (m, 2H), 1.78 (m, 1H). m/z (APCI-pos) M+1=372.

Upon further structural analysis, it was determined by X-ray crystallography that the relative stereochemistry of Example 1 was (4R*,4a′S*,10a′S*)-8′-(5-chloropyridin-3-yl)-3′,4′,4a′,10a′-tetrahydro-1′H,5H-spiro[oxazole-4,10′-pyrano[4,3-b]chromen]-2-amine:

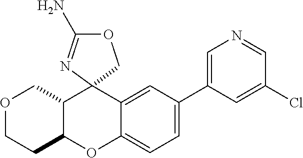

### Example 2

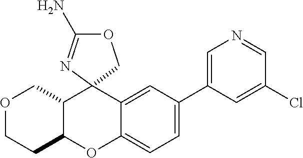

### (4R*,4a′S*,10a′S*)-8′-(5-chloropyridin-3-yl)-3′,4′,4a′,10a′-tetrahydro-1′H,5H-spiro[oxazole-4,10′-pyrano[4, 3-1)]chromen]-2-amine

(4R*,4a′S*,10a′S*)-8′-(5-Chloropyridin-3-yl)-3′,4′,4a′,10a′-tetrahydro-1′H,5H-spiro[oxazole-4,10′-pyrano[4,3-b]chromen]-2-amine (9 mg, 40%) was prepared from (4R*,4a′S*,10a′S*)-8′-bromo-3′,4′,4a′,10a′-tetrahydro-1′H,5H-spiro[oxazole-4,10′-pyrano[4,3-b]chromen]-2-amine (20 mg, 0.059 mmol; Example 1, Step F) according to the procedure in Example 1, Step G. ¹H NMR (400 MHz, CDCl₃+MeOD) δ 8.49 (d, J=2 Hz, 1H), 8.36 (d, J=2 Hz, 1H), 7.74 (t, J=2 Hz, 1H), 7.29 (m, 2H), 6.85 (d, J=9 Hz, 1H), 4.54 (d, J=9 Hz, 1H), 4.32 (d, J=9 Hz, 1H), 4.17 (td, J=5, 11 Hz, 1H), 3.97 (m, 2H), 3.44 (m, 1H), 3.30 (d, J=12 Hz, 1H), 2.09 (m, 1H), 1.85 (m, 2H). m/z (APCI-pos) M+1=372.

Upon further structural analysis, it was determined by X-ray crystallography that the relative stereochemistry of Example 2 was (4R*,4a′R*,10a′R*)-8′-(5-chloropyridin-3-yl)-3′,4′,4a′,10a′-tetrahydro-1′H,5H-spiro[oxazole-4,10′-pyrano[4,3-b]chromen]-2-amine:

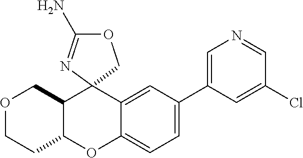

### Example 3

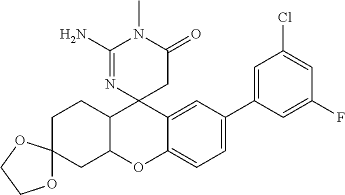

### 2″-amino-7′-(3-chloro-5-fluorophenyl)-1″-methyl-2′,4′,4′a,5″,6″,9′a-hexahydro-1′H,1″H-dispiro[1,3-dioxolane-2,3′-xanthene-9′,4″-pyrimidine]-6″-one

Step A: 7′-Bromo-1′,4′,4a′,9a′-tetrahydrospiro[[1,3]dioxolane-2,3′-xanthen]-9′(2′H)-one (1.00 g, 2.95 mmol) was combined with 2-methylpropane-2-sulfinamide (2.0 g, 16.5 mmol) and tetraethoxytitanium (3.08 mL, 14.7 mmol) in dry THF (10 mL) and heated under N₂ at 70° C. After 14 hours, starting material remained and cis and trans isomers spots are observed by TLC analysis (40% EtOAc/hexanes). The reaction was cooled and poured into saturated NaHCO₃ and vigourously stirred. After 5 minutes, EtOAc was added, and the solids were filtered through a pad of Celite®. The Celite® was extracted exhaustively until no yellow color remained (about 400 mL EtOAc). The organic phase was washed with brine and dried (MgSO₄) and concentrated. The crude material was purified by flash column chromatography eluting with 20% EtOAc/hexanes to provide as the major isomer the trans ring product, N-(7′-bromo-1′,4′,4a′,9a′-tetrahydrospiro[[1,3]dioxolane-2,3′-xanthene]-9′(2′H)-ylidene)-2-methylpropane-2-sulfinamide (0.67 g, 51%) as a solid.

Step B: Diisopropylamine (0.75 mL, 5.3 mmol) and dry THF (15 mL) were added to a flame dried flask and cooled to 0° C. Butyllithium (1.8 mL, 4.5 mmol) was added, and the reaction was stirred 30 minutes. The reaction was then cooled to −78° C., and methyl acetate (0.40 mL, 5.0 mmol) was added dropwise and allowed to stir for 30 minutes. A solution of (N-((4a′R*,9a′S*)-7′-bromo-1′,4′,4a′,9a′-tetrahydrospiro[[1,3]dioxolane-2,3′-xanthene]-9′(2′H)-ylidene)-2-methylpropane-2-sulfinamide (0.67 g, 1.5 mmol) was added in dry THF (28 mL) slowly over 10 minutes. The reaction was stirred for 4 hours and then quenched with saturated NaHCO₃ (30 mL). The reaction was then extracted with EtOAc, washed with NH₄Cl, brine and dried over MgSO₄. The reaction was then concentrated and purified with a gradient of 15% EtOAc/CH₂Cl₂ to 60% EtOAc/CH₂Cl₂ on a 125 g silica column to provide methyl 2-(7′-bromo-9′-(1,1-dimethylethylsulfinamido)-1′,2′,4′,4a′,9′,9a′-hexahydrospiro[[1,3]dioxolane-2,3′-xanthene]-9′-yl)acetate (0.54 g, 69%), as mostly one diastereomers by NMR analysis.

Step C: Methyl 2-(7′-bromo-9′-(1,1-dimethylethylsulfinamido)-1′,2′,4′,4a′,9′,9a′-hexahydrospiro[[1,3]dioxolane-2,3′-xanthene]-9′-yl)acetate (0.054 g, 0.10 mmol) was dissolved in CH₂Cl₂ (2 mL), and a 2M solution of hydrogen chloride in ether (0.10 mL, 0.21 mmol) was added dropwise. After 7 hours, the reaction was concentrated. The residue was partitioned between EtOAc (25 mL) and a small amount of saturated NaHCO₃ (about 2 mL). The organic phase was dried (MgSO₄) and concentrated to provide methyl 2-(9′-amino-7′-bromo-1′,2′,4′,4a′,9′,9a′-hexahydrospiro[[1,3]dioxolane-2,3′-xanthene]-9′-yl)acetate (41 mg, 95%).

Step D: Methyl 2-(9′-amino-7′-bromo-1′,2′,4′,4a′,9′,9a′-hexahydrospiro[[1,3]dioxolane-2,3′-xanthene]-9′-yl)acetate (0.108 g, 0.262 mmol), methylcarbamothioylcarbamate (60 mg, 0.314 mmol) and N1-((ethylimino)methylene)-N3,N3-dimethylpropane-1,3-diamine hydrochloride (70 mg, 0.367 mmol) were combined in DMF (2.5 mL), and N-ethyl-N-isopropylpropan-2-amine (0.232 mL, 1.31 mmol) was added dropwise. After stirring overnight, saturated NH₄Cl (8 mL) was added to quench the reaction. Then water (1 mL) was added, and the aqueous was extracted with EtOAc (2×). The organic phase was washed with brine, dried (MgSO₄) and concentrated. The residue was partially dissolved in CH₂Cl₂ and then concentrated to less than 1 mL. Et₂O (about 3 mL) was added, and the mixture was filtered and washed with Et₂O to provide tert-butyl N-{7′-bromo-1″-methyl-6″-oxo-2′,4′,4′a,5″,6″,9′a-hexahydro-1′H,1″H-dispiro[1,3-dioxolane-2,3′-xanthene-9′,4″-pyrimidine]-2″-yl}carbamate (80 mg, 57%) as a solid.

Step E: tert-Butyl N-{7′-bromo-1″-methyl-6″-oxo-2′,4′,4′a,5″,6″,9′a-hexahydro-1′H,1″H-dispiro[1,3-dioxolane-2,3′-xanthene-9′,4″-pyrimidine]-2″-yl}carbamate (0.041 g, 0.076 mmol) and 3-chloro-5-fluorophenylboronic acid (0.016 g, 0.092 mmol) were combined with dioxane (0.8 mL) and a saturated solution of sodium carbonate (0.097 mL, 0.18 mmol) and degassed with argon for 5 minutes. Dichloro[1,1′-bis(diphenylphosphino)ferrocene]palladium (II) dichloromethane adduct (0.0063 g, 0.0076 mmol) was added, and the reaction was heated in a sealed vial at 80° C. for 4 hours. The reaction was cooled and diluted with EtOAc, washed with water, brine and dried (MgSO₄). Column chromatography (0.5 to 2% MeOH/CH₂Cl₂ with 4% NH₄OH in MeOH) provided the N-Boc protected material, tert-butyl N-[7′-(3-chloro-5-fluorophenyl)-1″-methyl-6″-oxo-2′,4′,4′a,5″,6″,9′a-hexahydro-1′H,1″H-dispiro[1,3-dioxolane-2,3′-xanthene-9′,4″-pyrimidine]-2″-yl]carbamate (17 mg, 38%), and a smaller amount of the des-Boc material (7 mg). The des-Boc material was further triturated with MeOH to provide 2″-amino-7′-(3-chloro-5-fluorophenyl)-1″-methyl-2′,4′,4′a,5″,6″,9′a-hexahydro-1′H,1″H-dispiro[1,3-dioxolane-2,3′-xanthene-9′,4″-pyrimidine]-6″-one (5 mg, 13%) as a solid. ¹H NMR (CDCl₃) δ 7.34-7.33 (m, 2H), 7.23 (s, 1H), 7.06-7.03 (m, 1H), 7.01-6.98 (m, 1H), 6.89 (d, J=9.0 Hz, 1H), 4.35 (bs, 2H), 4.03-3.95 (m, 5H), 3.32 (s, 3H), 3.01 (bd, 1H), 2.80 (d, J=17 Hz, 1H), 2.40-2.35 (m, 1H), 1.84-1.54 (m, 6H). m/z (APCI-pos) M+1=486.

### Example 4

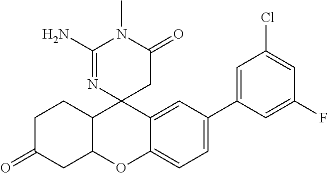

### 2-amino-7′-(3-chloro-5-fluorophenyl)-1-methyl-1′,4′,4a′,9a′-tetrahydro-1H-spiro[pyrimidine-4,9′-xanthene]-3′,6(2′H,5H)-dione

tert-Butyl N-[7′-(3-chloro-5-fluorophenyl)-1″-methyl-6″-oxo-2′,4′,4′a,5″,6″,9′a-hexahydro-1′H,1″H-dispiro[1,3-dioxolane-2,3′-xanthene-9′,4″-pyrimidine]-2″-yl]carbamate (17 mg, 0.029 mmol) was partially dissolved in acetone (1.5 mL), dioxane (0.2 mL) and 2N HCl (1.5 mL). The mixture was heated at 70° C. for 1 hour. The reaction was then concentrated and azeotroped twice from acetone to provide 2-amino-7′-(3-chloro-5-fluorophenyl)-1-methyl-1′,4′,4a′,9a′-tetrahydro-1H-spiro[pyrimidine-4,9′-xanthene]-3′,6(2′H,5H)-dione hydrochloride (14 mg, 0.029 mmol, 100%). ¹H NMR (CD₃OD) δ 7.93 (s, 1H), 7.62 (dd, J=8.6, 2.3 Hz, 1H), 7.50 (s, 1H), 7.37-7.34 (m, 1H), 7.17-7.13 (m, 1H), 7.01 (d, J=8.6 Hz, 1H), 4.11-4.04 (m, 1H), 3.87 (d, J=17 Hz, 1H), 3.36 (s, 3H), 3.02 (d, J=17 Hz, 1H), 2.48-2.43 (m, 1H), 2.07-2.00 (m, 1H), 1.91-1.87 (m, 1H), 1.79 (dd, J=12.5, 11.0, 1H), 1.74-1.66 (m, 2H), 1.52-1.45 (m, 1H). m/z (APCI-pos) M+1=442.

### Example 5

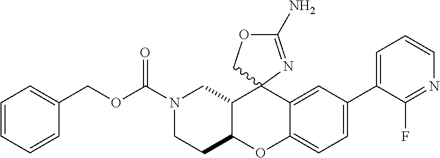

### rac-trans-(4a,10a)-benzyl 2′-amino-8-(2-fluoropyridin-3-yl)-1,4,4a,10a-tetrahydro-5′H-spiro[chromeno[3,2-c]pyridine-10,4′-oxazole]-2(3H)-carboxylate

Step A: Ethyl 4-chloronicotinate was prepared from 4-chloronicotinic acid as described in WO 2008/024725.

Step B: Cs₂CO₃ (25.5 g, 78.2 mmol) was added to a solution of ethyl 4-chloronicotinate (12.1 g, 65.2 mmol) and 4-bromophenol (11.8 g, 68.5 mmol) in DMF (217 mL). The reaction mixture was heated in an 80° C. sand bath and stirred for 20 hours. The reaction mixture was concentrated in vacuo, and the residue was partitioned between water and EtOAc. The mixture was extracted with EtOAc (2×), and the combined extracts were washed with brine, dried (Na₂SO₄), filtered, and concentrated. The crude was purified on silica gel (5-40% EtOAc in DCM gradient) to give ethyl 4-(4-bromophenoxy)nicotinate (19.2 g, 91.4%) as an oil that solidified on standing.

Step C: NaOH (3.58 g, 89.4 mmol) was added to a 0° C. solution of ethyl 4-(4-bromophenoxy)nicotinate (19.2 g, 59.6 mmol) in THF (300 mL) and H₂O (150 mL). The reaction mixture was warmed to room temperature and stirred for 7 hours. The THF was removed in vacuo, ice water (100 mL) was added, and the pH adjusted to about pH 3 by the addition of formic acid (3.60 mL, 95.4 mmol). Solid NaCl was added, and the mixture was extracted with EtOAc (2×). The combined extracts were dried (Na₂SO₄), filtered, and concentrated to give 4-(4-bromophenoxy)nicotinic acid (18.1 g, 103%) as a powder.

Step D: Concentrated sulfuric acid (123 mL, 2308 mmol) was added to a 1 L round-bottomed flask containing 4-(4-bromophenoxy)nicotinic acid (18.1 g, 61.5 mmol). The mixture was stirred until all of the solids dissolved, and the reaction mixture was heated in a 150° C. sand bath and stirred for 16 hours. The reaction mixture was then cooled to room temperature and poured slowly/portionwise into a 0° C. solution of NaOH (187 g, 4677 mmol) in 2 L of ice water, causing precipitation. The solids were isolated by vacuum filtration through qualitative filter paper on a Buchner funnel, rinsed with water, and air dried. The filtrate was extracted with DCM (2×), and the extracts were dried (Na₂SO₄), filtered, and concentrated. The resulting solids were combined with the solids above to give 8-bromo-10H-chromeno[3,2-c]pyridin-10-one (15.0 g, 88.3%) as a powder.

Step E: 1-(Chloromethyl)-4-methoxybenzene (5.90 mL, 43.5 mmol) was added into a thick-walled sealable pressure tube containing a mixture of 8-bromo-10H-chromeno[3,2-c]pyridin-10-one (3.0 g, 10.9 mmol) and TBAI (0.201 g, 0.543 mmol) in DCE (50 mL). The reaction mixture was sealed tightly and heated in a 90° C. sand bath and stirred for 22 hours. The reaction mixture was cooled to room temperature, diluted with DCM, and the solids were isolated by vacuum filtration through a 0.45 micron nylon filter membrane, rinsed with DCM and ether, and dried in vacuo to give 8-bromo-2-(4-methoxybenzyl)-10-oxo-10H-chromeno[3,2-c]pyridin-2-ium chloride (3.80 g, 80.8% yield) as a powder.

Step F: NaBH₄ (1.33 g, 35.1 mmol) was added in portions to a 0° C. mixture of 8-bromo-2-(4-methoxybenzyl)-10-oxo-10H-chromeno[3,2-c]pyridin-2-ium chloride (3.80 g, 8.78 mmol) in 1:1 EtOH:THF (80 mL). The reaction mixture was stirred at 0° C. for 45 minutes, another 1 equivalent NaBH₄ was added, and the reaction mixture continued to stir at 0° C. After 2 hours, another 1 equivalent NaBH₄ was added, and the reaction mixture was allowed to warm to room temperature and stirred. After 4 hours, the reaction mixture was concentrated to 1/3 volume, and this mixture was poured into a solution of ice saturated NH₄Cl, causing a precipitate to form. The solids were isolated by vacuum filtration through qualitative filter paper on a Buchner funnel, rinsed with water, air dried, and dried in vacuo to give 8-bromo-2-(4-methoxybenzyl)-2,3,4,4a,10,10a-hexahydro-1H-chromeno[3,2-c]pyridin-10-ol (3.12 g, 88.0% yield) as a powder as a mixture of diastereomers.

Step G: A solution of DMSO (1.65 mL, 23.2 mmol) in DCM (10 mL) was added to a −78° C. solution of 2M oxalyl chloride in DCM (5.80 mL, 11.6 mmol) in DCM (50 mL). The reaction mixture was stirred 10 minutes, then a sonicated suspension of rac-8-bromo-2-(4-methoxybenzyl)-2,3,4,4a,10,10a-hexahydro-1H-chromeno[3,2-c]pyridin-10-ol (3.125 g, 7.73 mmol) in THF (30 mL) was added slowly by syringe. The reaction mixture was stirred at −78° C. for 1 hour, then neat TEA (6.46 mL, 46.4 mmol) was added, and the reaction mixture was allowed to warm to room temperature and stirred for 1 hour. Brine was then added, the mixture was extracted with DCM (2×), and the combined extracts were dried (Na₂SO₄), filtered, concentrated, and dried in vacuo to give rac-(4a)-8-bromo-2-(4-methoxybenzyl)-2,3,4,4a-tetrahydro-1H-chromeno[3,2-c]pyridin-10(10aH)-one (3.11 g, 100.0% yield) as a syrup as a mixture of diastereomers.

Step H: K₂CO₃ (0.214 g, 1.55 mmol) was added to a solution of rac-(4a)-8-bromo-2-(4-methoxybenzyl)-2,3,4,4a-tetrahydro-1H-chromeno[3,2-c]pyridin-10(10aH)-one (3.11 g, 7.73 mmol) in MeOH (50 mL) and DCM (30 mL). The reaction mixture was stirred at room temperature for 3 hours, after which it was concentrated. The crude was purified on silica gel (5-40% EtOAc in DCM gradient) to give rac-trans-(4a,10a)-8-bromo-2-(4-methoxybenzyl)-2,3,4,4a-tetrahydro-1H-chromeno[3,2-c]pyridin-10(10aH)-one (1.03 g, 33%) as a foam.

Step I: 0.5M Tebbe reagent in toluene (5.62 mL, 2.81 mmol) was added to a 0° C. solution of rac-trans-(4a,10a)-8-bromo-2-(4-methoxybenzyl)-2,3,4,4a-tetrahydro-1,1-chromeno[3,2-c]pyridin-10(10aH)-one (0.514 g, 1.28 mmol) in THF (11 mL). The reaction mixture was stirred at 0° C. for 10 minutes, then warmed to room temperature and stirred for 4 hours. THF (20 mL) was added, the reaction mixture was cooled to 0° C., then MeOH was added very slowly/dropwise (vigorous bubbling) until bubbling ceased, causing gelatinous solids to form. The mixture was warmed to room temperature, stirred 15 minutes, then it was vacuum filtered through glass microfibre filter (“GF/F”) paper topped with compressed Celite®, rinsed with THF, and the filtrate was dried (Na₂SO₄), filtered, and concentrated. The crude was purified on silica gel (0-15% EtOAc in DCM, then 0-10% MeOH in DCM gradient) to give rac-trans-(4a,10a)-8-bromo-2-(4-methoxybenzyl)-10-methylene-2,3,4,4a,10,10a-hexahydro-1H-chromeno[3,2-c]pyridine (0.278 g, 54.4% yield) as a residue.

Step J: Benzyl carbonochloridate (0.548 mL, 3.84 mmol) was added to a thick-walled sealable pressure tube containing a solution of rac-trans-(4a,10a)-8-bromo-2-(4-methoxybenzyl)-10-methylene-2,3,4,4a,10,10a-hexahydro-1H-chromeno[3,2-c]pyridine (0.256 g, 0.640 mmol) in acetonitrile (2.5 mL) and THF (1 mL). The reaction mixture was heated in a 90° C. sand bath and stirred for 21 hours. The reaction mixture was then cooled to room temperature and concentrated. The resulting residue was combined with EtOAc and poured into saturated NaHCO₃. The mixture was extracted with EtOAc (2×), and the combined extracts were dried (Na₂SO₄), filtered, and concentrated. The crude was purified on silica gel (5-35% EtOAc in hexanes gradient) to give rac-trans-(4a,10a)-benzyl 8-bromo-10-methylene-4,4a,10,10a-tetrahydro-1H-chromeno[3,2-c]pyridine-2(3H)-carboxylate (0.202 g, 76.2% yield) as a residue.

Step K: Silver cyanate (0.290 g, 1.93 mmol) was added to a solution of iodine (0.245 g, 0.967 mmol) in 1:1 acetonitrile:TRF (1 mL). This mixture was sonicated for 1 minute, and it was added by pipette to a 50 mL round bottom flask containing a 0° C. solution of rac-trans-(4a,10a)-benzyl 8-bromo-10-methylene-4,4a,10,10a-tetrahydro-1H-chromeno[3,2-c]pyridine-2(3H)-carboxylate (0.267 g, 0.644 mmol) in THF (3.5 mL; rinsed over with acetonitrile (0.3 mL)). The reaction mixture was stirred at 0° C. for 15 minutes, then warmed to room temperature and stirred. After 4 hours, the reaction mixture was filtered through a pipette plugged with GF/F paper using nitrogen pressure, rinsed with THF, and the filtrate was concentrated to give rac-trans-(4a,10a)-benzyl 8-bromo-10-(iodomethyl)-10-(isocyanato)-4,4a,10,10a-tetrahydro-1H-chromeno[3,2-c]pyridine-2(3H)-carboxylate as a residue, which was used immediately without further purification.

Step L: Concentrated NH₄OH (1.33 mL, 10.2 mmol) was added to a solution of rac-trans-(4a,10a)-benzyl 8-bromo-10-(iodomethyl)-10-(isocyanato)-4,4a,10,10a-tetrahydro-1H-chromeno[3,2-c]pyridine-2(3H)-carboxylate (0.298 g, 0.511 mmol) in THF (5 mL), and the reaction mixture was stirred at room temperature for 14 hours. The reaction mixture was then concentrated, and the residue was partitioned between EtOAc and brine. The mixture was extracted with EtOAc (2×), and the combined extracts were dried (Na₂SO₄), filtered, and concentrated. The crude was purified on silica gel (0-8% MeOH in DCM gradient) to give rac-trans-(4a,10a)-benzyl 2′-amino-8-bromo-1,4,4a,10a-tetrahydro-5′H-spiro[chromeno[3,2-c]pyridine-10,4′-oxazole]-2(3H)-carboxylate (0.219 g, 90.7% yield) as a foam as a 1:1 mixture of diastereomers at the spirocycle.

Step M: rac-trans-(4a,10a)-Benzyl 2′-amino-8-bromo-1,4,4a,10a-tetrahydro-5′H-spiro[chromeno[3,2-c]pyridine-10,4′-oxazole]-2(3H)-carboxylate (0.219 g, 0.464 mmol), 2-fluoropyridin-3-ylboronic acid (0.0849 g, 0.603 mmol), and Pd(PPh₃)₄ (0.0536 g, 0.0464 mmol) were combined with dioxane (3 mL) and 2M Na₂CO₃ (0.695 mL, 1.39 mmol) (both degassed with nitrogen 20 minutes prior to use), the headspace was purged with nitrogen, the mixture was sonicated, and the reaction mixture was heated in a 90° C. reaction block and stirred for 16 hours. The reaction mixture was then concentrated, and the resulting residue was partitioned between EtOAc and water. The mixture was extracted with EtOAc (2×), and the combined extracts dried (Na₂SO₄), filtered, and concentrated. The crude was purified on silica gel (0-8% MeOH in DCM gradient) to give rac-trans-(4a,10a)-benzyl 2′-amino-8-(2-fluoropyridin-3-yl)-1,4,4a,10a-tetrahydro-5′H-spiro[chromeno[3,2-c]pyridine-10,4′-oxazole]-2(3H)-carboxylate (0.154 g, 68.0% yield) as a solid as a 1:1 mixture of diastereomers at the spirocycle. MS m/z (APCI-pos) M+1=489.

### Example 6

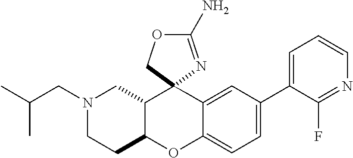

### rac-trans-(4a,4′S*,10a)-8-(2-fluoropyridin-3-yl)-2-isobutyl-1,2,3,4,4a,10a-hexahydro-5′H-spiro[chromeno[3,2-c]pyridine-10,4′-oxazol]-2′-amine

Step A: rac-trans-(4a,10a)-Benzyl 2′-amino-8-(2-fluoropyridin-3-yl)-1,4,4a,10a-tetrahydro-5′H-spiro[chromeno[3,2-c]pyridine-10,4′-oxazole]-2(3H)-carboxylate (0.152 g, 0.311 mmol) was dissolved in THF (2 mL) and EtOH (1 mL), 5% Degussa type Pd/C (0.0662 g, 0.0311 mmol) was added, then hydrogen was bubbled through the reaction mixture for 5 minutes, and the reaction mixture was stirred at room temperature under a hydrogen balloon for 6 hours. The reaction mixture was purged with nitrogen and diluted with MeOH. Celite® was added, and the mixture was vacuum filtered through compressed Celite®. The mixture was rinsed with 1:1 MeOH/THF, and the filtrate was concentrated to give rac-trans-(4a,10a)-8-(2-fluoropyridin-3-yl)-1,2,3,4,4a,10a-hexahydro-5′H-spiro[chromeno[3,2-c]pyridine-10,4′-oxazol]-2′-amine (0.100 g, 90.7% yield) as a powder as a 1:1 mixture of diastereomers at the spirocycle.

Step B: Isobutyraldehyde (0.018 g, 0.248 mmol), rac-trans-(4a,10a)-8-(2-fluoropyridin-3-yl)-1,2,3,4,4a,10a-hexahydro-5′H-spiro[chromeno[3,2-c]pyridine-10,4′-oxazol]-2′-amine (0.020 g, 0.0564 mmol), and Na(OAc)₃BH (0.072 g, 0.339 mmol) were combined with THF (0.3 mL), DMF (0.1 mL) and acetic acid (0.019 mL, 0.338 mmol), and the slightly cloudy mixture was heated to 50° C. and stirred for 22 hours. The reaction mixture was concentrated under a nitrogen stream, diluted with minimal DCM/MeOH, and loaded directly onto a preparative TLC plate for purification (1 mm plate, mobile phase 9:1 DCM:7N NH₃ in MeOH) to give rac-trans-(4a,4′S*,10a)-8-(2-fluoropyridin-3-yl)-2-isobutyl-1,2,3,4,4a,10a-hexahydro-5′H-spiro[chromeno[3,2-c]pyridine-10,4′-oxazol]-2′-amine (0.0003 g, 1.29% yield) as a residue (MS m/z (APCI-pos) M+1=411).

### Example 7

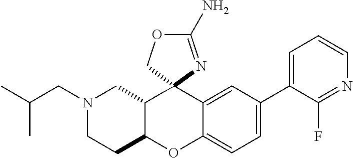

### rac-trans-(4a,4′R*,10a)-8-(2-fluoropyridin-3-yl)-2-isobutyl-1,2,3,4,4a,10a-hexahydro-5′H-spiro[chromeno[3,2-c]pyridine-10,4′-oxazol]-2′-amine

rac-trans-(4a,4′R*,10a)-8-(2-Fluoropyridin-3-yl)-2-isobutyl-1,2,3,4,4a,10a-hexahydro-5′H-spiro[chromeno[3,2-c]pyridine-10,4′-oxazol]-2′-amine (0.0008 g, 3.45% yield) was obtained as a residue from Example 6, Step B (MS m/z (APCI-pos) M+1=411).

### Example 8

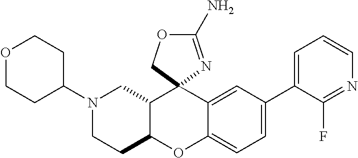

### rac-trans-(4a,4′S*,10a)-8-(2-fluoropyridin-3-yl)-2-(tetrahydro-2H-pyran-4-yl)-1,2,3A,4a,10a-hexahydro-5′H-spiro[chromeno[3,2-c]pyridine-10,4′-oxazol]-2′-amine

rac-trans-(4a,4′S*,10a)-8-(2-Fluoropyridin-3-yl)-2-(tetrahydro-2H-pyran-4-yl)-1,2,3,4,4a,10a-hexahydro-5′H-spiro[chromeno[3,2-c]pyridine-10,4′-oxazol]-2′-amine (0.001 g) was prepared as described in Example 6, Step B, substituting dihydro-2H-pyran-4(3H)-one for isobutyraldehyde. MS m/z (APCI-pos) M+1=439.

### Example 9

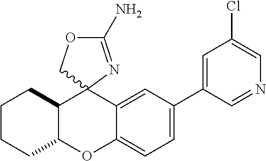

### 7′-(5-chloropyridin-3-yl)-1′,2′,3′,4′,4a′,9a′-hexahydro-5H-spiro[oxazole-4,9′-xanthen]-2-amine (Diastereomer 1)

Step A: 5-Bromo-2-hydroxybenzaldehyde (30 g, 149 mmol) and 4-cyclohexenylmorpholine (25 g, 149 mmol) were combined in dry toluene (600 mL) in a one liter round bottom flask. This mixture was stirred at ambient temperature for 16 hours, during which time a solid had precipitated. The solids were collected by filtration, rinsed with toluene and dried under vacuum to give 7-bromo-4a-morpholino-2,3,4,4a,9,9a-hexahydro-1H-xanthen-9-ol (25.6 g, 47%).

Step B: 7-Bromo-4a-morpholino-2,3,4,4a,9,9a-hexahydro-1H-xanthen-9-ol (25.6 g, 69.5 mmol) and dry 1,2-dichloroethane (280 mL) were added to a one liter round bottom flask. This mixture was chilled to 0° C., and then Dess-Martin periodane (35.4 g, 83.4 mmol) was added. Once addition was complete, the reaction mixture was stirred at 0° C. for 10 minutes and then allowed to warm to ambient temperature over a 16 hour period. 2M aqueous sodium hydroxide (100 mL) was then added, followed by water (300 mL). This mixture was extracted two times with dichloromethane, and the extracts were dried over sodium sulfate and concentrated under reduced pressure. Column chromatography (100% dichloromethane as the eluant) afforded 7-bromo-3,4-dihydro-1H-xanthen-9(2H)-one (14.7 g, 76%) as a solid.

Step C: 7-Bromo-3,4-dihydro-1H-xanthen-9(2H)-one (10 g, 35.8 mmol) and dry THF (360 mL) were added to a one liter round bottom flask. This solution was chilled to −78° C., and L-selectride (39.4 mL, 39.4 mmol, 1M in THF) was then added slowly by syringe. Once the addition was complete, the mixture was stirred at −78° C. for two hours. The mixture was then quenched with methanol (50 mL) and allowed to warm to room temperature. 2M aqueous sodium hydroxide (100 mL) was then added and stirred vigorously for 20 minutes. Water (200 mL) was added, and this mixture was extracted two times with EtOAc, extracts dried over sodium sulfate and concentrated under reduced pressure. Column chromatography (5% EtOAc/Hexane) afforded rac-trans-7-bromo-2,3,4,4a-tetrahydro-1H-xanthen-9(9aH)-one (3.2 g, 32%).

Step D: A 250 mL round bottom flask was charged with trans-7-bromo-2,3,4,4a-tetrahydro-1H-xanthen-9(9aH)-one (2.18 g, 7.75 mmol) in dry THF (70 mL). This solution was chilled to 0° C., and Tebbe's reagent (31 mL, 15.5 mmol, 0.5M in toluene) was then added by syringe over a 5 minute period. Once addition was complete, the reaction mixture was removed from the cooling bath and brought to ambient temperature. After stirring at room temperature for several hours, the mixture was then quenched with 2M aqueous sodium hydroxide solution (100 mL) and stirred vigorously for 30 minutes. The resulting solids were filtered away, and the filtrate was extracted with EtOAc (2×), extracts dried over sodium sulfate and concentrated under reduced pressure. Column chromatography (100% dichloromethane as the eluant) provided rac-trans-7-bromo-9-methylene-2,3,4,4a,9,9a-hexahydro-1H-xanthene (1.27 g, 58%).

Step E: A suspension of iodine/AgOCN (1.15 g, 4.55 mmol/1.02 g, 6.82 mmol; prepared by dissolving iodine in 1:1 acetonitrile:THF (4 mL) and sonicating for 2 minutes) was added to a 0° C. solution of 7-bromo-9-methylene-2,3,4,4a,9,9a-hexahydro-1H-xanthene (1.27 g, 4.55 mmol) in THF (10 mL). The suspension was stirred at 0° C. for 30 minutes and then allowed to warm to room temperature and stirred at ambient temperature for 16 hours. The solids were collected by filtration through GF/F filter paper and rinsed with acetonitrile, and the filtrate was concentrated under reduced pressure. The resulting crude material was then taken up in THF (20 mL) and chilled to 0° C., and concentrated ammonium hydroxide (10 mL) was added. After stirring at 0° C. for 15 minutes, the mixture was vigorously stirred at room temperature for 5 hours. The mixture was then diluted with water (100 mL) and extracted two times with 25% isopropyl alcohol/dichloromethane. The extracts were dried over sodium sulfate and concentrated under reduced pressure to a foam. This material was then triturated with MTBE/DCM to give diastereomer 1 of 7′-bromo-1′,2′,3′,4′,4a′,9a′-hexahydro-5H-spiro[oxazole-4,9′-xanthen]-2-amine (240 mg, 16%) as a solid. The filtrate was purified by column chromatography (1:1 EtOAc:hexane to 100% EtOAc) to give the diastereomer 2 of 7′-bromo-1′,2′,3′,4′,4a′,9a′-hexahydro-5H-spiro[oxazole-4,9′-xanthen]-2-amine (291 mg, 19%).

Step F: A 15 mL pressure tube was charged with diastereomer 1 of 7′-bromo-1′,2′,3′,4′,4a′,9a′-hexahydro-5H-spiro[oxazole-4,9′-xanthen]-2-amine (0.075 g, 0.22 mmol), 5-chloropyridin-3-ylboronic acid (0.105 g, 0.667 mmol), Pd(PPh₃)₄ (0.026 g, 0.022 mmol), 2M aqueous potassium carbonate (0.334 mL, 0.667 mmol) in dioxane (2 mL). This mixture was purged with argon for 5 minutes, and the tube was sealed. The mixture was heated at 90° C. for 16 hours. The mixture was then diluted with water (20 mL), extracted two times with EtOAc, extracts dried over sodium sulfate and concentrated under reduced pressure. Flash chromatography (5% MeOH/DCM) afforded diastereomer 1 of 7′-(5-chloropyridin-3-yl)-1′,2′,3′,4′,4a′,9a′-hexahydro-5H-spiro[oxazole-4,9′-xanthen]-2-amine (33 mg, 40%). m/z (APCI-pos) M+1=370.1.

### Example 10

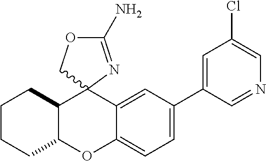

### 7′-(5-chloropyridin-3-yl)-1′,2′,3′,4′,4a′,9a′-hexahydro-5H-spiro[oxazole-4,9′-xanthen]-2-amine (Diastereomer 2)

Diastereomer 2 of 7′-(5-chloropyridin-3-yl)-1′,2′,3′,4′,4a′,9a′-hexahydro-5H-spiro[oxazole-4,9′-xanthen]-2-amine (24 mg, 29%) was prepared in the same manner as outlined in Example 9, Step F, using diastereomer 2 of 7′-bromo-1′,2′,3′,4′,4a′,9a′-hexahydro-5H-spiro[oxazole-4,9′-xanthen]-2-amine. m/z (APCI-pos) M+1=370.1.

### Example 11

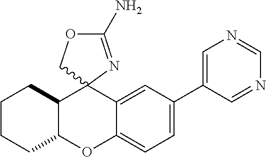

### 7′-(pyrimidin-5-yl)-1′,2′,3′,4′,4a′,9a′-hexahydro-5H-spiro[oxazole-4,9′-xanthen]-2-amine (Diastereomer 1)

Diastereomer 1 of 7′-(pyrimidin-5-yl)-1′,2′,3′,4′,4a′,9a′-hexahydro-5H-spiro[oxazole-4,9′-xanthen]-2-amine (17 mg, 23% yield) was prepared according to Example 9, Steps A-F, substituting pyrimidin-5-ylboronic acid for 5-chloropyridin-3-ylboronic acid in Step F. m/z (APCI-pos) M+1=337.1.

### Example 12

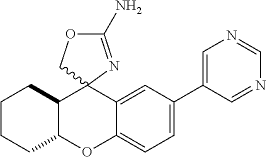

### 7′-(pyrimidin-5-yl)-1′,2′,3′,4′,4a′,9a′-hexahydro-5H-spiro[oxazole-4,9′-xanthen]-2-amine (Diastereomer 2)

Diastereomer 2 of 7′-(pyrimidin-5-yl)-1′,2′,3′,4′,4a′,9a′-hexahydro-5H-spiro[oxazole-4,9′-xanthen]-2-amine (17 mg, 23% yield) was prepared according to Example 9, Steps A-F, and Example 10, substituting pyrimidin-5-ylboronic acid for 5-chloropyridin-3-ylboronic acid in Step F. m/z (APCI-pos) M+1=337.1.

### Example 13

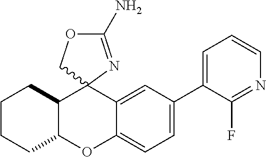

### 7′-(2-fluoropyridin-3-yl)-1′,2′,3′,4′,4a′,9a′-hexahydro-5H-spiro[oxazole-4,9′-xanthen]-2-amine (Diastereomer 1)

Diastereomer 1 of 7′-(2-fluoropyridin-3-yl)-1′,2′,3′,4′,4a′,9a′-hexahydro-5H-spiro[oxazole-4,9′-xanthen]-2-amine (4 mg, 9%) was prepared according to Example 1, Steps A-F, substituting 2-fluoropyridin-3-ylboronic acid for 5-chloropyridin-3-ylboronic acid in Step F. m/z (APCI-pos) M+1=354.1.

### Example 14

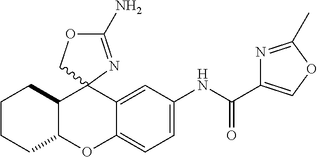

### N-(2-amino-1′,2′,3′,4′,4a′,9a′-hexahydro-5H-spiro[oxazole-4,9′-xanthene]-7′-yl)-2-methyloxazole-4-carboxamide (Diastereomer 1)

Step A: 7′-Bromo-1′,2′,3′,4′,4a′,9a′-hexahydro-5H-spiro[oxazole-4,9′-xanthen]-2-amine (150 mg, 0.445 mmol; mixture of diastereomers) was dissolved in dry THF (5 mL). Triethylamine (0.124 mL, 0.890 mmol) was added, followed by BOC₂O (0.116 g, 0.534 mmol), and the mixture was stirred at ambient temperature for 16 hours. The reaction mixture was then diluted with EtOAc, washed two times with 10% aqueous potassium carbonate, dried over sodium sulfate and concentrated under reduced pressure. Column chromatography (3:1 hexane:EtOAc) afforded tert-butyl 7′-bromo-1′,2′,3′,4′,4a′,9a′-hexahydro-5H-spiro[oxazole-4,9′-xanthene]-2-ylcarbamate (61 mg, 31%) as a mixture of diastereomers.

Step B: A reaction vial was charged with the diastereomeric mixture of tert-butyl 7′-bromo-1′,2′,3′,4′,4a′,9a′-hexahydro-5H-spiro[oxazole-4,9′-xanthene]-2-ylcarbamate (0.0614 g, 0.140 mmol), Pd₂ dba₃ (0.006 g, 0.006 mmol), and biphenyl-2-yldicyclohexylphosphine (0.004 g, 0.013 mmol). This was purged with nitrogen for 5 minutes and LiHMDS (1M in toluene, 0.351 mL, 0.351 mmol) was then added followed by dry toluene (0.5 mL). The vial was capped and heated to 80° C. for 16 hours. The mixture was then diluted with EtOAc and 1M aqueous HCl added (about 1 mL). This mixture was vigorously stirred for 10 minutes, then neutralized with 10% aqueous potassium carbonate, extracted with EtOAc (2×), extracts dried over sodium sulfate and concentrated under reduced pressure. Column chromatography (1:1 ethyl acetate:hexane, visualize with 12 on silica gel) afforded tert-butyl 7′-amino-1′,2′,3′,4′,4a′,9a′-hexahydro-5H-spiro[oxazole-4,9′-xanthene]-2-ylcarbamate (21 mg, 40%) as a mixture of diastereomers.

Step C: A reaction vial was charged with the diastereomeric mixture of tert-butyl 7′-amino-1′,2′,3′,4′,4a′,9a′-hexahydro-5H-spiro[oxazole-4,9′-xanthene]-2-ylcarbamate (0.021 g, 0.056 mmol), 2-methyloxazole-4-carboxylic acid (0.008 g, 0.062 mmol), and 4-(4,6-dimethoxy-1,3,5-triazin-2-yl)-4-methylmorpholin-4-ium chloride hydrate (0.025 g, 0.084 mmol) in methanol (1 mL). This mixture was stirred at ambient temperature for 16 hours, diluted with EtOAc, washed with 10% aqueous potassium carbonate, dried over sodium sulfate and concentrated under reduced pressure. Preparative thin layer chromatography (0.5 mm plate, 80% ethyl acetate:hexane) afforded 2 diastereomers of tert-butyl 7′-(2-methyloxazole-4-carboxamido)-1′,2′,3′,4′,4a′,9a′-hexahydro-5H-spiro[oxazole-4,9′-xanthene]-2-ylcarbamate (4 mg of diastereomer 1 (less polar) and 5.5 mg of diastereomer 2 (more polar)).

Step D: A reaction vial containing diastereomer 1 of tert-butyl 7′-(2-methyloxazole-4-carboxamido)-1′,2′,3′,4′,4a′,9a′-hexahydro-5H-spiro[oxazole-4,9′-xanthene]-2-ylcarbamate (0.004 g, 0.008 mmol) was charged with 2 ml of TFA (2 mL), and the mixture was stirred at room temperature for 1 hour, and then concentrated under reduced pressure and dried under vacuum to give diastereomer 1 of N-(2-amino-1′,2′,3′,4′,4a′,9a′-hexahydro-5H-spiro[oxazole-4,9′-xanthene]-7′-yl)-2-methyloxazole-4-carboxamide as the TFA salt. m/z (APCI-pos) M+1=383.0.

### Example 15

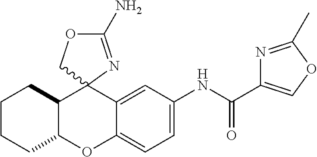

### N-(2-amino-1′,2′,3′,4′,4a′,9a′-hexahydro-5H-spiro[oxazole-4,9′-xanthene]-7′-yl)-2-methyloxazole-4-carboxamide (Diastereomer 2)

Diastereomer 2 of N-(2-amino-1′,2′,3′,4′,4a′,9a′-hexahydro-5H-spiro[oxazole-4,9′-xanthene]-7′-yl)-2-methyloxazole-4-carboxamide TFA salt was prepared according to the procedure of Example 14, substituting diastereomer 2 of tert-butyl 7′-(2-methyloxazole-4-carboxamido)-1′,2′,3′,4′,4a′,9a′-hexahydro-5H-spiro[oxazole-4,9′-xanthene]-2-ylcarbamate. m/z (APCI-pos) M+1=383.0.

### Example 16

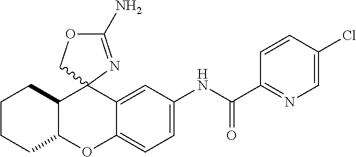

### N-(2-amino-1′,2′,3′,4′,4a′,9a′-hexahydro-5H-spiro[oxazole-4,9′-xanthene]-7′-yl)-5-chloropicolinamide

N-(2-Amino-1′,2′,3′,4′,4a′,9a′-hexahydro-5H-spiro[oxazole-4,9′-xanthene]-7′-yl)-5-chloropicolinamide (3 mg, 8.4%) was prepared according to Example 14, Steps A-D (in TFA step, product was free based using 10% aqueous potassium carbonate and preparative TLC purification) using diastereomer 2 of 7′-bromo-1′,2′,3′,4′,4a′,9a′-hexahydro-5H-spiro[oxazole-4,9′-xanthen]-2-amine (Example 9, Step E), and substituting 5-chloropicolinic acid for 2-methyloxazole-4-carboxylic acid. m/z (APCI-pos) M+1=413.0.

### Example 17

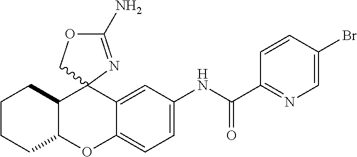

### N-(2-amino-1′,2′,3′,4′,4a′,9a′-hexahydro-5H-spiro[oxazole-4,9′-xanthene]-7′-yl)-5-bromopicolinamide

N-(2-Amino-1′,2′,3′,4′,4a′,9a′-hexahydro-5H-spiro[oxazole-4,9′-xanthene]-7′-yl)-5-bromopicolinamide (3 mg, 7%) was prepared according to Example 14, Steps A-D (in TFA step, product was free based using 10% aqueous potassium carbonate and preparative TLC purification) using diastereomer 2 of 7′-bromo-1′,2′,3′,4′,4a′,9a′-hexahydro-5H-spiro[oxazole-4,9′-xanthen]-2-amine (Example 9, Step E), and substituting 5-bromopicolinic acid for 2-methyloxazole-4-carboxylic acid. m/z (APCI-pos) M+1=457.0, 459.0.

### Example 18

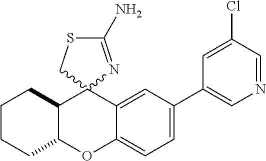

### 7′-(5-chloropyridin-3-yl)-1′,2′,3′,4′,4a′,9a′-hexahydro-5H-spiro[thiazole-4,9′-xanthen]-2-amine

7′-(5-Chloropyridin-3-yl)-1′,2′,3′,4′,4a′,9a′-hexahydro-5H-spiro[thiazole-4,9′-xanthen]-2-amine (22 mg, 27%) was prepared according to Example 9, Steps A-F, substituting silver thiocyanate for silver cyanate in Step E. m/z (APCI-pos) M+1=386.1.

### Example 19

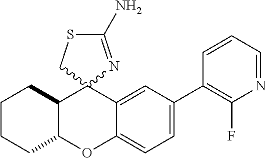

### 7′-(2-fluoropyridin-3-yl)-1′,2′,3′,4′,4a′,9a′-hexahydro-5H-spiro[thiazole-4,9′-xanthen]-2-amine

7′-(2-Fluoropyridin-3-yl)-1′,2′,3′,4′,4a′,9a′-hexahydro-5H-spiro[thiazole-4,9′-xanthen]-2-amine (8 mg, 15%) was prepared according to Example 9, Steps A-F, substituting silver thiocyanate for silver cyanate in Step E and 2-fluoropyridin-3-ylboronic acid for 5-chloropyridin-3-ylboronic acid in Step F. m/z (APCI-pos) M+1=370.1.

### Example 20

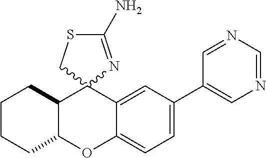

### 7′-(pyrimidin-5-yl)-1′,2′,3′,4′,4a′,9a′-hexahydro-5H-spiro[thiazole-4,9′-xanthen]-2-amine

7′-(Pyrimidin-5-yl)-1′,2′,3′,4′,4a′,9a′-hexahydro-5H-spiro[thiazole-4,9′-xanthen]-2-amine (4 mg, 5%) was prepared according to Example 9, Steps A-F, substituting silver thiocyanate for silver cyanate in Step E and pyrimidin-5-ylboronic acid for 5-chloropyridin-3-ylboronic acid in Step F. m/z (APCI-pos) M+1=353.1.

### Example 21

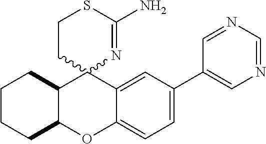

### rac-cis-7′-(pyrimidin-5-yl)-1′,2′,3′,4′,4a′,5,6,9a′-octahydrospiro[[1,3]thiazine-4,9′-xanthen]-2-amine

Step A: rac-trans-7-Bromo-2,3,4,4a-tetrahydro-1H-xanthen-9(9aH)-one (1.30 g, 4.62 mmol) was dissolved in dry THF (40 mL) and chilled to 0° C. Vinyl magnesium bromide (13.9 mL, 13.9 mmol, 1M in THF) was added by syringe over a 5 minute period. Once the addition was complete, the mixture was allowed to warm to room temperature, and then quenched with saturated ammonium chloride solution. Water (100 mL) was then added, and the mixture was extracted two times with EtOac, extracts dried over sodium sulfate and concentrated to rac-trans-7-bromo-9-vinyl-2,3,4,4a,9,9a-hexahydro-1H-xanthen-9-ol (1.45 g, 100%) as a mixture of diastereomers. This material was of sufficient purity to carry on to next step.

Step B: Thionyl chloride (1.12 g, 9.38 mmol) was added to rac-trans-7-bromo-9-vinyl-2,3,4,4a,9,9a-hexahydro-1H-xanthen-9-ol (1.45 g, 4.69 mmol) in dry dichloromethane (40 mL) chilled to 0° C. This mixture was stirred at 0° C. for 10 minutes and then allowed to warm to room temperature and stirred for 48 hours. The reaction mixture was then concentrated under reduced pressure, and the resulting crude material was taken up in acetonitrile (40 mL) and DCM (10 mL). Thiourea (0.357 g, 4.69 mmol) was added to this mixture and was stirred at ambient temperature for 30 minutes, then at 50° C. for 2 hours. A precipitate formed, which was collected by filtration, rinsed with acetonitrile, and dried under vacuum to obtained (E)-2-(7-bromo-2,3,4,4a-tetrahydro-1H-xanthen-9(9aH)-ylidene)ethyl carbamimidothioate (1.47 g, 85%).

Step C: (E)-2-(7-Bromo-2,3,4,4a-tetrahydro-1H-xanthen-9(9aH)-ylidene)ethyl carbamimidothioate (1.4 g, 3.81 mmol) was dissolved in TFA (12 mL, 152 mmol) and chilled to 0° C. Methanesulfonic acid (5 mL, 76.2 mmol) was added, and the mixture was stirred at 0° C. for 15 minutes and then allowed to warm to room temperature over 18 hours. The reaction mixture was then poured into ice cold 10% aqueous potassium carbonate (200 mL) and stirred vigorously for 10 minutes, then extracted two times with EtOAc. The extracts were dried over sodium sulfate and concentrated under reduced pressure to a foam, which was triturated with methanol, filtered, and the filtrate concentrated to 7′-bromo-1′,2′,3′,4′,4a′,5,6,9a′-octahydrospiro[[1,3]thiazine-4,9′-xanthen]-2-amine (0.766 g, 55%) as a mixture of cis/trans diastereomers.

Step D: tert-Butyl 7′-bromo-1′,2′,3′,4′,4a′,5,6,9a′-octahydrospiro[[1,3]thiazine-4,9′-xanthene]-2-ylcarbamate (0.461 g, 47%) as a mixture of diastereomers was prepared according to Example 14, Step A, substituting 7′-bromo-1′,2′,3′,4′,4a′,5,6,9a′-octahydrospiro[[1,3]thiazine-4,9′-xanthen]-2-amine for 7′-bromo-1′,2′,3′,4′,4a′,9a′-hexahydro-5H-spiro[oxazole-4,9′-xanthen]-2-amine.

Step E: A reaction vial was charged with tert-butyl 7′-bromo-1′,2′,3′,4′,4a′,5,6,9a′-octahydrospiro[[1,3]thiazine-4,9′-xanthene]-2-ylcarbamate (0.050 g, 0.107 mmol), PdCl₂(dppf) dichloromethane adduct (0.004 g, 0.005 mmol), pyrimidin-5-ylboronic acid (0.02 g, 0.160 mmol), sodium carbonate (0.187 mL, 0.374 mmol, 2M aqueous solution) in dioxanethe (2 mL) and was purged with nitrogen for 5 minutes. The vial was capped and heated to 90° C. for 16 hours. The mixture was diluted with water, extracted with EtOAc, extracts dried over sodium sulfate and concentrated under reduced pressure. Column chromatography (10% MeOH/DCM) afforded rac-cis-7′-(pyrimidin-5-yl)-1′,2′,3′,4′,4a′,5,6,9a′-octahydrospiro[[1,3]thiazine-4,9′-xanthen]-2-amine (2.5 mg, 6%). m/z (APCI-pos) M+1=367.0.

Upon further structural analysis, it was determined by X-ray crystallography that the relative stereochemistry of Example 21 was rac-trans-7′-(pyrimidin-5-yl)-1′,2′,3′,4′,4a′,5,6,9a′-octahydrospiro[[1,3]thiazine-4,9′-xanthen]-2-amine:

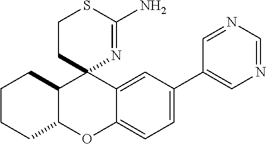

### Example 22

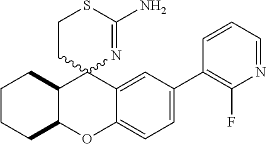

### rac-cis-7′-(2-fluoropyridin-3-yl)-1′,2′,3′,4′,4a′,5,6,9a′-octahydrospiro[[1,3]thiazine-4,9′-xanthen]-2-amine

rac-cis-7′-(2-Fluoropyridin-3-yl)-1′,2′,3′,4′,4a′,5,6,9a′-octahydrospiro[[1,3]thiazine-4,9′-xanthen]-2-amine (15 mg, 37%) was prepared according to Example 21, Steps A-E, substituting 2-fluoropyridin-3-ylboronic acid for pyrimidin-5-ylboronic acid in Step E. m/z (APCI-pos) M+1=384.0.

Upon further structural analysis, it was determined by X-ray crystallography that the relative stereochemistry of Example 22 was rac-trans-7′-(2-fluoropyridin-3-yl)-1′,2′,3′,4′,4a′,5,6,9a′-octahydrospiro[[1,3]thiazine-4,9′-xanthen]-2-amine:

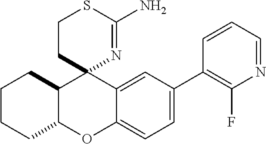

### Example 23

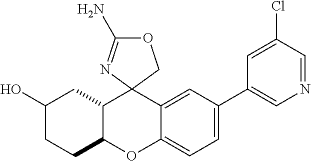

### (4a′S,9a′R)-2-amino-7′-(5-chloropyridin-3-yl)-1′,2′,3′,4′,4a′,9a′-hexahydro-5H-spiro[oxazole-4,9′-xanthen]-2′-ol

Step A: A solution of 1,4-cyclohexanedione monoethylene ketal (50 g, 320 mmol), morpholine (30.7 mL, 352 mmol), and 4-methylbenzenesulfonic acid-monohydrate (1.22 g, 6.40 mmol) in toluene (320 mL, 1M) in a 500 mL round bottom flask was fitted with a Dean-Stark trap and a condensor, and then the reaction mixture was heated at 132° C. (bath temperature) for 12 hours. The reaction was cooled to ambient temperature, concentrated in vacuo, and dried under high vacuum for greater than 24 hours to provide 4-(1,4-dioxaspiro[4.5]dec-7-en-8-yl)morpholine (70 g, 280 mmol, 87% yield) as an oil.

Step B: A solution 4-(1,4-dioxaspiro[4.5]dec-7-en-8-yl)morpholine (70 g, 261 mmol), 5-bromo-2-hydroxybenzaldehyde (52 g, 261 mmol) in toluene (131 mL, 261 mmol) was stirred at room temperature for 24 hours. A solid precipitated after 10 minutes of reaction. After 1 day, the mixture was filtered and washed with a minimal amount of toluene. The solid was dried in a vacuum oven at 50° C. overnight. The solid was confirmed to be 7′-bromo-4a′-morpholino-1′,3′,4′,4a′,9′,9a′-hexahydrospiro[[1,3]dioxolane-2,2′-xanthen]-9′-ol (73 g, 171 mmol, 66% yield), and was taken onto the next step without further purification.

Step C: A solution of 7′-bromo-4a′-morpholino-1′,3′,4′,4a′,9′,9a′-hexahydrospiro[[1,3]dioxolane-2,2′-xanthen]-9′-ol (50 g, 117 mmol) in DCM (586 mL, 117 mmol) was cooled to 0° C., and Dess-Martin reagent (59.7 g, 141 mmol) was added. The mixture was stirred at room temperature for 2 hours, monitoring by TLC. The reaction mixture was diluted with DCM (1 L) and then slowly quenched with 2N NaOH. The mixture was poured into a separatory funnel, rinsing the flask with DCM and water. The organic layer was washed successively with 2N NaOH (2×400-500 mL), 2N HCl (2×300-400 mL), water (1×300-400 mL), brine (1×400-500 mL), dried (Na₂SO₄) and concentrated to afford a solid, which was triturated with ether to afford 7′-bromo-3′,4′-dihydrospiro[[1,3]dioxolane-2,2′-xanthen]-9′(1′H)-one (38 g, 113 mmol, 96%).

Step D: A solution of 7′-bromo-3′,4′-dihydrospiro[[1,3]dioxolane-2,2′-xanthen]-9′(1′H)-one (34 g, 101 mmol) in THF (1008 mL, 101 mmol) (2 L round bottom flask) was cooled to −78° C., and L-Selectride (151 mL, 151 mmol; 1.0M in THF) was added dropwise. The reaction was stirred at −78° C. for 2 hours and then quenched at −78° C. with NH₄Cl (250 mL, saturated). The suspension was stirred vigorously while warming to room temperature. The reaction mixture was diluted with ethyl acetate (500 mL) and water (500 mL) with constant stirring. The mixture was transferred to a separatory funnel, and the aqueous layer was extracted with ethyl acetate (3×). The combined organic layers were dried (Na₂SO₄) and concentrated. The residue was purified by flash chromatography, eluting with a gradient of 40% DCM/hexanes to 40% DCM/ethyl acetate gradient to afford 7′-bromo-1′,4′,4a′,9a′-tetrahydrospiro[[1,3]dioxolane-2,2′-xanthen]-9′(3′H)-one (15.7 g, 46.3 mmol, 45.9% yield). 2:1 trans:cis by NMR.

Step E: Tebbe reagent (36 mL, 18 mmol) was added to a solution of (4a′S*,9a′S*)-7′-bromo-1′,4′,4a′,9a′-tetrahydrospiro[[1,3]dioxolane-2,2′-xanthen]-9′(3′H)-one (5.5 g, 16 mmol) in THF (162 mL, 16 mmol) at 0° C., and the resulting mixture was stirred at 0° C. for 1 hour, warming to room temperature and stirring for an additional 1 hour. The reaction mixture was cooled to 0° C., and methanol was slowly added until bubbling slowed down. Then 2N NaOH was added dropwise to precipitate salts, and addition was discontinued when drops of an aqueous phase appeared on sides of the flask. Na₂SO₄ was then added to this stirring mixture, and the reaction mixture was filtered, washing thoroughly with ether. The filtrate was concentrated. The residue was purified by flash chromatography, eluting with a gradient of 5%-15% ethyl acetate/hexanes to afford (4a′S*,9a′R*)-7′-bromo-9′-methylene-1′,3′,4′,4a′,9′,9a′-hexahydrospiro[[1,3]dioxolane-2,2′-xanthene]. Cis elutes first, trans last.

Step F: In a 20-dram vial, a solution of iodine (1.66 g, 6.52 mmol) in THF (3.26 mL, 3.26 mmol) was added to a suspension of silver cyanate (1.96 g, 13.0 mmol) in acetonitrile (3.26 mL, 3.26 mmol). The resulting mixture was shaken for 60 seconds. This mixture was quickly poured into a solution of (4a′S*,9a′R*)-7′-bromo-9′-methylene-1′,3′,4′,4a′,9′,9a′-hexahydrospiro[[1,3]dioxolane-2,2′-xanthene] (1.10 g, 3.26 mmol) in ether (32.6 mL, 3.26 mmol) at 0° C., rinsing the vial with acetonitrile (1×). After 1 hour, the reaction mixture was filtered through GF/F paper, and to the filtrate was added a stir bar and NR₄OH (1.63 mL, 3.26 mmol). The resulting dark solution was stirred at room temperature overnight. The reaction mixture was partitioned between ethyl acetate and 2N NaOH, and the aqueous layer was extracted with ethyl acetate (3×). The combined organic layers were dried and concentrated to afford a residue that was purified by flash chromatography, eluting with DCM/MeOH gradient (0-15%) to give (4′aS*,9′aR*)-7′-bromo-3′,4′,4′a,9′a-tetrahydro-1′H,2″H-dispiro[1,3-dioxolane-2,2′-xanthene-9′,3″-[1,4]oxazole]-5″-amine (0.850 g, 2.15 mmol, 66%).

Step G: A solution of (4′aS*,9′aR*)-7′-bromo-3′,4′,4′a,9′a-tetrahydro-1′H,2″H-dispiro[1,3-dioxolane-2,2′-xanthene-9′,3″-[1,4]oxazole]-5″-amine (1.20 g, 3.04 mmol) in 2N HCl (8.0 mL) and acetone (15 mL, 3.04 mmol) was heated at 55° C. overnight. The mixture was basified with NaOH until a pH of grater than 10, and the mixture was extracted with ethyl acetate (3×). The combined organic layers were dried and concentrated. The solid was purified by trituration with ether to give a solid corresponding to 2-amino-7′-bromo-1′,4′,4a′,9a′-tetrahydro-5H-spiro[oxazole-4,9′-xanthen]-2′(3′H)-one (480 mg, 1.37 mmol, 45.0% yield).

Step H: NaBH₄ (0.0808 g, 2.14 mmol) was added to a solution of 2-amino-7′-bromo-1′,4′,4a′,9a′-tetrahydro-5H-spiro[oxazole-4,9′-xanthen]-2′(3′H)-one (0.500 g, 1.42 mmol) in THF (14.2 mL, 1.42 mmol) and MeOH (1.42 mL, 1.42 mmol; d. 0.791), and the resulting mixture was stirred for 1 day at room temperature. The mixture was quenched with water and extracted with ethyl acetate (3×). The combined organic layers were dried and concentrated to afford 2-amino-7′-bromo-1′,2′,3′,4′,4a′,9a′-hexahydro-5H-spiro[oxazole-4,9′-xanthen]-2′-ol (0.400 g, 1.13 mmol, 80%).

Step I: A solution of 2-amino-7′-bromo-1′,2′,3′,4′,4a′,9a′-hexahydro-5H-spiro[oxazole-4,9′-xanthen]-2′-ol (88.3 mg, 0.250 mmol), 5-chloropyridin-3-ylboronic acid (41.3 mg, 0.262 mmol), Pd(PPh₃)₄ (14.4 mg, 0.0125 mmol), Na₂CO₃ (375 μL, 0.750 mmol) (2M aqueous) in dioxane (1250 μL, 0.250 mmol) was degassed with nitrogen for 5 minutes, sealed in a vial and stirred at 80° C. for 1 day. The reaction mixture was filtered through an inline syringe filter, washing with methanol. The filtrate was purified by C18 semi-prep HPLC eluting with 5-95% ACN/H₂O+0.1% TFA. The product containing fractions were concentrated, the residue was partitioned between ethyl acetate/2N NaOH, extracted with ethyl acetate (3×), the combined organic layers were dried and concentrated to afford (4a′S*,9a′R*)-2-amino-7′-(5-chloropyridin-3-yl)-1′,2′,3′,4′,4a′,9a′-hexahydro-5H-spiro[oxazole-4,9′-xanthen]-2′-ol (35 mg, 0.09 mmol, 36%). m/z (APCI-pos) M+1=386.1 (100%), 388.1 (30%).

Diastereomer 1: ¹H NMR (CD₃OD) δ 8.67 (d, J=2.0 Hz, 1H), 8.46 (d, J=2.3 Hz, 1H), 8.06 (t, J=2.3 Hz, 1H), 7.60 (d, J=2.3 Hz, 1H), 7.48 (t, J=2.3 Hz, 1H), 6.91 (d, J=8.2 Hz, 1H), 4.64 (d, J=9.4 Hz, 1H), 4.40 (d, J=9.0 Hz, 1H), 3.99 Hz (td, J=10.6, 4.7 Hz, 1H), 3.74 (m, 1H), 2.46 (m, 1H), 2.10-1.96 (m, 2H), 1.95 (m, 2H), 1.50-1.30 (m, 2H).

Diastereomer 2: ¹H NMR (CD₃OD) δ 8.65 (d, J=2.0 Hz, 1H), 8.45 (d, J=2.3 Hz, 1H), 8.04 (t, J=2.3 Hz, 1H), 7.52 (d, J=2.3 Hz, 1H), 7.46 (t, J=2.3 Hz, 1H), 6.87 (d, J=8.6 Hz, 1H), 4.61 (d, J=9.0 Hz, 4.04 (d, J=9.0 Hz, 1H), 3.92 (td, J=11.0, 5.9 Hz, 1H), 3.68 (m, 1H), 2.46 (m, 1H), 2.10-1.96 (m, 2H), 1.62 (m, 2H), 1.30-1.20 (m, 2H).

### Example 24

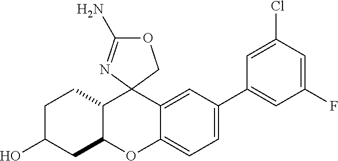

### (4a′S*,9a′R*)-2-amino-7′-(3-chloro-5-fluorophenyl)-1′,2′,3′,4′,4a′,9a′-hexahydro-5H-spiro[oxazole-4,9′-xanthen]-3′-ol

Step A: A solution of 6-bromo-4-oxo-4H-chromene-3-carbaldehyde (25.0 g, 98.8 mmol) in CH₂Cl₂ (988 mL) was stirred at room temperature until homogeneous (additional CH₂Cl₂ was added until completely dissolved). Zinc (II) iodide (4.73 g, 14.8 mmol) was added to this mixture, and the mixture was cooled to 0° C. (Buta-1,3-dien-2-yloxy)trimethylsilane (21.1 g, 148 mmol) was then added to this mixture, and the ice bath was removed. The reaction was stirred for 1.5 hours, or until complete by HPLC (if necessary, additional diene was added to drive reaction). Celite® (25 g) and HCl (1 mL; concentrated) were added to the reaction mixture, and the resulting mixture was stirred at room temperature for 15 minutes. The mixture was filtered through GF/F paper, and the filtrate was transferred to a separatory funnel and washed with water. The organic layer was dried and concentrated to give crude 7-bromo-3,9-dioxo-2,3,4,4a,9,9a-hexahydro-1H-xanthene-9a-carbaldehyde (28.0 g, 86.7 mmol, 88%) as a racemic mixture of diastereomers.

Step B: A mixture of 7-bromo-3,9-dioxo-2,3,4,4a,9,9a-hexahydro-1H-xanthene-9a-carbaldehyde (17.1 g, 52.9 mmol) and 4N HCl (132 mL) in ethanol (265 mL) was heated at 100° C. for 18 hours. The reaction mixture was concentrated to remove ethanol, dissolved in CH₂Cl₂, and then the layers were separated. The organic layer was washed with brine, dried and concentrated. The residue was dissolved with CH₂Cl₂ to load onto a silica chromatography column then eluting with 10-50% ethyl acetate/hexane (+10% CH₂Cl₂) gradient to afford (4aS*,9aS*)-7-bromo-4,4a-dihydro-1H-xanthene-3,9(2H,9aH)-dione (5.0 g, 17 mmol, 67%) and (4aS*,9aR*)-7-bromo-4,4a-dihydro-1H-xanthene-3,9(2H,9aH)-dione (1.7 g, 5.8 mmol, 23%).

Step C: A solution of (4aS*,9aS*)-7-bromo-4,4a-dihydro-1H-xanthene-3,9(2H,9aH)-dione (5.00 g, 16.9 mmol), ethane-1,2-diol (1.04 mL, 18.6 mmol) and TsOH—H₂O (0.322 g, 1.69 mmol) in toluene (84.7 mL, 16.9 mmol) was heated to 130-135° C. (Dean-Stark apparatus) for 4 hours. Additional ethane-1,2-diol was added as necessary to drive the reaction to completion, because at 130-135° C., ethylene glycol was also collected in the Dean-Stark trap. Bis-ketal was formed in substantial amounts when the reaction was run at temperatures below 130° C. The reaction mixture was diluted with ethyl acetate and washed with water. The organic layer was washed with sodium carbonate, brine, dried and concentrated to give (4a′S*,9a′S*)-7′-bromo-1′,4′,4a′,9a′-tetrahydrospiro[[1,3]dioxolane-2,3′-xanthen]-9′(2′H)-one (4.95 g, 14.6 mmol, 86%).

Step D: (4a′S*,9a′R*)-7′-Bromo-9′-methylene-1′,2′,4′,4a′,9′,9a′-hexahydrospiro[[1,3]dioxolane-2,3′-xanthene] (0.83 g, 2.46 mmol, 81%) was prepared from (4a′S*,9a′S*)-7′-bromo-1′,4′,4a′,9a′-tetrahydrospiro[[1,3]dioxolane-2,3′-xanthen]-9′(2′H)-one according to the procedure in Example 23, Step E.

Step E: (4a′S*,9a′ R*)-7′-Bromo-2′,4′,4′a,9′a-tetrahydro-1′H,2″H-dispiro[1,3-dioxolane-2,3′-xanthene-9′,3″-[1,4]oxazole]-5″-amine (0.42 g, 1.06 mmol, >99% yield, about 80% pure) was prepared from (4a′S*,9a′R*)-7′-bromo-9′-methylene-1′,2′,4′,4a′,9′,9a′-hexahydrospiro[[1,3]dioxolane-2,3′-xanthene] according to the procedure in Example 23, Step F.

Step F: (4a′S*,9a′ R*)-2-Amino-7′-bromo-1′,4′,4a′,9a′-tetrahydro-5H-spiro[oxazole-4,9′-xanthen]-3′(2′H)-one (371 mg, 1.06 mmol, 90%) was prepared from (4a′S*,9a′R*)-7′-bromo-2′,4′,4′a,9′a-tetrahydro-1′H,2″H-dispiro[1,3-dioxolane-2,3′-xanthene-9′,3″-[1,4]oxazole]-5″-amine according to the procedure in Example 23, Step G.

Step G: (4a′S*,9a′R*)-2-Amino-7′-bromo-1′,2′,3′,4′,4a′,9a′-hexahydro-5H-spiro[oxazole-4,9′-xanthen]-3′-ol (82 mg, 0.23 mmol, 22%) was prepared from (4a′S*,9a′R*)-2-amino-7′-bromo-1′,4′,4a′,9a′-tetrahydro-5H-spiro[oxazole-4,9′-xanthen]-3′(2′H)-one according to the procedure in Example 23, Step H.

Step H: (4a′S*,9a′R*)-2-Amino-7′-(3-chloro-5-fluorophenyl)-1′,2′,3′,4′,4a′,9a′-hexahydro-5H-spiro[oxazole-4,9′-xanthen]-3′-ol (7 mg, 0.017 mmol, 55%) was prepared from (4a′S*,9a′R*)-2-amino-7′-bromo-1′,2′,3′,4′,4a′,9a′-hexahydro-5H-spiro[oxazole-4,9′-xanthen]-3′-ol according to the procedure in Example 23, Step I. m/z (APCI-pos) M+1=403.1 (100%), 405.1 (35%).

Major diastereomer: ¹H NMR (CD₃OD) δ 7.82 (d, J=2.3 Hz, 1H), 7.60 (d, J=2.3 Hz, 1H), 7.51 (m, 1H), 7.36 (m, 1H), 7.15 (m, 1H), 6.96 (d, J=8.6 Hz, 1H), 5.12 (d, J=9.5 Hz, 1H), 4.64 (d, J=9.5 Hz, 1H), 4.09 Hz (td, J=10.9, 4.7 Hz, 1H), 3.76 (m, 1H), 2.55 (m, 1H), 2.10-1.96 (m, 2H), 1.95 (m, 2H), 1.50-1.30 (m, 2H).

### Example 25

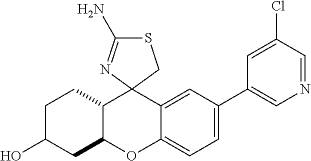

### (4a′S*,9aR*)-2-amino-7′-(5-chloropyridin-3-yl)-1′,2′,3′,4′,4a′,9a′-hexahydro-5H-spiro[thiazole-4,9′-xanthen]-3′-ol

Step A: 7′-Bromo-2′,4′,4′a,9′a-tetrahydro-1′H,2″H-dispiro[1,3-dioxolane-2,3′-xanthene-9′,3″-[1,4]thiazole]-5″-amine (0.25 g, 0.61 mmol, 99%, about 80% pure) was prepared from 7′-bromo-9′-methylene-1′,2′,4′,4a′,9′,9a′-hexahydrospiro[[1,3]dioxolane-2,3′-xanthene] according to the procedure in Example 23, Step F, substituting silver thiocyanate for silver cyanate.

Step B: (4a′S*,9a′R*)-2-Amino-7′-bromo-1′,4′,4a′,9a′-tetrahydro-5H-spiro[thiazole-4,9′-xanthen]-3′(2H)-one (0.22 g, 0.61 mmol, 99%, 80% pure) was prepared from 7′-bromo-2′,4′,4′a,9′a-tetrahydro-1′H,2″H-dispiro[1,3-dioxolane-2,3′-xanthene-9′,3″-[1,4]thiazole]-5″-amine according to the procedure in Example 23, Step G.

Step C: (4a′S*,9a′R*)-2-Amino-7′-bromo-1′,2′,3′,4′,4a′,9a′-hexahydro-5H-spiro[thiazole-4,9′-xanthen]-3′-ol (55 mg, 0.149 mmol, 24%) was prepared from (4a′S*,9a′R*)-2-amino-7′-bromo-1′,4′,4a′,9a′-tetrahydro-5H-spiro[thiazole-4,9′-xanthen]-3′(2′H)-one according to the procedure in Example 23, Step H.

Step D: To a solution of (4a′S*,9a′R*)-2-amino-7′-bromo-1′,2′,3′,4′,4a′,9a′-hexahydro-5H-spiro[thiazole-4,9′-xanthen]-3′-ol (55 mg, 0.149 mmol) and TEA (104 μL, 0.745 mmol; d. 0.726) in DCM (1489 pt, 0.149 mmol) was added Boc₂O (81.3 mg, 0.372 mmol), and the resulting solution was stirred at room temperature for 4 hours. The mixture was diluted with DCM and washed with brine, dried and concentrated. The residue was purified by flash chromatography eluting with an ethyl acetate/hexane gradient to afford tert-butyl (4a′S*,9a′R*)-7′-bromo-3′-hydroxy-1′,2′,3′,4′,4a′,9a′-hexahydro-5H-spiro[thiazole-4,9′-xanthene]-2-ylcarbamate (47.2 mg, 0.101 mmol, 68%).

Step E: A solution of tert-butyl 7′-bromo-3′-hydroxy-1′,2′,3′,4′,4a′,9a′-hexahydro-5H-spiro[thiazole-4,9′-xanthene]-2-ylcarbamate (9.1 mg, 0.0194 mmol), 5-chloropyridin-3-ylboronic acid (3.20 mg, 0.0204 mmol), Pd(PPh₃)₄ (1.12 mg, 0.001 mmol), Na₂CO₃ (29 μL, 0.058 mmol; 2M aqueous) in dioxane (100 μL, 0.0194 mmol) was degassed with nitrogen for 5 minutes, sealed in a vial and stirred at 80° C. for 1 day. The reaction mixture was diluted with ethyl acetate and brine. The aqueous layer was extracted with ethyl acetate (2×). The combined organic layers were concentrated. The residue was diluted with TFA (2 mL) and stirred at room temperature for 1 hour. The TFA was removed in vacuo, and the residue was purified by C18 semi-prep HPLC eluting with 5-95% ACN/H₂O+0.1% TFA to afford 2-amino-7′-(5-chloropyridin-3-yl)-1′,2′,3′,4′,4a′,9a′-hexahydro-5H-spiro[thiazole-4,9′-xanthen]-3′-ol (5.4 mg, 0.0134 mmol, 69.3% yield). m/z (APCI-pos) M+1=402.1 (100%), 404.1 (30%).

Major diastereomer: ¹H NMR (CD₃OD) δ 8.73 (br s, 1H), 8.53 (d, J=2.3 Hz, 1H), 8.16 (br s, 1H), 7.89 (br s, 1H), 7.65 (d, J=8.6 Hz, 1H), 7.05 (d, J=8.6 Hz, 1H), 4.25 (d, J=12.0 Hz, 1H), 4.40 (d, J=11 Hz, 1H), 3.99 Hz (td, J=11.0, 4.7 Hz, 1H), 3.65 (m, 1H), 2.55 (m, 1H), 2.10-1.96 (m, 2H), 1.95 (m, 2H), 1.50-1.20 (m, 2H).

### Example 26

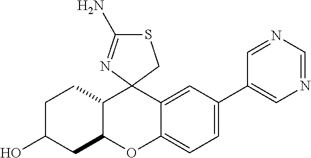

### (4a′S*,9a′R*)-2-amino-7′-(pyrimidin-5-yl)-1′,2′,3′,4′,4a′,9a′-hexahydro-5H-spiro[thiazole-4,9′-xanthen]-3′-ol

(4a′S*,9a′R*)-2-Amino-7′-(pyrimidin-5-yl)-1′,2′,3′,4′,4a′,9a′-hexahydro-5H-spiro[thiazole-4,9′-xanthen]-3′-ol (6.2 mg, 0.0151 mmol, 64%) was prepared according to Example 25 Step D, substituting pyrimidine-5-boronic acid. m/z (APCI-pos) M+1=369.1 (100%), 370.1 (20%).

Major diastereomer: ¹H NMR (CD₃OD) δ 9.16 (br s, 1H), 9.13 (br s, 2H), 7.95 (br s, 1H), 7.70 (d, J=8.6 Hz, 1H), 7.08 (d, J=8.6 Hz, 1H), 4.25 (d, J=12.5 Hz, 1H), 3.99 (d, J=12.5 Hz, 1H), 3.80-3.55 (m, 2H), 2.55 (m, 1H), 2.25 (m, 1H), 2.10-1.96 (m, 2H), 1.95 (m, 1H), 1.50-1.20 (m, 2H).

### Example 27

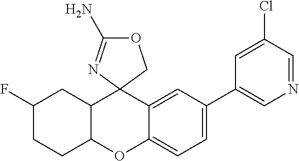

### 7′-(5-chloropyridin-3-yl)-2′-fluoro-1′,2′,3′,4′,4a′,9a′-hexahydro-5H-spiro[oxazole-4,9′-xanthen]-2-amine

A mixture of 2-amino-7′-(5-chloropyridin-3-yl)-1′,2′,3′,4′,4a′,9a′-hexahydro-5H-spiro[oxazole-4,9′-xanthen]-2′-ol (Example 23, Step I, 0.015 g, 0.039 mmol) and deoxofluor (0.0086 mL, 0.047 mmol) in DCM (0.19 mL, 0.039 mmol) in a plastic vial at −78° C. was stirred for 2 hours and then quenched with saturated NaHCO₃ solution. The organics were extracted with DCM twice, washed with brine and dried with Na₂SO₄. The organics were concentrated and purified by preparative HPLC to give 7′-(5-chloropyridin-3-yl)-2′-fluoro-1′,2′,3′,4′,4a′,9a′-hexahydro-5H-spiro[oxazole-4,9′-xanthen]-2-amine (0.0038 g, 0.0098 mmol, 25%). ¹H NMR (400 MHz, CDCl₃+CD₃OD) δ 8.76 (s, 1H), 8.60 (s, 1H), 8.04 (m, 1H), 7.51 (dd, 1H), 7.47 (m, 1H), 7.05 (d, 1H), 4.99 (d, 1H), 4.75 (d, 1H), 3.66 (m, 1H), 3.3 (m, 1H), 2.35 (m, 2H), 2.24 (m, 2H), 1.64 (m, 3H). MS m/z (APCI-pos) M+1=388.1.

### Example 28

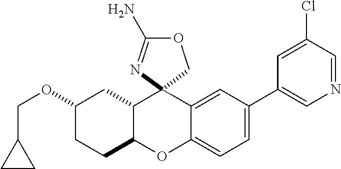

### (4R*,4a′S*,9a′R*)-7′-(5-chloropyridin-3-yl)-2′-(cyclopropylmethoxy)-1′,2′,3′,4′,4a′,9a′-hexahydro-5H-spiro[oxazole-4,9′-xanthen]-2-amine

Step A: Cyclopropylmethanol (5.0 g, 69.3 mmol) was added to a solution of n-BuLi (27.7 mL, 69.3 mmol; 2.5M in THF) in ether (139 mL, 0.5M) at 0° C., and the resulting slurry was stirred at 0° C. for 1 hour. TMS-Cl (8.77 mL, 69.3 mmol) was added to this mixture, and the mixture was stirred at room temperature for 2 hours. The mixture was filtered through GF/F paper, and the filtrate was carefully concentrated to afford (cyclopropylmethoxy)trimethylsilane (8.0 g, 55.4 mmol, 80%).

Step B: (Cyclopropylmethoxy)trimethylsilane (1068 mg, 7.403 mmol) and triethylsilane (1182 μL, 7.403 mmol) were added to a solution of 2-amino-7′-bromo-1′,4′,4a′,9a′-tetrahydro-5H-spiro[oxazole-4,9′-xanthen]-2′(3′H)-one (65 mg, 0.1851 mmol) in acetonitrile (1.85 mL, 0.1M), and then tert-butyldimethylsilyl trifluoromethanesulfonate (850.1 μL, 3.702 mmol) was added. The mixture was stirred at room temperature for 1 day. The solvents were evaporated, and the residue was purified by flash chromatography eluting with DCM/MeOH 0-10% to afford (4a′S*,9a′R*)-7′-bromo-2′-(cyclopropylmethoxy)-1′,2′,3′,4′,4a′,9a′-hexahydro-5H-spiro[oxazole-4,9′-xanthen]-2-amine (39 mg, 0.096 mmol, 52%).

Step C: (4a′S*,9a′R*)-7′-(5-Chloropyridin-3-yl)-2′-(cyclopropylmethoxy)-1′,2′,3′,4′,4a′,9a′-hexahydro-5H-spiro[oxazole-4,9′-xanthen]-2-amine was prepared according to Example 23, Step I, using (4a′S*,9a′R*)-7′-bromo-2′-(cyclopropylmethoxy)-1′,2′,3′,4′,4a′,9a′-hexahydro-5H-spiro[oxazole-4,9′-xanthen]-2-amine. Purification by semi-prep C18 chromatography (ACN/H₂O+0.1% TFA) gave two diastereomers of product. (4R*,4a′S*,9a′R*)-7′-(5-chloropyridin-3-yl)-2′-(cyclopropylmethoxy)-1′,2′,3′,4′,4a′,9a′-hexahydro-5H-spiro[oxazole-4,9′-xanthen]-2-amine. m/z (APCI-pos) M+1=440.1 (100%), 442.1 (40%). ¹H NMR (CD₃OD) δ 8.75 (d, J=2.0 Hz, 1H), 8.52 (d, J=2.0 Hz, 1H), 8.19 (t, J=2.3 Hz, 1H), 7.87 (d, J=2.0 Hz, 1H), 7.66 (dd, J=8.6, 2.3 Hz, 1H), 7.03 (d, J=8.6 Hz), 5.25 (d, J=10.1 Hz, 1H), 5.05 (d, J=10.1 Hz, 1H), 3.90 (td, J=11.0, 5.1 Hz, 1H), 3.58 (tt, J=11.0, 3.9 Hz, 1H), 3.40 (m, 2H), 2.40-2.20 (m, 2H), 2.15 (td, J=12.5, 3.5 Hz, 2H), 1.70-1.60 (m, 1H), 1.40-1.15 (m, 2H), 1.10-0.95 (m, 1H), 0.55 (m, 2H), 0.23 (m, 2H).

### Example 29

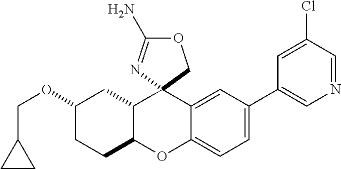

### (4S*,4a′S*,9a′R*)-7′-(5-chloropyridin-3-yl)-2′-(cyclopropylmethoxy)-1′,2′,3′,4′,4a′,9a′-hexahydro-5H-spiro[oxazole-4,9′-xanthen]-2-amine

(4S*,4a′S*,9a′R*)-7′-(5-Chloropyridin-3-yl)-2′-(cyclopropylmethoxy)-1′,2′,3′,4′,4a′,9a′-hexahydro-5H-spiro[oxazole-4,9′-xanthen]-2-amine was the other diastereomer prepared in Example 28, Step C. m/z (APCI-pos) M+1=440.1 (100%), 442.1 (40%). ¹H NMR (CD₃OD) δ 8.75 (d, J=2.0 Hz, 1H), 8.51 (d, J=2.0 Hz, 1H), 8.18 (t, J=2.3 Hz, 1H), 7.91 (d, J=2.3 Hz, 1H), 7.64 (dd, J=8.6, 2.3 Hz, 1H), 6.96 (d, J=8.6 Hz, 1H), 5.22 (d, J=9.8 Hz, 1H), 4.68 (d, J=9.8 Hz, 1H), 4.04 (td, J=11.0, 5.1 Hz, 1H), 3.58 (m, 1H), 3.41 (d, J=7.0 Hz, 1H), 3.37 (d, J=7.0 Hz, 1H), 2.38-2.30 (m, 1H), 2.25-2.15 (m, 3H), 1.70-1.60 (m, 1H), 1.50-1.35 (m, 2H), 1.10-0.95 (m, 1H), 0.54 (m, 2H), 0.22 (m, 2H).

### Example 30

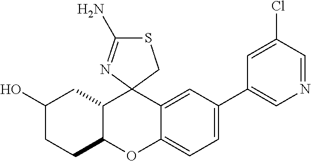

### (4a′S*,9a′R*)-2-amino-7′-(5-chloropyridin-3-yl)-1′,2′,3′,4′,4a′,9a′-hexahydro-5H-spiro[thiazole-4,9′-xanthen]-2′-ol

Step A: (4aS*,9a′R*)-7′-Bromo-3′,4′,4′a,9′a-tetrahydro-1′H,2″H-dispiro[1,3-dioxolane-2,2′-xanthene-9′,3″-[1,4]thiazole]-5″-amine (0.26 g, 0.63 mmol, 99%, about 75% pure) was prepared from (4a′S*,9a′R*)-7′-bromo-9′-methylene-1′,3′,4′,4a′,9′,9a′-hexahydrospiro[[1,3]dioxolane-2,2′-xanthene] according to the procedure in Example 23, Step F, substituting silver thiocyanate for silver cyanate.

Step B: (4a′S*,9a′R*)-2-amino-7′-bromo-1′,4′,4a′,9a′-tetrahydro-5H-spiro[thiazole-4,9′-xanthen]-2′(3′H)-one (0.23 g, 0.63 mmol, 99%, 70% pure) was prepared from (4a′S*,9′aR*)-7′-bromo-3′,4′,4′a,9′a-tetrahydro-1′H,2″H-dispiro[1,3-dioxolane-2,2′-xanthene-9′,3″-[1,4]thiazole]-5″-amine according to the procedure in Example 23, Step G.

Step C: (4a′S*,9a′R*)-2-Amino-7′-bromo-1′,2′,3′,4′,4a′,9a′-hexahydro-5H-spiro[thiazole-4,9′-xanthen]-2′-ol (74 mg, 0.20 mmol, 32%, >99% pure) was prepared from (4a′S*,9a′R*)-2-amino-7′-bromo-1′,4′,4a′,9a′-tetrahydro-5H-spiro[thiazole-4,9′-xanthen]-2′(3′H)-one according to the procedure in Example 23, Step H.

Step D: tert-Butyl (4a′S*,9a′R*)-7′-bromo-2′-hydroxy-1′,2′,3′,4′,4a′,9a′-hexahydro-5H-spiro[thiazole-4,9′-xanthene]-2-ylcarbamate (77 mg, 0.16 mmol, 71%) was prepared from (4a′S*,9a′R*)-2-amino-7′-bromo-1′,2′,3′,4′,4a′,9a′-hexahydro-5H-spiro[thiazole-4,9′-xanthen]-2′-ol according to the procedure in Example 25, Step D.

Step E: (4a′S*,9a′R*)-2-Amino-7′-(5-chloropyridin-3-yl)-1′,2′,3′,4′,4a′,9a′-hexahydro-5H-spiro[thiazole-4,9′-xanthen]-2′-ol (8.4 mg, 0.019 mmol, 49%) was prepared from tert-butyl (4a′S*,9a′R*)-7′-bromo-2′-hydroxy-1′,2′,3′,4′,4a′,9a′-hexahydro-5H-spiro[thiazole-4,9′-xanthene]-2-ylcarbamate according to the procedure in Example 25, Step E. m/z (APCI-pos) M+1=402.1 (100%), 404.1 (40%).

Major diastereomer: ¹H NMR (CD₃OD) δ 8.66 (d, J=2.0 Hz, 1H), 8.48 (d, J=2.3 Hz, 1H), 8.05 (t, J=2.0 Hz, 1H), 7.83 (d, J=2.3 Hz, 1H), 7.52 (dd, J=8.6, 2.3 Hz, 1H), 6.96 (d, J=8.6 Hz, 1H), 4.05-3.95 (m, 2H), 3.83-3.70 (m, 2H), 2.25-2.19 (m, 2H), 2.10-1.96 (m, 1H), 1.95 (m, 1H), 1.60 (m, 1H), 1.50-1.20 (m, 2H).

### Example 31

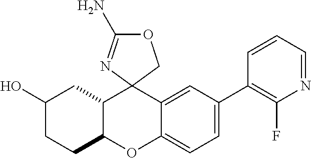

### (4a′S*,9a′R*)-2-amino-7′-(2-fluoropyridin-3-yl)-1′,2′,3′,4′,4a′,9a′-hexahydro-5H-spiro[oxazole-4,9′-xanthen]-2′-ol

(4a′S*,9a′R*)-2-Amino-7′-(2-fluoropyridin-3-yl)-1′,2′,3′,4′,4a′,9a′-hexahydro-5H-spiro[oxazole-4,9′-xanthen]-2′-ol (17 mg, 0.046 mmol, 83%) was prepared according to Example 23, Step I, substituting 2-fluoro-3-pyridine boronic acid. Two diastereomers were observed: m/z (APCI-pos) M+1=370.1 (100%), 371.1 (20%).

Diastereomer 1: ¹H NMR (CD₃OD) δ 8.16 (m, 1H), 8.05 (m, 1H), 7.74 (m, 1H), 7.54 (m, 1H), 7.40 (m, 1H), 7.00 (m, 1H), 5.21 (d, J=10.0 Hz, 1H), 4.99 (d, J=12.5 Hz, 1H), 4.04 (td, J=11, 4.7 Hz, 1H), 3.83 (m, 1H), 2.31 (m, 1H), 2.10 (m, 2H), 1.65 (m, 2H), 1.20 (m, 2H).

Diastereomer 2: ¹H NMR (CD₃OD) δ 8.16 (m, 1H), 8.05 (m, 1H), 7.74 (m, 1H), 7.54 (m, 1H), 7.40 (m, 1H), 7.00 (m, 1H), 5.13 (d, J=12.5 Hz, 1H), 4.65 (d, J=12.5 Hz, 1H), 3.91 (td, J=11, 4.7 Hz, 1H), 3.74 (m, 1H), 2.19 (m, 1H), 1.92 (m, 2H), 1.40 (m, 2H), 1.20 (m, 2H).

### Example 32

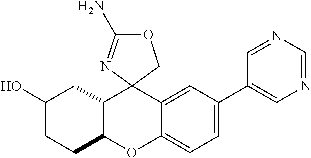

### (4a′S*,9a′R*)-2-amino-7′-(pyrimidin-5-yl)-1′,2′,3′,4′,4a′,9a′-hexahydro-5H-spiro[oxazole-4,9′-xanthen]-2′-ol

(4a′S*,9a′R*)-2-Amino-7′-(pyrimidin-5-yl)-1′,2′,3′,4′,4a′,9a′-hexahydro-5H-spiro[oxazole-4,9′-xanthen]-2′-ol (13 mg, 0.037 mmol, 53%) was prepared according to Example 23, Step I, substituting pyrimidin-5-yl boronic acid. m/z (APCI-pos) M+1=353.1 (100%), 354.1 (20%).

Major diastereomer: ¹H NMR (CD₃OD) δ 9.11 (br s, 1H), 9.07 (br s, 2H), 7.93 (m, 1H), 7.68 (m, 1H), 7.05 (m, 1H), 5.21 (d, J=12.5 Hz, 1H), 5.01 (d, J=12.5 Hz, 1H), 4.10 (m, 1H), 3.81 (m, 1H), 2.31 (m, 1H), 2.10-1.96 (m, 2H), 1.95 (m, 2H), 1.50-1.20 (m, 2H).

### Example 33

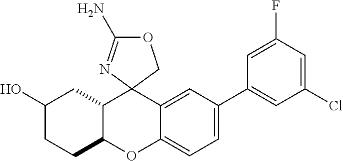

### (4a′S*,9a′R*)-2-amino-7′-(3-chloro-5-fluorophenyl)-1′,2′,3′,4′,4a′,9a′-hexahydro-5H-spiro[oxazole-4,9′-xanthen]-2′-ol

(4a′S*,9a′R*)-2-Amino-7′-(3-chloro-5-fluorophenyl)-1′,2′,3′,4′,4a′,9a′-hexahydro-5H-spiro[oxazole-4,9′-xanthen]-2′-ol (10 mg, 0.025 mmol, 39%) was prepared according to Example 23, Step I, substituting 3-chloro-5-fluorophenyl boronic acid. m/z (APCI-pos) M+1=403.1 (100%), 405.1 (35%).

Major diastereomer: ¹H NMR (CD₃OD) δ 7.84 (d, 1H), 7.59 (br s, 2H), 7.51 (br s, 1H), 7.36 (m, 1H), 7.15 (m, 1H), 6.94 (d, 1H), 5.21 (d, J=12.5 Hz, 1H), 4.67 (d, J=12.5 Hz, 1H), 4.03 (m, 1H), 3.71 (m, 1H), 2.31 (m, 1H), 2.10-1.96 (m, 2H), 1.95 (m, 2H), 1.50-1.20 (m, 2H).

### Example 34

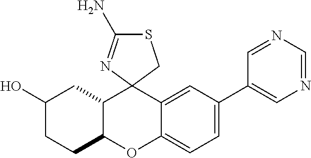

### (4a′S*,9aR*)-2-amino-7′-(pyrimidin-5-yl)-1′,2′,3′,4′,4a′,9a′-hexahydro-5,4-spiro[thiazole-4,9′-xanthen]-2′-ol

(4a′S*,9a′R*)-2-Amino-7′-(pyrimidin-5-yl)-1′,2′,3′,4′,4a′,9a′-hexahydro-5H-spiro[thiazole-4,9′-xanthen]-2′-ol (4.5 mg, 0.012 mmol, 53%) was prepared according to Example 30, Step E, substituting pyrimidin-5-yl boronic acid. m/z (APCI-pos) M+1=369.1 (100%), 370.1 (20%).

### Example 35

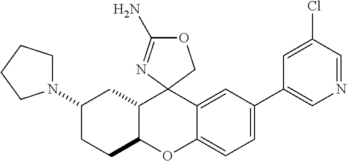

### (2′S*,4a′S*,9a′R*)-7′-(5-chloropyridin-3-yl)-2′-(pyrrolidin-1-yl)-1′,2′,3′,4′,4a′,9a′-hexahydro-5H-spiro[oxazole-4,9′-xanthen]-2-amine

Step A: (4′aS*,9′aR*)-7′-Bromo-3′,4′,4′a,9′a-tetrahydro-1′H,2″H-dispiro[1,3-dioxolane-2,2′-xanthene-9′,3″-[1,4]oxazole]-5″-amine (Example 23, Step F) was subjected to Suzuki coupling conditions as described in Example 23, Step I, to afford (4′aS*,9′aR*)-7′-(5-chloropyridin-3-yl)-3′,4′,4′a,9′a-tetrahydro-1′H,2″H-dispiro[1,3-dioxolane-2,2′-xanthene-9′,3″-[1,4]oxazole]-5″-amine (158 mg, 0.369 mmol, 60.1% yield).

Step B: (4′aS*,9′aR*)-7′-(5-Chloropyridin-3-yl)-3′,4′,4′a,9′a-tetrahydro-1′H,2″H-dispiro[1,3-dioxolane-2,2′-xanthene-9′,3″-[1,4]oxazole]-5″-amine was hydrolyzed as described in Example 23, Step G to afford (4a′S*,9a′R*)-2-amino-7′-(5-chloropyridin-3-yl)-1′,4′,4a′,9a′-tetrahydro-5H-spiro[oxazole-4,9′-xanthen]-2′(3′H)-one (76 mg, 0.198 mmol, 53.6% yield).

Step C: A solution of 2-amino-7′-(5-chloropyridin-3-yl)-1′,4′,4a′,9a′-tetrahydro-5H-spiro[oxazole-4,9′-xanthen]-2′(3′H)-one (5 mg, 0.013 mmol), pyrrolidine (1.9 mg, 0.026 mmol) and NaBH(OAc)₃ (5.5 mg, 0.026 mmol) in DCE(bp83) (1.3 mg, 0.013 mmol) was stirred at room temperature for 5 minutes, then AcOH (5 drops) was added, and the resulting solution was stirred at room temperature for 1 day. The crude reaction mixture was diluted with MeOH and filtered, then purified by Gilson C18 semi-prep HPLC, eluting with ACN/H₂O 0.1% TFA to afford 7′-(5-chloropyridin-3-yl)-2′-(pyrrolidin-1-yl)-1′,2′,3′,4′,4a′,9a′-hexahydro-5H-spiro[oxazole-4,9′-xanthen]-2-amine (3.1 mg, 0.0071 mmol, 54%). m/z (APCI-pos) M+1=439.1 (100%), 440.2 (25%).

### Example 36

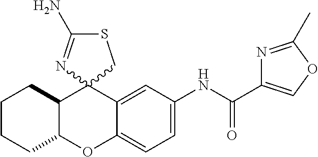

### N-(2-amino-1′,2′,3′,4′,4a′,9a′-hexahydro-5H-spiro[thiazole-4,9′-xanthene]-7′-yl)-2-methyloxazole-4-carboxamide

N-(2-Amino-1′,2′,3′,4′,4a′,9a′-hexahydro-5H-spiro[thiazole-4,9′-xanthene]-7′-yl)-2-methyloxazole-4-carboxamide (6%) was prepared according to the procedure of Example 14, substituting 7′-bromo-1′,2′,3′,4′,4a′,9a′-hexahydro-5H-spiro[thiazole-4,9′-xanthen]-2-amine for 7′-bromo-1′,2′,3′,4′,4a′,9a′-hexahydro-5H-spiro[oxazole-4,9′-xanthen]-2-amine. m/z (APCI-pos) M+1=399.1.

The following compounds in Table 1 were prepared according to the above procedures using appropriate intermediates.

TABLE 1

| Ex. | | | NMR/ |
|---|---|---|---|
| # | Structure | Name | MS |
| 37 | 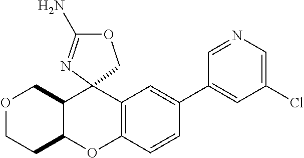 | (4R*,4a′S*,10a′R*)-8′-(5- chloropyridin-3-yl)- 3′,4′,4a′,10a′-tetrahydro- 1′H,5H-spiro[oxazole-4,10′- pyrano[4,3-b]chromen]-2- amine | 372 |
| 38 | 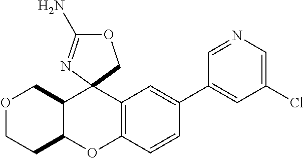 | (4S*,4a′S*,10a′R*)-8′-(5- chloropyridin-3-yl)- 3′,4′,4a′,10a′-tetrahydro- 1′H,5H-spiro[oxazole-4,10′- pyrano[4,3-b]chromen]-2- amine | 372 |
| 39 | 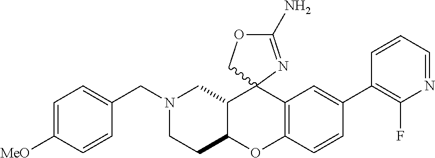 | rac-trans-(4a,10a)-8-(2- fluoropyridin-3-yl)-2-(4- methoxybenzyl)- 1,2,3,4,4a,10a-hexahydro- 5′H-spiro[chromeno[3,2- c]pyridine-10,4′-oxazol]-2′- amine bis trifluoroacetate salt | 475 |
| 40 | 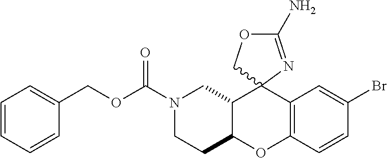 | rac-trans-(4a,10a)-benzyl 2′- amino-8-bromo-1,4,4a,10a- tetrahydro-5′H- spiro[chromeno[3,2- c]pyridine-10,4′-oxazole]- 2(3H)-carboxylate | 473/475 |
| 41 | 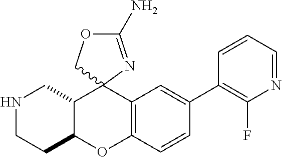 | rac-trans-(4a,10a)-8-(2- fluoropyridin-3-yl)- 1,2,3,4,4a,10a-hexahydro- 5′H-spiro[chromeno[3,2- c]pyridine-10,4′-oxazol]-2′- amine | 355 |
| 42 | 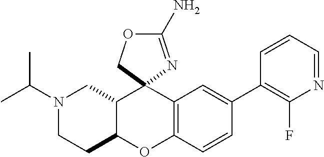 | rac-trans-(4a,4′S*,10a)-8- (2-fluoropyridin-3-yl)-2- isopropyl-1,2,3,4,4a,10a- hexahydro-5′H- spiro[chromeno[3,2- c]pyridine-10,4′-oxazol]-2′- amine | 397 |
| 43 | 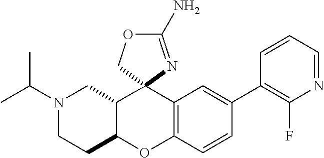 | rac-trans-(4a,4′R*,10a)-8- (2-fluoropyridin-3-yl)-2- isopropyl-1,2,3,4,4a,10a- hexahydro-5′H- spiro[chromeno[3,2- c]pyridine-10,4′-oxazol]-2′- amine | 397 |
| 44 | 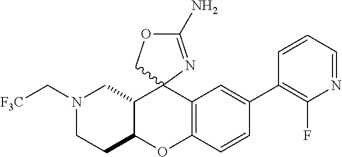 | rac-trans-(4a,10a)-8-(2- fluoropyridin-3-yl)-2-(2,2,2- trifluoroethyl)- 1,2,3,4,4a,10a-hexahydro- 5′H-spiro[chromeno[3,2- c]pyridine-10,4′-oxazol]-2′- amine | 437 |
| 45 | 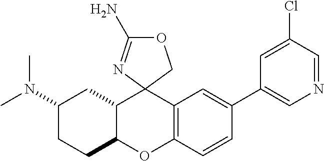 | (2′S,4a′S,9a′R)-7′-(5- chloropyridin-3-yl)-N2′,N2′- dimethyl-1′,2′,3′,4′,4a′,9a′- hexahydro-5H- spiro[oxazole-4,9′- xanthene]-2,2′-diamine | 413.1 |
| 46 | 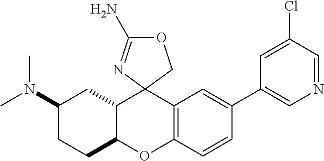 | (2′R,4a′S,9a′R)-7′-(5- chloropyridin-3-yl)-N2′,N2′- dimethyl-1′,2′,3′,4′,4a′,9a′- hexahydro-5H- spiro[oxazole-4,9′- xanthene]-2,2′-diamine | 413.1 |
| 47 | 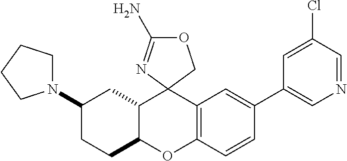 | (2′R,4a′S,9a′R)-7′-(5- chloropyridin-3-yl)-2′- (pyrrolidin-1-yl)- 1′,2′,3′,4′,4a′,9a′-hexahydro- 5H-spiro[oxazole-4,9′- xanthen]-2-amine | 439.1 |
| 48 | 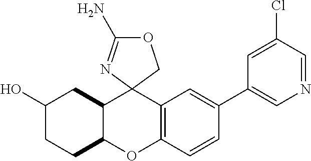 | (4a′S,9a′S)-2-amino-7′-(5- chloropyridin-3-yl)- 1′,2′,3′,4′,4a′,9a′-hexahydro- 5H-spiro[oxazole-4,9′- xanthen]-2′-ol | 386.1 |
| 49 | 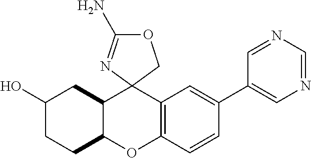 | (4a′S,9a′S)-2-amino-7′- (pyrimidin-5-yl)- 1′,2′,3′,4′,4a′,9a′-hexahydro- 5H-spiro[oxazole-4,9′- xanthen]-2′-ol | 353.1 |
| 50 | 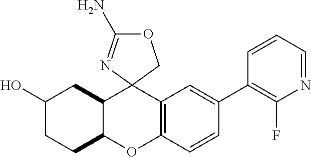 | (4a′S,9a′S)-2-amino-7′-(2- fluoropyridin-3-yl)- 1′,2′,3′,4′,4a′,9a′-hexahydro- 5H-spiro[oxazole-4,9′- xanthen]-2′-ol | 370.1 |
| 51 | 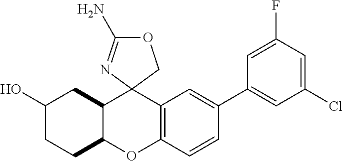 | (4a′S,9a′S)-2-amino-7′-(3- chloro-5-fluorophenyl)- 1′,2′,3′,4′,4a′,9a′-hexahydro- 5H-spiro[oxazole-4,9′- xanthen]-2′-ol | 403.1 |
| 52 | 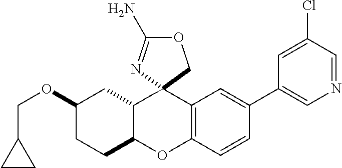 | (2′R,4S,4a′S,9a′R)-7′-(5- chloropyridin-3-yl)-2′- (cyclopropylmethoxy)- 1′,2′,3′,4′,4a′,9a′-hexahydro- 5H-spiro[oxazole-4,9′- xanthen]-2-amine | 440.1 |
| 53 | 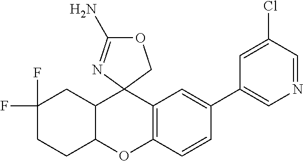 | 7′-(5-chloropyridin-3-yl)- 2′,2′-difluoro- 1′,2′,3′,4′,4a′,9a′-hexahydro- 5H-spiro[oxazole-4,9′- xanthen]-2-amine | 373.1 |
| 54 | 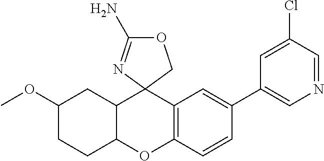 | 7′-(5-chloropyridin-3-yl)-2′- methoxy-1′,2′,3′,4′,4a′,9a′- hexahydro-5H- spiro[oxazole-4,9′-xanthen]- 2-amine | 400.1 |
| 55 | 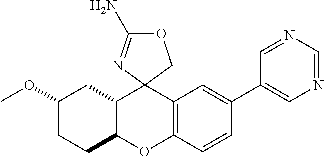 | (2′S*,4a′S*,9a′R*)-2′- methoxy-7′-(pyrimidin-5- yl)-1′,2′,3′,4′,4a′,9a′- hexahydro-5H- spiro[oxazole-4,9′-xanthen]- 2-amine TFA salt | 367.1 |
| 56 | 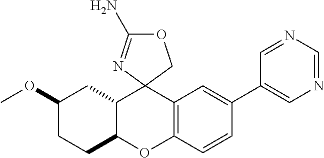 | (2′R,4a′S,9a′R)-2′-methoxy- 7′-(pyrimidin-5-yl)- 1′,2′,3′,4′,4a′,9a′-hexahydro- 5H-spiro[oxazole-4,9′- xanthen]-2-amine TFA salt (axial ether, mixture at warhead, racemic) | 367.1 |
| 57 | 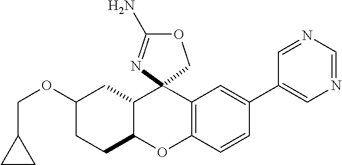 | (4R,4a′S,9a′R)-2′- (cyclopropylmethoxy)-7′- (pyrimidin-5-yl)- 1′,2′,3′,4′,4a′,9a′-hexahydro- 5H-spiro[oxazole-4,9′- xanthen]-2-amine | 407.1 |
| 58 | 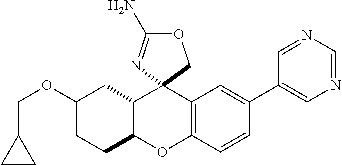 | (4S,4a′S,9a′R)-2′- (cyclopropylmethoxy)-7′- (pyrimidin-5-yl)- 1′,2′,3′,4′,4a′,9a′-hexahydro- 5H-spiro[oxazole-4,9′- xanthen]-2-amine | 407.2 |
| 59 | 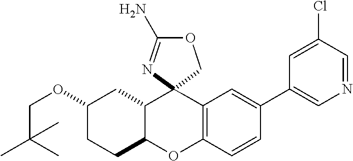 | (2′S,4R,4a′S,9a′R)-7′-(5- chloropyridin-3-yl)-2′- (neopentyloxy)- 1′,2′,3′,4′,4a′,9a′-hexahydro- 5H-spiro[oxazole-4,9′- xanthen]-2-amine | 456.1 |
| 60 | 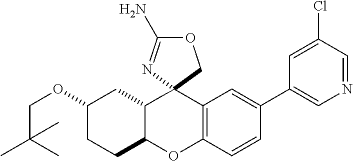 | (2′S,4S,4a′S,9a′R)-7′-(5- chloropyridin-3-yl)-2′- (neopentyloxy)- 1′,2′,3′,4′,4a′,9a′-hexahydro- 5H-spiro[oxazole-4,9′- xanthen]-2-amine | 456.1 |
| 61 | 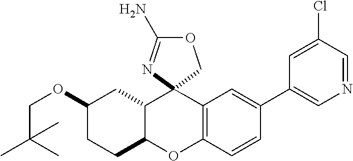 | (2′R,4R,4a′S,9a′R)-7′-(5- chloropyridin-3-yl)-2′- (neopentyloxy)- 1′,2′,3′,4′,4a′,9a′-hexahydro- 5H-spiro[oxazole-4,9′- xanthen]-2-amine | 456.1 |
| 62 | 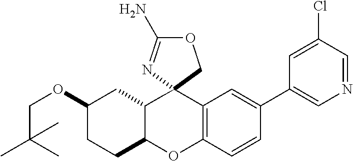 | (2′R,4S,4a′S,9a′R)-7′-(5- chloropyridin-3-yl)-2′- (neopentyloxy)- 1′,2′,3′,4′,4a′,9a′-hexahydro- 5H-spiro[oxazole-4,9′- xanthen]-2-amine | 456.1 |

### Example 63

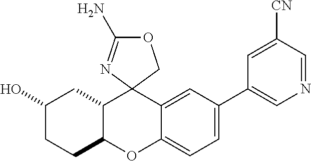

### 5-((2′S*,4a′S*,9a′R*)-2-amino-2′-hydroxy-1′,2′,3′,4′,4a′,9a′-hexahydro-5H-spiro[oxazole-4,9′-xanthen]-7′-yl)nicotinonitrile

Step A: A solution of (4a′S,9a′R)-2-amino-7′-bromo-1′,4′,4a′,9a′-tetrahydro-5H-spiro[oxazole-4,9′-xanthen]-2′(3′H)-one (25.0 mg, 0.071 mmol; Example 23, Step G), 5-(4,4,5,5-tetramethyl-1,3,2-dioxaborolan-2-yl)nicotinonitrile (17.2 mg, 0.075 mmol), Pd(PPh₃)₄ (4.1 mg, 0.0036 mmol), 2M Na₂CO₃ (107 μL, 0.214 mmol) in dioxane (356 μL) was degassed with nitrogen for 5 minutes, sealed in a vial and stirred at 80° C. for 1 day. The reaction mixture was partitioned between 4N HCl and ethyl acetate. The organic layer was extracted with 4N HCl, and the combined aqueous layers were cooled to 0° C. and basified with KOH pellets. The basic (greater than pH 10) aqueous layer was extracted with ethyl acetate (5×), and the combined organic layers were dried and concentrated to afford 5-((4a′S*,9a′R*)-2-amino-2′-oxo-1′,2′,3′,4′,4a′,9a′-hexahydro-5H-spiro[oxazole-4,9′-xanthene]-7′-yl)nicotinonitrile (17 mg, 0.045 mmol, 64% yield).

Step B: NaBH₄ (11 mg, 0.29 mmol) and MeOH (1 drop) were added to a solution of 5-((4a′S*,9a′R*)-2-amino-2′-oxo-1′,2′,3′,4′,4a′,9a′-hexahydro-5H-spiro[oxazole-4,9′-xanthene]-7′-yl)nicotinonitrile (27 mg, 0.072 mmol) in THF (721 μL) at −78° C. The resulting mixture was stirred at −78° C. for 1 hour while warming to ambient temperature. The reaction was quenched with water and then partitioned between 1N NaOH and ethyl acetate. The aqueous layer was extracted with ethyl acetate (3×), and the combined organic layers were dried and concentrated to give a residue which was purified by C18 chromatography to afford 5-((2′S*,4a′S*,9a′R*)-2-amino-7′-hydroxy-1′,2′,3′,4′,4a′,9a′-hexahydro-5H-spiro[oxazole-4,9′-xanthen]-7′-yl)nicotinonitrile trifluoroacetic acid salt as a diastereomeric mixture at the oxazoline (16 mg, 0.043 mmol, 59%). m/z (APCI-pos) M+1=377.1 (100%); Both diastereomers: ¹H NMR (CD₃OD) δ 9.08 (s, 2H), 8.85 (s, 2H), 8.50 (s, 2H), 7.94 (d, J=17 Hz, 2H), 7.69 (m, 2H), 7.03 (dd, J=17, 9 Hz, 2H), 5.23 (m, 2H), 5.01 (d, J=9 Hz, 1H), 4.68 (d, J=9 Hz, 1H), 4.05 (m, 1H), 3.92 (m, 1H), 3.83 (m, 1H), 3.74 (m, 1H), 2.30 (m, 2H), 2.17 (m, 4H), 2.09 (m, 2H), 1.70 (m, 2H), 1.57 (m, 2H), 1.40 (m, 1H), 1.26 (m, 1H).

### Example 64

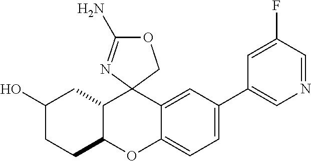

### (4a′S*,9a′R*)-2-amino-7′-(5-fluoropyridin-3-yl)-1′,2′,3′,4′,4a′,9a′-hexahydro-5,1-spiro[oxazole-4,9′-xanthen]-2′-ol

Step A: 2-Amino-7′-(5-fluoropyridin-3-yl)-1′,4′,4a′,9a′-tetrahydro-5H-spiro[oxazole-4,9′-xanthen]-2′(3′H)-one (90 mg, 0.245 mmol, 86%) was prepared according to the procedure in Example 63, Step A, substituting 5-fluoropyridin-3-ylboronic acid.

Step B: (4a′S*,9a′R*)-2-Amino-7′-(5-fluoropyridin-3-yl)-1′,2′,3′,4′,4a′,9a′-hexahydro-5H-spiro[oxazole-4,9′-xanthen]-2′-ol (100 mg, 0.271 mmol, 95%; racemic, mixture of diastereomers at oxazoline and hydroxyl) was prepared according to the procedure in Example 63, Step B, substituting 2-amino-7′-(5-fluoropyridin-3-yl)-1′,4′,4a′,9a′-tetrahydro-5H-spiro[oxazole-4,9′-xanthen]-2′(31-1)-one. m/z (APCI-pos) M+1=370.1 (100%); 4 diastereomers: ¹H NMR (CD₃OD) δ 8.58 (m, 4H), 8.36 (m, 4H), 7.58 (m, 4H), 7.39 (m, 8H), 6.94 (m, 4H), 5.23 (m, 4H), 4.68-4.05 (m, 8H), 4.05-3.70 (m, 8H), 2.60-2.30 (m, 4H), 2.17 (m, 8H), 1.70 (m, 4H), 1.40 (m, 4H), 1.40 (m, 8H).

### Example 65

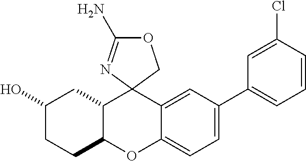

### (2′S*,4a′S*,9a′R*)-2-amino-7′-(3-chlorophenyl)-1′,2′,3′,4′,4a′,9a′-hexahydro-5H-spiro[oxazole-4,9′-xanthen]-2′-ol

(2′S*,4a′S*,9a′R*)-2-Amino-7′-(3-chlorophenyl)-1′,2′,3′,4′,4a′,9a′-hexahydro-5H-spiro[oxazole-4,9′-xanthen]-2′-ol trifluoroacetic acid salt (9.2 mg, 0.024 mmol, 24%; racemic, mixture of diastereomers at oxazoline) was prepared according to the procedure in Example 23, Step I, substituting 3-chlorophenylboronic acid. m/z (APCI-pos) M+1=385.1 (100%); Major diastereomer: ¹H NMR (CD₃OD) δ 7.79 (d, J=2.0 Hz, 1H), 7.64 (t, J=2.0 Hz, 1H), 7.54 (m, 2H), 7.40 (t, J=7.8 Hz, 1H), 7.32 (m, 1H), 6.93 (d, J=8.6 Hz, 1H), 5.20 (d, J=9.8 Hz, 1H), 4.67 (d, J=9.8 Hz, 1H), 4.01 (td, J=11, 5 Hz, 1H), 3.73 (m, 1H), 2.31 (m, 1H), 2.16 (m, 1H), 2.06 (m, 2H), 1.69 (m, 1H), 1.50 (m, 1H), 1.38 (m, 1H).

### Example 66

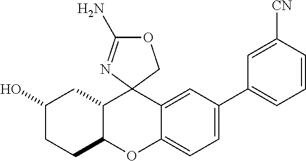

### 3-((2′S*,4a′S*,9a′R*)-2-amino-2′-hydroxy-1′,2′,3′,4′,4a′,9a′-hexahydro-5H-spiro[oxazole-4,9′-xanthen]-7′-yl)benzonitrile

3-((2′S*,4a′S*,9a′R*)-2-Amino-2′-hydroxy-1′,2′,3′,4′,4a′,9a′-hexahydro-5H-spiro[oxazole-4,9′-xanthen]-7′-yl)benzonitrile trifluoroacetic acid salt (7.8 mg, 0.021 mmol, 21%; racemic, mixture of diastereomers at oxazoline) was prepared according to the procedure in Example 23, Step I, substituting 3-cyanophenylboronic acid. m/z (APCI-pos) M+1=376.1 (100%); Major diastereomer: ¹H NMR (CDCl₃) δ 7.80 (s, 1H), 7.74 (m, 1H), 7.59 (m, 1H), 7.53 (m, 2H), 7.44 (m, 1H), 6.92 (d, J=8.6 Hz, 1H), 4.86 (d, J=9.6 Hz, 1H), 4.50 (d, J=9.4 Hz, 1H), 3.86 (td, J=11, 5 Hz, 1H), 2.31 (m, 1H), 2.21 (m, 1H), 2.11 (m, 1H), 2.06 (m, 2H), 1.64 (m, 1H), 1.43 (m, 1H), 1.25 (m, 1H).

### Example 67

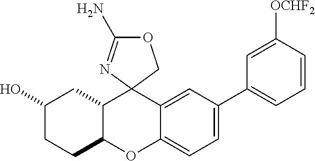

### (2′S*,4a′S*,9aR*)-2-amino-7′-(3-(difluoromethoxy)phenyl)-1′,2′,3′,4′,4a′,9a′-hexahydro-5H-spiro[oxazole-4,9′-xanthen]-2′-ol

(2′S*,4a′S*,9a′R*)-2-Amino-7′-(3-(difluoromethoxy)phenyl)-1′,2′,3′,4′,4a′,9a′-hexahydro-5H-spiro[oxazole-4,9′-xanthen]-2′-ol trifluoroacetic acid salt (17 mg, 0.041 mmol, 41%; racemic, mixture of diastereomers at oxazoline) was prepared according to the procedure in Example 23, Step I, substituting 3-(difluoromethoxy)phenylboronic acid. m/z (APCI-pos) M+1=417.1 (100%); Major diastereomer: ¹H NMR (CD₃OD) δ 7.78 (d, J=2.3 Hz, 1H), 7.56 (dd, J=8.2, 2.0 Hz, 1H), 7.46 (m, 3H), 7.37 (m, 1H), 7.10 (m, 1H), 6.93 (d, J=8.6 Hz, 1H), 5.20 (d, J=9.8 Hz, 1H), 4.67 (d, J=9.8 Hz, 1H), 4.01 (td, J=11, 5.1 Hz, 1H), 3.73 (m, 1H), 2.31 (m, 1H), 2.15 (m, 1H), 2.04 (m, 2H), 1.69 (m, 1H), 1.53 (m, 1H), 1.38 (m, 1H).

### Example 68

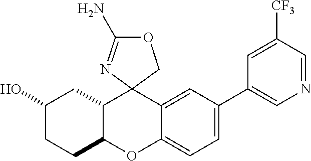

### (2′S*,4a′S*,9a′R*)-2-amino-7′-(5-(trifluoromethyl)pyridin-3-yl)-1′,2′,3′,4′,4a′,9a′-hexahydro-5H-spiro[oxazole-4,9′-xanthen]-2′-ol

(2′S*,4a′S*,9a′R*)-2-Amino-7′-(5-(trifluoromethyl)pyridin-3-yl)-1′,2′,3′,4′,4a′,9a′-hexahydro-5H-spiro[oxazole-4,9′-xanthen]-2′-ol trifluoroacetic acid salt (4.5 mg, 0.011 mmol, 11%; racemic, mixture of diastereomers at oxazoline) was prepared according to the procedure in Example 23, Step I, substituting 5-(trifluoromethyl)pyridin-3-ylboronic acid. m/z (APCI-pos) M+1=420.1 (100%); Two diastereomers: ¹H NMR (CD₃OD) δ 9.07 (s, 2H), 8.82 (s, 2H), 8.38 (s, 2H), 7.97 (d, J=2.3 Hz, 1H), 7.93 (d, J=2.0 Hz, 1H), 7.69 (m, 2H), 7.05 (d, J=8.6 Hz, 1H), 7.00 (d, J=8.6 Hz, 1H), 5.24 (d, J=10.2 Hz, 1H), 5.21 (d, J=10.2 Hz, 1H), 4.99 (d, J=10.2 Hz, 1H), 4.69 (d, J=9.9 Hz, 1H), 4.04 (td, J=9.8, 5.0 Hz, 1H), 3.90 (td, J=9.8, 5.0 Hz, 1H), 3.82 (m, 1H), 3.73 (m, 1H), 2.31 (m, 2H), 2.15 (m, 4H), 2.04 (m, 2H), 1.69 (m, 2H), 1.50 (m, 1H), 1.39 (m, 2H), 1.25 (m, 1H).

### Example 69

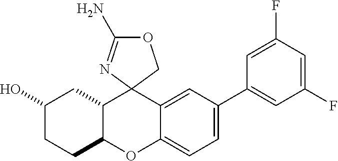

### (2′S*,4a′S*,9a′R*)-2-amino-7′-(3,5-difluorophenyl)-1′,2′,3′,4′,4a′,9a′-hexahydro-5H-spiro[oxazole-4,9′-xanthen]-2′-ol

(2′S*,4a′S*,9a′R*)-2-Amino-7′-(3,5-difluorophenyl)-1′,2′,3′,4′,4a′,9a′-hexahydro-5H-spiro[oxazole-4,9′-xanthen]-2′-ol trifluoroacetic acid salt (7.5 mg, 0.016 mmol, 16%; racemic, mixture of diastereomers at oxazoline) was prepared according to the procedure in Example 23, Step I, substituting 3,5-difluorophenylboronic acid. m/z (APCI-pos) M+1=387.1 (100%).

### Example 70

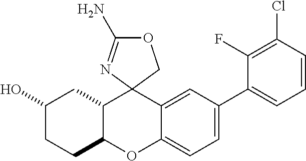

### (2′S*,4a′S*,9a′R*)-2-amino-7′-(3-chloro-2-fluorophenyl)-1′,2′,3′,4′,4a′,9a′-hexahydro-5H-spiro[oxazole-4,9′-xanthen]-2′-ol

(2′S*,4a′S*,9a′R*)-2-Amino-7′-(3-chloro-2-fluorophenyl)-1′,2′,3′,4′,4a′,9a′-hexahydro-5H-spiro[oxazole-4,9′-xanthen]-2′-ol trifluoroacetic acid salt (4.0 mg, 0.008 mmol, 8%; racemic, mixture of diastereomers at oxazoline) was prepared according to the procedure in Example 23, Step I, substituting 3-chloro-2-fluorophenylboronic acid. m/z (APCI-pos) M+1=403.1 (100%); Two diastereomers: ¹H NMR (CD₃OD) δ 7.69 (m, 1H), 7.64 (m, 1H), 7.50-7.38 (m, 6H), 7.23 (m, 2H), 7.00 (d, J=8.6 Hz, 1H), 6.95 (d, J=8.6 Hz, 1H), 5.20 (d, J=10.2 Hz, 1H), 5.11 (d, J=9.8 Hz, 1H), 4.99 (d, J=9.8 Hz, 1H), 4.64 (d, J=9.8 Hz, 1H), 4.03 (td, J=10.5, 5.0 Hz, 1H), 3.89 (td, J=10.5, 5.0 Hz, 1H), 3.82 (m, 1H), 3.73 (m, 1H), 2.32 (m, 2H), 2.16 (m, 4H), 2.04 (m, 2H), 1.69 (m, 2H), 1.50 (m, 1H), 1.38 (m, 2H), 1.25 (m, 1H).

### Example 71

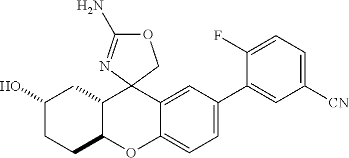

### 3-((2′S*,4a′S*,9a′R*)-2-amino-2′-hydroxy-1′,2′,3′,4′,4a′,9a′-hexahydro-5H-spiro[oxazole-4,9′-xanthen]-7′-yl)-4-fluorobenzonitrile

3-((2′S*,4a′S*,9a′R*)-2-amino-7′-hydroxy-1′,2′,3′,4′,4a′,9a′-hexahydro-5H-spiro[oxazole-4,9′-xanthen]-7′-yl)-4-fluorobenzonitrile trifluoroacetic acid salt (5 mg, 0.01 mmol, 10%; racemic, mixture of diastereomers at oxazoline) was prepared according to the procedure in Example 23, Step I, substituting 5-cyano-2-fluorophenylboronic acid. m/z (APCI-pos) M+1=394.1 (100%); Single diastereomer: ¹H NMR (CD₃OD) δ 7.94 (dd, J=7.4, 2.3 Hz, 1H), 7.76 (octet, J=2.0 Hz, 1H), 7.71 (m, 1H), 7.53 (dt, J=8.6, 2.0 Hz, 1H), 7.43 (dd, J=10.6, 8.6 Hz, 1H), 7.01 (d, J=8.6 Hz, 1H), 5.14 (d, J=9.8 Hz, 1H), 4.99 (d, J=10.2 Hz, 1H), 3.90 (td, J=11, 5.0 Hz, 1H), 3.82 (m, 1H), 2.32 (m, 1H), 2.18 (m, 2H), 2.10 (m, 1H), 1.69 (m, 1H), 1.41 (m, 1H), 1.25 (m, 1H).

### Example 72

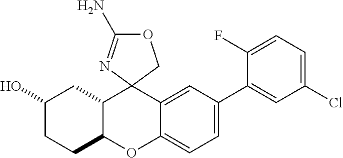

### (2′S*,4a′S*,9a′R*)-2-amino-7′-(5-chloro-2-fluorophenyl)-1′,2′,3′,4′,4a′,9a′-hexahydro-5H-spiro[oxazole-4,9′-xanthen]-2′-ol

(2′S*,4a′S*,9a′R*)-2-Amino-7′-(5-chloro-2-fluorophenyl)-1′,2′,3′,4′,4a′,9a′-hexahydro-5H-spiro[oxazole-4,9′-xanthen]-2′-ol trifluoroacetic acid salt (3 mg, 0.006 mmol, 6%; racemic, mixture of diastereomers at oxazoline) was prepared according to the procedure in Example 23, Step I, substituting 5-chloro-2-fluorophenylboronic acid. m/z (APCI-pos) M+1=403.1 (100%); Major diastereomer: ¹H NMR (CD₃OD) δ 7.65 (m, 1H), 7.50 (m, 2H), 7.35 (m, 1H), 7.19 (dd, J=10.2, 8.6 Hz, 1H), 6.99 (d, J=8.6 Hz, 1H), 5.12 (d, J=9.8 Hz, 1H), 4.99 (d, J=9.8 Hz, 1H), 3.88 (td, J=11, 5.0 Hz, 1H), 3.82 (m, 1H), 2.31 (m, 1H), 2.17 (m, 2H), 2.10 (m, 1H), 1.69 (m, 1H), 1.41 (m, 1H), 1.24 (m, 1H).

### Example 73

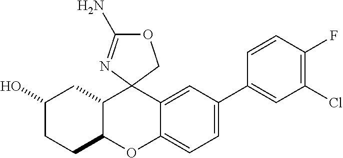

### (2′S*,4a′S*,9a′R*)-2-amino-7′-(3-chloro-4-fluorophenyl)-1′,2′,3′,4′,4a′,9a′-hexahydro-5H-spiro[oxazole-4,9′-xanthen]-2′-ol

(2′S*,4a′S*,9a′R*)-2-Amino-7′-(3-chloro-4-fluorophenyl)-1′,2′,3′,4′,4a′,9a′-hexahydro-5H-spiro[oxazole-4,9′-xanthen]-2′-ol trifluoroacetic acid salt (6 mg, 0.012 mmol, 12%; racemic, mixture of diastereomers at oxazoline) was prepared according to the procedure in Example 23, Step I, substituting 3-chloro-4-fluorophenylboronic acid. m/z (APCI-pos) M+1=403.1 (100%); Two diastereomers: ¹H NMR (CD₃OD) δ 7.74 (m, 4H), 7.55 (m, 2H), 7.29 (m, 2H), 6.95 (m, 2H), 5.12 (m, 2H), 4.99 (d, J=9.8 Hz, 1H), 4.67 (d, J=9.8 Hz, 1H), 4.01 (td, J=11, 5.0 Hz, 1H), 3.87 (td, J=11, 5.0 Hz, 1H), 3.81 (m, 1H), 3.73 (m, 1H), 2.31 (m, 2H), 2.14 (m, 4H), 2.03 (m, 2H), 1.69 (m, 2H), 1.52 (m, 1H), 1.38 (m, 2H), 1.25 (m, 1H).

### Example 74

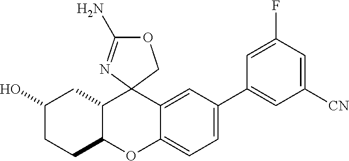

### 3-((2′S,4a′S,9a′R)-2-amino-2′-hydroxy-1′,2′,3′,4′,4a′,9a′-hexahydro-5H-spiro[oxazole-4,9′-xanthen]-7′-yl)-5-fluorobenzonitrile

3-((2′S,4a′S,9a′R)-2-Amino-2′-hydroxy-1′,2′,3′,4′,4a′,9a′-hexahydro-5H-spiro[oxazole-4,9′-xanthen]-7′-yl)-5-fluorobenzonitrile trifluoroacetic acid salt (8 mg, 0.016 mmol, 16%; 5:1 mixture of diastereomers at oxazoline, racemic) was prepared according to the procedure in Example 23, Step I, substituting 3-cyano-5-fluorophenylboronic acid. m/z (APCI-pos) M+1=394.1 (100%). Major diastereomer: ¹H NMR (CD₃OD) δ 7.93 (d, J=2.3 Hz, 1H), 7.90 (t, J=1.6 Hz, 1H), 7.77 (ddd, J=10.2, 2.3, 1.6 Hz, 1H), 7.64 (dd, J=8.6, 2.3 Hz, 1H), 7.50 (dq, J=8.6, 1.2 Hz, 1H), 6.97 (d, J=8.6 Hz, 1H), 5.20 (d, J=9.8 Hz, 1H), 4.68 (d, J=9.8 Hz, 1H), 4.04 (td, J=11, 5.1 Hz, 1H), 3.73 (m, 1H), 2.32 (m, 1H), 2.13 (m, 2H), 2.03 (m, 1H), 1.69 (m, 1H), 1.50 (m, 1H), 1.39 (m, 1H).

### Example 75

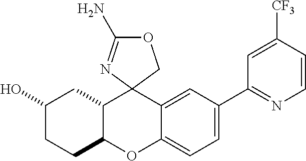

### (2′S,4a′S,9a′R)-2-amino-7′-(4-(trifluoromethyl)pyridin-2-yl)-1′,2′,3′,4′,4a′,9a′-hexahydro-5H-spiro[oxazole-4,9′-xanthen]-2′-ol

Step A: A solution of (4a′S,9a′R)-2-amino-7′-bromo-1′,2′,3′,4′,4a′,9a′-hexahydro-5H-spiro[oxazole-4,9′-xanthen]-2′-ol (Example 23, Step H, 0.38 g, 1.08 mmol) in DMF (5.4 mL) was treated with 4,4,4′,4′,5,5,5′,5′-octamethyl-2,2′-bi(1,3,2-dioxaborolane) (0.68 g, 2.69 mmol), PdCl₂(dppf)*DCM (0.044 g, 0.054 mmol) and KOAc (0.32 g, 3.23 mmol) while purging with nitrogen for 15 minutes in a pressure tube. The mixture was soniccated, then further purged with nitrogen, and then sealed and heated at 90° C. for 4 hours. The mixture was concentrated in vacuo, diluted with DCM, and filtered to remove solids, washing with DCM. The filtrate was purified by flash chromatography eluting with a gradient of 2-10% MeOH in DCM. The major product-containing fraction was concentrated to afford (4a′S,9a′R)-2-amino-7′-(4,4,5,5-tetramethyl-1,3,2-dioxaborolan-2-yl)-1′,2′,3′,4′,4a′,9a′-hexahydro-5H-spiro[oxazole-4,9′-xanthen]-2′-ol (103 mg, 0.257 mmol, 24%).

Step B: A solution of (4a′S,9a′R)-2-amino-7′-(4,4,5,5-tetramethyl-1,3,2-dioxaborolan-2-yl)-1′,2′,3′,4′,4a′,9a′-hexahydro-5H-spiro[oxazole-4,9′-xanthen]-2′-ol (0.025 g, 0.062 mmol), 2-bromo-4-(trifluoromethyl)pyridine (0.017 g, 0.075 mmol), Pd(PPh₃)₄ (0.004 g, 0.003 mmol), Na₂CO₃ (2.0 M aq, 0.10 mL, 0.19 mmol) in dioxane (0.62 mL) was degassed with nitrogen for 5 minutes, sealed in a vial and stirred at 80° C. for 1 day. The reaction mixture was filtered through GF/F paper, washing with methanol. The filtrate was concentrated, redissolved in MeOH and purified by C18 semi-prep HPLC eluting with 5-75% ACN/H₂O+0.1% TFA to afford (2′S,4a′S,9a′R)-2-amino-7′-(4-(trifluoromethyl)pyridin-2-yl)-1′,2′,3′,4′,4a′,9a′-hexahydro-5H-spiro[oxazole-4,9′-xanthen]-2′-ol trifluoroacetic acid salt: (6 mg, 0.010 mmol, 16%; racemic mixture of 2:1 mixture of diastereomers at oxazoline). m/z (APCI-pos) M+1=420.1 (100%).

Major diastereomer: ¹H NMR (CD₃OD) δ 8.83 (m, 1H), 8.33 (m, 1H), 8.04 (m, 1H), 7.59 (m, 1H), 6.99 (d, J=8.6 Hz, 1H), 5.23 (d, J=9.8 Hz, 1H), 4.69 (d, J=9.8 Hz, 1H), 4.06 (td, J=11, 5.0 Hz, 1H), 3.74 (m, 1H), 2.33 (m, 1H), 2.17 (m, 2H), 2.10 (m, 1H), 1.70 (m, 1H), 1.51 (m, 1H), 1.41 (m, 1H).

Minor diastereomer: ¹H NMR (CD₃OD) δ 8.84 (m, 1H), 8.30 (m, 1H), 8.04 (m, 1H), 7.59 (m, 1H), 7.03 (d, J=8.6 Hz, 1H), 5.23 (d, J=9.8 Hz, 1H), 5.02 (d, J=9.8 Hz, 1H), 3.92 (td, J=11, 5.0 Hz, 1H), 3.83 (m, 1H), 2.33 (m, 1H), 2.17 (m, 2H), 2.10 (m, 1H), 1.70 (m, 1H), 1.41 (m, 1H), 1.26 (m, 1H).

### Example 76

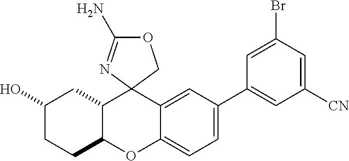

### 3-((2′S,4a′S,9a′R)-2-amino-2′-hydroxy-1′,2′,3′,4′,4a′,9a′-hexahydro-5H-spiro[oxazole-4,9′-xanthene]-7′-yl)-5-bromobenzonitrile

3-((2′S,4a′S,9a′R)-2-Amino-2′-hydroxy-1′,2′,3′,4′,4a′,9a′-hexahydro-5H-spiro[oxazole-4,9′-xanthene]-7′-yl)-5-bromobenzonitrile trifluoroacetic acid salt (3 mg, 0.006 mmol, 6%; racemic mixture of 2:1 mixture of diastereomers at oxazoline) was prepared according to the procedure in Example 75, Step B, substituting 3,5-dibromobenzonitrile. m/z (APCI-pos) M+1=454.0 (100%), 456.0 (100%). Major diastereomer: ¹H NMR (CD₃OD) δ 8.14 (m, 1H), 8.03 (m, 1H), 7.90 (m, 1H), 7.62 (m, 1H), 6.97 (d, J=8.6 Hz, 1H), 5.22 (d, J=9.8 Hz, 1H), 4.68 (d, J=9.8 Hz, 1H), 4.04 (td, J=11, 5.0 Hz, 1H), 3.72 (m, 1H), 2.32 (m, 1H), 2.17 (m, 2H), 2.10 (m, 1H), 1.69 (m, 1H), 1.50 (m, 1H), 1.39 (m, 1H). Minor diastereomer: ¹H NMR (CD₃OD) δ 8.14 (m, 1H), 8.03 (m, 1H), 7.88 (m, 1H), 7.64 (m, 1H), 7.01 (d, J=8.6 Hz, 1H), 5.23 (d, J=9.8 Hz, 1H), 5.00 (d, J=9.8 Hz, 1H), 3.89 (td, J=11, 5.0 Hz, 1H), 3.81 (m, 1H), 2.32 (m, 1H), 2.17 (m, 2H), 2.10 (m, 1H), 1.69 (m, 1H), 1.50 (m, 1H), 1.25 (m, 1H).

### Example 77

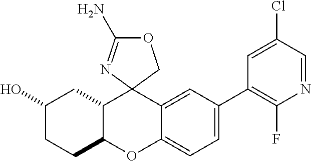

### (2′S,4a′S,9a′R)-2-amino-7′-(5-chloro-2-fluoropyridin-3-yl)-1′,2′,3′,4′,4a′,9a′-hexahydro-5H-spiro[oxazole-4,9′-xanthen]-2′-ol

(2′S,4a′S,9a′R)-2-Amino-7′-(5-chloro-2-fluoropyridin-3-yl)-1′,2′,3′,4′,4a′,9a′-hexahydro-5H-spiro[oxazole-4,9′-xanthen]-2′-ol trifluoroacetic acid salt (4 mg, 0.007 mmol, 7%; racemic, single diastereomer) was prepared according to the procedure in Example 23, Step I, substituting 5-chloro-2-fluoropyridin-3-ylboronic acid. m/z (APCI-pos) M+1=404.0 (100%). Single diastereomer: ¹H NMR (CD₃OD) [symbol] 08.16 (m, 1H), 8.12 (dd, J=8.2, 2.3 Hz, 1H), 7.77 (m, 1H), 7.56 (dt, J=8.6, 2.0 Hz, 1H), 7.01 (d, J=9.0 Hz, 1H), 5.14 (d, J=10.2 Hz, 1H), 4.99 (d, J=10.2 Hz, 1H), 3.90 (td, J=11, 5.1 Hz, 1H), 3.82 (m, 1H), 2.32 (m, 1H), 2.18 (m, 2H), 2.10 (m, 1H), 1.70 (m, 1H), 1.42 (m, 1H), 1.25 (m, 1H).

### Example 78

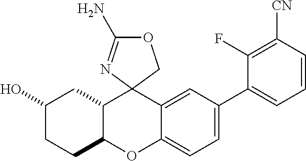

### 3-((2′S,4a′S,9a′R)-2-amino-2′-hydroxy-1′,2′,3′,4′,4a′,9a′-hexahydro-5H-spiro[oxazole-4,9′-xanthen]-7′-yl)-2-fluorobenzonitrile

3-((2′S,4a′S,9a′R)-2-Amino-2′-hydroxy-1′,2′,3′,4′,4a′,9a′-hexahydro-5H-spiro[oxazole-4,9′-xanthen]-7′-yl)-2-fluorobenzonitrile trifluoroacetic acid salt (7 mg, 0.013 mmol, 14%; racemic, 1:1 mixture of diastereomers at oxazoline) was prepared according to the procedure in Example 23 Step I, substituting 3-cyano-2-fluorophenylboronic acid. m/z (APCI-pos) M+1=394.1 (100%). Two diastereomers: ¹H NMR (CD₃OD) δ 7.82 (m, 2H), 7.73 (m, 2H), 7.51 (m, 1H), 7.43 (m, 1H), 7.22 (m, 2H), 7.13 (m, 2H), 7.02 (d, J=8.6 Hz, 1H), 6.98 (d, J=8.6 Hz, 1H), 5.21 (d, J=9.8 Hz, 1H), 5.12 (d, J=9.8 Hz, 1H), 5.00 (d, J=9.8 Hz, 1H), 4.64 (d, J=9.8 Hz, 1H), 4.04 (td, J=11, 5.0 Hz, 1H), 3.90 (td, J=11, 5.0 Hz, 1H), 3.82 (m, 1H), 3.73 (m, 1H), 2.32 (m, 2H), 2.17 (m, 4H), 2.05 (m, 2H), 1.70 (m, 2H), 1.51 (m, 1H), 1.41 (m, 2H), 1.24 (m, 1H).

### Example 79

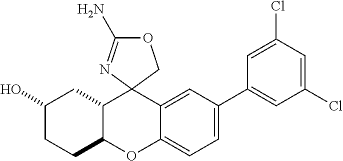

### (2′S,4a′S,9a′R)-2-amino-7′-(3,5-dichlorophenyl)-1′,2′,3′,4′,4a′,9a′-hexahydro-5H-spiro[oxazole-4,9′-xanthen]-2′-ol

Step A: (2′S,4a′S,9a′R)-2-amino-7′-(3,5-dichlorophenyl)-1′,2′,3′,4′,4a′,9a′-hexahydro-5H-spiro[oxazole-4,9′-xanthen]-2′-ol trifluoroacetic acid salt (7 mg, 0.013 mmol, 14%) was prepared according to the procedure in Example 23, Step I, substituting 3,5-dichlorophenylboronic acid. m/z (APCI-pos) M+1=419.1 (100%), 421.0 (60%). Major diastereomer: ¹H NMR (CD₃OD) δ 7.78 (d, J=2.3 Hz, 1H), 7.60 (d, J=2.0 Hz, 2H), 7.58 (m, 1H), 7.40 (t, J=2.0 Hz, 1H), 6.99 (d, J=8.6 Hz, 1H), 5.21 (d, J=10 Hz, 1H), 4.99 (d, J=10 Hz, 1H), 3.88 (td, J=11, 5.0 Hz, 1H), 3.82 (m, 1H), 2.31 (m, 1H), 2.17 (m, 2H), 2.05 (m, 1H), 1.69 (m, 1H), 1.41 (m, 1H), 1.25 (m, 1H). Minor diastereomer: ¹H NMR (CD₃OD) δ 7.85 (d, J=2.3 Hz, 1H), 7.61 (d, J=2.0 Hz, 2H), 7.58 (m, 1H), 7.39 (t, J=2.0 Hz, 1H), 6.94 (d, J=8.6 Hz, 1H), 5.20 (d, J=10 Hz, 1H), 4.67 (d, J=10 Hz, 1H), 4.03 (td, J=11, 5.0 Hz, 1H), 3.73 (m, 1H), 2.31 (m, 1H), 2.17 (m, 2H), 2.05 (m, 1H), 1.69 (m, 1H), 1.52 (m, 1H), 1.41 (m, 1H).

### Example 80

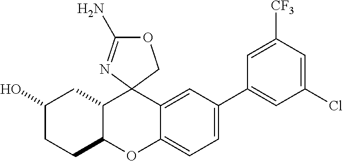

### (2′S*,4a′S*,9a′R*)-2-amino-7-(3-chloro-5-(trifluoromethyl)phenyl)-1′,2′,3′,4′,4a′,9a′-hexahydro-5H-spiro[oxazole-4,9′-xanthen]-2′-ol

(2′S*,4a′S*,9a′R*)-2-Amino-7′-(3-chloro-5-(trifluoromethyl)phenyl)-1′,2′,3′,4′,4a′,9a′-hexahydro-5H-spiro[oxazole-4,9′-xanthen]-2′-ol trifluoroacetic acid salt (4 mg, 0.007 mmol, 7%) was prepared according to the procedure in Example 23, Step I, substituting 3-chloro-5-(trifluoromethyl)phenylboronic acid. m/z (APCI-pos) M+1=453.1 (100%).

### Example 81

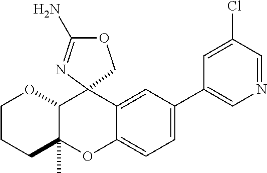

### (4R*,4a′S*,10a′R*)-8′-(5-chloropyridin-3-yl)-4a′-methyl-3′,4′,4a′,10a′-tetrahydro-2′H,5H-spiro[oxazole-4,10′-pyrano[3,2-b]chromen]-2-amine

Step A: A solution of 5-hydroxypentan-2-one (65.7 mL, 644 mmol) and imidazole (65.7 g, 965 mmol) in DCM (600 mL) was cooled in an ice bath and treated dropwise by addition funnel with a solution of TBDMS-Cl (97 g, 644 mmol) in DCM (500 mL) over a 1 hour time period. The ice bath was removed, and the reaction was allowed to come to room temperature and stirring continued for 1 hour. The reaction was washed with 1N aqueous HCl (1 L), water (1 L), then saturated aqueous. NaHCO₃ (1 L) and dried over Na₂SO₄. The product, 5-((tert-butyldimethylsilyl)oxy)pentan-2-one (116.7 g, 67%), was clean enough crude to take forward without further purification.

Step B: A round bottomed flask plus stir bar was charged with 1-(2-hydroxy-5-methoxyphenyl)ethanone (72.9 g, 439 mmol), 5-((tert-butyldimethylsilyl)oxy)pentan-2-one (86.3 g, 399 mmol), EtOH (500 mL) and pyrrolidine (31.2 g, 439 mmol). The reaction mixture was heated to 80° C. for 18 hours with stirring, with attached water reflux condenser. After cooling to room temperature, the reaction mixture was transferred to a separatory funnel with diethyl ether (500 mL). The mixture was washed with 1N aqueous NaOH (500 mL), and the aqueous phase was re-extracted with diethyl ether (150 mL). The combined organics were washed with 1N aqueous HCl (500 mL), re-extracting aqueous with diethyl ether (150 mL), then washed combined organics with saturated aqueous NaHCO₃ (500 mL), dried (MgSO₄), filtered, and concentrated to yield 2-(3-((tert-butyldimethylsilyl)oxy)propyl)-6-methoxy-2-methylchroman-4-one (117 g, 65%).

Step C: A round bottomed flask plus stir bar was charged with ethyl formate (155 mL, 1926 mmol), diethyl ether (600 mL) and sodium methoxide (86.7 g, 1605 mmol) at 0° C. The reaction mixture was stirred for 20 minutes. Next, 2-(3-((tert-butyldimethylsilyl)oxy)propyl)-6-methoxy-2-methylchroman-4-one (117 g, 321 mmol) dissolved in diethyl ether (200 mL) was added by canula over a 30 minutes period with vigorous stirring. The mixture was removed from the bath and stirred at room temperature for 3 hours. The reaction mixture was worked up by recooling to 0° C., and carefully adding saturated aqueous NH₄Cl (500 mL) in small portions maintaining internal temperature below 15° C. The contents were transferred to a separatory funnel, rinsing with diethyl ether. The phases were separated, and the aqueous was re-extracting with diethyl ether (200 mL). The combined organics were dried (MgSO₄), filtered, and concentrated to yield (E)-2-(3-((tert-butyldimethylsilyl)oxy)propyl)-3-(hydroxymethylene)-6-methoxy-2-methylchroman-4-one (130 g, 62%).

Step D: Diethylamine (45.1 g, 616 mmol) was added to a solution of (E)-2-(3-((tert-butyldimethylsilyl)oxy)propyl)-3-(hydroxymethylene)-6-methoxy-2-methylchroman-4-one (121 g, 308 mmol) and naphthalene-2-sulfonyl azide (79.1 g, 339 mmol, prepared according to the procedure described for 4-methylbenzenesulfonyl azide in WO 2010/011147, but replacing 4-methylbenzenesulfonyl chloride with naphthalene-2-sulfonyl chloride, and replacing DCM with EtOAc during the workup) in Et₂O (600 mL) while cooled in an ice bath. The reaction mixture was left in the ice bath to warm up slowly, while stirring under N₂. The reaction mixture was then stirred at room temperature for 18 hours. The reaction mixture was filtered to remove most of the sulfonamide by-product, and concentrated in vacuo. The crude was partially purified by Biotage Flash 75 L silica gel chromatography, eluting with DCM, then 2% MeOH/DCM. The mixed fractions were pooled and chromatographed as before to yield 2-(3-((tert-butyldimethylsilyl)oxy)propyl)-3-diazo-6-methoxy-2-methylchroman-4-one (58 g, 29%).

Step E: A round bottomed flask plus stir bar was charged with 2-(3-((tert-butyldimethylsilyl)oxy)propyl)-3-diazo-6-methoxy-2-methylchroman-4-one (58 g, 149 mmol), THF (150 mL), and TBAF (1M in THF, 223 mL, 223 mmol). The mixture was cooled in an ice bath during addition of TBAF, which was added quickly with no visible exotherm. The mixture was stirred at room temperature for 3 hours. As unreacted starting material remained by TLC analysis, more TBAF (75 mL) was added and continued stirring for 2 hours at room temperature. The mixture was worked up by pardoning between EtOAc (250 mL) and water (250 mL). The phases were separated, and the aqueous was re-extracted with EtOAc (250 mL). The combined organic phases were washed again with water (250 mL), brine (250 mL), dried (MgSO₄), filtered, and concentrated. The crude was purified by Biotage Flash 75 L silica gel chromatography eluting with DCM, then 3% MeOH/DCM to fully elute products and yield 3-diazo-2-(3-hydroxypropyl)-6-methoxy-2-methylchroman-4-one (33.3 g, 61%).

Step F: A round bottomed flask plus stir bar was charged with 3-diazo-2-(3-hydroxypropyl)-6-methoxy-2-methylchroman-4-one (17.7 g, 64.1 mmol) and anhydrous toluene (180 mL). The mixture was degassed with N₂ for 10 minutes, and then rhodium(II) acetate dimer (1.02 g, 2.31 mmol) was added. Immediately submerged reaction vessel into a pre-heated oil bath at 90° C. with stirring under a stream of N₂. The oil bath was removed after gas evolution ceased (approximately 5-10 minutes). After cooling to room temperature, combined this reaction crude with previous reaction crudes obtained in the same manner which totaled 23.9 g. The combined crudes were filtered through Celite®, rinsing with DCM. The filtrate was concentrated. The crudes were purified by Biotage Flash 75 L silica gel chromatography eluting with 30% EtOAc/hexanes, then 1:1 EtOAc/hexanes to fully elute product and yield (4aS*,10aS*)-8-methoxy-4a-methyl-2,3,4,4a-tetrahydropyrano[3,2-b]chromen-10(10aH)-one (15.2 g, 32%).

Step G: A round bottomed flask plus stir bar was charged with (4aS*,10aS*)-8-methoxy-4a-methyl-2,3,4,4a-tetrahydropyrano[3,2-b]chromen-10(10aH)-one (5.0 g, 20 mmol) and anhydrous THF (30 mL). The mixture was cooled to 0° C. under N₂, and Tebbe reagent (60 mL, 30 mmol) was added dropwise. The mixture was stirred for 1 hour in the ice bath. Aqueous 30% Rochelle's salt (75 mL) was very slowly added to the mixture while stirring in an ice bath. The internal temperature was maintained below 35° C. DCM (50 mL) was added to the mixture to maintain stirring during the addition. The biphasic suspension was stirred for 18 hours at room temperature. The biphase was filtered to remove solids through Celite®, rinsing with DCM. The phases were separated, and the aqueous phase was re-extracting with DCM (100 mL). The combined organics were washed with brine (100 mL), dried (MgSO₄), filtered, and concentrated. The crude was purified by Biotage Flash 65 silica gel chromatography, eluting with a gradient of 10% EtOAc/hexanes to 1:1 EtOAc/hexanes to yield (4aS*,10aR*)-8-methoxy-4a-methyl-10-methylene-2,3,4,4a,10,10a-hexahydropyrano[3,2-b]chromene (3.2 g, 63%).

Step H: Silver cyanate (5.84 g, 39.0 mmol), CH₃CN (10 mL) and THF (10 mL) were added to a round bottomed flask plus stir bar was added. The reaction mixture was cooled in an ice bath under N₂. Iodine (8.24 g, 32.5 mmol) was added. The mixture was stirred for 15 minutes at 0° C. Then (4aS*,10aR*)-8-methoxy-4a-methyl-10-methylene-2,3,4,4a,10,10a-hexahydropyrano[3,2-b]chromene (3.2 g, 13.0 mmol) in THF (30 mL) was added. The mixture was stirred in the ice bath for 5 minutes and then at room temperature for 2 hours. The suspension was filtered, rinsing with THF. The mixture was recooled in an ice bath, and then aqueous NH₄OH (9 mL) was added directly to the filtrate. The filtrate was stirred at room temperature for 20 hours. The reaction mixture was partioned between EtOAc (50 mL) and aqueous saturated sodium thiosulfate solution (50 mL). After shaking to remove dark color, the phases were separated. The aqueous phase was reextracted with EtOAc (50 mL). The combined organics were washed with brine (50 mL), dried (MgSO₄), filtered, and concentrated. The crude was then stirred with 2N aqueous HCl (50 mL) for 15 minutes, and the insoluble solids (isourea side product) were filtered. The solids were suspended a second time in 2N aqueous HCl (30 mL), stirred for 15 minutes again, and re-filtered, rinsing with 2N aqueous HCl. The combined HCl extracts (approx 100 mL volume) were cooled in an ice bath and were neutralized to a pH of about 7 to 8 while stirring with NaOH pellets added in portions over a 30 minute period. Substantial solid formed. The desired product was extracted with DCM (3×50 mL). The combined organic phases were washed with brine (100 mL), dried (MgSO₄), filtered and concentrated to yield (4R*,4a′S*,10a′R*)-2-amino-4a′-methyl-3′,4′,4a′,10a′-tetrahydro-2′H,5H-spiro[oxazole-4,10′-pyrano[3,2-b]chromen]-8′-yl trifluoromethanesulfonate (1.6 g, 38%), which was not separated from its diastereomer (1:2 ratio of desired diastereomer to undesired) at this step.

Step I: A round bottomed flask plus stir bar was charged with (4R*,4a′S*,10a′R*)-2-amino-4a′-methyl-3′,4′,4a′,10a′-tetrahydro-2′H,5H-spiro[oxazole-4,10′-pyrano[3,2-b]chromen]-8′-yl trifluoromethanesulfonate and its diastereomer (1.6 g, 5.3 mmol) and DCM (20 mL). The mixture was chilled in an acetonitrile bath, that was chilled to minus 20 degrees with addition of dry ice, under N₂. BBr₃ (11 ml, 11 mmol, 1M in DCM) was added dropwise. The reaction mixture continued stirring, monitoring by TLC, and maintaining temperature of bath between negative 10-15 degrees by addition of dry ice as needed. Stirred at this temperature for 3 hours. The reaction was worked up by adding ice chips to reaction at −10° C. The mixture was poured into saturated aqueous NaHCO₃ (30 mL). The aqueous phase was saturated with NaCl powder. The product was extracted with 10% MeOH/EtOAc (3×50 mL). The combined organics were dried (MgSO₄), filtered, and concentrated to yield (4R*,4a′S*,10a′R*)-2-amino-4a′-methyl-3′,4′,4a′,10a′-tetrahydro-2′H,5H-spiro[oxazole-4,10′-pyrano[3,2-b]chromen]-8′-ol (1.4 g, 87%), which was not separated from its diastereomer (1:2 ratio of desired diastereomer to undesired) at this step.

Step J: A stirred solution of (4R*,4a′S*,10a′R*)-2-amino-4a′-methyl-3′,4′,4a′,10a′-tetrahydro-2′H,5H-spiro[oxazole-4,10′-pyrano[3,2-b]chromen]-8′-ol and its diastereomer (1.4 g, 4.8 mmol) in DCM (20 mL) was treated with triethylamine (1.34 mL, 9.64 mmol) and 1,1,1-trifluoro-N-phenyl-N-(trifluoromethylsulfonyl)methanesulfonamide (2.07 g, 5.79 mmol). The reaction was sealed in a vial and stirred for 5 hours at room temperature. The reaction was washed with water, brine and dried over MgSO₄. The crude was purified by Biotage Flash 65 silica gel chromatography, eluting with a gradient of 1:1 EtOAc/hexanes to neat EtOAc, then 2%-6% MeOH/EtOAc to elute both diastereomers. The two diastereomers were separated to obtain a yield of (4R*,4a′S*,10a′R*)-2-amino-4a′-methyl-3′,4′,4a′,10a′-tetrahydro-2′H,5H-spiro[oxazole-4,10′-pyrano[3,2-b]chromen]-8′-yl trifluoromethanesulfonate (300 mg, 14%), and its diastereomer (4R*,4a′R*,10a′S*)-2-amino-4a′-methyl-3′,4′,4a′,10a′-tetrahydro-2′H,5H-spiro[oxazole-4,10′-pyrano[3,2-b]chromen]-8′-yl trifluoromethanesulfonate (588 mg, 26%).

Step K: A vial plus stir bar was charged with (4R*,4a′S*,10a′R*)-2-amino-4a′-methyl-3′,4′,4a′,10a′-tetrahydro-2′H,5H-spiro[oxazole-4,10′-pyrano[3,2-b]chromen]-8′-yl trifluoromethanesulfonate (20 mg, 0.047 mmol), dioxane (0.5 mL), 5-chloropyridin-3-ylboronic acid (11 mg, 0.071 mmol), Pd(PPh₃)₄ (5.5 mg, 0.0047 mmol), and 2N aqueous Na₂CO₃ (71 μL, 0.14 mmol). The mixture was sparged with N₂ for 2 minutes and then heated to 90° C. for 2 hours with stirring. After cooling to room temperature, the mixture was loaded directly on to a preparative TLC plate (0.5 mm thickness R_{f}=0.62), eluting with 10% MeOH (containing 7N NH₃)/DCM to yield (4R*,4a′S*,10a′R*)-8′-(5-chloropyridin-3-yl)-4a′-methyl-3′,4′,4a′,10a′-tetrahydro-2′H,5H-spiro[oxazole-4,10′-pyrano[3,2-b]chromen]-2-amine (6 mg 31%). ¹H NMR (CDCl₃+MeOD) δ 8.61 (d, J=2 Hz, 1H), 8.46 (d, J=2 Hz, 1H), 7.89 (t, J=2 Hz, 1H), 7.40 (m, 2H), 6.91 (d, J=9 Hz, 1H), 4.90 (d, J=8 Hz, 1H), 4.19 (d, J=8 Hz, 1H), 4.16 (m, 1H), 3.71 (s, 1H), 3.61 (m, 1H), 2.05 (m, 1H), 1.92 (m, 2H), 1.77 (m, 1H), 1.25 (s, 3H), m/z (APCI-pos) M+1=386.

### Example 82

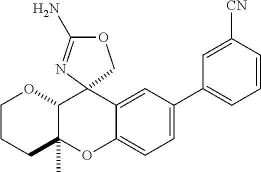

### 3-((4R*,4a′S*,10a′R*)-2-amino-4a′-methyl-3′,4′,4a′,10a′-tetrahydro-2H,5H-spiro[oxazole-4,10′-pyrano[3,2-b]chromen]-8′-yl)benzonitrile

3-((4R*,4a′S*,10a′R*)-2-Amino-4a′-methyl-3′,4′,4a′,10a′-tetrahydro-2′H,5H-spiro[oxazole-4,10′-pyrano[3,2-b]chromen]-8′-yl)benzonitrile (29 mg, 67%) was prepared from (4R*,4a′S*,10a′R*)-2-amino-4a′-methyl-3′,4′,4a′,10a′-tetrahydro-2′H,5H-spiro[oxazole-4,10′-pyrano[3,2-b]chromen]-8′-yl trifluoromethanesulfonate (synthesized as described in Example 81, Step J) according to the procedure for Example 81, Step K, substituting 3-cyanophenylboronic acid for 5-chloropyridin-3-ylboronic acid. ¹H NMR (CDCl₃+MeOD) δ 7.81 (m, 1H), 7.77 (m, 1H), 7.60 (m, 1H), 7.55 (m, 1H), 7.39 (m, 2H), 6.89 (d, J=9 Hz, 1H), 4.89 (d, J=8 Hz, 1H), 4.19 (d, J=8 Hz, 1H), 4.13 (m, 1H), 3.71 (s, 1H), 3.61 (m, 1H), 2.06 (m, 1H), 1.90 (m, 2H), 1.77 (m, 1H), 1.25 (s, 3H); m/z (APCI-pos) M+1=376.

### Example 83

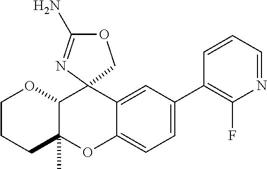

### (4R*,4a′S*,10a′R*)-8′-(2-fluoropyridin-3-yl)-4a′-methyl-3′,4′,4a′,10a′-tetrahydro-2′H,5H-spiro[oxazole-4,10′-pyrano[3,2-b]chromen]-2-amine

(4R*,4a′S*,10a′R*)-8′-(2-Fluoropyridin-3-yl)-4a′-methyl-3′,4′,4a′,10a′-tetrahydro-2′H,5H-spiro[oxazole-4,10′-pyrano[3,2-b]chromen]-2-amine (50 mg, 56%) was prepared from (4R*,4a′S*,10a′R*)-2-amino-4a′-methyl-3′,4′,4a′,10a′-tetrahydro-2′H,5H-spiro[oxazole-4,10′-pyrano[3,2-b]chromen]-8′-yl trifluoromethanesulfonate (synthesized as described in Example 81, Step J) according to the procedure for Example 81, Step K, substituting 2-fluoropyridin-3-ylboronic acid for 5-chloropyridin-3-ylboronic acid. ¹H NMR (400 MHz, CDCl₃) δ 8.14 (d, J=5 Hz, 1H), 7.85 (m, 1H), 7.30 (m, 2H), 7.27 (m, 1H), 6.89 (m, 1H), 4.88 (d, J=8 Hz, 1H), 4.17 (d, J=8 Hz, 1H), 4.13 (m, 1H), 3.73 (s, 1H), 3.61 (m, 1H), 2.03 (m, 1H), 1.89 (m, 2H), 1.75 (m, 1H), 1.25 (s, 3H); m/z (APCI-pos) M+1=370.

### Example 84

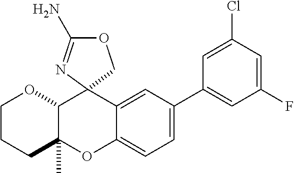

### (4R*,4a′S*, 10a′R*)-8′-(3-chloro-5-fluorophenyl)-4a′-methyl-3′,4′,4a′,10a′-tetrahydro-2′H,5H-spiro[oxazole-4,10′-pyrano[3,2-b]chromen]-2-amine

(4R*,4a′S*,10a′R*)-8′-(3-Chloro-5-fluorophenyl)-4a′-methyl-3′,4′,4a′,10a′-tetrahydro-2H,5H-spiro[oxazole-4,10′-pyrano[3,2-b]chromen]-2-amine (6 mg, 28%) was prepared from (4R*,4a′S*,10a′R*)-2-amino-4a′-methyl-3′,4′,4a′,10a′-tetrahydro-2′H,5H-spiro[oxazole-4,10′-pyrano[3,2-b]chromen]-8′-yl trifluoromethanesulfonate (synthesized as described in Example 81, Step J) according to the procedure for Example 81, Step K, substituting 3-chloro-5-fluorophenylboronic acid for 5-chloropyridin-3-ylboronic acid. ¹H NMR (CDCl₃+MeOD) δ 7.36 (m, 2H), 7.30 (m, 1H), 7.12 (m, 1H), 7.03 (m, 1H), 6.86 (d, J=9 Hz, 1H), 4.89 (d, J=8 Hz, 1H), 4.18 (d, J=8 Hz, 1H), 4.15 (m, 1H), 3.70 (s, 1H), 3.60 (m, 1H), 2.05 (m, 1H), 1.91 (m, 2H), 1.76 (m, 1H), 1.25 (s, 3H); m/z (APCI-pos) M+1=403.

### Example 85

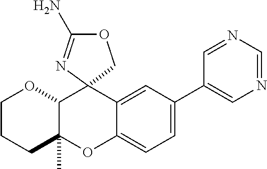

### (4R*,4a′S*,10a′R*)-4a′-methyl-8′-(pyrimidin-5-yl)-3′,4′,4a′,10a′-tetrahydro-2′H,5H-spiro[oxazole-4,10′-pyrano[3,2-b]chromen]-2-amine

(4R*,4a′S*,10a′R*)-4a′-Methyl-8′-(pyrimidin-5-yl)-3′,4′,4a′,10a′-tetrahydro-2′H,5H-spiro[oxazole-4,10′-pyrano[3,2-b]chromen]-2-amine (8 mg, 46%) was prepared from (4R*,4a′S*,10a′R*)-2-amino-4a′-methyl-3′,4′,4a′,10a′-tetrahydro-2′H,5H-spiro[oxazole-4,10′-pyrano[3,2-b]chromen]-8′-yl trifluoromethanesulfonate (synthesized as described in Example 81, Step J) according to the procedure for Example 81, Step K, substituting pyrimidin-5-ylboronic acid for 5-chloropyridin-3-ylboronic acid. ¹H NMR (CDCl₃+MeOD) δ 9.11 (s, 1H), 8.92 (s, 2H), 7.42 (m, 2H), 6.94 (d, J=9 Hz, 1H), 4.89 (d, J=8 Hz, 1H), 4.19 (d, J=8 Hz, 1H), 4.16 (m, 1H), 3.71 (s, 1H), 3.61 (m, 1H), 2.06 (m, 1H), 1.93 (m, 2H), 1.77 (m, 1H), 1.26 (s, 3H); m/z (APCI-pos) M+1=353.

### Example 86

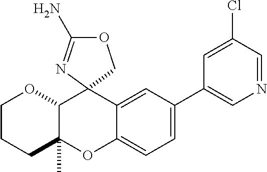

### 5-((4R*,4a′S*,10a′R*)-2-amino-4a′-methyl-3′,4′,4a′,10a′-tetrahydro-2′H,5H-spiro[oxazole-4,10′-pyrano[3,2-b]chromen]-8′-yl)nicotinonitrile

5-((4R*,4a′S*,10a′R*)-2-Amino-4a′-methyl-3′,4′,4a′,10a′-tetrahydro-2′H,5H-spiro[oxazole-4,10′-pyrano[3,2-b]chromen]-8′-yl)nicotinonitrile (7 mg, 37%) was prepared from (4R*,4a′S*,10a′R*)-2-amino-4a′-methyl-3′,4′,4a′,10a′-tetrahydro-2′H,5H-spiro[oxazole-4,10′-pyrano[3,2-b]chromen]-8′-yl trifluoromethanesulfonate (synthesized as described in Example 81, Step J) according to the procedure for Example 81, substituting 5-(4,4,5,5-tetramethyl-1,3,2-dioxaborolan-2-yl)nicotinonitrile for 5-chloropyridin-3-ylboronic acid. ¹H NMR (CDCl₃+MeOD) δ 8.96 (d, J=2 Hz, 1H), 8.79 (d, J=2 Hz, 1H), 8.18 (t, J=2 Hz, 1H), 7.41 (s, 2H), 6.94 (d, J=9 Hz, 1H), 4.90 (d, J=8 Hz, 1H), 4.18 (d, J=8 Hz, 1H), 4.14 (m, 1H), 3.71 (s, 1H), 3.61 (m, 1H), 2.06 (m, 1H), 1.92 (m, 2H), 1.77 (m, 1H), 1.26 (s, 3H); m/z (APCI-pos) M+1=377.

### Example 87

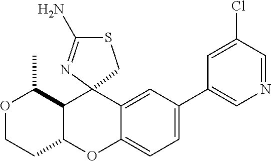

### (1R*,4aR*,4′R*,10aR*)-8-(5-chloropyridin-3-yl)-1-methyl-3,4,4a,10a-tetrahydro-1H,5′H-spiro[pyrano[4,3-b]chromene-10,4′-thiazol]-2′-amine

Step A: Similar to a procedure described in Phosphorus, Sulfur, and Silicon 2009, 184, 179-196, a mixture of 1-(5-bromo-2-hydroxyphenyl)ethanone (50 g, 233 mmol) and DMF-dimethylacetal (42 g, 349 mmol) in dry toluene (250 mL) was refluxed for 3 hours. After cooling to room temperature, the mixture was concentrated to half volume, and the resulting suspension was cooled in an ice bath. The solids were then filtered, washing with a minimal amount of toluene to yield (E)-1-(5-bromo-2-hydroxyphenyl)-3-(dimethylamino)prop-2-en-1-one (56 g, 87%).

Step B: Similar to a procedure described in Phosphorus, Sulfur, and Silicon 2009, 184, 179-196, acetic anhydride (196 mL) was added to a solution of (E)-1-(5-bromo-2-hydroxyphenyl)-3-(dimethylamino)prop-2-en-1-one (56 g, 207 mmol) in dry pyridine (84 mL), and the mixture was shirred at room temperature for 18 hours. The mixture was concentrated on the rotovap to one half volume at 80° C. The resulting suspension was cooled to room temperature, and then the solids were filtered. The solids were washed with hexanes and dried under high vacuum to yield 3-acetyl-6-bromo-4H-chromen-4-one (48 g, 85%).

Step C: A stainless steel bomb plus stir bar was charged with ethyl vinyl ether (169 mL, 1760 mmol) and 3-acetyl-6-bromo-4H-chromen-4-one (47 g, 176 mmol). The mixture was heated to 100° C. for 15 hours. After cooling to room temperature, the reaction mixture was filtered, washing the solids with a minimal amount of EtOAc to yield (3R*,4aR*)-8-bromo-3-ethoxy-1-methyl-4,4a-dihydropyrano[4,3-b]chromen-10(3H)-one (44 g, 72%).

Step D: A round bottomed flask plus stir bar was charged with (3R*,4aR*)-8-bromo-3-ethoxy-1-methyl-4,4a-dihydropyrano[4,3-b]chromen-10(3H)-one (43 g, 127 mmol), THF (500 mL), and the mixture was cooled to −78° C. in a dry ice/acetone bath. DIBAL (1.5M in toluene, 101 mL, 152 mmol) was added dropwise and stirred at −78° C. for 1 hour. The reaction remained a suspension the entire time. The reaction mixture was quenched by inverse addition (via canula) to Rochelle's salt (500 mL) that was stirred at room temperature. The mixture was worked up by extraction with EtOAc (2×500 mL). The combined organics were washed with brine (500 mL), dried (MgSO₄), filtered, and concentrated. The crude was purified by Biotage Flash 75 silica gel chromatography, eluting with 5%-10% EtOAc/hexanes to yield (1R*,4aR*,10aR*)-8-bromo-3-ethoxy-1-methyl-1,4,4a,10a-tetrahydropyrano[4,3-b]chromen-10(3H)-one (22.4 g, 36%).

Step E: A round bottomed flask plus stir bar was charged with (1R*,4aR*,10aR*)-8-bromo-3-ethoxy-1-methyl-1,4,4a,10a-tetrahydropyrano[4,3-b]chromen-10(3H)-one (22.2 g, 65.1 mmol), DCM (200 mL), and triethylsilane (51.8 mL, 325 mmol). The mixture was cooled in an ice bath under N₂. Then, BF₃-etherate (24.7 mL, 195 mmol) was added dropwise. The mixture was stirred overnight at room temperature. The mixture was Carefully quenched with saturated aqueous NaHCO₃ (200 mL). The mixture was stirred for 1 hour. The phases were separated phases, and the aqueous phase was re-extracted with DCM (2×75 mL). The combined organic phases were washed with brine (200 mL), dried (MgSO₄), filtered, and concentrated. The crude was purified Biotage Flash 65 silica gel chromatography, eluting with 10%-20% EtOAc/hexanes to yield (1R*,4aR*,10aR*)-8-bromo-1-methyl-1,4,4a,10a-tetrahydropyrano[4,3-b]chromen-10(3H)-one (13.6 g, 60%).

Step F: A round bottomed flask plus stir bar was charged with (1R*,4aR*,10aR*)-8-bromo-1-methyl-1,4,4a,10a-tetrahydropyrano[4,3-b]chromen-10(3H)-one (2.5 g, 8.4 mmol) and anhydrous THF (50 mL). The mixture was cooled to 0° C. under N₂, and Tebbe reagent (25 mL, 13 mmol) was added dropwise. The mixture was removed from the ice bath allowing to warm to room temperature. The mixture was stirred for 18 hours. 30% Aqueous Rochelle's salt (100 mL) was very slowly added to the mixture, while stirring in an ice bath. The mixture was stirred for 2 hours at room temperature. The reaction mixture was filtered, rinsing with EtOAc. The phases were separated. The aqueous phase was re-extracted with EtOAc (3×50 mL). The combined organics were washed with brine (100 mL), dried (MgSO₄), filtered, and concentrated. The crude was purified by Biotage Flash 40 silica gel chromatography, eluting with neat hexanes, followed by 5% EtOAc/hexanes to elute (1R*,4aR*,10aR*)-8-bromo-1-methyl-10-methylene-1,3,4,4a,10,10a-hexahydropyrano[4,3-b]chromene (1.4 g, 55%).

Step G: AgSCN (2.36 g, 14.2 mmol), CH₃CN (5 mL) and THF (5 mL) was added to a round bottomed flask plus stir bar. The mixture was cooled in an ice bath under N₂. Iodine (3.01 g, 11.9 mmol) was added, and the mixture was stirred for 15 minutes at 0° C. Then, (1R*,4aR*,10aR*)-8-bromo-1-methyl-10-methylene-1,3,4,4a,10,10a-hexahydropyrano[4,3-b]chromene (1.4 g, 4.74 mmol) in THF (10 mL) was added. The mixture was stirred in the ice bath for 5 minutes, then at room temperature for 4 hours. Aqueous NH₄OH (3 mL) was added directly to the suspension. The suspension was stirred at room temperature overnight. The reaction mixture was partioned between EtOAc (30 mL) and aqueous saturated sodium thiosulfate solution (30 mL). After shaking to remove dark color, the phases were separated. The aqueous phases was reextracted with EtOAc (2×10 mL). The combined organics were washed with brine (30 mL), dried (MgSO₄), filtered, and concentrated. The crude was purified by Biotage Flash 40 silica gel chromatography, eluting with 5%-7% MeOH/DCM to yield (1R*,4aR*,4′R*,10aR*)-8-bromo-1-methyl-3,4,4a,10a-tetrahydro-1H,5′H-spiro[pyrano[4,3-b]chromene-10,4′-thiazol]-2′-amine (1.0 g, 56%), obtained as a 60:40 mixture of diastereomers.

Step H: A vial plus stir bar was charged with (1R*,4aR*,4′R*,10aR*)-8-bromo-1-methyl-3,4,4a,10a-tetrahydro-1H,5′H-spiro[pyrano[4,3-b]chromene-10,4′-thiazol]-2′-amine and its diastereomer (200 mg, 0.542 mmol), dioxane (4 mL), 5-chloropyridin-3-ylboronic acid (128 mg, 0.812 mmol), Pd(PPh₃)₄ (63 mg, 0.054 mmol), and 2N aqueous Na₂CO₃ (812 μL, 1.62 mmol). The mixture was sparged with N₂ for 3 minutes and then heated to 90° C. for 2 hours with stirring. The crude reaction mixture was loaded on to 2 different preparative TLC plates (2 mm thickness each, R_{f}=0.50) eluting with 10% MeOH/DCM+1% HOAc. The diastereomers were separated. (1R*,4aR*,4′R*,10aR*)-8-(5-Chloropyridin-3-yl)-1-methyl-3,4,4a,10a-tetrahydro-1H,5′H-spiro[pyrano[4,3-b]chromene-10,4′-thiazol]-2′-amine was triturated with 1:1 DCM/Et₂O and filtered to yield 86 mg (39%). This amine was then diluted with DCM/MeOH, and TFA (0.1 mL) was added. It was concentrated in vacuo, using DCM to azeotrope away residual TFA, multiple times to provide (1R*,4aR*,4′R*,10aR*)-8-(5-chloropyridin-3-yl)-1-methyl-3,4,4a,10a-tetrahydro-1H,5′H-spiro[pyrano[4,3-b]chromene-10,4′-thiazol]-2′-amine bis(2,2,2-trifluoroacetate). m/z (APCI-pos) M+1=402.

### Example 88

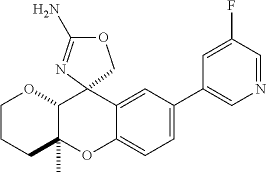

### (4R*,4a′S*,10a′R*)-8′-(5-fluoropyridin-3-yl)-4a′-methyl-3′,4′,4a′,10a′-tetrahydro-2′H,5H-spiro[oxazole-4,10′-pyrano[3,2-b]chromen]-2-amine

(4R*,4a′S*,10a′R*)-8′-(5-fluoropyridin-3-yl)-4a′-Methyl-3′,4′,4a′,10a′-tetrahydro-2′H,5H-spiro[oxazole-4,10′-pyrano[3,2-b]chromen]-2-amine (10 mg, 51%) was prepared from (4R*,4a′S*,10a′R*)-2-amino-4a′-methyl-3′,4′,4a′,10a′-tetrahydro-2′H,5H-spiro[oxazole-4,10′-pyrano[3,2-b]chromen]-8′-yl trifluoromethanesulfonate (synthesized as described in Example 81, Step J) according to the procedure for Example 81, Step K, substituting 5-fluoropyridin-3-ylboronic acid for 5-chloropyridin-3-ylboronic acid. ¹H NMR (CDCl₃+MeOD) δ 8.56 (t, J=2 Hz, 1H), 8.36 (d, J=3 Hz, 1H), 7.62 (m, 1H), 7.40 (m, 2H), 6.91 (d, J=9 Hz, 1H), 4.89 (d, J=8 Hz, 1H), 4.18 (d, J=8 Hz, 1H), 4.15 (m, 1H), 3.71 (s, 1H), 3.61 (m, 1H), 2.06 (m, 1H), 1.90 (m, 2H), 1.77 (m, 1H), 1.25 (s, 3H); m/z (APCI-pos) M+1=370.

### Example 89

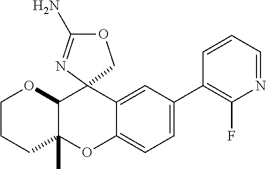

### (4R*,4′R*,10a′S*)-8′-(2-fluoropyridin-3-yl)-4a′-methyl-3′,4′,4a′,10a′-tetrahydro-2H,5H-spiro[oxazole-4,10′-pyrano[3,2-b]chromen]-2-amine

(4R*,4a′R*,10a′S*)-8′-(2-Fluoropyridin-3-yl)-4a′-methyl-3′,4′,4a′,10a′-tetrahydro-2′H,5H-spiro[oxazole-4,10′-pyrano[3,2-b]chromen]-2-amine (24 mg, 26%) was prepared from (4R*,4a′R*,10a′S*)-2-amino-4a′-methyl-3′,4′,4a′,10a′-tetrahydro-2′H,5H-spiro[oxazole-4,10′-pyrano[3,2-b]chromen]-8′-yl trifluoromethanesulfonate (synthesized as described in Example 81, Step J) according to the procedure for Example 81, Step K, substituting 2-fluoropyridin-3-ylboronic acid for 5-chloropyridin-3-ylboronic acid and substituting (4R*,4a′R*,10a′S*)-2-amino-4a′-methyl-3′,4′,4a′,10a′-tetrahydro-2′H,5H-spiro[oxazole-4,10′-pyrano[3,2-b]chromen]-8′-yl trifluoromethanesulfonate for its diastereomer (4R*,4a′S*,10a′R*)-2-amino-4a′-methyl-3′,4′,4a′,10a′-tetrahydro-2′H,5H-spiro[oxazole-4,10′-pyrano[3,2-b]chromen]-8′-yl trifluoromethanesulfonate. ¹H NMR (400 MHz, CDCl₃) δ 8.13 (d, J=5 Hz, 1H), 7.86 (m, 1H), 7.40 (m, 2H), 7.30 (m, 1H), 6.90 (d, J=9 Hz, 1H), 4.49 (d, J=8 Hz, 1H), 4.46 (d, J=8 Hz, 1H), 4.19 (m, 1H), 3.58 (m, 1H), 3.44 (s, 1H), 2.07 (m, 1H), 1.95 (m, 1H), 1.85 (m, 1H), 1.72 (m, 1H), 1.48 (s, 3H); m/z (APCI-pos) M+1=370.

### Example 90

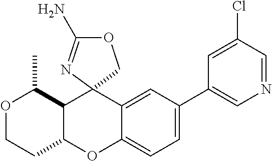

### (1′R*4R*,4a′R*,10a′R*)-8′-(5-chloropyridin-3-yl)-1′-methyl-3′,4′,4a′,10a′-tetrahydro-1′H,5H-spiro[oxazole-4,10′-pyrano[4,3-b]chromen]-2-amine

Step A: Silver cyanate (1.83 g, 12.2 mmol), CH₃CN (5 mL) and THF (5 mL) were added to a round bottomed flask plus stir bar. The mixture was cooled in an ice bath under N₂. Iodine (2.58 g, 10.2 mmol) was added. The mixture was stirred for 15 minutes at 0° C. Then, (1R*,4aR*,10aR*)-8-bromo-1-methyl-10-methylene-1,3,4,4a,10,10a-hexahydropyrano[4,3-b]chromene (1.2 g, 4.07 mmol; prepared as described in Example 87, Step F) in THF (10 mL) was added. The mixture was stirred in the ice bath for 5 minutes, then at room temperature for 4 hours. The reaction mixture was filtered, rinsing with THF. Aqueous NH₄OH (3 mL) to the filtrate in a round bottomed flask plus stir bar. The mixture was stirred at room temperature overnight. The reaction mixture was partioned between EtOAc (30 mL) and aqueous saturated sodium thiosulfate solution (30 mL). After shaking to remove dark color, the phases were separated. The aqueous phase was reextracted with EtOAc (3×10 mL). The combined organics were washed with brine (30 mL), dried (MgSO₄), filtered, and concentrated to yield (1′R*,4R*,4a′R*,10a′R*)-8′-bromo-1′-methyl-3′,4′,4a′,10a′-tetrahydro-1′H,5H-spiro[oxazole-4,10′-pyrano[4,3-b]chromen]-2-amine and its diastereomer (1′S*,4R*,4a′S*,10a′S*)-8′-bromo-1′-methyl-3′,4′,4a′,10a′-tetrahydro-1′H,5H-spiro[oxazole-4,10′-pyrano[4,3-b]chromen]-2-amine (1.5 g, 99%). The diastereomers were carried forward to the next step without separation.

Step B: A vial plus stir bar was charged with (1′R*,4R*,4a′R*,10a′R*)-8′-bromo-1′-methyl-3′,4′,4a′,10a′-tetrahydro-1′H,5H-spiro[oxazole-4,10′-pyrano[4,3-b]chromen]-2-amine and its diastereomer (1′S*,4R*,4a′S*,10a′S*)-8′-bromo-1′-methyl-3′,4′,4a′,10a′-tetrahydro-1′H,5H-spiro[oxazole-4,10′-pyrano[4,3-b]chromen]-2-amine (100 mg, 0.283 mmol), dioxane (2 mL), 5-chloropyridin-3-ylboronic acid (49 mg, 0.31 mmol), Pd(PPh₃)₄ (33 mg, 0.028 mmol), and 2N aqueous Na₂CO₃ (354 μL, 0.708 mmol). The mixture was sparged with N₂ for 3 minutes and then heated to 90° C. for 2 hours. The reaction mixture was loaded directly on to a preparative TLC plate (1 mm thickness) eluting with 10% MeOH/DCM. Each diastereomer was separately subjected to a second purification by preparative TLC (1 mm thickness) eluting with 7.5% MeOH (containing 7N NH₃)/DCM to yield (1′R*,4R*,4a′R*,10a′R*)-8′-(5-chloropyridin-3-yl)-1′-methyl-3′,4′,4a′,10a′-tetrahydro-1′H,5H-spiro[oxazole-4,10′-pyrano[4,3-b]chromen]-2-amine (9 mg, 7%). NMR (400 MHz, CDCl₃+MeOD) δ 8.59 (m, 1H), 8.47 (m, 1H), 7.83 (m, 1H), 7.35 (m, 2H), 6.95 (m, 1H), 4.53 (m, 2H), 4.07 (m, 2H), 3.60 (m, 2H), 2.18 (m, 1H), 1.96 (m, 1H), 1.75 (m, 1H), 1.36 (m, 3H); m/z (APCI-pos) M+1=386.

### Example 91

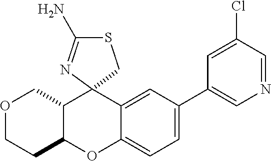

### (4aS*,4′R*,10aS*)-8-(5-chloropyridin-3-yl)-3,4,4a,10a-tetrahydro-1H,5′H-spiro[pyrano[4,3-b]chromene-10,4′-thiazol]-2′-amine

Step A: A mixture of dihydro-2H-pyran-4(3H)-one (100 g, 999 mmol) and morpholine (131 mL, 1498 mmol) in toluene (333 mL) was refluxed under Dean-Stark trap overnight. More than 1 equivalent of water was collected. This reaction mixture was then concentrated down to give 4-(3,6-dihydro-2H-pyran-4-yl)morpholine (169 g, 100% yield) as an oil.

Step B: A mixture of 4-(3,6-dihydro-2H-pyran-4-yl)morpholine (178.1 g, 1052 mmol) and 5-bromo-2-hydroxybenzaldehyde (211.6 g, 1052 mmol) in toluene (351 mL) was stirred overnight at room temperature. A solid crashed out and was filtered off. This was washed with toluene (50 mL). The solid product was collected and dried to give 8-bromo-4a-morpholino-1,3,4,4a,10,10a-hexahydropyrano[4,3-b]chromen-10-ol (306.8 g, 79% yield).

Step C: DMSO (204 mL, 2878 mmol) was added dropwise to oxalyl chloride (470 mL, 939 mmol) in DCM (8 L) at −78° C. This was added such that the temperature did not rise above −65° C. The mixture was then stirred for 40 minutes at −78° C. 8-Bromo-4a-morpholino-1,3,4,4a,10,10a-hexahydropyrano[4,3-b]chromen-10-ol (533 g, 1439 mmol) was added as a solid (temperature did not rise), and this was stirred for 2 hours at −78° C. The solid did not fully go into solution. Triethylamine (602 mL, 4317 mmol) was added dropwise (some exotherm was seen, however the reaction temperature did not get above −65° C.). This was stirred for 30 minutes at −78° C. During the entire course of the reaction, the mixture was continually purged with N₂ which exited the flask via a line fed into a bleach trap. The mixture was then concentrated down. Glacial acetic acid (1000 mL) was added to the mixture. The material went into solution initially, however after 5 minutes of stirring, the product began to crash out. The material was stirred overnight at room temperature. A solid had crashed out and was filtered. The solid was washed with glacial acetic acid (200 mL). This gave 8-bromo-3,4-dihydropyrano[4,3-b]chromen-10(1H)-one (340.8 g, 84% yield) as a solid.

Step D: 1-Selectride (587 mL, 587 mmol, 1M in THF) was added to a mixture of 8-bromo-3,4-dihydropyrano[4,3-b]chromen-10(1H)-one (150 g, 534 mmol) in DCM (2809 mL) at −78° C. The mixture was stirred for 45 minutes. TLC showed that the reaction was complete. The mixture was placed in an ice bath. Aqueous Rochelle's salt (0.5M) was added to the mixture as it was warming to 0° C. This was then worked up with EtOAc/water. The organics were extracted twice, washed with brine, dried (Na₂SO₄), and concentrated. The crude was then triturated with hexanes to give (4aS*,10aS*)-8-bromo-1,4,4a,10a-tetrahydropyrano[4,3-b]chromen-10(3H)-one (100 g, 66% yield).

Step E: A round bottomed flask plus stir bar was charged with (4aS*,10aS*)-8-bromo-1,4,4a,10a-tetrahydropyrano[4,3-b]chromen-10(3H)-one (544 mg, 1.92 mmol) and anhydrous THF (5 mL). The mixture was cooled to 0° C. under N₂, and added Tebbe reagent (5.7 mL, 2.88 mmol) was added. The mixture was stirred for 2 hours at room temperature. The mixture was cooled in an ice bath and added MeOH (5 mL) very carefully (vigorous exotherm and bubbling), then aqueous 2N NaOH (5 mL) was added dropwise. To enable stirring, DCM (10 mL) was added. The biphasic suspension was stirred for 15 minutes at room temperature. The biphase was filtered to remove solids through Celite®, rinsing with DCM. The phases were separated, and the aqueous phase was re-extracted with DCM (5 mL). The combined organics were washed with brine (20 mL), dried (MgSO₄), filtered, and concentrated. The crude was purified crude by Biotage Flash 40 silica gel chromatography, eluting with 10%-15% EtOAc/hexanes to yield of (4aS*,10aS*)-8-bromo-10-methylene-1,3,4,4a,10,10a-hexahydropyrano[4,3-b]chromene (168 mg, 30%).

Step F: A stirred solution of (4aS*,10aS*)-8-bromo-10-methylene-1,3,4,4a,10,10a-hexahydropyrano[4,3-b]chromene (168 mg, 0.598 mmol) in diethyl ether (2 mL) was cooled to 0° C. under N₂. In a separate flask, silver thiocyanate (397 mg, 2.39 mmol) was suspended in CH₃CN (1 mL), and iodine (303 mg, 1.20 mmol) in THF (1 mL) was added to this suspension. The resulting mixture was shaken for 30 seconds. The suspension was then poured into the alkene solution at 0° C., and the vial was rinsed with CH₃CN and added to the reaction mixture. The reaction mixture was stirred at room temperature for 4 hours. Aqueous NH₄OH (1 mL) was added directly to the reaction mixture, and it was stirred at room temperature for 2 hours, then left sitting for 18 hours at room temperature. The salts were filtered through Celite®, rinsing with EtOAc. The filtrate was washed with aqueous saturated Na₂S₂O₃ (20 mL). After shaking and then separating the phases, the aqueous layer was re-extracted with EtOAc (20 mL). The combined organic layers were washed with brine (20 mL), dried (MgSO₄), filtered, and concentrated. The crude was purified by preparative TLC (2 mm thickness) eluting with 10% MeOH/DCM to yield (4aS*,4′R*,10aS*)-8-bromo-3,4,4a,10a-tetrahydro-1H,5′H-spiro[pyrano[4,3-b]chromene-10,4′-thiazol]-2′-amine (72 mg, 32%), obtained as a mixture of diastereomers which were carried forward without separation at this step.

Step G: A vial plus stir bar was charged with (4aS*,4′R*,10aS*)-8-bromo-3,4,4a,10a-tetrahydro-1H,5′H-spiro[pyrano[4,3-b]chromene-10,4′-thiazol]-2′-amine as a diastereomeric mixture (38 mg, 0.11 mmol), dioxane (0.5 mL), 5-chloropyridin-3-ylboronic acid (25 mg, 0.16 mmol), Pd(PPh₃)₄ (12 mg, 0.011 mmol), and 2N aqueous Na₂CO₃ (160 μL, 0.32 mmol). The mixture was sparged with N₂ for 1 min and then heated to 90° C. for 2 hours with stirring. The mixture was loaded directly on to a preparative TLC plate (1 mm thickness, R_{f}=0.66), eluting with 10% MeOH (containing 7N NH₃)/DCM. The desired diastereomer was repurified by preparative TLC (0.5 mm thickness, R_{f}=0.45) eluting with 10% MeOH/DCM to yield (4aS*,4′R*,10aS*)-8-(5-chloropyridin-3-yl)-3,4,4a,10a-tetrahydro-1H,5′H-spiro[pyrano[4,3-b]chromene-10,4′-thiazol]-2′-amine (10 mg, 21%). m/z (APCI-pos) M+1=388.

### Example 92

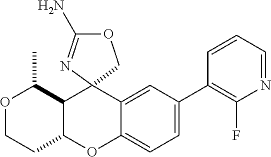

### (1′R*,4R*,4a′R*,10a′R*)-8′-(2-fluoropyridin-3-yl)-1′-methyl-3′,4′,4a′,10a′-tetrahydro-1′H,5H-spiro[oxazole-4,10′-pyran[4,3-b]chromen]-2-amine

(1′R*,4R*,4a′R*,10a′R*)-8′-(2-Fluoropyridin-3-yl)-1′-methyl-3′,4′,4a′,10a′-tetrahydro-1′H,5H-spiro[oxazole-4,10′-pyrano[4,3-b]chromen]-2-amine (13 mg, 12%) was prepared from (1′R*,4R*,4a′R*,10a′R*)-8′-bromo-1′-methyl-3′,4′,4a′,10a′-tetrahydro-1′H,5H-spiro[oxazole-4,10′-pyrano[4,3-b]chromen]-2-amine (synthesized as described in Example 90, Step A) according to the procedure for Example 90, Step B, substituting 2-fluoropyridin-3-ylboronic acid for 5-chloropyridin-3-ylboronic acid. ¹H NMR (400 MHz, CDCl₃+MeOD) 8.14 (m, 1H), 8.13 (m, 1H), 7.86 (m, 1H), 7.38 (m, 1H), 7.31 (m, 1H), 6.92 (d, J=8 Hz, 1H), 4.56 (d, J=9 Hz, 1H), 4.51 (d, J=9 Hz, 1H), 4.07 (m, 2H), 3.59 (m, 2H), 2.19 (m, 1H), 1.95 (m, 1H), 1.76 (t, J=10 Hz, 1H), 1.35 (d, J=6 Hz, 3H); m/z (APCI-pos) M+1=370.

### Example 93

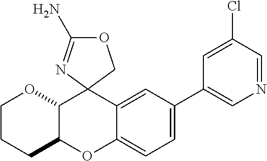

### (4a′S*,10a′R*)-8′-(5-chloropyridin-3-yl)-3′,4′,4a′,10a′-tetrahydro-2′H,5H-spiro[oxazole-4,10′-pyrano[3,2-b]chromen]-2-amine

Step A: Charged a 1-liter round bottom flask with dihydro-2H-pyran-3(4H)-one (18.5 g, 185 mmol) and morpholine (24.1 g, 277 mmol) in toluene (500 mL). The mixture was heated to reflux with azeotropic removal of water for 4 hours, then concentrated under reduced pressure to give a quantitative yield 4-(3,4-dihydro-2H-pyran-5-yl)morpholine.

Step B: To a round bottom flask containing 4-(3,4-dihydro-2H-pyran-5-yl)morpholine (15.8 g, 78.6 mmol) in toluene (500 mL) was added 5-bromo-2-hydroxybenzaldehyde (13.3 g, 78.6 mmol). This mixture was stirred at room temperature for 16 hours, and then concentrated under reduced pressure to an oil. The crude material was passed through a 220 g Redi Sep column, eluting with 25% ethyl acetate/DCM, to give 8-bromo-4a-morpholino-2,3,4,4a,10,10a-hexahydropyrano[3,2-b]chromen-10-01 (18 g, 61%) as a mixture of diastereomers.

Step C: Charged a round bottom flask with oxalyl chloride (27.6 mL, 53.5 mmol, 2M in DCM) and dry DCM (200 mL). This was chilled to −78° C., and DMSO (7.6 g, 97.2 mmol) was then added by syringe and gas evolution was observed. After about 15 minutes, a DCM solution (200 mL) of 8-bromo-4a-morpholino-2,3,4,4a,10,10a-hexahydropyrano[3,2-b]chromen-10-ol (18 g, 48.6 mmol) was added, and the mixture was stirred at −78° C. for 1 hour. Triethylamine (14.8 g, 146 mmol) was added, and the reaction mixture was allowed to warm to ambient temperature, while a stream of nitrogen gas was bubbled through into a beaker with bleach. The mixture was concentrated under reduced pressure, and the resulting crude material was taken up in AcOH (100 mL) and stirred at room temperature overnight. Water (500 mL) was then added, resulting in solids forming. The solids were collected by filtration, but were taken up in EtOAc and combined with the filtrate. The organics were isolated, washed with 10% aqueous potassium carbonate (2×), dried and concentrated under reduced pressure to give 8-bromo-3,4-dihydropyrano[3,2-b]chromen-10(2H)-one (12 g, 88%) as a solid. This material was used without further purification.

Step D: Charged a round bottom flask with 8-bromo-3,4-dihydropyrano[3,2-b]chromen-10(2H)-one (12 g, 42.7 mmol) and dry THF (400 mL). This mixture was chilled to −78° C., and L-Selectride (51.2 mL, 51.2 mmol, 1M in THF) was then added slowly by syringe. The mixture was stirred at −78° C. for 30 minutes, and then allowed to warm to −50° C. for 30 minutes. The mixture was quenched with 30% aqueous Rochelle's salt solution (250 mL), and the mixture was allowed to warm to room temperature. The mixture was then extracted with EtOAc (2×), extracts dried over sodium sulfate and concentrated under reduced pressure. The crude product was passed through a 220 g Redi Sep column (1:3 ethyl acetate:hexanes) to give (4aR,10aR)-8-bromo-2,3,4,4a-tetrahydropyrano[3,2-b]chromen-10(10aH)-one (5.5 g, 46%) as a pure trans diastereomer.

Step E: Charged a round bottom flask with (4aR,10aR)-8-bromo-2,3,4,4a-tetrahydropyrano[3,2-b]chromen-10(10aH)-one (4.0 g, 14.1 mmol) and dry THF (140 mL). This mixture was chilled to 0° C. To this was added Tebbe's reagent (56.5 mL, 28.2 mmol, 0.5M in toluene), and the mixture was stirred at 0° C. for 20 minutes, and then allowed to warm to ambient temperature. The mixture was then chilled back to 0° C. and quenched by the slow addition of methanol (15 mL), followed by 3M aqueous NaOH (200 mL). This mixture was stirred at room temperature overnight, followed by the addition of EtOAc (100 mL). The mixture was filtered through GF/F paper. The organics were isolated from the filtrate, dried and concentrated. The crude was passed through an 80 g Redi Sep column (100% DCM) to give (4aS,10aR)-8-bromo-10-methylene-2,3,4,4a,10,10a-hexahydropyrano[3,2-b]chromene (3 g, 76%) as an oil.

Step F: (4aS,10aR)-8-Bromo-10-methylene-2,3,4,4a,10,10a-hexahydropyrano[3,2-b]chromene (2.0 g, 7.11 mmol) was taken up in dry THF (70 mL), and to this was added a pre-sonicated (2 minutes) mixture of I₂/AgNCO in 1:1 THF:acetonitrile (2.13 g, 14.2 mmol/5.42 g, 21.3 mmol). This mixture was stirred at ambient temperature for 4 hours. This was then filtered through GF/F filter paper, and the filtrate was then chilled to 0° C. and ammonium hydroxide (20 mL) added. The mixture was allowed to warm to room temperature and stirred for 16 hours. The mixture was then diluted with water, extracted with EtOAc (2×), extracts washed with 10% aqueous sodium thiosulfate solution, brine, dried and concentrated to (4a′S,10a′R)-8′-bromo-3′,4′,4a′,10a′-tetrahydro-2′H,5H-spiro[oxazole-4,10′-pyrano[3,2-b]chromen]-2-amine (1.38 g, 57%) as a solid.

Step G: A reaction vial was charged with (4a′S,10a′R)-8′-bromo-3′,4′,4a′,10a′-tetrahydro-2H,5H-spiro[oxazole-4,10′-pyrano[3,2-b]chromen]-2-amine (100 mg, 0.295 mmol), 5-chloropyridin-3-ylboronic acid (55.7 mg, 0.354 mmol), Pd(PPh₃)₄ (34.1 mg, 0.0295 mmol), aqueous potassium carbonate (0.442 mL, 0.884 mmol), and dioxane (3 mL). This mixture was purged with argon for 5 minutes. The vial was sealed, and reaction mixture was heated to 100° C. for 4 hours, then diluted with EtOAc, washed with brine, dried and concentrated. The crude was passed through a 40 g Redi Sep column (7% MeOH/DCM), then through reverse phase prep HPLC to give (4a′S*,10a′R*)-8′-(5-chloropyridin-3-yl)-3′,4′,4a′,10a′-tetrahydro-2′H,5H-spiro[oxazole-4,10′-pyrano[3,2-b]chromen]-2-amine (15 mg, 14%). NMR (400 MHz, CDCl₃) δ 8.70-8.65 (m, 1H); 8.52-8.48 (m, 1H); 7.85-7.81 (m, 1H); 7.51-7.47 (m, 1H); 7.41-7.34 (m, 1H); 6.93-6.88 (m, 1H), 4.75-4.69 (m, 1H); 4.11-4.02 (m, 2H); 3.79-3.70 (m, 1H); 3.61-3.51 (m, 2H); 2.43-2.29 (m, 1H); 1.88-1.69 (m, 3H); m/z (APCI-pos) M+1=372.1, 374.1.

### Example 94

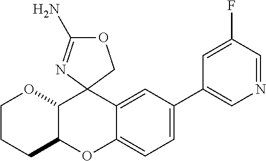

### (4a′S*,10a′R*)-8′-(5-fluoropyridin-3-yl)-3′,4′,4a′,10a′-tetrahydro-2H,5H-spiro[oxazole-4,10′-pyrano[3,2-b]chromen]-2-amine

(4a′S*,10a′R*)-8′-(5-Fluoropyridin-3-yl)-3′,4′,4a′,10a′-tetrahydro-2′H,5H-spiro[oxazole-4,10′-pyrano[3,2-b]chromen]-2-amine (14%) was prepared according to Example 93, Step G, substituting 5-fluoropyridin-3-ylboronic acid for 5-chloropyridin-3-ylboronic acid. ¹H NMR (400 MHz, CDCl₃) δ 8.65-8.61 (m, 1H); 8.42-8.38 (m, 1H); 7.58-7.52 (m, 1H); 7.51-7.47 (m, 1H); 7.40-7.35 (m, 1H); 6.92-6.88 (m, 1H), 4.75-4.70 (m, 1H); 4.12-4.02 (m, 2H); 3.79-3.70 (m, 1H); 3.60-3.49 (m, 2H); 2.40-2.31 (m, 1H); 1.88-1.70 (m, 3H); m/z (APCI-pos) M+1=356.1.

### Example 95

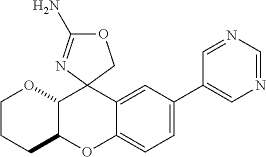

### (4aS*,10a′R*)-8′-(pyrimidin-5-yl)-3′,4′,4a′,10a′-tetrahydro-2′H,5H-spiro[oxazole-4,10′-pyrano[3,2-b]chromen]-2-amine

(4a′S*,10a′R*)-8′-(Pyrimidin-5-yl)-3′,4′,4a′,10a′-tetrahydro-2′H,5H-spiro[oxazole-4,10′-pyrano[3,2-b]chromen]-2-amine (10%) was prepared according to Example 93, Step G, substituting pyrimidin-5-ylboronic acid for 5-chloropyridin-3-ylboronic acid. m/z (APCI-pos) M+1=339.1.

### Example 96

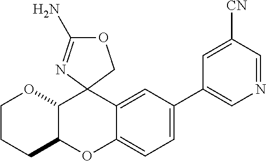

### 5-((4a′S*,10a′R*)-2-amino-3′,4′,4a′,10a′-tetrahydro-2′H,5H-spiro[oxazole-4,10′-pyrano[3,2-b]chromen]-8′-yl)nicotinonitrile

5-((4a′S*,10a′R*)-2-Amino-3′,4′,4a′,10a′-tetrahydro-2′H,5H-spiro[oxazole-4,10′-pyrano[3,2-b]chromen]-8′-yl)nicotinonitrile (9%) was prepared according to Example 93, Step G, substituting 5-(4,4,5,5-tetramethyl-1,3,2-dioxaborolan-2-yl)nicotinonitrile for 5-chloropyridin-3-ylboronic acid. m/z (APCI-pos) M+1=364.0.

### Example 97

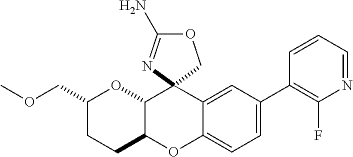

### (2′R*,4R*,4a′S*,10a′R*)-8′-(2-fluoropyridin-3-yl)-2′-(methoxymethyl)-3′,4′,4a′,10a′-tetrahydro-2′H,5H-spiro[oxazole-4,10′-pyrano[3,2-b]chromen]-2-amine

Step A: A round bottom flask was charged with (3,4-dihydro-2H-pyran-2-yl)methanol (19.5 g, 171 mmol) and dry DMF (600 mL). This solution was chilled to 0° C. and NaH (8.9 g, 221 mmol, 60% dispersion in mineral oil) was added (hydrogen evolution observed). This mixture was stirred at 0° C. for 30 minutes, then MeI (36.4 g, 256 mmol) was added. The mixture was allowed to warm to ambient temperature. The mixture was quenched by pouring into brine (350 mL), extracted with ether (2×), extracts washed with brine, dried over magnesium sulfate and concentrated under reduced pressure. The crude product was passed through a 120 g Redi Sep column (20% ethyl acetate:hexane) to give 2-(methoxymethyl)-3,4-dihydro-2H-pyran (12.8 g, 58%) as a liquid.

Step B: To a round bottom flask containing 2-(methoxymethyl)-3,4-dihydro-2H-pyran (6.8 g, 53.1 mmol) in dry THF (100 mL), at 0° C., was added 9-BBN (127 mL, 63.7 mmol, 0.5M in THF) over a 30 minute period. Once the addition was complete, the mixture was stirred at room temperature for 16 hours. The mixture was quenched, with cooling (0° C.), by the addition sodium perborate tetrahydrate (50 g in 300 mL of water). This was vigorously stirred for 1 hour, filtered, filtrate extracted with ether (2×), extracts dried over magnesium sulfate and concentrated under reduced pressure. The crude was passed through a 120 g Redi Sep column (eluting with 3:1 hexanes:ethyl acetate then 100% ethyl acetate) to give 6-(methoxymethyl)tetrahydro-2H-pyran-3-ol (2.8 g, 36%) as a mixture of diastereomers.

Step C: Charged a round bottom flask with 6-(methoxymethyl)tetrahydro-2H-pyran-3-ol (2.8 g, 19.2 mmol) and dry DCM (190 mL) and cooled to 0° C. To this was added Dess-Martin reagent (10.6 g, 24.9 mmol), and the mixture was allowed to warm to room temperature overnight. IPA (20 mL) was added to quench the reaction mixture, and the mixture was concentrated under reduced pressure. The resulting crude material was triturated with ether, filtered, and the filtrate concentrated. This material was passed through an 80 g Redi Sep column, eluting with 1:1 ethyl acetate:hexanes, to give 6-(methoxymethyl)dihydro-2H-pyran-3(4H)-one (700 mg, 25%) as an oil.

Step D: 4-(2-(Methoxymethyl)-3,4-dihydro-2H-pyran-5-yl)morpholine (100%) was prepared according to Example 93, Step A, substituting 6-(methoxymethyl)dihydro-2H-pyran-3(4H)-one for dihydro-2H-pyran-3(4H)-one.

Step E: 8-Bromo-2-(methoxymethyl)-4a-morpholino-2,3,4,4a,10,10a-hexahydropyrano[3,2-b]chromen-10-ol (35%) was prepared according to Example 93, Step B, substituting 6-(methoxymethyl)dihydro-2H-pyran-3(4H)-one for dihydro-2H-pyran-3(4H)-one.

Step F: 8-Bromo-2-(methoxymethyl)-3,4-dihydropyrano[3,2-b]chromen-10(2H)-one (27%) was prepared according to Example 93, Step C, substituting 8-bromo-2-(methoxymethyl)-4a-morpholino-2,3,4,4a,10,10a-hexahydropyrano[3,2-b]chromen-10-ol for 8-bromo-4a-morpholino-2,3,4,4a,10,10a-hexahydropyrano[3,2-b]chromen-10-ol.

Step G: (4aS,10aS)-8-Bromo-2-(methoxymethyl)-2,3,4,4a-tetrahydropyrano[3,2-b]chromen-10(10aH)-one (42%) was prepared according to Example 93, Step D, substituting 8-bromo-2-(methoxymethyl)-3,4-dihydropyrano[3,2-b]chromen-10(2H)-one for 8-bromo-3,4-dihydropyrano[3,2-b]chromen-10(2H)-one.

Step H: (4aS,10aR)-8-Bromo-2-(methoxymethyl)-10-methylene-2,3,4,4a,10,10a-hexahydropyrano[3,2-b]chromene (72%) was prepared according to Example 93, Step E, substituting (4aS,10aS)-8-bromo-2-(methoxymethyl)-2,3,4,4a-tetrahydropyrano[3,2-b]chromen-10(10aH)-one for (4aR,10aR)-8-bromo-2,3,4,4a-tetrahydropyrano[3,2-b]chromen-10(10aH)-one.

Step I: (4a′S,10a′R)-8′-Bromo-2′-(methoxymethyl)-3′,4′,4a′,10a′-tetrahydro-2′H,5H-spiro[oxazole-4,10′-pyrano[3,2-b]chromen]-2-amine (55%) was prepared according to Example 93, Step F, substituting (4aS,10aR)-8-bromo-2-(methoxymethyl)-10-methylene-2,3,4,4a,10,10a-hexahydropyrano[3,2-b]chromene for (4aS,10aR)-8-bromo-10-methylene-2,3,4,4a,10,10a-hexahydropyrano[3,2-b]chromene.

Step J: (2′R*,4R*,4a′S*,10a′R*)-8′-(2-Fluoropyridin-3-yl)-2′-(methoxymethyl)-3′,4′,4a′,10a′-tetrahydro-2′H,5H-spiro[oxazole-4,10′-pyrano[3,2-b]chromen]-2-amine (32%) was prepared according to Example 93, Step G, substituting (4a′S,10a′R)-8′-bromo-2′-(methoxymethyl)-3′,4′,4a′,10a′-tetrahydro-2′H,5H-spiro[oxazole-4,10′-pyrano[3,2-b]chromen]-2-amine for (4a′S,10a′R)-8′-bromo-3′,4′,4a′,10a′-tetrahydro-2′H,5H-spiro[oxazole-4,10′-pyrano[3,2-b]chromen]-2-amine, and 2-fluoropyridin-3-ylboronic acid for 5-chloropyridin-3-ylboronic acid. Relative stereochemistry was determined by X-ray crystallography. ¹H NMR (400 MHz, CDCl₃) δ 8.17-8.13 (m, 1H); 7.87-7.78 (m, 1H); 7.49-7.44 (m, 1H); 7.42-7.35 (m, 1H); 7.27-7.22 (m, 1H); 6.93-6.87 (m, 1H), 4.78-4.75 (m, 1H); 4.69-4.66 (m, 1H); 4.31-4.23 (m, 1H); 4.18-4.14 (m, 1H); 3.75-3.66 (m, 2H); 3.56-3.43 (m, 1H); 3.41 (s, 3H), 2.44-2.33 (m, 1H), 1.93-1.53 (m, 3H); m/z (APCI-pos) M+1=400.1.

### Example 98

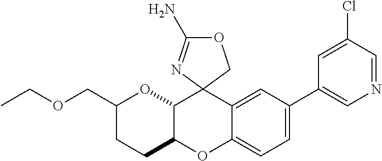

### (4a′S*,10a′R*)-8′45-chloropyridin-3-yl)-2′-(ethoxymethyl)-3′,4′,4a′,10a′-tetrahydro-2H,5H-spiro[oxazole-4,10′-pyrano[3,2-b]chromen]-2-amine

Step A: 2-(Ethoxymethyl)-3,4-dihydro-2H-pyran (72%) was prepared according to Example 97, Step A, substituting EtI for MeI.

Step B: 6-(Ethoxymethyl)tetrahydro-2H-pyran-3-ol (24%) was prepared according to Example 97, Step B, substituting 2-(ethoxymethyl)-3,4-dihydro-2H-pyran for 2-(methoxymethyl)-3,4-dihydro-2H-pyran.

Step C: 6-(Ethoxymethyl)dihydro-2H-pyran-3(4H)-one (26%) was prepared according to Example 97, Step C, substituting 6-(ethoxymethyl)tetrahydro-2H-pyran-3-ol for 6-(methoxymethyl)tetrahydro-2H-pyran-3-ol.

Step D: 4-(2-(Ethoxymethyl)-3,4-dihydro-2H-pyran-5-yl)morpholine (100%) was prepared according to Example 93, Step A, substituting 6-(ethoxymethyl)dihydro-2H-pyran-3(4H)-one for dihydro-2H-pyran-3(4H)-one.

Step E: 8-Bromo-2-(ethoxymethyl)-4a-morpholino-2,3,4,4a,10,10a-hexahydropyrano[3,2-b]chromen-10-ol (53%) was prepared according to Example 93, Step B, substituting 4-(2-(ethoxymethyl)-3,4-dihydro-2H-pyran-5-yl)morpholine for 4-(3,4-dihydro-2H-pyran-5-yl)morpholine.

Step F: 8-Bromo-2-(ethoxymethyl)-3,4-dihydropyrano[3,2-b]chromen-10(2H)-one (51%) was prepared according to Example 93, Step C, substituting 8-bromo-2-(ethoxymethyl)-4a-morpholino-2,3,4,4a,10,10a-hexahydropyrano[3,2-b]chromen-10-ol for 8-bromo-4a-morpholino-2,3,4,4a,10,10a-hexahydropyrano[3,2-b]chromen-10-ol.

Step G: (4aS,10aS)-8-Bromo-2-(ethoxymethyl)-2,3,4,4a-tetrahydropyrano[3,2-b]chromen-10(10aH)-one (50%) was prepared according to Example 93, Step D, substituting 8-bromo-2-(ethoxymethyl)-3,4-dihydropyrano[3,2-b]chromen-10(2H)-one for 8-bromo-3,4-dihydropyrano[3,2-b]chromen-10(2H)-one.

Step H: (4aS,10aR)-8-Bromo-2-(ethoxymethyl)-10-methylene-2,3,4,4a,10,10a-hexahydropyrano[3,2-b]chromene (51%) was prepared according to Example 93, Step E, substituting (4aS,10aS)-8-bromo-2-(ethoxymethyl)-2,3,4,4a-tetrahydropyrano[3,2-b]chromen-10(10aH)-one for (4aR,10aR)-8-bromo-2,3,4,4a-tetrahydropyrano[3,2-b]chromen-10(10aH)-one.

Step I: (4a′S,10a′R)-8′-Bromo-2′-(ethoxymethyl)-3′,4′,4a′,10a′-tetrahydro-2′H,5H-spiro[oxazole-4,10′-pyrano[3,2-b]chromen]-2-amine (95%) was prepared according to Example 93, Step F, substituting (4aS,10aR)-8-bromo-2-(ethoxymethyl)-10-methylene-2,3,4,4a,10,10a-hexahydropyrano[3,2-b]chromene for (4aS,10aR)-8-bromo-10-methylene-2,3,4,4a,10,10a-hexahydropyrano[3,2-b]chromene.

Step J: (4a′S*,10a′R*)-8′-(5-Chloropyridin-3-yl)-2′-(ethoxymethyl)-3′,4′,4a′,10a′-tetrahydro-2′H,5H-spiro[oxazole-4,10′-pyrano[3,2-b]chromen]-2-amine (22%) was prepared according to Example 93, Step G, substituting (4a′S,10a′R)-8′-bromo-2′-(ethoxymethyl)-3′,4′,4a′,10a′-tetrahydro-2′H,5H-spiro[oxazole-4,10′-pyrano[3,2-b]chrome]2-amine for (4a′S,10a′R)-8′-bromo-3′,4′,4a′,10a′-tetrahydro-2′H,5H-spiro[oxazole-4,10′-pyrano[3,2-b]chromen]-2-amine. ¹H NMR (400 MHz, CDCl₃) δ 8.72-8.60 (m, 1H); 8.55-8.47 (m, 1H); 7.85-7.77 (m, 1H); 7.55-7.34 (m, 2H); 6.94-6.87 (m, 1H), 4.76-4.70 (m, 1H); 4.68-4.63 (m, 1H); 4.33-4.24 (m, 1H); 4.13-4.08 (m, 1H); 3.75-3.41 (m, 5H); 2.45-2.33 (m, 1H), 1.93-1.53 (m, 3H); 1.30-1.12 (m, 3H); m/z (APCI-pos) M+1=430.1, 432.1.

### Example 99

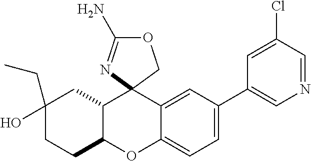

### (4R*,4a′S*,9a′R*)-2-amino-7′-(5-chloropyridin-3-yl)-2′-ethyl-1′,2′,3′,4′,4a′,9a′-hexahydro-5,1-spiro[oxazole-4,9′-xanthen]-2′-ol

Step A: A mixture of (4a′S,9a′R)-2-amino-7′-bromo-1′,4′,4a′,9a′-tetrahydro-5H-spiro[oxazole-4,9′-xanthen]-2′(3′H)-one (0.516 g, 1.47 mmol), Boc₂O (0.962 g, 4.41 mmol) and TEA (d. 0.726) (0.614 mL, 4.41 mmol) in DCM (10 mL) was stirred at room temperature overnight. The mixture was worked up with DCM and water. The organics were extracted with DCM twice, washed with brine and dried with Na₂SO₄. This was then purified on prep HPLC to give the product (0.285 g, 0.517 mmol, 35.2% yield).

Step B: Ethylmagnesium bromide (0.172 mL, 0.517 mmol) was added to a mixture of the product from Step A (0.114 g, 0.207 mmol) in THF (1 mL) at −78° C. The mixture was then allowed to stir at room temperature for an hour. The mixture was worked up the water and DCM. The organics were extracted with DCM twice, washed with brine and dried with Na₂SO₄. This was then concentrated down and purified on prep HPLC to give (4a′S,9a′R)-2-amino-7′-bromo-2′-ethyl-1′,2′,3′,4′,4a′,9a′-hexahydro-5H-spiro[oxazole-4,9′-xanthen]-2′-ol (0.025 g, 0.0656 mmol, 31.7% yield).

Step C: A mixture of (4a′S,9a′R)-2-amino-7′-bromo-2′-ethyl-1′,2′,3′,4′,4a′,9a′-hexahydro-5H-spiro[oxazole-4,9′-xanthen]-2′-ol (0.025 g, 0.0656 mmol), 5-chloropyridin-3-ylboronic acid (0.0114 g, 0.0721 mmol), Na₂CO₃ (0.105 mL, 0.210 mmol) and Pd(PPh₃)₄ (0.00758 g, 0.00656 mmol) in dioxane (1.2 mL) was stirred overnight at 90° C. The mixture was concentrated down and purified on prep HPLC to give (4S*,4a′S*,9a′R*)-2-amino-7′-(5-chloropyridin-3-yl)-2′-ethyl-1′,2′,3′,4′,4a′,9a′-hexahydro-5H-spiro[oxazole-4,9′-xanthen]-2′-ol (0.0045 g, 0.0109 mmol, 16.6% yield, m/z (APCI-pos) M+1=414.1) and (4R*,4a′S*,9a′R*)-2-amino-7′-(5-chloropyridin-3-yl)-2′-ethyl-1′,2′,3′,4′,4a′,9a′-hexahydro-5H-spiro[oxazole-4,9′-xanthen]-2′-ol (0.0115 g, 0.0278 mmol, 42.4% yield, m/z (APCI-pos) M+1=414.1).

### Example 100

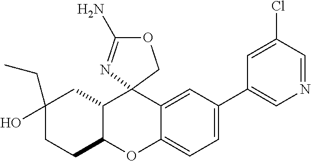

### (4S*,4a′S*,9a′R*)-2-amino-7′-(5-chloropyridin-3-yl)-2′-ethyl-1′,2′,3′,4′,4a′,9a′-hexahydro-5H-spiro[oxazole-4,9′-xanthen]-2′-ol

(4S*,4a′S*,9aR*)-2-Amino-7′-(5-chloropyridin-3-yl)-2′-ethyl-1′,2′,3′,4′,4a′,9a′-hexahydro-5H-spiro[oxazole-4,9′-xanthen]-2′-ol was prepared in Example 99, Step C.

### Example 101

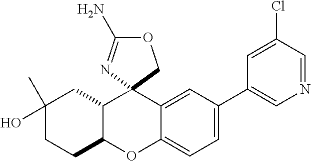

### (4S*,4a′S*,9a′R*)-2-amino-7′-(5-chloropyridin-3-yl)-2′-methyl-1′,2′,3′,4′,4a′,9a′-hexahydro-5H-spiro[oxazole-4,9′-xanthen]-2′-ol

Step A: A mixture of 2-amino-7′-bromo-1′,4′,4a′,9a′-tetrahydro-5H-spiro[oxazole-4,9′-xanthen]-2′(3′H)-one (0.145 g, 0.413 mmol), 5-chloropyridin-3-ylboronic acid (0.0715 g, 0.454 mmol), Na₂CO₃ (0.454 mL, 0.908 mmol) and Pd(PPh₃)₄ (0.0477 g, 0.0413 mmol) in dioxane (1.2 mL) was stirred overnight at 90° C. The mixture was concentrated down and purified on a column using DCM:MeOH:NH₄OH (90:10:1) to give 2-amino-7′-(5-chloropyridin-3-yl)-1′,4′,4a′,9a′-tetrahydro-5H-spiro[oxazole-4,9′-xanthen]-2′(3′H)-one (0.090 g, 0.234 mmol, 56.8% yield).

Step B: A mixture of (4a′S,9a′R)-2-amino-7′-(5-chloropyridin-3-yl)-1′,4′,4a′,9a′-tetrahydro-5H-spiro[oxazole-4,9′-xanthen]-2′(3′H)-one (0.090 g, 0.23 mmol), Boc₂O (0.056 g, 0.26 mmol) and TEA (d. 0.726) (0.098 mL, 0.70 mmol) in DCM (1 mL) was stirred overnight at room temperature. This was then worked up using DCM and water. The organics were extracted with DCM twice, washed with brine and dried with Na₂SO₄. This was then concentrated down and purified on a column using EtOAc:hexane to give the product (0.031 g, 0.053 mmol, 23% yield).

Step C: MeMgBr (0.044 mL, 0.13 mmol) was added to the product of Step B (0.031 g, 0.053 mmol) in THF (0.27 mL) at −78° C. The mixture was stirred at room temperature for 1 hour. The mixture was quenched with water and worked up with DCM. The organics were concentrated down and purified on prep HPLC to give (4R*,4a′S*,9a′R*)-2-amino-7′-(5-chloropyridin-3-yl)-7′-methyl-1′,2′,3′,4′,4a′,9a′-hexahydro-5H-spiro[oxazole-4,9′-xanthen]-2′-ol (0.007 g, 0.014 mmol, 27% yield, m/z (APCI-pos) M+1=400.1) and (4S*,4a′S*,9a′R*)-2-amino-7′-(5-chloropyridin-3-yl)-2′-methyl-1′,2′,3′,4′,4a′,9a′-hexahydro-5H-spiro[oxazole-4,9′-xanthen]-2′-ol (0.0035 g, 0.0070 mmol, 13% yield, m/z (APCI-pos) M+1=400.1).

### Example 102

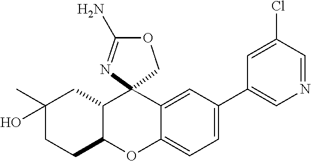

### (4R*,4a′S*,9a′R*)-2-amino-7′-(5-chloropyridin-3-yl)-2′-methyl-1′,2′,3′,4′,4a′,9a′-hexahydro-5H-spiro[oxazole-4,9′-xanthen]-2′-ol

(4R*,4a′S*,9a′R*)-2-Amino-7′-(5-chloropyridin-3-yl)-2′-methyl-1′,2′,3′,4′,4a′,9a′-hexahydro-5H-spiro[oxazole-4,9′-xanthen]-2′-ol was prepared in Example 101, Step C.

### Example 103

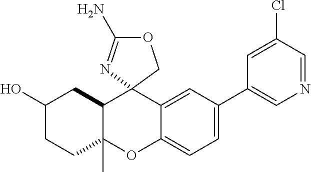

### (4S*,4a′R*,9a′S*)-2-amino-7′-(5-chloropyridin-3-yl)-4a′-methyl-1′,2′,3′,4′,4a′,9a′-hexahydro-5H-spiro[oxazole-4,9′-xanthen]-2′-ol

Step A: 5-Bromo-2-fluorobenzoic acid (18.0 g, 82.2 mmol) was dissolved in DCM (411 mL), cooled to 0° C., and then treated with oxalyl chloride (7.89 mL, 90.4 mmol) and 3 drops of DMF. The reaction mixture was stirred at ambient temperature for 1 hour then concentrated in vacuo and azeotroped with toluene (3×). In a separate flask, lithium bis(trimethylsilyl)amide (181 mL, 181 mmol) was dissolved in THF (411 mL), cooled to −78° C., and treated portion wise with 1,4-dioxaspiro[4.5]decan-8-one (12.8 g, 82.2 mmol). The reaction mixture was then stirred at −78° C. for 1 hour and then treated with the acid chloride, warmed to ambient temperature, and stirred for 2 hours. The reaction mixture was quenched with water and then extracted with EtOAc (2×). The organics were combined and washed with water (2×) and brine (1×), dried over Na₂SO₄, filtered, and concentrated in vacuo. The resulting residue was triturated with EtOAc to provide 7-(5-bromo-2-fluorobenzoyl)-1,4-dioxaspiro[4.5]decan-8-one (19.0 g, 53.2 mmol, 64.7% yield). ¹H NMR (400 MHz, CDCl₃) δ 7.55-7.48 (m, 2H), 7.03-6.99 (t, 1H), 3.97-3.92 (m, 4H), 2.71-2.68 (t, 2H), 2.43 (s, 2H), 1.94-1.91 (t, 2H).

Step B: In an oven dried flask, 7-(5-bromo-2-fluorobenzoyl)-1,4-dioxaspiro[4.5]decan-8-one (11.8 g, 33.0 mmol) was dissolved in THF (165 mL), cooled to 0° C., and degassed with N₂. The reaction mixture was then slowly treated with methylmagnesium bromide (3.0M diethyl ether solution) via cannula and then heated to reflux for 3 hours. The reaction mixture was then cooled to ambient temperature, poured onto ice, filtered, and concentrated in vacuo. The resulting residue was diluted with EtOAc and washed with water (1×) and brine (1×), dried over Na₂SO₄, filtered and concentrated in vacuo. Silica gel chromatography (Hexanes/EtOAc) provided (5-bromo-2-fluorophenyl)(8-hydroxy-8-methyl-1,4-dioxaspiro[4.5]decan-7-yl)methanone (8.03 g, 21.5 mmol, 65.1% yield) as a mixture of diastereomers.

Step C: (5-Bromo-2-fluorophenyl)(8-hydroxy-8-methyl-1,4-dioxaspiro[4.5]decan-7-yl)methanone (7.54 g, 20.2 mmol) was dissolved in THF (100 mL), treated with sodium hydride (848 mg, 21.2 mmol), and then heated to reflux for 3 hours. The reaction mixture was then cooled to ambient temperature, treated with water, and concentrated in vacuo. The resulting residue was then dissolved in EtOAc and washed with water (2×) and brine (1×), dried over Na₂SO₄, filtered and concentrated in vacuo. The crude reaction mixture was then dissolved in MeOH (100 mL), treated with potassium carbonate (5.58 g, 40.4 mmol) and stirred at ambient temperature for 1 hour. The reaction mixture was then concentrated in vacuo, dissolved in EtOAc and washed with water (2×) and brine (1×), dried over Na₂SO₄, filtered and concentrated in vacuo. Silica gel chromatography (Hexanes/EtOAc) provided 7′-bromo-4a′-methyl-1′,4′,4a′,9a′-tetrahydrospiro[[1,3]dioxolane-2,2′-xanthen]-9′(3′H)-one (3.37 g, 9.54 mmol, 47.2% yield) as a 5:1 cis:trans mixture of diastereomers.

Step D: 7′-Bromo-4a′-methyl-1′,4′,4a′,9a′-tetrahydrospiro[[1,3]dioxolane-2,2′-xanthen]-9′(3′H)-one (2.68 g, 7.59 mmol) was dissolved in THF (20 mL), cooled to −20° C., and then slowly treated with lithium bis(trimethylsilyl)amide (7.97 mL, 7.97 mmol., 1.0 M) and stirred at −20° C. for 2 hours. The reaction mixture was then added drop wise via cannula to a −78° C. 20 mL THF solution of ethyl salicylate (4.46 mL, 30.35 mmol) and stirred at −78° C. for 30 minutes. The reaction mixture was poured into a saturated NH₄Cl solution, concentrated off organic solvent in vacuo, and then extracted with EtOAc (2×). The organics were combined and washed with water (1×) and brine (1×), dried over Na₂SO₄, filtered and concentrated in vacuo. Silica gel chromatography (Hexanes/EtOAc) provided 7′-bromo-4a′-methyl-1′,4′,4a′,9a′-tetrahydrospiro[[1,3]dioxolane-2,2′-xanthen]-9′(3′H)-one (2.44 g, 6.91 mmol, 91.0% yield) as a 3:1 trans:cis mixture of diastereomers.

Step E: 7′-Bromo-4a′-methyl-1′,4′,4a′,9a′-tetrahydrospiro[[1,3]dioxolane-2,2′-xanthen]-9′(3′H)-one (2.25 g, 6.37 mmol) was dissolved in THF (32 mL), cooled to 0° C., and slowly treated with Tebbe reagent (19.1 mL, 9.56 mmol, 0.5 M). The reaction mixture was warmed to ambient temperature and stirred for 3 hours then cooled to 0° C. and slowly treated with 0.5 M Rochelle's salt solution until bubbling ceased. The reaction mixture was warmed to ambient temperature and stirred for 30 minutes then diluted with EtOAc, filtered, and concentrated in vacuo. The resulting residue was dissolved in EtOAc and washed with water (2×) and brine (1×), dried over Na₂SO₄, filtered, and concentrated in vacuo. Silica gel chromatography provided both (4a′R,9a′S)-7′-bromo-4a′-methyl-9′-methylene-1′,3′,4′,4a′,9′,9a′-hexahydrospiro[[1,3]dioxolane-2,2′-xanthene] (689.5 mg, 1.96 mmol, 30.8% yield) as pure trans diastereomer and (4a′R,9a′R)-7′-bromo-4a′-methyl-9′-methylene-1′,3′,4′,4a′,9′,9a′-hexahydrospiro[[1,3]dioxolane-2,2′-xanthene] (164.3 mg, 0.468 mmol, 7.3% yield) as pure cis diastereomer. ¹H NMR (400 MHz, CDCl₃) δ 7.93-7.92 (d, J=2.2 Hz, 1H), 7.55-7.52 (dd, J=2.7 Hz, 2.4 Hz, 1H), 6.85-6.83 (d, J=9.0 Hz, 1H), 4.03-3.94 (m, 4H), 3.17-3.13 (dd, J=4.1 Hz, 3.5 Hz, 1H), 2.32-2.20 (m, 2H), 2.00-1.95 (m, 1H), 1.87-1.81 (m, 1H), 1.77-1.69 (m, 1H), 1.57-1.51 (m, 1H), 1.29 (m, 3H).

Step F: Silver cyanate (882.7 mg, 5.89 mmol) was dissolved in 1:1 THF:ACN (12 mL), cooled to 0° C., and then treated with iodine (1.25 g, 4.91 mmol) followed by a 6.0 mL THF solution of (4a′R,9a′S)-7′-bromo-4a′-methyl-9′-methylene-1′,3′,4′,4a′,9′,9a′-hexahydrospiro[[1,3]dioxolane-2,2′-xanthene] (689.5 mg, 1.96 mmol) and stirred at ambient temperature for 24 hours. The reaction mixture was then filtered through GF/F paper and the filtrate was then treated with ammonium hydroxide (132.6 μL, 1.96 mmol) and stirred at ambient temperature for 24 hours. The reaction mixture was then treated with a 10% sodium thiosulfate solution, concentrated in vacuo, and the residue extracted with EtOAc (2×). The organics were combined and washed with water (2×) and brine (1×), dried over Na₂SO₄, filtered, and concentrated in vacuo. Silica gel chromatography (DCM/IPA) provided (4aR,9a′S)-2-amino-7′-bromo-4a′-methyl-1′,4′,4a′,9a′-tetrahydro-5H-spiro[oxazole-4,9′-xanthen]-2′(3′H)-[1,3]dioxolane (709.7 mg, 1.73 mmol, 88.3% yield). m/z (APCI-pos) M+1=409.0, 412.0.

Step G: (4a′R,9a′S)-2-Amino-7′-bromo-4a′-methyl-1′,4′,4a′,9a′-tetrahydro-5H-spiro[oxazole-4,9′-xanthen]-2′(3′H)-[1,3]dioxolane (682.1 mg, 1.67 mmol) was dissolved in 4:1 ACN:water (16.5 mL) and treated with 5-chloropyridine-3-boronic acid (314.7 mg, 2.00 mmol) and potassium carbonate (691.0 mg, 5.00 mmol). The reaction mixture was then degassed with argon, treated with tetrakis(triphenylphospine)palladium(0) (192.6 mg, 0.167 mmol), sparged with argon for an additional 5 minutes, sealed, and heated to 85° C. for 4 hours. The reaction mixture was then cooled to ambient temperature and concentrated in vacuo. The residue was dissolved in 20% IPA/DCM and washed with water (2×), dried over Na₂SO₄, filtered, and concentrated. Silica gel chromatography (DCM/IPA) provided (4a′R,9a′S)-2-amino-7′-(5-chloropyridin-3-yl)-4a′-methyl-1′,4′,4a′,9a′-tetrahydro-5H-spiro[oxazole-4,9′-xanthen]-2′(3′H)-[1,3]dioxolane (420.8 mg, 0.952 mmol, 57.1% yield) as a 1:1 mixture of diastereomers. m/z (APCI-pos) M+1=442.1, 444.1.

Step H: (4a′R,9a′S)-2-Amino-7′-(5-chloropyridin-3-yl)-4a′-methyl-1′,4′,4a′,9a′-tetrahydro-5H-spiro[oxazole-4,9′-xanthen]-2′(3′H)-[1,3]dioxolane (420.8 mg, 0.952 mmol) was dissolved in acetone (10 mL), treated with aqueous HCl (4.0 M), and heated to reflux for 2 hours. The reaction mixture was then cooled to ambient temperature, neutralized with saturated NaHCO₃, and concentrated in vacuo. The residue was dissolved in 20% IPA/DCM and washed with NaHCO₃ (1×) and brine (1×), dried over Na₂SO₄, filtered and concentrated in vacuo. Trituration with DCM provided (4S,4aR,9a′S)-2-amino-7′-(5-chloropyridin-3-yl)-4a′-methyl-1′,4′,4a′,9a′-tetrahydro-5H-spiro[oxazole-4,9′-xanthen]-2′(3′H)-one (70.1 mg, 0.176 mmol, 18.5% yield). M+1=398.1, 400.1. The filtrate was then concentrated in vacuo, followed by C18 chromatography (water-ACN) to provide (4R,4a′R,9a′S)-2-amino-7′-(5-chloropyridin-3-yl)-4a′-methyl-1′,4′,4a′,9a′-tetrahydro-5H-spiro[oxazole-4,9′-xanthen]-2′(3′H)-one (97.9 mg, 0.246 mmol, 25.8% yield). m/z (APCI-pos) M+1=398.1, 400.1.

Step I: (4S,4a′R,9a′S)-2-Amino-7′-(5-chloropyridin-3-yl)-4a′-methyl-1′,4′,4a′,9a′-tetrahydro-5H-spiro[oxazole-4,9′-xanthen]-2′(3′H)-one (45.3 mg, 0.114 mmol) was dissolved in 4:1 DCM:IPA (10 mL), treated with sodium borohydride (12.9 mg, 0.342 mmol), and stirred at ambient temperature for 2 hours. The reaction mixture was concentrated in vacuo then triturated with 3 mL 4:1 DCM:IPA to provide (4S*,4a′R*,9a′S*)-2-amino-7′-(5-chloropyridin-3-yl)-4a′-methyl-1′,2′,3′,4′,4a′,9a′-hexahydro-5H-spiro[oxazole-4,9′-xanthen]-2′-ol (9.3 mg, 0.023 mmol, 20.4% yield). ¹H NMR (400 MHz, (CD₃)₂SO-d6) δ 8.74 (d, J=1.9 Hz, 1H), 8.56-8.55 (d, J=2.3 Hz, 1H), 8.14-8.13 (m, 1H), 7.52-7.50 (dd, J=2.4 Hz, 2.0 Hz, 1H), 7.40 (d, J=2.3 Hz, 1H), 6.85-6.83 (d, J=8.5 Hz, 1H), 6.02 (s, 2H), 4.69-4.68 (d, J=4.6 Hz, 1H), 4.42-4.40 (d, J=8.2 Hz, 1H), 4.17-4.15 (d, J=8.6 Hz, 1H), 3.61 (m, 1H), 1.87-1.60 (m, 5H), 1.37-1.24 (m, 5H); m/z (APCI-pos) M+1=400.1, 402.1.

### Example 104

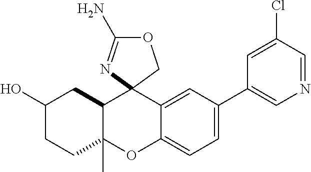

### (4R*,4aR*,9a′S*)-2-amino-7′-(5-chloropyridin-3-yl)-4a′-methyl-1′,2′,3′,4′,4a′,9a′-hexahydro-5H-spiro[oxazole-4,9′-xanthen]-2′-ol

(4R,4a′R,9a′S)-2-amino-7′-(5-chloropyridin-3-yl)-4a′-methyl-1′,4′,4a′,9a′-tetrahydro-5H-spiro[oxazole-4,9′-xanthen]-2′(3′H)-one (97.9 mg, 0.246 mmol) was dissolved in 4:1 DCM:IPA (2.5 mL), treated with sodium borohydride (27.9 mg, 0.738 mmol), and stirred at ambient temperature for 1 hour. The reaction mixture was diluted with additional 4:1 DCM:IPA and washed with water (2×) then dried over Na₂SO₄, filtered, and concentrated in vacuo. Silica gel chromatography (DCM/IPA with 2% NH₄OH) provided (4R*,4a′R*,9a′S*)-2-amino-7′-(5-chloropyridin-3-yl)-4a′-methyl-1′,2′,3′,4′,4a′,9a′-hexahydro-5H-spiro[oxazole-4,9′-xanthen]-2′-ol (26.6 mg, 0.067 mmol, 27.0% yield). ¹H NMR (400 MHz, (CD₃)₂SO) δ 8.76 (s, 1H), 8.56-8.55 (d, J=1.8 Hz, 1H), 8.13 (s, 1H), 7.55-7.48 (m, 2H), 6.84-6.82 (d, J=8.6 Hz, 1H), 4.82-4.81 (d, J=3.9 Hz, 1H), 4.56 (s, 1H), 4.17 (s, 1H), 3.56 (m, 1H), 1.99-1.61 (m, 5H), 1.42-1.24 (m, 2H), 1.18 (s, 3H).m/z (APCI-pos) M+1=400.1, 402.1.

### Example 105

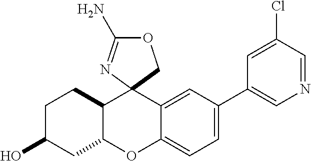

### (3 S*,4aR*,4′S*,9aR*)-2′-amino-7-(5-chloropyridin-3-yl)-2,3,4,4a,9a,10-hexahydro-1H,5′H-spiro[anthracene-9,4′-oxazol]-3-ol

Step A: To a solution of 1,4-dioxaspiro[4.5]decan-8-one (14.4 g, 92.2 mmol) in toluene (300 mL) was added pyrrolidine (8.08 mL, 96.8 mmol), followed by TsOH—H₂O (0.175 g, 0.922 mmol). The reaction mixture was refluxed using a Dean-stark trap for 3 hours and then cooled to room temperature. 1-Bromo-4-(bromomethyl)benzene (24.2 g, 96.8 mmol) was added and the mixture was refluxed for 16 hours. The reaction mixture was cooled to room temperature and to this was added acetate buffer (NaOAc:AcOH:H₂O, 1:2:2, 300 mL) and the mixture was refluxed for 1 hour. After cooling to room temperature, the layers were separated. The aqueous layer was extracted with EtOAc (2×). The combined organics were basified with saturated NaHCO₃ until the pH of the aqueous layer is about 7. The layers were separated and the aqueous layer was extracted with EtOAc (2×). The combined organics were dried, filtered and concentrated. The crude product was purified via column chromatography, eluting with hexanes/EtOAc (3:1) to give 7-(4-bromobenzyl)-1,4-dioxaspiro[4.5]decan-8-one as a solid (27 g, 90%). ¹H NMR (400 MHz, CDCl₃) δ7.38 (m, 2H), 7.02 (m, 2H), 4.00-3.90 (m, 4H), 3.14 (m, 1H), 2.93-2.83 (m, 1H), 2.66 (m, 1H), 2.45-2.36 (m, 2H), 2.07-1.89 (m, 3H), 1.67 (t, J=13.1 Hz, 1H).

Step B: To a cold (0° C.) suspension of (methoxymethyl)triphenylphosphonium chloride (58.8 g, 172 mmol) in dry THF (250 mL) was added 2.5M butyllithium (61.8 mL, 154 mmol) in hexanes dropwise over 30 minutes. The resulting solution was stirred at room temperature for 15 minutes and then cooled back down to −78° C. To this was added a solution of 7-(4-bromobenzyl)-1,4-dioxaspiro[4.5]decan-8-one (27.9 g, 85.8 mmol) in THF (100 mL) via cannula. The reaction mixture was allowed to warm up to room temperature overnight. The reaction was carefully quenched with water and concentrated to remove THF. The resulting residue was extracted with ether (2×). The combined organics were dried, filtered and concentrated. The crude product was purified via column chromatography, eluting with hexanes/EtOAc (20:1) to give 7-(4-bromobenzyl)-8-(methoxymethylene)-1,4-dioxaspiro[4.5]decane (21.9 g, 72%) as a mixture of E/Z isomers.

Step C: A solution of 7-(4-bromobenzyl)-8-(methoxymethylene)-1,4-dioxaspiro[4.5]decane (22.5 g, 63.7 mmol) in THF/2N HCl (1:1, 956 mL) was stirred at room temperature for 16 hours. The reaction mixture was concentrated to remove THF and the residue was taken up in EtOAc. The aqueous layer was extracted with EtOAc (1×) and the combined organics were dried, filtered and concentrate. The crude product was purified via column chromatography, eluting with hexanes/EtOAc (10:1) to give 2-(4-bromobenzyl)-4-oxocyclohexanecarbaldehyde (17.2 g, 92%) as a mixture of cis and trans isomers.

Step D: A solution of 2-(4-bromobenzyl)-4-oxocyclohexanecarbaldehyde (17.2 g, 58.3 mmol) and t-BuOH (8.36 mL, 87.4 mmol) in THF (200 mL) and water (200 mL) was cooled to 0° C. To this was added 2.0M 2-methylbut-2-ene (87.4 mL, 175 mmol) in THF, followed by NaH₂PO₄ (83.9 g, 699 mmol) and sodium chlorite (11.9 g, 105 mmol). The reaction mixture was allowed to warm up to room temperature overnight. The reaction mixture was diluted with EtOAc and water. The aqueous layer was extracted with EtOAc (2×). The combined organics were dried, filtered and concentrated to give the crude 2-(4-bromobenzyl)-4-oxocyclohexanecarboxylic acid as a mixture of cis and trans isomers (16.8 g, 93%). m/z (APCI-nega) M−1=309.0, 311.0. The acid was used without purification in step E.

Step E: 2-(4-Bromobenzyl)-4-oxocyclohexanecarboxylic acid (17.4 g, 55.8 mmol) was treated with polyphosphoric acid (140 g) and H₂O (14 g) at 90° C. overnight. The next morning, the reaction mixture was cooled to room temperature and carefully quenched with ice cold water. The aqueous layer was extracted with EtOAc (2×). The combined organics were washed with saturated NaHCO₃, dried, filtered and concentrated. The crude product was purified via column chromatography, eluting with hexanes/EtOAc (5:1) to give 6-bromo-1,4,4a,9a-tetrahydroanthracene-2,10(3H,9H)-dione (2.1 g, 13%) as a mixture of cis and trans isomers.

Step F: A suspension of 6-bromo-4,4a,9,9a-tetrahydroanthracene-2,10(1H,3H)-dione (2.1 g, 7.163 mmol), ethane-1,2-diol (0.4406 mL, 7.880 mmol) and TsOH—H₂O (0.1363 g, 0.7163 mmol) in toluene (50 mL) was heated to reflux using a Dean-stark trap overnight. The reaction mixture was cooled to room temperature and concentrates. The residue was taken up in EtOAc and washed with water (1×). The organics were dried, filtered and concentrated. The crude product was purified via column chromatography, eluting with hexanes/EtOAc (9:1) to give (4aR,9a′R)-6-bromo-4,4a,9,9a-tetrahydro-1H-spiro[anthracene-2,2′-[1,3]dioxolan]-10(3H)-one (1.26 g, 53%) as a mixture of cis and trans (major) isomer.

Step G: To a solution of (4aR,9a′R)-6-bromo-4,4a,9,9a-tetrahydro-1H-spiro[anthracene-2,2′-[1,3]-dioxolan]-10(3H)-one (1.61 g, 4.77 mmol) in THF (50 mL) at 0° C. was added Tebbe reagent (0.5M in THF, 38.2 mL, 19.1 mmol) and the resulting mixture was stirred at 0° C. for 2 hours. The reaction was quenched slowly with 0.5 M Rochelle's salt solution until bubbling ceased and stirred for 30 minutes. The reaction mixture was extracted with EtOAc (3×) and the combined organics were dried, filtered and concentrated. The crude product was purified by flash chromatography, eluting with hexanes/EtOAc (20:1) to give (4aR,9aR)-6-bromo-10-methylene-3,4,4a,9,9a,10-hexahydro-1H-spiro[anthracene-2,2′-[1,3]dioxolane] (1.16 g, 73%) as pure trans isomer. ¹H NMR (400 MHz, CDCl₃) δ 7.70 (d, 2.0 Hz, 1H), 7.28-7.25 (m, 1H) 6.93 (d, J=7.8 Hz, 1H), 5.53 (d, J=2.2 Hz, 1H), 5.07 (d, 2.2 Hz, 1H), 4.00-3.93 (m, 4H), 2.83-2.77 (m, 1H), 2.53-2.46 (m, 1H), 2.17-2.09 (m, 1H), 2.00-1.80 (m, 4H), 1.70-1.65 (m, 2H), 1.48-1.42 (m, 1H).

Step H: To a pre-sonicated (2 minutes) mixture of diiodine (1.46 g, 5.75 mmol) and AgNCO (0.575 g, 3.84 mmol) in THF/ACN (1:1, 8 mL) was added a solution of (4aR,9a′R)-6-bromo-10-methylene-3,4,4a,9,9a,10-hexahydro-1H-spiro[anthracene-2,2′-[1,3]dioxolane] (0.643 g, 1.92 mmol) in THF (15 mL). The reaction mixture was stirred at room temperature overnight. The next morning, the solids were removed by filtration and the filter cake was washed with THF. The filtrate was treated with concentrate aqueous NH₂OH (9.07 mL, 134 mmol) at room temperature for 16 hours. The reaction mixture was concentrated and the residue was partitioned between EtOAc and water. The solids were removed by filtration and washed with EtOAc. The filtrate was transferred into a separation funnel and the layers were separated. The aqueous layer was extracted with EtOAc (2×). The combined organics were washed with saturate Na₂S₂O₃, dried, filtered and concentrated. The crude product was purified via column chromatography, eluting with EtOAc to give (4aR,9aR)-2′-amino-7-bromo-4,4a,9a,10-tetrahydro-1H,5′H-spiro[anthracene-9,4′-oxazol]-3(2H)-[1,3]dioxolane (0.566 g, 75%) as a mixture of diastereomers. m/z (APCI-pos) M+1=393.1, 395.1.

Step I: A solution of (4aR,9aR)-2′-amino-7-bromo-4,4a,9a,10-tetrahydro-1H,5′H-spiro[anthracene-9,4′-oxazol]-3(2H)-[1,3]dioxolane (0.169 g, 0.430 mmol) and 2 N HCl (2.15 mL, 4.30 mmol) in acetone (5 mL) was heated to reflux overnight. After cooling to room temperature, the reaction mixture was concentrated to remove acetone. The pH of the aqueous layer was adjusted to about 7 with saturated NaHCO₃ and then extracted with DCM (2×). The combined organics were dried, filtered and concentrated. The crude product was purified via column chromatography (dry loading), eluting with hexanes/EtOAc (1:4), EtOAc to give (4aR,9aR)-2′-amino-7-bromo-4,4a,9a,10-tetrahydro-1H,5H-spiro[anthracene-9,4′-oxazol]3(2H)-one (0.090 g, 60%) as a mixture of diastereomers. m/z (APCI-pos) M+1=349.0, 351.0.

Step J: A mixture of (4aR,9aR)-2′-amino-7-bromo-4,4a,9a,10-tetrahydro-1H,5′H-spiro[anthracene-9,4′-oxazol]-3(2H)-one (0.051 g, 0.146 mmol), 5-chloropyridin-3-ylboronic acid (0.024 g, 0.153 mmol), 2N Na₂CO₃ (0.219 mL, 0.438 mmol) and Pd(PPh₃)₄ (0.00844 g, 0.00730 mmol) in dioxane (4 mL) was degassed with Ar for 10 minutes. The reaction mixture was heated to 90° C. under Ar for 16 hours. After cooling to room temperature, the reaction mixture was partitioned between EtOAc and water. The layers were separated and the aqueous layer was extracted with EtOAc (1×). The combined organics were dried, filtered and concentrated. The crude product was purified via column chromatography, eluting with EtOAc/MeOH (50:1), EtOAc/MeOH (25:1) to give (4aR,9aR)-2′-amino-7-(5-chloropyridin-3-yl)-4,4a,9a,10-tetrahydro-1H,5H-spiro[anthracene-9,4′-oxazol]-3(2H)-one (0.005 g, 9%) as a single diastereomer. m/z (APCI-pos) M+1=382.1, 384.1.

Step K: To a −78° C. solution of (4aR,9aR)-2′-amino-7-(5-chloropyridin-3-yl)-4,4a,9a,10-tetrahydro-1H,5′H-spiro[anthracene-9,4′-oxazol]-3(2H)-one (0.005 g, 0.013 mmol) in a mixture of 1:1 THF/MeOH (4.0 mL) was added NaBH₄ (0.0020 g, 0.052 mmol). The cold bath was removed and the reaction mixture was allowed to warm up to room temperature slowly over 1 hour. The reaction was quenched with water (1 mL) and then concentrated. The residue was partitioned between EtOAc and water. The aqueous layer was extracted with EtOAc (1×). The combined organics were dried, filtered and concentrated. The crude product was purified reverse phase HPLC to give (3S*,4aR*,4′S*,9aR*)-2′-amino-7-(5-chloropyridin-3-yl)-2,3,4,4a,9a,10-hexahydro-1H,5′H-spiro[anthracene-9,4′-oxazol]-3-ol (0.0024 g, 38%) as a single diastereomer. ¹H NMR (400 MHz, (CD₃)OD) δ 8.77 (d, J=2.1 Hz, 1H), 8.56 (d, J=2.0 Hz, 1H), 8.21 m, 1H), 7.86 (d, J=1.8 Hz, 1H), 7.65-7.62 (m, 1H), 7.33 (d, J=7.8 Hz, 1H), 5.12 (d, J=9.8 Hz, 1H), 4.49 (d, J=9.8 Hz, 1H), 3.74-3.64 (m, 1H), 3.05-2.99 (m, 1H), 2.68-2.60 (m, 1H), 2.24-2.10 (m, 2H), 1.98-1.73 (m, 3H), 1.54-1.42 (m, 1H), 1.39-1.29 (m, 1H), 1.24-1.15 (m, 1H). m/z (APCI-pos) M+1=384.1, 386.1.

### Example 106

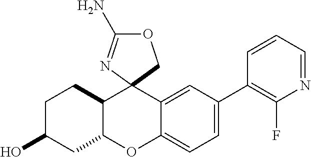

### (3 S*,4aR*,4′S*,9aR*)-2′-amino-7-(2-fluoropyridin-3-yl)-2,3,4,4a,9a,10-hexahydro-1H,5′H-spiro[anthracene-9,4′-oxazol]-3-ol

Step A: A mixture of (4aR,9aR)-2′-Amino-7-bromo-4,4a,9a,10-tetrahydro-1H,5′H-spiro[anthracene-9,4′-oxazol]-3(2H)-[1,3]dioxolane (0.064 g, 0.16 mmol), 2-fluoropyridin-3-ylboronic acid (0.030 g, 0.21 mmol), 2N Na₂CO₃ (0.24 mL, 0.49 mmol) and Pd(PPh₃)₄ (0.0094 g, 0.0081 mmol) in dioxane (4 mL) was degassed with Ar for 10 minutes. The reaction mixture was heated to 90° C. under Ar overnight. After cooling to room temperature, the reaction mixture was partitioned between EtOAc and water. The layers were separated and the aqueous layer was extracted with EtOAc (1×). The combined organics were dried, filtered and concentrated. The crude (4aR,9aR)-2′-amino-7-(2-fluoropyridin-3-yl)-4,4a,9a,10-tetrahydro-1H,5′H-spiro[anthracene-9,4′-oxazol]-3(2H)-[1,3]dioxolane was used as it is in Step B. m/z (APCI-pos) M+1=410.1.

Step B: A solution of (4aR,9aR)-2′-amino-7-(2-fluoropyridin-3-yl)-4,4a,9a,10-tetrahydro-1H,5′H-spiro[anthracene-9,4′-oxazol]-3(2H)-[1,3]dioxolane (0.067 g, 0.164 mmol) and 2N HCl (0.818 mL, 1.64 mmol) in acetone (10 mL) was heated to reflux overnight. After cooling to room temperature, the reaction mixture was concentrated to remove acetone. The pH of the aqueous layer was adjusted to about 7 with saturated NaHCO₃ and then extracted with DCM (2×). The combined organics were dried, filtered and concentrated. The crude product was purified via column chromatography, eluting with EtOAc, EtOAc/MeOH (20:1) to give (4aR,9aR)-2′-amino-7-(2-fluoropyridin-3-yl)-4,4a,9a,10-tetrahydro-1H,5′H-spiro[anthracene-9,4′-oxazol]-3(2H)-one (0.028, 47%). m/z (APCI-pos) M+1=366.1.

Step C: (3 S*,4aR*,4′S*,9aR*)-2′-Amino-7-(2-fluoropyridin-3-yl)-2,3,4,4a,9a,10-hexahydro-1H,5′H-spiro[anthracene-9,4′-oxazol]-3-ol (0.020 g, 56% over 2 steps) was prepared as a single diastereomer according the general procedure described in Example 105, Step D, substituting (4aR,9aR)-2′-amino-7-(2-fluoropyridin-3-yl)-4,4a,9a,10-tetrahydro-1H,5′H-spiro[anthracene-9,4′-oxazol]-3(2H)-one for (4aR,9aR)-2′-amino-7-(5-chloropyridin-3-yl)-4,4a,9a,10-tetrahydro-1H,5′H-spiro[anthracene-9,4′-oxazol]-3(2H)-one. ¹H NMR (400 MHz, (CD₃)OD) δ 8.19 (m, 1H), 8.11-8.05 (m, 1H), 7.73 (s, 1H), 7.53 (m, 1H), 7.44-7.40 (m, 1H), 7.34-7.30 (m, 1H), 5.12 (d, J=9.8 Hz, 1H), 4.49 (d, J=9.8 Hz, 1H), 3.74-3.64 (m, 1H), 3.05-2.99 (m, 1H), 2.68-2.60 (m, 1H), 2.24-2.10 (m, 2H), 1.98-1.73 (m, 3H), 1.54-1.42 (m, 1H), 1.39-1.29 (m, 1H), 1.24-1.15 (m, 1H). m/z (APCI-pos) M+1=368.1.

### Example 107

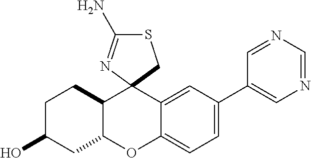

### (3 S*,4aR*,4′S*,9aR*)-2′-amino-7-(pyrimidin-5-yl)-2,3,4,4a,9a,10-hexahydro-1H,5′H-spiro[anthracene-9,4′-thiazol]-3-ol

Step A: (4aR,9aR)-2′-Amino-7-bromo-4,4a,9a,10-tetrahydro-1H,5′H-spiro[anthracene-9,4′-thiazol]-3(2H)-[1,3]dioxolane (0.151 g, 24%) was prepared as a mixture of diastereomers according the general procedure described in Example 105, Step A, substituting AgNCS for AgNCO. m/z (APCI-pos) M+1=409.0, 411.0.

Step B: (4aR,9aR)-2′-Amino-7-(pyrimidin-5-yl)-4,4a,9a,10-tetrahydro-1H,5′H-spiro[anthracene-9,4′-thiazol]-3(2H)-[1,3]dioxolane was prepared as a mixture of diastereomers according the general procedure described in Example 106, Step A, substituting (4aR,9aR)-2′-amino-7-bromo-4,4a,9a,10-tetrahydro-1H,5′H-spiro[anthracene-9,4′-thiazol]-3(2H)-[1,3]dioxolane for (4aR,9aR)-2′-amino-7-bromo-4,4a,9a,10-tetrahydro-1H,5′H-spiro[anthracene-9,4′-oxazol]-3(2H)-[1,3]dioxolane, and substituting pyrimidin-5-ylboronic acid for 2-fluoropyridin-3-ylboronic acid. m/z (APCI-pos) M+1=409.2.

Step C: (4aR,9aR)-2′-Amino-7-(pyrimidin-5-yl)-4,4a,9a,10-tetrahydro-1H,5′H-spiro[anthracene-9,4′-thiazol]-3(2H)-one (0.030, 44% over 2 steps) was prepared as a mixture of diastereomers according the general procedure described in Example 106, Step B, substituting (4aR,9aR)-2′-amino-7-(pyrimidin-5-yl)-4,4a,9a,10-tetrahydro-1H,5′H-spiro[anthracene-9,4′-thiazol]-3(2H)-[1,3]dioxolane for (4aR,9aR)-2′-amino-7-(2-fluoropyridin-3-yl)-4,4a,9a,10-tetrahydro-1H,5′H-spiro[anthracene-9,4′-oxazol]-3(2H)-[1,3]dioxolane. m/z (APCI-pos) M+1=365.1.

Step D: (3 S*,4aR*,4′S*,9aR*)-2′-amino-7-(pyrimidin-5-yl)-2,3,4,4a,9a,10-hexahydro-1H,5′H-spiro[anthracene-9,4′-thiazol]-3-ol (0.002 g, 5%) was prepared as a single diastereomer according the general procedure described in Example 105, Step D, substituting (4aR,9aR)-2′-amino-7-(pyrimidin-5-yl)-4,4a,9a,10-tetrahydro-1H,5′H-spiro[anthracene-9,4′-thiazol]-3(2H)-one for (4aR,9aR)-2′-amino-7-(5-chloropyridin-3-yl)-4,4a,9a,10-tetrahydro-1H,5′H-spiro[anthracene-9,4′-oxazol]-3(2H)-one. ¹H NMR (400 MHz, (CD₃)OD) δ 9.15 (s, 1H), 9.06 (s, 2H), 7.87 (d, J=2.1 Hz, 1H), 7.69-7.65 (m, 1H), 7.37 d, J=7.7 Hz, 1H), 4.22 (d, J=12.1 Hz, 1H), 3.99 (d, J=12.6 Hz, 1H), 3.65-3.57 (m, 1H), 2.96-2.90 (m, 1H), 2.68-2.60 (m, 1H), 2.25-2.12 (m, 3H), 1.88-1.71 (m, 2H), 1.50-1.32 (m, 2H), 1.25-1.16 (m, 1H). m/z (APCI-pos) M+1=366.8.

### Example 108

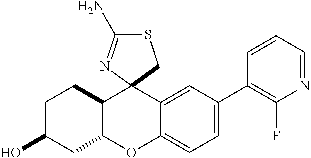

### (3S*,4aR*,4′S*,9aR*)-2′-amino-7-(2-fluoropyridin-3-yl)-2,3,4,4a,9a,10-hexahydro-1H,5′H-spiro[anthracene-9,4′-thiazol]-3-ol

Step A: (4aR,9aR)-2′-Amino-7-(2-fluoropyridin-3-yl)-4,4a,9a,10-tetrahydro-1H,5′H-spiro[anthracene-9,4′-thiazol]-3(2H)-[1,3]dioxolane was prepared as a mixture of diastereomers according the general procedure described in Example 106, Step A, substituting (4aR,9aR)-2′-Amino-7-bromo-4,4a,9a,10-tetrahydro-1H,5′H-spiro[anthracene-9,4′-thiazol]-3(2H)-[1,3]dioxolane for (4aR,9aR)-2′-amino-7-bromo-4,4a,9a,10-tetrahydro-1H,5′H-spiro[anthracene-9,4′-oxazol]-3(2H)-[1,3]dioxolane. m/z (APCI-pos) M+1=426.1.

Step B: (4aR,9aR)-2′-Amino-7-(2-fluoropyridin-3-yl)-4,4a,9a,10-tetrahydro-1H,5H-spiro[anthracene-9,4′-thiazol]-3(2H)-one (0.045, 75% over 2 steps) was prepared as a mixture of diastereomers according the general procedure described in Example 106, Step B, substituting (4aR,9aR)-2′-amino-7-(2-fluoropyridin-3-yl)-4,4a,9a,10-tetrahydro-1H,5′H-spiro[anthracene-9,4′-thiazol]-3(2H)-[1,3]dioxo lane for (4aR,9aR)-2′-amino-7-(2-fluoropyridin-3-yl)-4,4a,9a,10-tetrahydro-1H,5′H-spiro[anthracene-9,4′-oxazol]-3(2H)-[1,3]dioxolane. m/z (APCI-pos) M+1=382.1.

Step C: (3 S*,4aR*,4′S*,9aR*)-2′-Amino-7-(2-fluoropyridin-3-yl)-2,3,4,4a,9a,10-hexahydro-1H,5′H-spiro[anthracene-9,4′-thiazol]-3-01 (0.0037 g, 7%) was prepared as a single diastereomer according the general procedure described in Example 105, Step D, substituting (4aR,9aR)-2′-amino-7-(2-fluoropyridin-3-yl)-4,4a,9a,10-tetrahydro-1H,5′H-spiro[anthracene-9,4′-thiazol]-3(2H)-one for (4aR,9aR)-2′-amino-7-(5-chloropyridin-3-yl)-4,4a,9a,10-tetrahydro-1H,5′H-spiro[anthracene-9,4′-oxazol]-3(2H)-one. ¹H NMR (400 MHz, (CD₃)OD) δ 8.21-8.18 (m, 1H), 8.09-8.04 (m, 1H), 7.82 (s, 1H), 7.55-7.51 (m, 1H), 7.44-7.40 (m, 1H), 7.31 (d, J=8.4 Hz, 1H), 4.11 (d, J=12.1 Hz, 1H), 3.99 (d, J=12.6 Hz, 1H), 3.65-3.57 (m, 1H), 2.96-2.90 (m, 1H), 2.68-2.60 (m, 1H), 2.25-2.12 (m, 3H), 1.88-1.71 (m, 2H), 1.50-1.32 (m, 2H), 1.25-1.16 (m, 1H). m/z (APCI-pos) M+1=384.1.

### Example 109

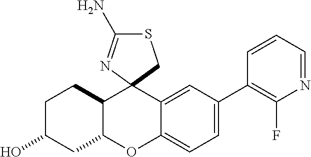

### (3R*,4aR*,4′S*,9aR*)-2′-amino-7-(2-fluoropyridin-3-yl)-2,3,4,4a,9a,10-hexahydro-1H,5′H-spiro[anthracene-9,4′-thiazol]-3-ol

(3R*,4aR*,4′S*,9aR*)-2′-Amino-7-(2-fluoropyridin-3-yl)-2,3,4,4a,9a,10-hexahydro-1H,5′H-spiro[anthracene-9,4′-thiazol]-3-ol was prepared as a single diastereomer according the general procedure described in Example 105, Step D, substituting (4aR,9aR)-2′-amino-7-(2-fluoropyridin-3-yl)-4,4a,9a,10-tetrahydro-1H,5′H-spiro[anthracene-9,4′-thiazol]-3(2H)-one for (4aR,9aR)-2′-amino-7-(5-chloropyridin-3-yl)-4,4a,9a,10-tetrahydro-1H,5H-spiro[anthracene-9,4′-oxazol]-3(2H)-one. ¹H NMR (400 MHz, (CD₃)OD) δ 8.21-8.18 (m, 1H), 8.09-8.04 μm, 1H), 7.82 (s, 1H), 7.55-7.51 (m, 1H), 7.44-7.40 (m, 1H), 7.31 (d, J=8.4 Hz, 1H), 4.12-4.08 (m, 1H), 3.99 (d, J=12.6 Hz, 1H), 2.88-2.82 (m, 1H), 2.59-2.52 (m, 1H), 2.16-1.94 (m, 3H), 1.87-1.70 (m, 4H), 1.45-1.37 (m, 1H). m/z (APCI-pos) M+1=384.1.

### Example 110

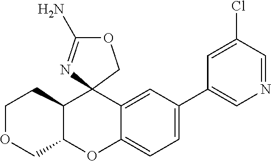

### (4R,4a′S,10a′S)-7′-(5-chloropyridin-3-yl)-3′,4′,4a′,10a′-tetrahydro-1′H,5H-spiro[oxazole-4,5′-pyrano[3,4-b]chromen]-2-amine

Step A: (3S,4S)-3,4-Dihydro-2H-pyran-3,4-diyl diacetate was prepared according to the procedure of Tetrahedron 67 (2011) 971-975.

Step B: (3S,4S)-3,4-Dihydro-2H-pyran-3,4-diyl diacetate (27.3 g, 136 mmol) was dissolved in methylene chloride (220 mL) followed by the sequential addition of triethylsilane (26.7 mL, 164 mmol) and boron trifluoride ether complex (18.0 mL, 143 mmol), and the reaction mixture was stirred for one hour. After one hour the mixture was diluted with methylene chloride and quenched slowly with saturated sodium bicarbonate solution. The organic layer was then dried over solid sodium sulfate, filtered and concentrated. The crude material was purified by silica gel eluting with a linear gradient of 0-30% ethyl acetate/heptane to yield (R)-3,6-dihydro-2H-pyran-3-yl acetate (15.0 g, 106 mmol, 77% yield). ¹H NMR (400 MHz, CDCl₃), d: 6.08 (m, 1H), 5.93 (m, 1H), 5.09 (m, 1H), 4.06-4.24 (m, 2H), 3.78-3.94 (m, 2H), 2.09 (s, 3H).

Step C: (R)-3,6-dihydro-2H-pyran-3-yl acetate (15.0 g, 106 mmol) was dissolved in methanol (350 mL) followed by the addition of triethylamine (50 mL). The reaction mixture was heated to 55° C. for 40 hours. The reaction mixture was cooled and concentrated to provide (R)-3,6-dihydro-2H-pyran-3-ol (10.4 g, 104 mmol, 98% yield) of suitable purity to carry forward to the next reaction. ¹H NMR (400 MHz, CDCl₃), d: 5.90-6.01 (m, 2H), 4.04-4.19 (m, 2H), 3.98 (m, 1H), 3.73-3.86 (m, 2H).

Step D: (R)-3,6-Dihydro-2H-pyran-3-ol (10.0 g, 99.9 mmol) was dissolved in N,N-dimethylformamide (200 mL) followed by the addition of cesium carbonate (39.0 g, 119 mmol) and 5-bromo-2-fluoro-benzaldehyde (11.9 mL, 99.9 mmol). The reaction mixture was heated to 80° C. for 24 hours. After 24 hours the reaction mixture was cooled and diluted with ethyl acetate and saturated sodium bicarbonate. The organic layer was washed with brine, dried over sodium sulfate, filtered and concentrated. The crude material was purified by silica gel eluting with a linear gradient of 0-35% ethyl acetate/heptane to yield (R)-5-bromo-2-(3,6-dihydro-2H-pyran-3-yloxy)benzaldehyde (7.12 g, 25.1 mmol, 25% yield). ¹H NMR (400 MHz, CDCl₃) δ 10.41 (s, 1H), 7.94 (d, J=2.6 Hz, 1H), 7.61 (dd, J=8.9, 2.6 Hz, 1H), 6.93 (d, J=8.9 Hz, 1H), 6.16-6.09 (m, 1H), 6.06-5.99 (m, 1H), 4.84-4.75 (m, 1H), 4.30-4.21 (m, 1H), 4.19-4.10 (m, 1H), 3.99 (d, J=4.0 Hz, 3H).

Step E: (R)-5-Bromo-2-(3,6-dihydro-2H-pyran-3-yloxy)benzaldehyde (6.78 g, 23.9 mmol) and hydroxylamine hydrochloride (5.1 g, 71.8 mmol) were dissolved in ethanol (150 mL) followed by the addition of sodium acetate trihydrate (5.95 g, 71.8 mmol). The reaction mixture was heated at 70° C. overnight. The next morning the reaction was diluted with ethyl acetate and washed sequentially with a saturated sodium bicarbonate solution and brine. The organic layer was then dried over sodium sulfate, filtered, and concentrated to provide suitably pure (R)-5-bromo-2-(3,6-dihydro-2H-pyran-3-yloxy)benzaldehyde oxime (6.82 g, 22.9 mmol, 95% yield) for the next reaction. ¹H NMR (400 MHz, CDCl₃) δ 8.45 (s, 1H), 7.90 (d, J=2.3 Hz, 1H), 7.41 (dd, J=8.9, 2.3 Hz, 1H), 6.82 (d, J=8.9 Hz, 1H), 6.10-6.03 (m, 1H), 6.03-5.95 (m, 1H), 4.72-4.66 (m, 1H), 4.30-4.20 (m, 1H), 4.17-4.09 (m, 1H), 3.96 (dd, J=11.6, 3.9 Hz, 1H), 3.91 (dd, J=11.9, 3.9 Hz, 1H).

Step F: (R)-5-bromo-2-(3,6-dihydro-2H-pyran-3-yloxy)benzaldehyde oxime (6.82 g, 22.9 mmol) was dissolved in N,N-dimethylformamide (100 mL) and methanol (50 mL) and cooled to 0° C. To this solution was added solid [hydrozy(tosyloxy)-iodo]benzene (10.3 g, 25.2 mmol) over the course of 2 minutes. Shortly after the addition, the reaction mixture was warmed to room temperature. After 30 minutes, the reaction mixture was diluted with methylene chloride and washed sequentially with saturated sodium bicarbonate and brine. The organic layer was then dried over sodium sulfate, filtered and concentrated. The crude material was then purified by silica gel eluting with a linear gradient of 0-40% ethyl acetate/heptane to yield enantiopure dihydroisoxazole (5.97 g, 20.2 mmol, 88% yield). ¹H NMR (400 MHz, CDCl₃) δ 7.99 (d, J=2.5 Hz, 1H), 7.44 (dd, J=8.9, 2.5 Hz, 1H), 6.85 (d, J=8.9 Hz, 1H), 5.08 (dd, J=15.9, 8.0 Hz, 1H), 4.90 (dt, J=8.0, 3.9 Hz, 1H), 4.07-3.99 (m, 2H), 3.97-3.90 (m, 1H), 3.09 (dd, J=11.9, 11.9 Hz, 1H), 3.04 (d, J=11.9 Hz, 1H).

Step G: Enantiopure dihydroisoxazole (2.5 g, 8.4 mmol) was dissolved in ethanol (100 mL) followed by the sequential addition of ammonium chloride (4.5 g, 84 mmol), water (100 mL) and iron (4.7 g, 84 mmol). The reaction vessel was sealed at heated at 90° C. for 7 hours. The reaction mixture was then cooled, filtered through Celite®, and then diluted with methylene chloride and a brine solution. The organic layer was then dried over sodium sulfate, filtered and concentrate. The crude material was then purified by silica gel eluting with a linear gradient of 0-30% ethyl acetate/heptane to provide 7-bromo-4-hydroxy-1,4,4a,10a-tetrahydropyrano[3,4-b]chromen-5(3H)-one (1.05 g, 3.51 mmol, 42% yield) of unknown stereochemical purity.

Step H: 7-Bromo-4-hydroxy-1,4,4a,10a-tetrahydropyrano[3,4-b]chromen-5(3H)-one (426 mg, 1.42 mmol) was dissolved in benzene (1.0 mL) followed by the addition of Burgess reagent (367 mg, 1.49 mmol) as a solid. The reaction mixture was then heated to 80° C. for 45 minutes. The reaction mixture was then concentrated and purified by silica gel eluting with a linear gradient of 0-30% ethyl acetate/heptane to provide 7-bromo-1,10a-dihydropyrano[3,4-b]chromen-5(3H)-one (345 mg, 1.23 mmol, 86% yield). ¹H NMR (400 MHz, CDCl₃) δ 8.08 (d, J=2.5 Hz, 1H), 7.56 (dd, J=8.8, 2.5 Hz, 1H), 7.16-7.08 (m, 1H), 6.87 (d, J=8.8 Hz, 1H), 5.06-4.97 (m, 1H), 4.52-4.42 (m, 1H), 4.41-4.32 (m, 1H), 4.25 (dd, J=11.3, 7.9 Hz, 1H), 3.89-3.81 (dd, J=11.3, 7.6 Hz, 1H).

Step I: 7-Bromo-1,10a-dihydropyrano[3,4-b]chromen-5(3H)-one (4.5 g, 51 mmol) was dissolved in ethyl acetate (50 mL) followed by the addition of solid platinum (IV) oxide (16 mg, 0.07 mmol). The atmosphere of the reaction vessel was flushed with nitrogen, then hydrogen. A balloon filled with hydrogen was then fitted to the reaction vessel and the mixture was allowed to stir at room temperature for 1.5 hours. After 1.5 hours, the mixture was filtered through celite and concentrated. The crude material was then purified by silica gel eluting with a linear gradient from 0-30% ethyl acetate/heptane to provide (4aR*,10aS*)-7-bromo-1,4,4a,10a-tetrahydropyrano[3,4-b]chromen-5(3H)-one (414 mg, 1.46 mmol). ¹H NMR (400 MHz, CDCl₃) δ 7.99 (d, J=2.4 Hz, 1H), 7.56 (dd, J=8.9, 2.4 Hz, 1H), 6.87 (d, J=8.9 Hz, 1H), 4.29-4.17 (m, 2H), 4.14-4.06 (m, 1H), 3.57-3.51 (m, 1H), 3.50-3.42 (m, 1H), 2.73-2.63 (m, 1H), 2.30-2.21 (m, 1H), 1.75-1.61 (m, 1H).

Step J: (4aR*,10aS*)-7-Bromo-1,4,4a,10a-tetrahydropyrano[3,4-b]chromen-5(3H)-one (414 mg, 1.46 mmol) was dissolved in tetrahydrofuran (6.0 mL) and cooled to −78° C. A solution of 0.5M Tebbe reagent in toluene (4.39 mL, 2.19 mmol) was then added slowly via syringe. The reaction mixture was then allowed to slowly warm to room temperature overnight. The next morning the reaction mixture was cooled to 0° C. and slowly quenched with methanol (5 mL) followed by the addition of 3 mL of 1N NaOH solution. After stirring 15 minutes at room temperature, the mixture was diluted with methylene chloride (100 mL) and filtered through celite. The filtrate was diluted with additional methylene chloride and a saturated brine solution, and the organic layer was then dried over sodium sulfate, filtered and concentrated. The crude material was purified by silica gel eluting with a linear gradient of 0-25% ethyl acetate/heptane to provide (4aS*,10aS*)-7-bromo-5-methylene-1,3,4,4a,5,10a-hexahydropyrano[3,4-b]chromene (323 mg, 1.15 mmol). ¹H NMR (400 MHz, CDCl₃) δ 7.64 (d, J=2.2 Hz, 1H), 7.24 (dd, J=8.7, 2.2 Hz, 1H), 6.71 (d, J=8.7 Hz, 1H), 5.52 (d, J=2.5 Hz, 1H), 4.92 (d, J=2.1 Hz, 1H), 4.20 (dd, J=11.8, 3.9 Hz, 1H), 4.09 (dd, J=11.8, 3.6 Hz, 1H), 3.78 (ddd, J=11.8, 11.8, 3.9 Hz, 1H), 3.52 (ddd, J=11.8, 11.8, 3.6 Hz, 1H), 3.37 (dd, J=7.9, 7.9 Hz, 1H), 2.44-2.32 (m, 1H), 2.15-2.05 (m, 1H), 1.72-1.59 (m, 1H).

Step K: Iodine (321 mg, 1.26 mmol) dissolved in ethyl acetate (8.5 mL) was added slowly over 5 minutes to a suspension of (4aS*,10aS*)-7-bromo-5-methylene-1,3,4,4a,5,10a-hexahydropyrano[3,4-b]chromene (323 mg, 1.15 mmol) and silver cyanate (209 mg, 1.38 mmol) in ethyl acetate (1.5 mL) and acetonitrile (3.1 mL) at 0° C. The reaction mixture was then warmed to room temperature and allowed to stir for 1 hour. The heterogeneous reaction mixture was then filtered through Celite® and concentrated. The crude material was redissolved in tetrahydrofuran (3.0 mL) and ammonium hydroxide solution (2 mL). The mixture was then heated at 50° C. for 30 minutes. The reaction mixture was then diluted with methylene chloride and saturated brine solution, the organic layer dried over sodium sulfate, filtered and concentrated. The crude material was then purified by silica gel eluting with a linear gradient of 0-6% methylene chloride/methanol+1% ammonium hydroxide to provide 7′-bromo-3′,4′,4a′,10a′-tetrahydro-1′H,5H-spiro[oxazole-4,5′-pyrano[3,4-b]chromen]-2-amine (257 mg, 0.758 mmol, 66% yield) as a 2.5:1 mixture of diastereomers that was carried on to the next reaction without further purification.

Step L: 7′-Bromo-3′,4′,4a′,10a′-tetrahydro-1′H,5H-spiro[oxazole-4,5′-pyrano[3,4-b]chromen]-2-amine (54 mg, 0.159 mmol) as a mixture of diastereomers, (5-chloro-3-pyridyl)boronic acid (32.6 mg, 0.207 mmol), sodium carbonate (51.1 mg, 0.478 mmol), and palladium tetrakis(triphenylphosphine) (18.4 mg, 0.0159 mmol) were added as solids to a vial, followed by the simultaneous addition of dioxane (2.7 mL) and degassed water (0.287 mL). The vial was sealed and heated at 85° C. for 4 hours. The reaction mixture was then cooled and diluted with methylene chloride and a mixture of saturated brine and ammonium hydroxide. The organic layer was dried over sodium sulfate, filtered and concentrated. The crude material was purified by silica gel eluting with a linear gradient of 0-6% methylene chloride/methanol+1% ammonium hydroxide to provide 7′-(5-chloropyridin-3-yl)-3′,4′,4a′,10a′-tetrahydro-1′H,5H-spiro[oxazole-4,5′-pyrano[3,4-b]chromen]-2-amine (41 mg) as a 2:1 mixture of diastereomers. This material was further purified by chiral SFC on a Chiralpak AD (2×15 cm) column eluting with 25% methanol (0.1% NH₄OH)/CO₂ at 100 bar at a flow rate of 70 mL/min. The peaks isolated were analyzed on Chiralpak AD (50×0.46 cm) column eluting with 25% methanol (0.1% NH₄OH)/CO₂, at 120 bar (flow rate 5 mL/min, 220 nm). From this separation, (4R,4a′S,10a′S)-7′-(5-chloropyridin-3-yl)-3′,4′,4a′,10a′-tetrahydro-1′H,5H-spiro[oxazole-4,5′-pyrano[3,4-b]chromen]-2-amine (peak-3, 6.2 mg, 10% yield, chemical purity >99%, ee>99%) was isolated. ¹H NMR (400 MHz, (CD₃)₂SO) δ 8.78 (d, J=1.9 Hz, 1H), 8.55 (d, J=2.2 Hz, 1H), 8.17 (d, J=2.0 Hz, 1H), 7.54 (dd, J=8.4, 2.3 Hz, 1H), 7.51 (d, J=2.1 Hz, 1H), 6.88 (d, J=8.4 Hz, 1H), 5.98 (s, 2H), 4.70 (d, J=8.9 Hz, 1H), 4.26 (d, J=9.0 Hz, 1H), 4.19-4.10 (m, 1H), 4.10-4.00 (m, 1H), 3.96 (dd, J=11.0, 4.3 Hz, 1H), 3.42 (t, J=11.0 Hz, 1H), 3.22 (m, 1H), 1.88 (td, J=11.4, 4.3 Hz, 1H), 1.69 (d, J=13.7 Hz, 1H), 1.50 (td, J=12.7, 4.5 Hz, 1H). m/z (ESI-pos) M+1=372.1.

### Example 111

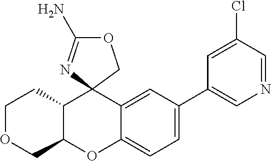

### (4R,4a′R,10a′R)-7′-(5-chloropyridin-3-yl)-3′,4′,4a′,10a′-tetrahydro-1′H,5H-spiro[oxazole-4,5′-pyrano[3,4-b]chromen]-2-amine

Step A: The title compound was prepared from 7′-bromo-3′,4′,4a′,10a′-tetrahydro-1′H,5H-spiro[oxazole-4,5′-pyrano[3,4-b]chromen]-2-amine (synthesized as described in Example 110, Step K) according to the procedure for Example 110, Step L. From the chiral SFC separation, (4R*,4a′R*,10a′R*)-7′-(5-chloropyridin-3-yl)-3′,4′,4a′,10a′-tetrahydro-1′H,5H-spiro[oxazole-4,5′-pyrano[3,4-b]chromen]-2-amine (peak-1, 7.9 mg, 13% yield, chemical purity >99%, ee>99%) was isolated. ¹H NMR (400 MHz, (CD₃)₂SO) δ 8.75 (d, J=1.9 Hz, 1H), 8.55 (d, J=2.3 Hz, 1H), 8.12 (t, J=2.1 Hz, 1H), 7.61-7.52 (m, 2H), 6.86 (d, J=8.4 Hz, 1H), 6.13 (s, 2H), 4.50 (d, J=8.9 Hz, 1H), 4.13 (dd, J=10.2, 5.1 Hz, 1H), 4.09-4.00 (m, 1H), 3.97 (dd, J=11.2, 4.1 Hz, 1H), 3.91 (d, J=8.9 Hz, 1H), 3.37 (t, J=10.8 Hz, 1H), 3.23 (m, 1H), 1.91-1.81 (m, 1H), 1.62 (d, J=9.7 Hz, 1H), 1.44 (dd, J=12.7, 4.4 Hz, 1H). m/z (ESI-pos) M+1=372.1.

### Example 112

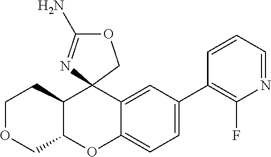

(4R,4a′S,10a′S)-7′-(2-fluoropyridin-3-yl)-3′,4′,4a′,10a′-tetrahydro-1′H,5H-spiro[oxazole-4,5′-pyrano[3,4-b]chromen]-2-amine

The title compound was prepared from 7′-bromo-3′,4′,4a′,10a′-tetrahydro-1′H,5H-spiro[oxazole-4,5′-pyrano[3,4-b]chromen]-2-amine (synthesized as described in Example 110, Step K) according to the procedure for Example 110, Step L, substituting (2-fluoro-3-pyridyl)boronic acid for (5-chloro-3-pyridyl)boronic acid. From the chiral SFC separation, (4R,4a′S,10a′S)-7′-(2-fluoropyridin-3-yl)-3′,4′,4a′,10a′-tetrahydro-1′H,5H-spiro[oxazole-4,5′-pyrano[3,4-b]chromen]-2-amine (peak-3, 4.2 mg, 7% yield, chemical purity >99%, ee>99%) was isolated. m/z (ESI-pos) M+1=356.0.

### Example 113

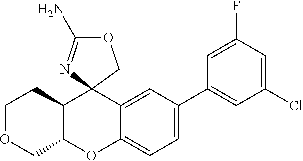

### (4R,4a′S,10a′S)-7′-(3-chloro-5-fluorophenyl)-3′,4′,4a′,10a′-tetrahydro-1′H,5H-spiro[oxazole-4,5′-pyrano[3,4-b]chromen]-2-amine

The title compound was prepared from 7′-bromo-3′,4′,4a′,10a′-tetrahydro-1′H,5H-spiro[oxazole-4,5′-pyrano[3,4-b]chromen]-2-amine (synthesized as described in Example 110, Step K) according to the procedure for Example 110, Step L, substituting (3-chloro-5-fluoro-phenyl)boronic acid for (5-chloro-3-pyridyl)boronic acid. From the chiral SFC separation, (4R,4a′S,10a′S)-7′-(3-chloro-5-fluorophenyl)-3′,4′,4a′,10a′-tetrahydro-1H,5H-spiro[oxazole-4,5′-pyrano[3,4-b]chromen]-2-amine (peak-3, 4.1 mg, 6% yield, chemical purity >99%, ee>99%) was isolated. m/z (ESI-pos) M+1=389.0.

### Example 114

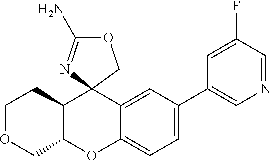

### (4R,4a′S,10a′S)-7′-(5-fluoropyridin-3-yl)-3′,4′,4a′,10a′-tetrahydro-1′H,5H-spiro[oxazole-4,5′-pyrano[3,4-b]chromen]-2-amine

The title compound was prepared from 7′-bromo-3′,4′,4a′,10a′-tetrahydro-1H-5H-spiro[oxazole-4,5′-pyrano[3,4-b]chromen]-2-amine (synthesized as described in Example 110, Step K) according to the procedure for Example 110, Step L, substituting (5-fluoro-3-pyridyl)boronic acid for (5-chloro-3-pyridyl)boronic acid. From the chiral SFC separation, (4R,4a′S,10a′S)-7′-(5-fluoropyridin-3-yl)-3′,4′,4a′,10a′-tetrahydro-1′H,5H-spiro[oxazole-4,5′-pyrano[3,4-b]chromen]-2-amine (peak-3, 3.4 mg, 5% yield, chemical purity >99%, ee>99%) was isolated. m/z (ESI-pos) M+1=356.0.

### Example 115

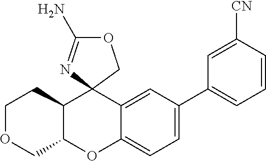

### 3-((4R,4a′S,10a′S)-2-amino-3′,4′,4a′,10a′-tetrahydro-1′H,5H-spiro[oxazole-4,5′-pyrano[3,4-b]chromen]-7′-yl)benzonitrile

The title compound was prepared from 7′-bromo-3′,4′,4a′,10a′-tetrahydro-1′H,5H-spiro[oxazole-4,5′-pyrano[3,4-b]chromen]-2-amine (synthesized as described in Example 110, Step K) according to the procedure for Example 110, Step L, substituting (3-cyano-phenyl)boronic acid for (5-chloro-3-pyridyl)boronic acid. From the chiral SFC separation, 3-((4R,4a′S,10a′S)-2-amino-3′,4′,4a′,10a′-tetrahydro-1′H,5H-spiro[oxazole-4,5′-pyrano[3,4-b]chromene]-7′-yl)benzonitrile (peak-3, 3.2 mg, 5% yield, chemical purity >99%, ee>99%) was isolated. m/z (ESI-pos) M+1=362.0.

### Example 116

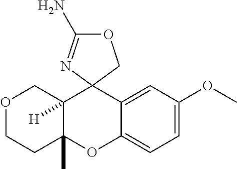

### (4aR*,10aR*)-8-methoxy-4a-methyl-1,3,4,4a,5,10a-hexahydro-5′H-spiro[benzo[g]isochromene-10,4′-oxazol]-2′-amine

Step A: To a solution of (5,6-dihydro-2H-pyran-3-yloxy)trimethylsilane (50.9 g, 296 mmol, prepared according to the method described in WO 2009/43883) and 4-methoxybenzyl acetate (35.5 g, 197 mmol) in dichloromethane (394 mL, 197 mmol) at 0° C. under N₂ was added dropwise a solution of 1,1,1-trifluoro-N-(trifluoromethylsulfonyl)methanesulfonamide (2.77 g, 9.85 mmol) in DCM (24 mL). The mixture was stirred at 0° C. for 10 minutes and quenched with ice water (30 mL). The organic layer was separated, washed with brine (50 mL), dried (MgSO₄) and concentrated in vacuo. The residue isolated was purified by flash chromatography on silica gel (Ready Sep 330 g) eluting with 10% EtOAc/hexane to provide 4-(4-methoxybenzyl)dihydro-2H-pyran-3(4H)-one (41.5 g, 96% yield) as a solid. ¹H NMR (400 MHz, CDCl₃) δ 7.07 (d, J=8.61 Hz, 2H), 6.83 (d, J=8.61 Hz, 2H), 4.06 (d, J=15.65 Hz, 1H), 3.98-3.94 (m, 2H), 3.79 (s, 3H), 3.76-3.70 (m, 1H), 3.29 (dd, J1=4.30 Hz, J2=14.08 Hz, 1H), 2.71-2.63 (m, 1H), 2.06-1.98 (m, 1H), 1.79-1.69 (m, 1H).

Step B: A solution of 4-(4-methoxybenzyl)dihydro-2H-pyran-3(4H)-one (10 g, 45.4 mmol) and iodomethane (3.11 mL, 50 mmol) in tetrahydrofuran (227 mL, 45.4 mmol) was cooled to −78° C. under a stream of N₂. The mixture was then treated dropwise with KOtBu 1M in THF (49.9 mL, 50 mmol) at −78° C. and allowed to warm to −40° C. The mixture was allowed to warm to ambient temperature overnight without removing the cooling bath. The mixture was poured into ice water (100 mL) and THF was removed in vacuo. The aqueous residue obtained was partitioned with EtOAc (3×80 mL). The organic layers were combined, washed with brine (60 mL), dried (MgSO₄) and concentrated in vacuo. The residue obtained was purified by flash chromatography (Ready Sep 120 g, silica gel) eluting with 10% EtOAc/hexane to provide 4-(4-methoxybenzyl)-4-methyldihydro-2H-pyran-3(4H)-one (6.84 g, 64.3% yield) as an oil. ¹H NMR (400 MHz, CDCl₃) δ 7.03 (d, J=8.61 Hz, 2H), 6.82 (d, J=8.99 Hz, 2H), 4.07 (d, J=6.65 Hz, 2H), 3.89-3.85 (m, 2H), 3.79 (s, 3H), 3.01 (d, J=13.694 Hz, 1H), 2.76 (d, J=13.694 Hz, 1H), 2.08-2.02 (m, 1H), 1.7-1.65 (m, 1H), 1.17 (s, 3H).

Step C: To a stirred suspension of (methoxymethyl)triphenylphosphonium chloride (13.5 g, 39.3 mmol) in tetrahydrofuran (78.5 mL, 19.6 mmol) at 0° C. under N₂ was added dropwise a solution of n-butyllithium 2.5 in hexanes (14.3 mL, 35.7 mmol). Once the addition was complete the ice bath was removed, and the mixture was stirred at ambient temperature for 15 minutes. The mixture was then cooled to −78° C. and treated dropwise with a solution of 4-(4-methoxybenzyl)-4-methyldihydro-2H-pyran-3(4H)-one (4.6 g, 19.6 mmol) THF (60 mL) over 30 minutes. The mixture was stirred at −78° C. for 2 hours and allowed to warm to −65° C. The mixture was then poured into saturated aqueous NaHCO₃ (100 mL) and partitioned with EtOAc (3×100 mL). The organic layers were combined, washed with brine (60 mL), dried (MgSO₄) and concentrated in vacuo. The residue obtained was triturated with DCM/hexane to remove some of the Ph₃P═O. The filtrate containing the product was concentrated in vacuo and the resulting residue was purified by flash chromatography on silica gel (Ready Sep 220 g) eluting with 10% EtOAc/hexane to provide (Z)-4-(4-methoxybenzyl)-3-(methoxymethylene)-4-methyltetrahydro-2H-pyran (3.6 g, 69.9% yield) as an oil. ¹H NMR (400 MHz, CDCl₃) δ 6.95 (d, 8.61 Hz, 2H), 6.79 (d, J=8.61 Hz, 2H), 5.51 (s, 1H), 4.64 (d, J=12.91 Hz, 1H), 4.11 (d, J=12.91 Hz, 1H), 3.90-3.79 (m, 2H), 3.78 (s, 3H), 3.50 (s, 3H), 2.96 (d, J=13.30 Hz, 1H), 2.56 (d, J=13.03 Hz, 1H), 1.64-1.57 (m, 1H), 1.48 (d, t, J1=13.13 Hz, J2=3.13 Hz, 1H), 0.94 (s, 3H).

Step D: A solution of (Z)-4-(4-methoxybenzyl)-3-(methoxymethylene)-4-methyltetrahydro-2H-pyran (3.6 g, 13.7 mmol) in THF:2N HCl (2:1, 20 mL) was stirred at ambient temperature. After 18 hours the mixture was treated with concentrated HCl (2 mL) and stirred for 24 hours. The mixture was then diluted with water (50 mL) and extracted with EtOAc (2×60 mL). The organic layers were combined, dried (MgSO₄), concentrated in vacuo and the residue obtained was purified by flash chromatography on silica gel (Ready Sep 80 g) eluting with 10% EtOAc/hexane to provide 4-(4-methoxybenzyl)-4-methyltetrahydro-2H-pyran-3-carbaldehyde (2.85 g, 83.6% yield) as an oil. ¹H NMR (400 MHz, CDCl₃) δ 10.01 (d, J=1.56 Hz, 1H), 7.02 (d, J=8.61 Hz, 2H), 6.81 (d, J=8.22, 2H), 3.95-3.92 (m, 2H), 3.89-3.82 (m, 1H), 3.78 (s, 3H), 3.77-3.75 (m, 1H), 2.85 (d, J=13.69 Hz, 1H), 2.58 (d, J=13.69 Hz, 1H), 2.34-2.30 (m, 1H), 1.80-1.73 (m, 1H), 1.39-1.33 (m, 1H), 1.17 (s, 3H).

Step E: A solution of 4-(4-methoxybenzyl)-4-methyltetrahydro-2H-pyran-3-carbaldehyde (500 mg, 2.01 mmol) in tert-BuOH (7744 μL, 2.01 mmol), tetrahydrofuran (7744 μL, 2.01 mmol) and water (7.7 mL) was cooled to 0° C. and sequentially added 2-methylbut-2-ene 2M in THF (3020 μL, 6.04 mmol) and NaH₂PO₄ (2899 mg, 24.2 mmol). Then NaClO₂ (228 mg, 2.01 mmol) was added in small portions and the mixture was stirred at 0° C. After 2 hours the reaction mixture was poured into saturated NH₄Cl (20 mL) and extracted into EtOAc (3×40 mL). The organic layers were combined, dried (MgSO₄) and concentrated in vacuo. The residue obtained was crystallized from EtOAc/DCM/hexane to provide 4-(4-methoxybenzyl)-4-methyltetrahydro-2H-pyran-3-carboxylic acid (450 mg, 1.70 mmol, 84.6% yield) as a solid. LCMS (APCI−) m/z 263 (M−H)−.

Step F: A mixture of crude 4-(4-methoxybenzyl)-4-methyltetrahydro-2H-pyran-3-carboxylic acid (3.02 g, 11.4 mmol) and PPA (4 mL) was warmed at 50° C. (oil bath) with slow stirring for 40 minutes. Then the mixture was cooled to ambient temperature and quenched with ice water. The resulting suspension was extracted with 5% MeOH/EtOAc (4×60 mL). The combined organic layers were washed with brine, saturated NaHCO₃ (2×30 mL), followed by brine (2×30 mL). The organic layer was separated, dried (MgSO₄) and concentrated in vacuo and the residue obtained was purified by silica gel flash chromatography (ready Sep 80 g) eluting with 20% EtOAc/hexane to provide a mixture of cis and trans (3:2 ratio) 8-methoxy-4a-methyl-3,4,4a,5-tetrahydro-1H-benzo[g]isochromen-10(10aH)-one (1.6 g, 57% yield) as an oil. LCMS: (APCI+) m/z 345, 346.

Step G: To a solution of cis and trans 8-methoxy-4a-methyl-3,4,4a,5-tetrahydro-1H-benzo[g]isochromen-10(10aH)-one (1.4 g, 5.68 mmol) in tetrahydrofuran (56.8 mL, 5.68 mmol) at −78° C. under N₂ was added dropwise a solution of lithium bis(trimethylsilyl)amide 1M in toluene (6.25 mL, 6.25 mmol). The mixture was stirred at −78° C. for 20 minutes and transferred via a cannula into a solution of ethyl 2-hydroxybenzoate (3.34 mL, 22.7 mmol) in THF (30 mL) at −78° C. under N₂. The resulting mixture was stirred at −78° C. for 30 minutes then poured into saturate NH₄Cl solution (50 mL). The mixture was partitioned with DCM (3×40 mL). The organic layers were combined, washed with brine (20 mL), dried (MgSO₄), concentrated in vacuo and the crude isolated was purified by flash chromatography (Ready Sep 80 g silica) eluting with 10% EtOAc/hexane to provide a mixture of trans and cis (30:1 ratio) (4aR,10aS)-8-methoxy-4a,10a-dimethyl-3,4,4a,5-tetrahydro-1H-benzo[g]isochromen-10(10aH)-one (1.33 g, 90% yield). (Trans diastereomer) NMR (400 MHz, CDCl₃) δ 7.45 (d, J=3.13 Hz, 1H), 7.14 (d, J=7.83 Hz, 1H), 7.08 (dd, J1=2.74 Hz, J2=8.22 Hz, 1H), 4.27 (dd, J1=4.30 Hz, J2=12.13 Hz, 1H), 3.88-3.84 (m, 1H), 3.84 (s, 3H), 3.72-3.62 (m, 2H), 2.97 (d, J=16.04 Hz, 1H), 2.80 (dd, J1=4.30 Hz, J2=10.56 Hz, 1H), 2.74 (d, J=16.04 Hz, 1H), 1.98 (dt, J1=5.08 Hz, J2=13.30 Hz, 1H), 1.50 (dt, J1=13.03 Hz, J2=1.56 Hz, 1H), 1.03 (s, 3H).

Step H: To a solution of (4aR,10aS)-8-methoxy-4a-methyl-3,4,4a,5-tetrahydro-1H-benzo[g]isochromen-10(10aH)-one (1.2 g, 4.87 mmol) in tetrahydrofuran (48.7 mL, 4.87 mmol) at 0° C. under N₂ was added dropwise Tebbe reagent 0.5M in toluene (14.6 mL, 7.31 mmol). The mixture was stirred at 0° C. After for 2 hours additional Tebbe Reagent (5 mL) was added and the mixture was stirred at 0° C. fax 30 minutes and at ambient temperature for 1 hour. The mixture was cooled back to 0° C. and carefully quenched with Rochell's salt (until gas evolution stopped). The mixture was stirred at 0° C. and allowed to warm to ambient temperature slowly. The solids formed were filtered off through a pad of Celite®. The filtrate collected was washed with brine (2×20 mL), dried (MgsO₄) and concentrated in vacuo and the residue obtained was by flash chromatography on silica gel (Ready Sep 80 g) eluting with 10% EtOAc/hexane to provide (4aR,10aR)-8-methoxy-4a-methyl-10-methylene-3,4,4a,5,10,10a-hexahydro-1H-benzo[g] isochromene (1.12 g, 94.1% yield). ¹H NMR (400 MHz, CDCl₃) [symbol] 7.12 (d, J=2.74 Hz, 1H), 6.99 (d, J=8.61 Hz, 1H), 6.80 (dd, J1=2.35 Hz, J2=8.26 Hz, 1H), 5.52 (d, J1=1.96 Hz, 1H), 4.62 (d, J=1.96 Hz, 1H), 4.09 (dd, J1=4.30, J2=11.35 Hz, 1H), 3.87-3.83 (m, 1H), 3.81 (s, 3H), 3.69-3.63 (m, 1H), 3.58 (t, J=10.96 Hz, 1H), 2.72 (d, J=15.65 Hz, 1H), 2.57 (d, J=16.04 Hz, 1H), 2.53-2.48 (m, 1H), 1.76 (dt, J1=4.69 Hz, J2=12.91 Hz, 1H), 1.50 (dt, J1=1.95 Hz, J2=13.30 Hz, 1H), 0.89 (s, 3H).

Step I: To a suspension of silver cyanate (1215 mg, 8.10 mmol) in a mixture of acetonitrile (5403 μL, 2.70 mmol) and THF (5403 μL, 2.70 mmol) at 0° C. was added iodine (1.7 g, 6.75 mmol), and the resulting suspension was stirred at 0° C. for 1 minute. To this suspension quickly added a solution of (4aR,10aR)-8-methoxy-4a-methyl-10-methylene-3,4,4a,5,10,10a-hexahydro-1H-benzo[g]isochromene (660 mg, 2.70 mmol) in THF (6 mL), and the resulting suspension was stirred at ambient temperature for one overnight. The mixture was cooled to 0° C., treated with NH₄OH (10805 μL, 2.70 mmol) and stirred at ambient temperature for 18 hours. The mixture was then filtered, and the filtrate collected was extracted with EtOAc (4×50 mL). The combined organics were dried (MgSO₄) and concentrated in vacuo. The crude isolated was purified by flash chromatography on silica gel (Ready Sep 120 g) eluting with a gradient of 2% IPA/DCM/NH₃ (0-30%, 12 CV) to provide (4aR*,10aR*)-8-methoxy-4a-methyl-1,3,4,4a,5,10a-hexahydro-5′H-spiro[benzo[g]isochromene-10,4′-oxazol]-2′-amine (100 mg, 12.2% yield). LCMS: (APCI+) m/z (M+H)+303.

### Example 117

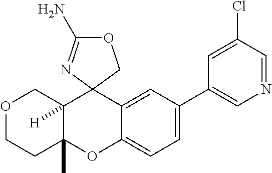

### (4aR*,10aR*)-8-(5-chloropyridin-3-yl)-4a-methyl-1,3,4,4a,5,10a-hexahydro-5′H-spiro[benzo[g]isochromene-10,4′-oxazol]-2′-amine

Step A: A solution of crude (4aR,10aR)-8-methoxy-4a-methyl-1,3,4,4a,5,10a-hexahydro-5′H-spiro[benzo[g]isochromene-10,4′-oxazol]-2′-amine (400 mg, 0.93 mmol) and hydrogen bromide 48% in water (4.630 mL, 0.93 mmol) was heated at 90° C. After 3 hours, the mixture was cooled to 0° C. and carefully basified with saturated NaHCO₃. The resulting mixture was extracted well with EtOAc (5×50 mL) and washed with brine (1×30 mL). The organic layer was dried (MgSO₄), concentrated in vacuo and the crude isolated was purified by flash chromatography on silica gel (Ready Sep 80) eluting with a gradient of 5-20% IPA/DCM+2% NH₄OH (13 CV) to provide (4aR,10aR)-2′-amino-4a-methyl-1,3,4,4a,5,10a-hexahydro-5′H-spiro[benzo[g]isochromene-10,4′-oxazol]-8-ol (100 mg, 0.347 mmol, 37.5% yield) as a solid. LCMS (APCI+) m/z 289 (M+H)+.

Step B: To a solution of (4aR)-2′-amino-4a-methyl-1,3,4,4a,5,10a-hexahydro-5′H-spiro[benzo[g]isochromene-10,4′-oxazol]-8-ol (100 mg, 0.347 mmol) in N,N-dimethylformamide (2312 μL, 0.35 mmol) was added DMF Dimethylacetal (209 μL, 1.73 mmol). The mixture was stirred at ambient temperature for 3 hours. The mixture was then poured in to ice water (20 mL) and partitioned with 5% MeOH/EtOAc (6×30 mL). The aqueous phase pH was adjusted to about 5 with 1M HCl and extracted with EtOAc until there was no product left in the aqueous layer. The organic layers were combined, dried (MgSO₄) and concentrated in vacuo to provide crude (E)-N′-((4aR)-8-hydroxy-4a-methyl-1,3,4,4a,5,10a-hexahydro-5′H-spiro[benzo[g] isochromene-10,4′-oxazole]-2′-yl)-N,N-dimethylformimidamide (126 mg, 105.8% yield) as a solid. LCMS (APCI+) m/z 344 (M+H)+.

Step C: To a solution of (E)-N′-((4aR)-8-hydroxy-4a-methyl-1,3,4,4a,5,10a-hexahydro-5′H-spiro[benzo[g]isochromene-10,4′-oxazole]-2′-yl)-N,N-dimethylformimidamide (120 mg, 0.349 mmol) in DCM was sequentially added triethylamine (97.4 μL, 0.7 mmol) and 1,1,1-trifluoro-N-phenyl-N-(trifluoromethylsulfonyl)methanesulfonamide (187 mg, 0.524 mmol). The resulting mixture was stirred at ambient temperature for 24 hours. The mixture was poured into brine and extracted with DCM (3×30 mL). The organic layers were combined, washed with brine (10 mL), dried (MgSO₄) and concentrated in vacuo. The crude isolated was purified by flash chromatography on silica gel (Ready Sep 80 g) on Biotage SP1 unit eluting with a gradient of 0-30% IPA/DCM+2% NH₃ (10CV) to provide (4aR)-2′-((E)-(dimethylamino)methyleneamino)-4a-methyl-1,3,4,4a,5,10a-hexahydro-5′H-spiro[benzo[g]sochromene-10,4′-oxazole]-8-yl trifluoromethanesulfonate. LCMS (APCI+) m/z 476 (M+H)+.

Step D: A resealable glass pressure tube was charged with (4aR)-2′-((E)-(dimethylamino)methyleneamino)-4a-methyl-1,3,4,4a,5,10a-hexahydro-5′H-spiro[benzo[g]isochromene-10,4′-oxazole]-8-yl trifluoromethanesulfonate (57 mg, 0.12 mmol), 5-chloropyridin-3-ylboronic acid (23 mg, 0.14 mmol), PdCl₂(dppf) dichloromethane adduct (9.8 mg, 0.012 mmol), 20% aqueous Na₂CO₃ (222 μL, 0.42 mmol), and 1,4-dioxane (480 μL, 0.12 mmol). The reaction mixture was sparged with N₂ for 5 minutes, capped, and stirred at 80° C. for 18 hours and allowed to cool to ambient temperature. The reaction mixture was diluted with EtOAc (6 mL) filtered (45 micron filter) and purified by flash chromatography on silica gel (Ready Sep 40 g) eluting with a IPA/DCM+2% NH₄OH (1-40% over 14 CV) to provide (E)-N′-((4aR)-8-(5-chloropyridin-3-yl)-4a-methyl-1,3,4,4a,5,10a-hexahydro-5′H-spiro[benzo[d]isochromene-10,4′-oxazole]-2′-yl)-N,N-dimethylformimidamide (37 mg, 70% yield). LCMS (APCI+) m/z 439 (M+H)+

Step E: A solution of crude (E)-N′-((4aR,10aR)-8-(5-chloropyridin-3-yl)-4a-methyl-1,3,4,4a,5,10a-hexahydro-5′H-spiro[benzo[g]isochromene-10,4′-oxazole]-2′-yl)-N,N-dimethylformimidamide (35 mg, 0.08 mmol) in THF/MeOH (1:1, 2 mL) was treated with 50% aqueous HCl. The mixture was stirred at ambient temperature for 18 hours. The mixture was then poured into ice cold saturated NaHCO₃ and extracted with 5% MeOH/DCM. The organic layers were combined, dried (MgSO₄) and concentrated in vacuo. The residue obtained was purified reverse phase C-18 prep HPLC (Gilson Unipoint) eluting with a gradient of 5-95% CH₃CN/water+0.1% TFA to provide (4aR*,10aR*)-8-(5-chloropyridin-3-yl)-4a-methyl-1,3,4,4a,5,10a-hexahydro-5′H-spiro[benzo[g] isochromene-10,4′-oxazol]-2′-amine 2,2,2-trifluoroacetate (15 mg, 38% yield) as a solid. ¹H NMR (400 MHz, CDCl₃) δ 11.42 (brs, 1H), 8.73 (s, 1H), 8.62 (s, 1H), 7.99 (s, 1H), 7.50-7.47 (m, 2H), 6.41 (s, 1H), 4.96 (d, J=9.39 Hz, 1H), 4.48 (d, J=9.39 Hz, 1H), 3.98-3.92 (m, 2H), 3.72-3.60 (m, 2H), 2.87 (d, J=16.43 Hz, 1H), 2.71 (d, J=16.82 Hz, 1H), 2.51 (dd, J1=3.91 Hz, J2=11.35 Hz, 1H), 1.89-1.81 (m, 1H), 1.55 (d, J=13.69 Hz, 1H), 1.08 (s, 3H). LCMS (APCI+) m/z 384 (M+H)+.

### Example 118

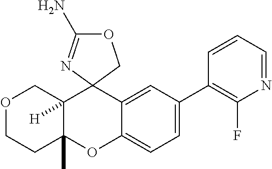

### (4aR*,10aR*)-8-(2-fluoropyridin-3-yl)-4a-methyl-1,3,4,4a,5,10a-hexahydro-5′H-spiro[benzo[g] isochromene-10,4′-oxazol]-2′-amine

(4aR,10aR)-2′-((E)-((Dimethylamino)methylene)amino)-4a-methyl-1,3,4,4a,5,10a-hexahydro-5′H-spiro[benzo[g] isochromene-10,4′-oxazol]-8-yl trifluoromethanesulfonate (40 mg, 0.084 mmol) was processed as described for the preparation of (4aR,10aR)-8-(5-chloropyridin-3-yl)-4a-methyl-1,3,4,4a,5,10a-hexahydro-5′H-spiro[benzo[g]isochromene-10,4′-oxazol]-2′-amine except substituting 5-chloropyridin-3-ylboronic acid with 2-fluoropyridin-3-ylboronic acid (24 mg, 0.17 mmol) to provide (4aR,10aR)-8-(2-fluoropyridin-3-yl)-4a-methyl-1,3,4,4a,5,10a-hexahydro-5′H-spiro[benzo[g]isochromene-10,4′-oxazol]-2′-amine (17 mg, 48% yield). ¹H NMR (400 MHz, CDCl₃) δ 8.63 (d, J=1.56 Hz, 1H), 8.51 (d, J=1.95 Hz, 1H), 7.85 (m, 1H), 7.39-7.36 (m, 2H), 7.18 (d, J=7.83 Hz, 1H), 4.52 (d, J=8.61 Hz, 1H), 4.30 (d, J=8.99 Hz, 1H), 3.90-3.86 (m, 1H), 3.82-3.76 (m, 2H), 2.65 (m, 2H), 1.93 (dd, J1=3.91 Hz, J2=10.96 Hz, 1H), 1.71 (dt, J1=5.48 Hz, J2=12.91 Hz, 1H), 1.53 (d, J=12.91 Hz, 1H), 1.31-1.25 (m, 1H), 1.12 (s, 3H). LCMS (APCI+) m/z 368 (M+H)+.

### Example 119

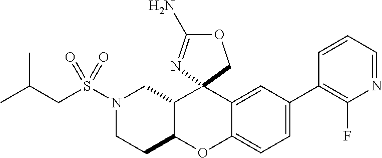

### (4aS*,4′S*,10aR*)-8-(2-fluoropyridin-3-yl)-2-(isobutylsulfonyl)-1,2,3,4,4a,10a-hexahydro-5′H-spiro[chromeno[3,2-c]pyridine-10,4′-oxazol]-2′-amine

Step A: Ethyl 4-chloronicotinate (12.1 g, 60.8% yield) was prepared from 4-chloronicotinic acid (16.9 g, 107 mmol) and thionyl chloride (200 mL, 2735 mmol) according to the method described in WO 2008/02472. LC/MS: APCI (+) m/z 186 (M+1)+.

Step B: To a solution of ethyl 4-chloronicotinate (12.1 g, 65.2 mmol) and 4-bromophenol (11.8 g, 68.5 mmol) in DMF (217 mL) was added Cs₂CO₃ (25.5 g, 78.2 mmol). The reaction mixture was heated at 80° C. with stirring for 20 hours. The reaction mixture was concentrated in vacuo, and the residue was combined with water and EtOAc. The mixture was extracted with EtOAc (2×), and the combined organics were washed brine (1×), dried (Na2SO4), filtered, and concentrated in vacuo. The crude isolated was purified by flash chromatography on silica gel to give an oil, which was chased with DCM/hexanes to give ethyl 4-(4-bromophenoxy)nicotinate (19.2 g, 91.4% yield) as an oil that solidified on standing. APCI (+) m/z 322/324 (M+1)+ with Br isotope.

Step C: To a solution of ethyl 4-(4-bromophenoxy)nicotinate (19.2 g, 59.6 mmol) in THF (300 mL at 0° C. and H₂O (150 mL) was added NaOH (3.58 g, 89.4 mmol). The mixture was lowed to warm to room temperature with stirring. After 7 hours, THF was removed in vacuo, ice water (100 mL), formic acid (3.60 mL, 95.4 mmol) (about pH 3) and saturated NaCl (enough to saturate mixture) were added, and the mixture was extracted with EtOAc (2×). The combined organic extracts were dried (Na₂SO₄), filtered, concentrated, and the resulting residue was concentrated from DCM to give 4-(4-bromophenoxy)nicotinic acid (18.1 g, 103% yield) as a solid. LC/MS APCI (+) m/z 294/296 (M+1)+Br isotope.

Step D: To a 1 L round bottom flask containing 4-(4-bromophenoxy)nicotinic acid (18.1 g, 61.5 mmol) was added concentrated sulfuric acid (123 mL, 2308 mmol). The mixture was stirred until all of the solids dissolved, and the reaction mixture was heated in a 150° C. for 16 hours. The reaction mixture was cooled to room temperature and poured slowly/portionwise into a 0° C. solution of NaOH (187 g, 4677 mmol) in ice water (2 L) (ice added periodically to maintain temp below 15° C.). The solids formed were filtered, rinsed with water, and air dried. The filtrated was extracted with DCM (2×), and the combined organic extracts were combined with the solids. The mixture was then concentrated and dried in vacuo to give 8-bromo-10H-chromeno[3,2-c]pyridin-10-one (15.0 g, 88.3% yield) as a solid. LC/MS APCI (+) m/z 276/278 (M+1)+ with Br isotope.

Step E: Resealable glass pressure tube was charged with 8-bromo-10H-chromeno[3,2-c]pyridin-10-one (3.0 g, 10.87 mmol), TBAI (0.2007 g, 0.5433 mmol) in DCE (50 mL) and 1-(chloromethyl)-4-methoxybenzene (5.899 mL, 43.46 mmol). The reaction mixture was capped tightly and heated at 90° C. After 22 hours, the reaction mixture was cooled to room temperature, diluted with DCM, and the solids were isolated by vacuum filtration. The solid collected was rinsed with DCM and ether, and dried in vacuo to give 8-bromo-2-(4-methoxybenzyl)-10-oxo-10H-chromeno[3,2-c]pyridin-2-ium chloride (3.80 g, 80.82% yield) as a solid. LC/MS APCI (+) m/z 398/400 (M+1)+ with Br isotope.

Step F: To a mixture of crude 8-bromo-2-(4-methoxybenzyl)-10-oxo-10H-chromeno[3,2-c]pyridin-2-ium chloride (3.80 g, 8.782 mmol) in EtOH:THF (80 mL, 1:1) at 0° C. was added NaBH₄ (1.329 g, 35.13 mmol) in portions. The mixture was stirred at 0° C. After 45 minutes, another 1 equivalent NaBH₄ was added and the reaction mixture was continued to stir at 0° C. and allowed to warm to room temperature without removing the ice bath. After 2 hours another 1 equivalent NaBH₄ and another 1 equivalent NaBH₄ was added after 3 hours. The reaction mixture was concentrated in vacuo to 1/3 volume, and this mixture was poured into a solution of ice saturated NH₄Cl. The solids formed were isolated by vacuum filtration, then rinsed with water, air dried, and dried in vacuo to give 8-bromo-2-(4-methoxybenzyl)-2,3,4,4a,10,10a-hexahydro-1H-chromeno[3,2-c]pyridin-10-ol (3.125 g, 88% yield) as a solid. LC/MS (APCI+) m/z 404/406 (M+1)+ with Br isotope.

Step G: To a solution of 2M oxalyl Chloride in DCM (5.797 mL, 11.59 mmol) in DCM (50 mL) at −78° C. was added a solution of DMSO (1.65 mL, 23.2 mmol) in DCM (10 mL). The reaction mixture was stirred at −78° C. for 10 minutes, then a sonicated suspension of 8-bromo-2-(4-methoxybenzyl)-2,3,4,4a,10,10a-hexahydro-1H-chromeno[3,2-c]pyridin-10-ol (3.125 g, 7.729 mmol) in THF (30 mL) was added slowly by syringe. The mixture was stirred at −78° C. for 1 hour, then Et₃N (6.464 mL, 46.38 mmol) was added, and the reaction mixture was allowed to warm to room temperature with stirring. After 1 hour, the mixture was quenched with brine and extracted with DCM (2×). The combined organic extracts were dried (Na₂SO₄), filtered, concentrated and dried in vacuo to give crude (4aS)-8-bromo-2-(4-methoxybenzyl)-2,3,4,4a-tetrahydro-1H-chromeno[3,2-c]pyridin-10(10aH)-one (3.11 g, 100.0% yield).

Step H: A solution of (4aS)-8-bromo-2-(4-methoxybenzyl)-2,3,4,4a-tetrahydro-1H-chromeno[3,2-c]pyridin-10(10aH)-one (3.50 g, 8.70 mmol) in MeOH (55 mL) and DCM (35 mL) was treated with K₂CO₃ (0.240 g, 1.74 mmol) and stirred at room temperature for 3 hours. The mixture was then concentrated in vacuo, and the residue obtained was purified on silica gel (340 g) eluting with 5-40% EtOAc/DCM (8 CV) on Biotage SP1 unit. The 1st major peak was identified as (4aS,10aS)-8-bromo-2-(4-methoxybenzyl)-2,3,4,4a-tetrahydro-1H-chromeno[3,2-c]pyridin-10(10aH)-one (1.40 g, 40.0% yield). LCMS (APCI+) m/z 402/404 (M+1)+ with Br isotope. The 2nd major peak was identified as (4aS,10aR)-8-bromo-2-(4-methoxybenzyl)-2,3,4,4a-tetrahydro-1H-chromeno[3,2-c]pyridin-10(10aH)-one (0.917 g, 2.28 mmol, 26.2% yield). LCMS (APCI+) m/z 402/404 (M+1)+ with Br isotope.

Step I: To a solution of (4aS,10aS)-8-bromo-2-(4-methoxybenzyl)-2,3,4,4a-tetrahydro-1H-chromeno[3,2-c]pyridin-10(10aH)-one (0.573 g, 1.42 mmol) in THF (8 mL) at 0° C. under N₂ was added 0.5M Tebbe reagent in toluene (8.55 mL, 4.27 mmol). The reaction mixture was stirred at 0° C. for 5 minutes and allowed to warm to ambient temperature. After 3 hours, the reaction mixture was diluted with THF, cooled to 0° C., and quenched carefully with MeOH. The resulting mixture was diluted with THF, stirred vigorously for 20 minutes, and Celite® was added. The mixture was filtered through a pad of compressed Celite®, rinsing with THF/MeOH. The filtrate collected was concentrated in vacuo, and the crude obtained was purified on silica gel (50 g) eluting with 10-20% EtoAc/hexanes (8 CV) on Biotage SP1 unit to give (4aS,10aS)-8-bromo-2-(4-methoxybenzyl)-10-methylene-2,3,4,4a,10,10a-hexahydro-1H-chromeno[3,2-c]pyridine (577 mg, 101% yield). LMS (APCI+) m/z 400/402 (M+1)+ with Br isotope.

Step J: A resealable glass pressure tube containing a solution of (4aS,10aS)-8-bromo-2-(4-methoxybenzyl)-10-methylene-2,3,4,4a,10,10a-hexahydro-1H-chromeno[3,2-c]pyridine (0.731 g, 1.826 mmol) in CH₃CN:THF (2:1, 7 mL) was treated with benzyl carbonochloridate (2.085 mL, 14.61 mmol), and the mixture was heated in a 90° C. for 18 hours. The mixture was cooled to ambient temperature then concentrated to dryness, and the residue was partitioned between saturated NaHCO₃ and DCM. The mixture was then extracted with DCM (2×). The combined organic extracts were dried (Na₂SO₄), filtered, and concentrated in vacuo. The crude isolated was purified by silica gel chromatography (100 g) eluting with a gradient of 10-40% EtOAc/hexanes (8CV) on Biotage SP1 unit provide (4aS,10aS)-benzyl 8-bromo-10-methylene-4,4a,10,10a-tetrahydro-1H-chromeno[3,2-c]pyridine-2(3H)-carboxylate (0.342 g, 45.21% yield).

Step K: To a solution of I2 (0.468 g, 1.84 mmol) in CH₃CN:THF (1:1, 1.4 mL) was added silver cyanate (0.552 g, 3.69 mmol). This mixture was sonicated for 1 minute, and it was added to a solution of solution of (4aS,10aS)-benzyl 8-bromo-10-methylene-4,4a,10,10a-tetrahydro-1H-chromeno[3,2-c]pyridine-2(3H)-carboxylate (0.509 g, 1.23 mmol) in THF (8 mL) at 0° C. The reaction mixture was stirred at 0° C. for 30 minutes and allowed to warm to room temperature with stirring. After 4 hours, Celite® was added, and the reaction mixture was vacuum filtered through GF/F paper topped with compressed Celite®, rinsed with THF, and concentrated. The resulting residue was dissolved in THF (6.5 mL) and treated with NH₄OH (3.19 mL, 24.6 mmol). The reaction mixture was stirred at ambient temperature for 17 hours and concentrated in vacuo. The residue obtained was partitioned with brine and EtOAc. The organic extracts were combined, dried (Na₂SO₄), filtered, and concentrated in vacuo. The crude obtained was purified by silica gel flash chromatography (50 g) eluting with a gradient of 0-10% MeOH/DCM (10CV) on Biotage SP1 unit to give (4aS,10aR)-benzyl 2′-amino-8-bromo-1,4,4a,10a-tetrahydro-5′H-spiro[chromeno[3,2-c]pyridine-10,4′-oxazole]-2(3H)-carboxylate (0.526 g, 1.11 mmol, 90.6% yield) as a solid. LCMS (APCI+) m/z 473/475 (M+1)+ with one Br isotope.

Step L: A resealable glass pressure tube was charged with (4aS,10aR)-benzyl 2′-amino-8-bromo-1,4,4a,10a-tetrahydro-5′H-spiro[chromeno[3,2-c]pyridine-10,4′-oxazole]-2(3H)-carboxylate (0.300 g, 0.635 mmol), 2-Fluoropyridin-3-ylboronic acid (0.116 g, 0.826 mmol), Pd(PPh₃)₄ (0.0587 g, 0.0508 mmol), 2M Na₂CO₃ (0.953 mL, 1.91 mmol) and 4.2 mL dioxane. The mixture was purged with nitrogen, sealed with a Teflon cap, and heated at 90° C. with stirring. After 16 hours, the mixture was concentrated in vacuo, and the residue obtained was partitioned between ethyl acetate and water. The organic layers were combined, dried (Na₂SO₄), filtered, and concentrated in vacuo. The crude isolated was purified on silica gel (50 g) eluting with 0-20% MeOH/DCM gradient to provide (4aS,10aR)-benzyl 2′-amino-8-(2-fluoropyridin-3-yl)-1,4,4a,10a-tetrahydro-5′H-spiro[chromeno[3,2-c]pyridine-10,4′-oxazole]-2(3H)-carboxylate (0.177 g, 57% yield) as a solid. LCMS (APCI+) m/z 489 (M+¹)+.

Step M: To a mixture of (4aS,10aR)-benzyl 2′-amino-8-(2-fluoropyridin-3-yl)-1,4,4a,10a-tetrahydro-5′H-spiro[chromeno[3,2-c]pyridine-10,4′-oxazole]-2(3H)-carboxylate (0.18 g, 0.362 mmol) and TEA (0.061 mL, 0.435 mmol) in DCM (3.6 mL and few drops of THF) was added Boc₂O (87 mg, 0.4 mmol). The mixture was stirred at room temperature. After 2 hours, additional 0.5 equivalents Boc₂O and few drops of IPA was added. After 5 hours, several drops MeOH were added to aid solubility, and the mixture was heated at 40° C. with stirring. After 20 hours, another 0.5 equivalents Boc₂O was added, and the reaction mixture stirred at 40° C. for further 6 hours. The reaction mixture was then concentrated in vacuo, and the residue obtained was purified by silica gel preparative TLC (2 plates, 2 mm) eluting with 1:1 DCM:ethyl acetate and then with 1:2 DCM:ethyl acetate followed by flash chromatography (50 g silica, 5-50% EtOAc/hexane) on Biotage SP1 unit to give (4aS,10aR)-benzyl 2′-(tert-butoxycarbonylamino)-8-(2-fluoropyridin-3-yl)-1,4,4a,10a-tetrahydro-5′H-spiro[chromeno[3,2-c]pyridine-10,4′-oxazole]-2(3H)-carboxylate (0.124 g, 58.1% yield) as solid. LCMS (APCI+) m/z 589 (M+1)+.

Step N: A solution of (4aS,10aR)-benzyl 2′-(tert-butoxycarbonylamino)-8-(2-fluoropyridin-3-yl)-1,4,4a,10a-tetrahydro-5′H-spiro[chromeno[3,2-c]pyridine-10,4′-oxazole]-2(3H)-carboxylate (0.124 g, 0.211 mmol) in THF:EtOH (2:1, 2 mL) was treated with 5% Degussa type Pd/C (0.045 g, 0.0211 mmol). Then H₂ was bubbled through the reaction mixture and stirred at ambient temperature under a H₂ balloon. After 16 hours, the mixture was purged with N₂, diluted with MeOH, filtered through a pad of Celite®, rinsing with MeOH/EtOAc. The filtrate collected was concentrated in vacuo and the residue was purified on silica gel to give tert-butyl (4aS,10aR)-8-(2-fluoropyridin-3-yl)-1,2,3,4,4a,10a-hexahydro-5′H-spiro[chromeno[3,2-c]pyridine-10,4′-oxazole]-2′-ylcarbamate (0.074 g, 77.3% yield) as a solid. LCMS (APCI+) m/z 455 (M+1)+.

Step O: To a solution of tert-butyl (4aS,10aR)-8-(2-fluoropyridin-3-yl)-1,2,3,4,4a,10a-hexahydro-5′H-spiro[chromeno[3,2-c]pyridine-10,4′-oxazole]-2′-ylcarbamate (0.01 g, 0.022 mmol) and TEA (0.0123 mL, 0.088 mmol) in 0.3 mL DCM was added 2-methylpropane-1-sulfonyl chloride (0.00574 ml, 0.0440 mmol). After 16 hours, the reaction mixture was concentrated in vacuo and purified by preparative TLC plate (0.5 mm, 2:1 DCM:EA). This purification gave tert-butyl (4aS,4′S,10aR)-8-(2-fluoropyridin-3-yl)-2-(isobutylsulfonyl)-1,2,3,4,4a,10a-hexahydro-5′H-spiro[chromeno[3,2-c]pyridine-10,4′-oxazole]-2′-ylcarbamate (0.003 g, 23.7% yield) APCI (+) m/z 575 (M+1) and tert-butyl (4aS,4′R,10aR)-8-(2-fluoropyridin-3-yl)-2-(isobutylsulfonyl)-1,2,3,4,4a,10a-hexahydro-5′H-spiro[chromeno[3,2-c]pyridine-10,4′-oxazole]-2′-ylcarbamate (0.003 g, 23.7% yield). LCMS (APCI+) m/z 575 (M+1).

Step P: To a solution of tert-butyl (4aS,4′S,10aR)-8-(2-fluoropyridin-3-yl)-2-(isobutylsulfonyl)-1,2,3,4,4a,10a-hexahydro-5′H-spiro[chromeno[3,2-c]pyridine-10,4′-oxazole]-2′-ylcarbamate (30 mg, 0.00522 mmol) in DCM (0.25 mL) was added neat TFA (0.02 mL, 0.261 mmol). The reaction mixture was stirred at ambient temperature for 7 hours. The reaction mixture was concentrated in vacuo. The resulting residue was dissolved in minimal DCM, and this solution was added to a solution of 2M HCl in ether (0.261 mL, 0.5220 mmol). The mixture was stirred 5 minutes, then concentrated under a nitrogen stream and dried in vacuo to provide (4aS*,4′S*,10aR*)-8-(2-fluoropyridin-3-yl)-2-(isobutylsulfonyl)-1,2,3,4,4a,10a-hexahydro-5′H-spiro[chromeno[3,2-c]pyridine-10,4′-oxazol]-2′-amine hydrochloride (0.0018 g, 67.47% yield) as a solid. LCMS (APCI+) m/z 475 (M+1)+.

### Example 120

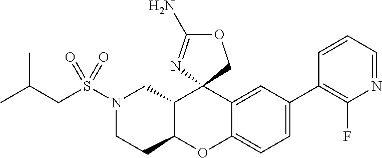

### (4aS,4′S,10aR)-8-(2-fluoropyridin-3-yl)-2-(isobutylsulfonyl)-1,2,3,4,4a,10a-hexahydro-5′H-spiro[chromeno[3,2-c]pyridine-10,4′-oxazol]-2′-amine

Step A: tert-Butyl (4aS,4′S,10aR)-8-(2-fluoropyridin-3-yl)-2-(isobutylsulfonyl)-1,2,3,4,4a,10a-hexahydro-5′H-spiro[chromeno[3,2-c]pyridine-10,4′-oxazole]-2′-ylcarbamate (0.007 g, 0.012 mmol) was separated by chiral HPLC (Chiral Tech 1A, 10% EtOH:90% hexane, 1 mL/min, 220 nM, 4.6 mm×250 mm, 5u) to give tert-butyl (4aS,4′S,10aR)-8-(2-fluoropyridin-3-yl)-2-(isobutylsulfonyl)-1,2,3,4,4a,10a-hexahydro-5′H-spiro[chromeno[3,2-c]pyridine-10,4′-oxazole]-2′-ylcarbamate (0.0025 g, 36% yield). LCMS (APCI+) m/z 575 (M+1) and tert-butyl (4aR,4′R,10aS)-8-(2-fluoropyridin-3-yl)-2-(isobutylsulfonyl)-1,2,3,4,4a,10a-hexahydro-5′H-spiro[chromeno[3,2-c]pyridine-10,4′-oxazole]-2′-ylcarbamate (0.0025 g, 36% yield). LCMS (APCI+) m/z 575 (M+1)+. The absolute configurations assigned arbitrarily.

Step B: To a stirring solution of tert-butyl (4aS,4′S,10aR)-8-(2-fluoropyridin-3-yl)-2-(isobutylsulfonyl)-1,2,3,4,4a,10a-hexahydro-5′H-spiro[chromeno[3,2-c]pyridine-10,4′-oxazole]-2′-ylcarbamate (0.0025 g, 0.004350 mmol) in DCM (0.15 mL) was added TFA (0.03352 mL, 0.4350 mmol). After 2 hours, the reaction mixture was concentrated in in vacuo. The residue obtained was dissolved in minimal DCM+2 drops MeOH, and this solution was added to 2M HCl in ether (0.5 mL). The mixture was stirred 5 minutes, then concentrated under a N₂ stream, chased 3× with ether, concentrated under N₂ stream and dried in vacuo to give (4aS,4′S,10aR)-8-(2-fluoropyridin-3-yl)-2-(isobutylsulfonyl)-1,2,3,4,4a,10a-hexahydro-5′H-spiro[chromeno[3,2-c]pyridine-10,4′-oxazol]-2′-amine hydrochloride (0.0023 g, 103.5% yield) as a solid. LCMS (APCI+) m/z 475 (M+1)+.

### Example 121

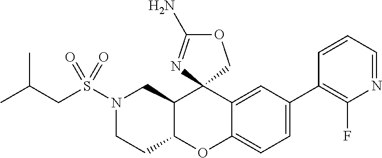

### (4aR,4′R,10aS)-8-(2-fluoropyridin-3-yl)-2-(isobutylsulfonyl)-1,2,3,4,4a,10a-hexahydro-5′H-spiro[chromeno[3,2-c]pyridine-10,4′-oxazol]-2′-amine

To a stirring solution of tert-butyl (4aR,4′R,10aS)-8-(2-fluoropyridin-3-yl)-2-(isobutylsulfonyl)-1,2,3,4,4a,10a-hexahydro-5′H-spiro[chromeno[3,2-c]pyridine-10,4′-oxazole]-2′-ylcarbamate (0.0025 g, 0.00435 mmol) in DCM (0.15 mL) was added neat TFA (0.034 mL, 0.435 mmol). After 2 hours, the mixture was concentrated in vacuo. The residue obtained was dissolved in minimal DCM, and this solution was added to 2M HCl in ether (0.5 mL). The mixture was stirred 5 minutes, then concentrated under a N₂ stream, chased 3× with ether and concentrated under N₂ stream and dried in vacuo to give (4aR,4′R,10aS)-8-(2-fluoropyridin-3-yl)-2-(isobutylsulfonyl)-1,2,3,4,4a,10a-hexahydro-5′H-spiro[chromeno[3,2-c]pyridine-10,4′-oxazol]-2′-amine hydrochloride (0.0023 g, 103% yield) as solid. LCMS (APCI+) m/z 475 (M+1)+.

### Example 122

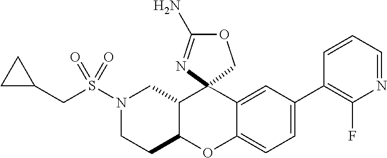

### (4aS,4′R,10aR)-2-((cyclopropylmethyl)sulfonyl)-8-(2-fluoropyridin-3-yl)-1,2,3,4,4a,10a-hexahydro-5′H-spiro[chromeno[3,2-c]pyridine-10,4′-oxazol]-2′-amine

(4aS,10aR)-8-(2-Fluoropyridin-3-yl)-1,2,3,4,4a,10a-hexahydro-5′H-spiro[chromeno[3,2-c]pyridine-10,4′-oxazol]-2′-amine was processed with cyclopropylmethanesulfonyl chlorideas described for the Example 119, Step O to provide (4aS,4′R,10aR)-2-((cyclopropylmethyl)sulfonyl)-8-(2-fluoropyridin-3-yl)-1,2,3,4,4a,10a-hexahydro-5′H-spiro[chromeno[3,2-c]pyridine-10,4′-oxazol]-2′-amine. LCMS (APCI+) m/z 473 (M+1)+.

### Example 123

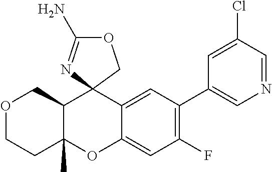

### (4R,4a′R,10a′R)-8′-(5-chloropyridin-3-yl)-7′-fluoro-4a′-methyl-3′,4′,4a′,10a′-tetrahydro-1′H,5H-spiro[oxazole-4,10′-pyrano[4,3-b]chromen]-2-amine

Step A: To a solution of 3-fluoro-phenol (100.0 g, 893 mmol) in anhydrous acetonitrile (1.0 L) was added MgCl₂ (254.7 g, 2.7 mol) portionwise at 0° C. Triethylamine (494 mL, 3.5 mol) was added dropwise to the mixture over 25 minutes, followed by portionwise addition of paraformaldehyde (160.7 g, 5.3 mol). After complete addition, the mixture was heated at reflux for 3 hours. The mixture was cooled and quenched by the addition of cold concentrated hydrochloric acid (347 mL) and extracted with EtOAc. The combined EtOAc layers were washed with water and brine, dried over Na₂SO₄, filtered and evaporated to give4-fluoro-2-hydroxybenzaldehyde (100.0 g, 80.0% yield) as an oil. ¹H NMR ((CD₃)₂SO, 400 MHz): δ 11.19 (d, J=0.4 Hz, 1H), 10.15 (s, 1H), 7.74-7.70 (dd, J=7.2, 8.8 Hz, 1H), 6.81-6.74 (m, 2H).

Step B: To a solution of 4-fluoro-2-hydroxy-benzaldehyde (100.0 g, 714 mmol) in acetic acid (1.0 L) was added bromine (118.5 g, 750 mmol) dropwise at 10° C. The mixture was stirred at room temperature overnight. To the reaction mixture was added dropwise saturated Na₂SO₃ slowly at 0° C. until the brown color disappeared. The reaction was then poured into ice-water, the solid was collected by filtration and dried to give 5-bromo-4-fluoro-2-hydroxybenzaldehyde (50.0 g, crude) as a solid. It was used in the next step without further purification. ¹H NMR (400 MHz, CDCl₃) δ 11.34 (br, 1H), 9.77 (s, 1H), 7.74 (dd, J=6.8, 2.8 Hz, 1H), 6.79 (d, J=9.6 Hz, 1H).

Step C: To a solution of 5-bromo-4-fluoro-2-hydroxybenzaldehyde (50.0 g, crude) and 3-methylbut-2-enal (19.2 g, 230 mmol) in dioxane (90 mL) and water (30 mL) was added triethylamine (16.5 mL, 12 mmol). The reaction mixture was heated at 60° C. for 16 hours. The reaction mixture was cooled to room temperature, extracted with ethyl acetate and water, the organic layer was concentrated to give the crude product which was purified by column chromatography on silica gel (hexanes/EtOAc=1:1) to give the cyclic hemiacetal as a single unknown diastereomer (39.0 g, 18%, two steps) which crystallized as a solid. ¹H NMR (400 MHz, (CD₃)₂SO) δ 7.57 (d, J=8.0 Hz, 1H), 6.83 (d, J=10.4 Hz, 1H), 6.40 (d, J=6.8 Hz, 1H), 4.89 (s, 1H), 4.59 (m, 1H), 2.06 (m, 1H), 1.94 (m, 1H), 1.71 (m, 1H), 1.55 (m, 1H), 1.37 (s, 3H).

Step D: To a solution of the cyclic hemiacetal (39.0 g, 129 mmol) in THF (300 mL) and methanol (30 mL) was added sodium borohydride (2.5 g, 65 mmol). The reaction mixture was stirred at 0° C. for one hour. The reaction mixture was quenched with water, and extracted with DCM. The organic layer was concentrated under vacuum to give 6-bromo-7-fluoro-2-(2-hydroxyethyl)-2-methylchroman-4-ol (35.0 g, 89.2% yield) as a single unknown diastereomer. ¹H NMR (400 MHz, (CD₃)₂SO) δ 7.62 (dd, J=0.8, 8.0 Hz 1H), 6.77 (d, J=10.4 Hz, 1H), 5.55 (d, J=6.0 Hz, 1H), 4.67 (m, 1H), 4.46 (t, J=5.0 Hz, 1H), 3.59 (m, 2H), 2.10 (m, 1H), 1.86 (m, 2H), 1.71 (m, 1H), 1.23 (s, 3H).

Step E: To a solution 6-bromo-7-fluoro-2-(2-hydroxyethyl)-2-methylchroman-4-ol (35.0 g, 115 mmol) in DCM (300 mL) was added manganese dioxide (50.0 g, 576 mmol). The reaction mixture was stirred at room temperature for 20 hours. The reaction mixture was filtered and then washed with DCM. The filtrate was concentrated to give the crude product which was purified by column chromatography on silica gel (hexanes/EtOAc=2:1) to give 6-bromo-7-fluoro-2-(2-hydroxyethyl)-2-methylchroman-4-one (32.0 g, 91.4%). ¹H NMR (400 MHz, (CD₃)₂SO): δ 7.93 (d, J=8.0 Hz, 1H), 7.15 (d, J=10.0 Hz, 1H), 4.54 (t, J=5.2 Hz, 1H), 3.57 (m, 2H), 2.96 (dd, J=16.8, 56.8 Hz, 2H), 1.90 (m, 2H), 1.38 (s, 3H).

Step F: To a solution of 6-bromo-7-fluoro-2-(2-hydroxyethyl)-2-methylchroman-4-one (10.0 g, 33 mmol) and triethylamine (9.2 mL, 66 mmol) in DCM (100 mL) was added dropwise MOMCl (4.0 g, 49 mmol) at 0° C. The reaction mixture was stirred at room temperature for 16 hours. The reaction mixture was then quenched with water, extracted with DCM. The organic layer was concentrated under vacuum to give the 6-bromo-7-fluoro-2-(2-(methoxymethoxy)ethyl)-2-methylchroman-4-one (8.8 g, 77.1%). ¹H NMR (400 MHz, (CD₃)₂SO): δ 7.94 (d, J=8.0 Hz, 1H), 7.16 (d, J=10.0 Hz, 1H), 4.53 (s, 2H), 3.63 (m, 2H), 3.23 (s, 3H), 2.97 (dd, J=16.8, 21.6 Hz, 2H), 2.02 (m, 2H), 1.39 (s, 3H).

Step G: To a solution of 6-bromo-7-fluoro-2-(2-(methoxymethoxy)ethyl)-2-methylchroman-4-one (8.8 g, 25 mmol) in THF (100 mL) was added dropwise LiHMDS (28 mL, 1M, 28 mmol) at −70° C. The reaction mixture was stirred at −70° C. for 10 minutes, then TMSCl (3.0 g, 28 mmol) was added dropwise. The mixture was stirred at −70° C. for additional 30 minutes. The reaction was quenched with 10% NaHCO₃ at −70° C., extracted with DCM. The organic layer was dried over Na₂SO₄, filtered, concentrated to afford crude (6-bromo-7-fluoro-2-(2-(methoxymethoxy)ethyl)-2-methyl-2H-chromen-4-yloxy)trimethylsilane. It was used in the next step without further purification.

Step H: To a solution of TiCl₄ (9.6 mL, 88 mmol) in DCM (100 mL) was added (6-bromo-7-fluoro-2-(2-(methoxymethoxy)ethyl)-2-methyl-2H-chromen-4-yloxy)trimethylsilane (crude) in DCM (50 mL) at −40° C., the mixture was stirred for 30 minutes, the mixture was poured into saturated NaHCO₃, extracted with DCM twice, washed with brine, the organic layer was dried over Na₂SO₄, filtered, concentrated to give the crude product which was purified by column chromatography on silica gel (hexanes/EtOAc=10:1) to give (4aS*,10aR*)-8-bromo-7-fluoro-4a-methyl-1,4,4a,10a-tetrahydropyrano[4,3-b]chromen-10(3H)-one (4.7 g, 59.9%, two steps). ¹H NMR (CDCl₃, 400 MHz) δ 8.00 (d, J=7.6 Hz, 1H), 6.71 (d, J=9.2 Hz, 1H), 3.82-3.75 (m, 3H), 3.51 (t, J=10.8 Hz, 1H), 2.71 (dd, J=5.2, 10.8 Hz, 1H), 1.93-1.89 (m, 1H), 1.76-1.68 (m, 1H), 1.31 (s, 3H).

Step I: (4aS*,10aR*)-8-Bromo-7-fluoro-4a-methyl-1,4,4a,10a-tetrahydropyrano[4,3-b]chromen-10(3H)-one (7.5 g, 23.8 mmol) as the pure cis ring junction material was dissolved in tetrahydrofuran (175 mL) and cooled to −78° C. Lithium bis(trimethylsilyl)amide (1 mol/L) in THF (25 mL, 25 mmol) was then added slowly via syringe. After stirring for 10 minutes at −78° C. the enolate solution was added slowly over 10 minutes via cannula to a solution of ethyl 2-hydroxybenzoate (14.1 mL, 95.2 mmol) in tetrahydrofuran (100 mL) also cooled to −78° C. After stirring 10 minutes the reaction mixture was quenched with a saturated solution of ammonium chloride, then diluted with methylene chloride and a saturated solution of brine. The organic layer was then dried over sodium sulfate, filtered and concentrated. The crude material was then purified by silica gel eluting with a linear gradient of 0-20% ethyl acetate/heptane to provide a 4.5:1 mixture of trans:cis ring junction (4aS*,10aS*)-8-bromo-7-fluoro-4a-methyl-1,4,4a,10a-tetrahydropyrano[4,3-b]chromen-10(3H)-one (6.6 g, 21 mmol, 88% yield).

Step J: (4aS*,10aS*)-8-Bromo-7-fluoro-4a-methyl-1,4,4a,10a-tetrahydropyrano[4,3-b]chromen-10(31-1)-one (6.6 g, 20.9 mmol) as a 4.5:1 trans:cis ring junction mixture was dissolved in tetrahydrofuran (60 mL) and cooled to −78° C. A solution of 0.5M Tebbe reagent in toluene (50 mL, 25 mmol) was then added slowly via syringe. The reaction mixture was allowed to slowly warm to room temperature overnight. The next morning, the reaction mixture was cooled to 0° C. and slowly quenched with methanol followed by the addition of 20 mL of 1N NaOH solution. After stirring 15 minutes at room temperature, the mixture was diluted with methylene chloride (300 mL) and filtered through Celite®. The filtrate was diluted with additional methylene chloride and a saturated brine solution, and the organic layer was then dried over sodium sulfate, filtered and concentrated. The crude material was purified by silica gel eluting with a linear gradient of 0-30% ethyl acetate/heptane to provide (4aS*,10aS*)-8-bromo-7-fluoro-4a-methyl-10-methylene-1,3,4,4a,10,10a-hexahydropyrano[4,3-b]chromene (2.84 g, 9.07 mmol, 43% yield) as a 2:1 mixture of trans:cis ring junction material.

Step K: Iodine (2.53 g, 9.97 mmol) dissolved in ethyl acetate (80 mL) was added slowly over 5 minutes to a suspension of (4aS*,10aS*)-8-bromo-7-fluoro-4a-methyl-10-methylene-1,3,4,4a,10,10a-hexahydropyrano[4,3-b]chromene (2.84 g, 9.07 mmol) as a 2:1 mixture of trans:cis ring junction material and silver cyanate (1.65 g, 10.9 mmol) in ethyl acetate (17 mL) and acetonitrile (24 mL) at 0° C. The reaction mixture was then warmed to room temperature and allowed to stir for 30 minutes and then heated at 40° C. for an additional 30 minutes. The heterogeneous reaction mixture was then filtered through Celite® and concentrated. The crude material was redissolved in tetrahydrofuran (30 mL) and ammonium hydroxide solution (20 mL). The mixture was then heated at 40° C. for 1 hour. The reaction mixture was then diluted with methylene chloride and saturated brine solution, the organic layer dried over sodium sulfate, filtered and concentrated. The crude material was then purified by silica gel eluting with a linear gradient of 0-6% methylene chloride/methanol+1% ammonium hydroxide to provide 1.84 g of a mixture of spirocyclic and ring diastereomers. This material was then further purified by semi-preparative C18 HPLC eluting with 30-70% acetonitrile/water+0.1% ammonium hydroxide to afford W-bromo-7′-fluoro-4a′-methyl-3′,4′,4a′,10a′-tetrahydro-1′H,5H-spiro[oxazole-4,10′-pyrano[4,3-b]chromen]-2-amine (484 mg, 1.30 mmol, 14% yield) as a 3:2 mixture of trans ring junction diastereomers which were taken on to the next reaction without further purification.

Step L: 8′-Bromo-7′-fluoro-4a′-methyl-3′,4′,4a′,10a′-tetrahydro-1′H,5H-spiro[oxazole-4,10′-pyrano[4,3-b]chromen]-2-amine (160 mg, 0.431 mmol), (5-chloro-3-pyridyl)boronic acid (88.2 mg, 0.560 mmol), sodium carbonate (138 mg, 1.29 mmol), and palladium tetrakis(triphenylphosphine) (49.9 mg, 0.0431 mmol) were added as solids to a vial, followed by the simultaneous addition of dioxane (7.4 mL) and degassed water (0.777 mL). The vial was sealed and heated at 85° C. for 2 hours. The reaction mixture was then cooled and diluted with methylene chloride and a mixture of saturated brine and ammonium hydroxide. The organic layer was dried over sodium sulfate, filtered and concentrated. The crude material was purified by silica gel eluting with a linear gradient of 0-6% methylene chloride/methanol+1% ammonium hydroxide to provide 7′-(5-chloropyridin-3-yl)-3′,4′,4a′,10a′-tetrahydro-1′H,5H-spiro[oxazole-4,5′-pyrano[3,4-b]chromen]-2-amine (42 mg) as a 2:1 mixture of diastereomers. This material was further purified by chiral SFC on a Chiralpak AD (2×15 cm) column eluting with 25% methanol (0.1% NH₄OH)/CO₂ at 100 bar at a flow rate of 70 mL/min. The peaks isolated were analyzed on Chiralpak AD (50×0.46 cm) column eluting with 25% methanol (0.1% NH₄OH)/CO₂, at 120 bar (flow rate 5 mL/min, 220 nm). From this separation, (4R,4a′R,10a′R)-8′-(5-chloropyridin-3-yl)-7′-fluoro-4a′-methyl-3′,4′,4a′,10a′-tetrahydro-1′H,5H-spiro[oxazole-4,10′-pyrano[4,3-b]chromen]-2-amine (peak-1, 12.6 mg, 7% yield, chemical purity >99%, ee>99%) was isolated. ¹H NMR (400 MHz, (CD₃)₂) δ 8.68-8.58 (m, 2H), 8.08 (s, 1H), 7.25 (d, J=8.8 Hz, 1H), 6.79 (d, J=11.9 Hz, 1H), 6.06 (s, 2H), 4.43 (d, J=8.9 Hz, 1H), 4.30 (d, J=8.9 Hz, 1H), 3.98-3.82 (m, 2H), 3.57 (m, 1H), 2.14-2.05 (m, 1H), 1.88 (dt, J=12.4, 6.1 Hz, 1H), 1.79 (d, J=12.3 Hz, 1H), 1.48 (s, 3H). m/z (ESI-pos) M+1=404.1.

### Example 124

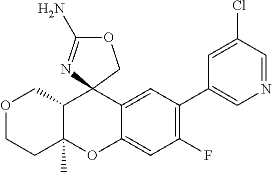

### (4R,4a′S,10a′S)-8′-(5-chloropyridin-3-yl)-7′-fluoro-4a′-methyl-3′,4′,4a′,10a′-tetrahydro-1′H,5H-spiro[oxazole-4,10′-pyrano[4,3-b]chromen]-2-amine

The title compound was prepared from 8′-bromo-7′-fluoro-4a′-methyl-3′,4′,4a′,10a′-tetrahydro-1′H,5H-spiro[oxazole-4,10′-pyrano[4,3-b]chromen]-2-amine (synthesized as described in Example 123 Step K) according to the procedure for Example 123, Step L. From the chiral SFC separation, (4R,4a′S,10a′S)-8′-(5-chloropyridin-3-yl)-7′-fluoro-4a′-methyl-3′,4′,4a′,10a′-tetrahydro-1′H,5H-spiro[oxazole-4,10′-pyrano[4,3-b]chromen]-2-amine (peak-3, 16.3 mg, 9% yield, chemical purity >99%, ee>99%) was isolated. m/z (ESI-pos) M+1=404.1.

### Example 125

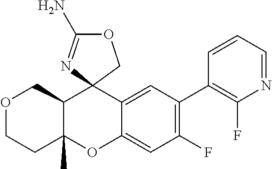

### (4R,4a′R,10aR)-7′-fluoro-8′-(2-fluoropyridin-3-yl)-4a′-methyl-3′,4′,4a′,10a′-tetrahydro-1′H,5H-spiro[oxazole-4,10′-pyrano[4,3-b]chromen]-2-amine

The title compound was prepared from 8′-bromo-7′-fluoro-4a′-methyl-3′,4′,4a′,10a′-tetrahydro-1′H,5H-spiro[oxazole-4,10′-pyrano[4,3-b]chromen]-2-amine (synthesized as described in Example 123, Step K) according to the procedure for Example 123, Step L, substituting (2-fluoro-3-pyridyl)boronic acid for (5-chloro-3-pyridyl)boronic acid. From the chiral SFC separation, (4R,4aR,10a′R)-7′-fluoro-8′-(2-fluoropyridin-3-yl)-4a′-methyl-3′,4′,4a′,10a′-tetrahydro-1′H,5H-spiro[oxazole-4,10′-pyrano[4,3-b]chromen]-2-amine (peak-1, 17.4 mg, 10% yield, chemical purity >99%, ee>99%) was isolated. ¹H NMR (400 MHz, (CD₃)₂SO) δ 8.27 (d, J=4.8 Hz, 1H), 8.16 (s, 1H), 8.01 (t, J=8.7 Hz, 1H), 7.47 (t, J=6.1 Hz, 1H), 7.17 (d, J=8.4 Hz, 1H), 6.77 (d, J=11.4 Hz, 1H), 6.09 (s, 2H), 4.30 (s, 2H), 3.92 (dd, J=11.8, 4.5 Hz, 1H), 3.85 (dd, J=11.6, 3.9 Hz, 1H), 3.63-3.54 (m, 1H), 3.37-3.29 (m, 2H), 2.11 (dd, J=10.8, 3.9 Hz, 1H), 1.93-1.83 (m, 1H), 1.79 (d, J=12.6 Hz, 1H), 1.49 (s, 2H). m/z (ESI-pos) M+1=388.1.

### Example 126

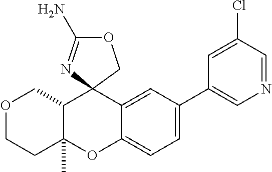

### (4R*,4a′S*,10a′S*)-8′45-chloropyridin-3-yl)-4W-methyl-3′,4′,4a′,10a′-tetrahydro-1′H,5H-spiro[oxazole-4,10′-pyrano[4,3-b]chromen]-2-amine

Step A: To a solution of 5-bromo-2-hydroxybenzaldehyde (30 g, 150 mmol) and Et₃N (10.5 mL, 75 mmol) in dioxane (56 mL) and H₂O (19 mL) was added methylbut-2-enal (12.6 g, 150 mmol). The mixture was stirred at 55° C. for 12 hours. Saturated NaHCO₃ was added to the mixture, extracted with EtOAc. The organic layer was dried over Na₂SO₄, filtered and concentrated to give to the crude product which was purified by column chromatography on silica gel (eluting with 50% ethyl acetate in hexanes) to give the cyclic hemiacetal (26.5 g, 62.6%) as a single unknown diastereomer and a solid. ¹H NMR (CDCl₃, 400 MHz) δ 7.30-7.26 (m, 2H), 6.69 (d, J=9.6 Hz, 1H), 4.85-4.80 (m, 2H), 3.17 (d, J=6.4 Hz, 1H), 2.20-2.10 (m, 2H), 1.81-1.77 (m, 1H), 1.60-1.55 (m, 1H), 1.45 (s, 3H).

Step B: To a solution of the cyclic hemiacetal (20.5 g, 71.8 mmol) in MeOH (60 mL) and THF (240 mL) was added NaBH₄ (1.4 g, 35.9 mmol). The mixture was stirred at 0° C. for 2 hours. The reaction was quenched with saturated NaHCO₃, extracted with DCM. The organic layer was dried over Na₂SO₄, filtered and concentrated to give 6-bromo-2-(2-hydroxyethyl)-2-methylchroman-4-ol (16.6 g, 80.6%) as a single unknown diastereomer and a solid. ¹H NMR (CDCl₃, 400 MHz) δ 7.58 (dd, J=0.8, 2.4 Hz, 1H), 7.28-7.25 (m, 1H), 6.68 (d, J=8.8 Hz, 1H), 4.84-4.81 (m, 1H), 3.90-3.82 (m, 2H), 2.16-1.90 (m, 4H), 1.35 (s, 1H).

Step C: To a solution of 6-bromo-2-(2-hydroxyethyl)-2-methylchroman-4-ol (16.6 g, 58 mmol) in DCM (300 mL) was added MnO₂ (25.2 g, 290 mmol). The reaction mixture was stirred at room temperature for 12 hours. The reaction was filtered, and the filtrate was concentrated to give the crude product which was purified by column chromatography on silica gel (hexanes/EtOAc=2:1) to give 6-bromo-2-(2-hydroxyethyl)-2-methylchroman-4-one (10.8 g, 65% yield) as a solid. ¹H NMR (400 MHz, CDCl₃) δ 7.96 (d, J=2.8 Hz, 1H), 7.56 (dd, J=2.8, 8.8 Hz, 1H), 6.84 (d, J=8.8 Hz, 1H), 3.89-3.86 (m, 2H), 2.93-2.64 (m, 2H), 2.11-1.95 (m, 2H), 1.45 (s, 3H).

Step D: A solution of 6-bromo-2-(2-hydroxyethyl)-2-methylchroman-4-one (10.8 g, 38 mmol) and DIPEA (13.5 mL, 76 mmol) in DCM (30 mL) was added MOMCl (4.4 mL, 57 mmol) at 0° C. The reaction mixture was stirred at room temperature for 12 hours. The reaction was then quenched with saturated NH₄Cl and then extracted with DCM. The organic layer was dried over Na₂SO₄, filtered and concentrated to give the crude product which was purified by column chromatography on silica gel (hexanes/EtOAc=10:1) to give 6-bromo-2-(2-(methoxymethoxy)ethyl)-2-methylchroman-4-one (10.1 g, 81%) as an oil. ¹H NMR (400 MHz, CDCl₃) δ 7.96 (d, J=2.4 Hz, 1H), 7.55 (dd, J=2.8, 8.8 Hz, 1H), 6.84 (d, J=8.8 Hz, 1H), 4.59 (d, J=0.8 Hz, 2H), 3.72-3.68 (m, 2H), 3.34 (s, 3H), 2.91-2.86 (m, 1H), 2.70-2.66 (m, 1H), 2.12-1.99 (m, 2H), 1.44 (s, 3H).

Step E: To a solution of 6-bromo-2-(2-(methoxymethoxy)ethyl)-2-methylchroman-4-one (20.0 g, 60.9 mmol) in THF (300 mL) was added dropwise LiHMDS (66.9 mL, 1M, 66.9 mmol) at −70° C. The mixture was stirred for 10 minutes. Then TMSCl (7.26 g, 66.8 mmol) was added to the mixture. After 30 minutes, the reaction was quenched with 10% NaHCO₃ at −70° C., extracted with DCM. The organic layer was dried over Na₂SO₄, filtered, concentrated to afford the crude product. To a solution of TiCl₄ (23.4 mL, 213 mmol) in DCM (200 mL) was added crude (6-bromo-2-(2-(methoxymethoxy)ethyl)-2-methyl-2H-chromen-4-yloxy)trimethylsilane in DCM (100 mL) at −40° C. The mixture was stirred at −40° C. for 30 minutes. The mixture was then poured into saturated NaHCO₃, extracted with DCM twice, washed with brine, the organic layer was dried over Na₂SO₄, filtered, concentrated to give the crude product which was purified by column chromatography on silica gel (hexanes/EtOAc=10:1) to give (4aS*,10aR*)-8-bromo-4a-methyl-1,4,4a,10a-tetrahydropyrano[4,3-b]chromen-10(3H)-one (12.0 g, 66.5% yield, two steps). ¹H NMR (CDCl₃, 400 MHz) δ 7.96 (d, J=2.4 Hz, 1H), 7.59 (dd, J=2.4, 8.4 Hz, 1H), 6.90 (d, J=8.8 Hz, 1H), 3.89-3.82 (m, 3H), 3.60 (t, J=11.2 Hz, 1H), 2.71 (q, J=5.2 Hz, 1H), 2.00-1.96 (m, 1H), 1.37 (s, 3H).

Step F: (4aS*,10aR*)-8-Bromo-4a-methyl-1,4,4a,10a-tetrahydropyrano[4,3-b]chromen-10(3H)-one (7.0 g, 24 mmol) as the pure cis ring junction material was dissolved in tetrahydrofuran (150 mL) and cooled to −78° C. Lithium bis(trimethylsilyl)amide (1 mol/L) in THF (25 mL, 25 mmol) was then added slowly via syringe. After stirring for 10 minutes at −78° C., the enolate solution was added slowly over 10 minutes via cannula to a solution of ethyl 2-hydroxybenzoate (14 mL, 94 mmol) in tetrahydrofuran (100 mL) also cooled to −78° C. After stirring 10 minutes the reaction mixture was quenched with a saturated solution of ammonium chloride, then diluted with methylene chloride and a saturated solution of brine. The organic layer was then dried over sodium sulfate, filtered and concentrated. The crude material was then purified by silica gel eluting with a linear gradient of 0-20% ethyl acetate/heptane to provide a 5:1 mixture of trans:cis ring junction 8-bromo-4a-methyl-1,4,4a,10a-tetrahydropyrano[4,3-b]chromen-10(3H)-one (6.3 g, 21 mmol, 90% yield).

Step G: 8-Bromo-4a-methyl-1,4,4a,10a-tetrahydropyrano[4,3-b]chromen-10(3H)-one (2.45 g, 8.25 mmol) as a 5:1 trans:cis ring junction mixture was dissolved in tetrahydrofuran (25 mL) and cooled to −78° C. A solution of 0.5 M Tebbe reagent in toluene (24.7 mL, 12.4 mmol) was then added slowly via syringe. The reaction mixture was allowed to slowly warm to room temperature overnight. The next morning, the reaction mixture was cooled to 0° C. and slowly quenched with methanol (20 mL) followed by the addition of 10 mL of 1N NaOH solution. After stirring 15 minutes at room temperature, the mixture was diluted with methylene chloride (200 mL) and filtered through Celite®. The filtrate was diluted with additional methylene chloride and a saturated brine solution, and the organic layer was then dried over sodium sulfate, filtered and concentrated. The crude material was purified by silica gel eluting with a linear gradient of 0-30% ethyl acetate/heptane to provide 8-bromo-4a-methyl-10-methylene-1,3,4,4a,10,10a-hexahydropyrano[4,3-b]chromene (1.47 g, 4.98 mmol, 60% yield) as a 3:1 mixture of trans:cis ring junction material.

Step H: Iodine (1.38 g, 5.44 mmol) dissolved in ethyl acetate (39 mL) was added slowly over 5 minutes to a suspension of 8-bromo-4a-methyl-10-methylene-1,3,4,4a,10,10a-hexahydropyrano[4,3-b]chromene (1.46 g, 4.95 mmol) as a 3:1 mixture of trans:cis ring junction material and silver cyanate (899 mg, 5.93 mmol) in ethyl acetate (6.0 mL) and acetonitrile (13.2 mL) at 0° C. The reaction mixture was then warmed to room temperature and allowed to stir for 30 minutes and then heated at 40° C. for an additional 30 minutes. The heterogeneous reaction mixture was then filtered through Celite® and concentrated. The crude material was redissolved in tetrahydrofuran (30 mL) and ammonium hydroxide solution (20 mL). The mixture was then heated at 40° C. for 1 hour. The reaction mixture was then diluted with methylene chloride and saturated brine solution, the organic layer dried over sodium sulfate, filtered and concentrated. The crude material was then purified by silica gel eluting with a linear gradient of 0-6% methylene chloride/methanol+1% ammonium hydroxide to provide 8′-bromo-4a′-methyl-3′,4′,4a′,10a′-tetrahydro-1′H,5H-spiro[oxazole-4,10′-pyrano[4,3-b]chromen]-2-amine (585 mg, 1.66 mmol, 33% yield) as a mixture of diastereomers which was carried on to the next reaction without additional purification.

Step I: 8′-Bromo-4a′-methyl-3′,4′,4a′,10a′-tetrahydro-1′H,5H-spiro[oxazole-4,10′-pyrano[4,3-b]chromen]-2-amine (95 mg, 0.269 mmol), (5-chloro-3-pyridyl)boronic acid (55 mg, 0.350 mmol), sodium carbonate (86 mg, 0.807 mmol), and palladium tetrakis(triphenylphosphine) (31.1 mg, 0.0269 mmol) were added as solids to a vial, followed by the simultaneous addition of dioxane (4.6 mL) and degassed water (0.485 mL). The vial was sealed and heated at 90° C. for 4 hours. The reaction mixture was then cooled and diluted with methylene chloride and a mixture of saturated brine and ammonium hydroxide. The organic layer was dried over sodium sulfate, filtered and concentrated. The crude material was purified by silica gel eluting with a linear gradient of 0-6% methylene chloride/methanol+1% ammonium hydroxide to provide (4R*,4a′S*,10a′S*)-8′-(5-chloropyridin-3-yl)-4a′-methyl-3′,4′,4a′,10a′-tetrahydro-1′H,5H-spiro[oxazole-4,10′-pyrano[4,3-b]chromen]-2-amine (17.5 mg) as the second eluting UV active fraction. ¹H NMR (400 MHz, (CD₃)₂SO) δ 8.74 (d, J=1.9 Hz, 1H), 8.56 (d, J=2.2 Hz, 1H), 8.12 (t, J=2.1 Hz, 1H), 7.55 (dd, J=8.5, 2.3 Hz, 1H), 7.42 (d, J=2.3 Hz, 1H), 6.85 (d, J=8.5 Hz, 1H), 6.13 (s, 2H), 4.46 (d, J=8.9 Hz, 1H), 4.13 (d, J=8.9 Hz, 1H), 3.92 (dd, J=11.4, 4.0 Hz, 1H), 3.71 (dd, J=11.1, 4.2 Hz, 1H), 3.55 (t, J=11.4 Hz, 2H), 2.19 (dd, J=11.3, 4.2 Hz, 1H), 1.88 (dd, J=12.8, 5.0 Hz, 1H), 1.80 (d, J=12.5 Hz, 1H), 1.32 (s, 3H). m/z (ESI-pos) M+1=386.1.

### Example 127

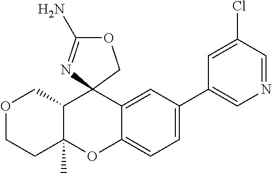

### (4R,4a′S,10a′S)-8′-(5-chloropyridin-3-yl)-4a′-methyl-3′,4′,4a′,10a′-tetrahydro-1′H,5H-spiro[oxazole-4,10′-pyrano[4,3-b]chromen]-2-amine

The title compound was prepared from (4R*,4a′S*,10a′S*)-8′-(5-chloropyridin-3-yl)-4a′-methyl-3′,4′,4a′,10a′-tetrahydro-1′H,5H-spiro[oxazole-4,10′-pyrano[4,3-b]chromen]-2-amine (synthesized as described in Example 126, Step I) and a portion of the material was further purified by chiral SFC on a Chiralpak AD (2×15 cm) column eluting with 25% methanol (0.1% NH₄OH)/CO₂ at 100 bar at a flow rate of 70 mL/minute. The peaks isolated were analyzed on Chiralpak AD (50×0.46 cm) column eluting with 25% methanol (0.1% NH₄OH)/CO₂, at 120 bar (flow rate 5 mL/minute, 220 nm). From the chiral SFC separation, (4R,4a′S,10a′S)-8′-(5-chloropyridin-3-yl)-4a′-methyl-3′,4′,4a′,10a′-tetrahydro-1′H,5H-spiro[oxazole-4,10′-pyrano[4,3-b]chromen]-2-amine (peak-2, 12.6 mg, 6% yield, chemical purity >99%, ee>99%) was isolated. ¹H NMR (400 MHz, (CD₃)₂SO) δ 8.74 (d, J=1.9 Hz, 1H), 8.56 (d, J=2.3 Hz, 1H), 8.12 (t, J=2.1 Hz, 1H), 7.55 (dd, J=8.5, 2.3 Hz, 1H), 7.42 (d, J=2.3 Hz, 1H), 6.85 (d, J=8.5 Hz, 1H), 6.13 (s, 2H), 4.46 (d, J=8.9 Hz, 1H), 4.13 (d, J=8.9 Hz, 1H), 3.92 (dd, J=11.7, 4.2 Hz, 1H), 3.71 (dd, J=11.2, 4.2 Hz, 1H), 3.55 (t, J=11.4 Hz, 2H), 2.19 (dd, J=11.3, 4.2 Hz, 1H), 1.90 (td, J=12.5, 4.9 Hz, 1H), 1.80 (d, J=12.5 Hz, 1H), 1.32 (s, 3H). m/z (ESI-pos) M+1=386.1.

### Example 128

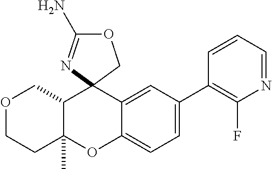

### (4R*,4a′S*,10a′S*)-8′-(2-fluoropyridin-3-yl)-4a′-methyl-3′,4′,4a′,10a′-tetrahydro-1′H,5H-spiro[oxazole-4,10′-pyrano[4,3-b]chromen]-2-amine

The title compound was prepared from 8′-bromo-4a′-methyl-3′,4′,4a′,10a′-tetrahydro-1′H,5H-spiro[oxazole-4,10′-pyrano[4,3-b]chromen]-2-amine (synthesized as described in Example 126, Step H) according to the procedure for Example 126, Step I, substituting (2-fluoro-3-pyridyl)boronic acid for (5-chloro-3-pyridyl)boronic acid. Yield: 23.0 mg (20%). m/z (ESI-pos) M+1=370.1.

### Example 130

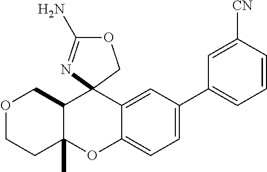

### 3-((4R*,4aR*,10a′R*)-2-amino-4a′-methyl-3′,4′,4a′,10a′-tetrahydro-1′H,5H-spiro[oxazole-4,10′-pyrano[4,3-b]chromen]-8′-yl)benzonitrile

The title compound was prepared from 8′-bromo-4a′-methyl-3′,4′,4a′,10a′-tetrahydro-1′H,5H-spiro[oxazole-4,10′-pyrano[4,3-b]chromen]-2-amine (synthesized as described in Example 126, Step H) according to the procedure for Example 126, Step I, substituting (3-cyano-phenyl)boronic acid for (5-chloro-3-pyridyl)boronic acid. Purification by silica gel provided a mixture of product diastereomers. The mixture was further purified by chiral SFC on a Chiralpak AD (2×15 cm) column eluting with 25% methanol (0.1% NH₄OH)/CO₂ at 100 bar at a flow rate of 70 mL/minute. The peaks isolated were analyzed on Chiralpak AD (50×0.46 cm) column eluting with 25% methanol (0.1% NH₄OH)/CO₂, at 120 bar (flow rate 5 mL/minute, 220 nm). From this purification 3-((4R*,4a′R*,10a′R*)-2-amino-4a′-methyl-3′,4′,4a′,10a′-tetrahydro-1′H,5H-spiro[oxazole-4,10′-pyrano[4,3-b]chromene]-8′-yl)benzonitrile (peak-1, 12.7 mg, 13% yield) was isolated as a racemic mixture. ¹H NMR (400 MHz, (CD₃)₂SO) δ 8.04 (s, 1H), 7.89 (d, J=8.0 Hz, 1H), 7.77 (d, J=7.7 Hz, 1H), 7.64 (t, J=7.8 Hz, 1H), 7.50 (dd, J=8.5, 2.3 Hz, 1H), 7.37 (d, J=2.2 Hz, 1H), 6.85 (d, J=8.5 Hz, 1H), 6.07 (s, 2H), 4.41 (d, J=8.8 Hz, 1H), 4.30 (d, J=8.9 Hz, 1H), 3.89 (ddd, J=24.9, 11.7, 4.2 Hz, 2H), 3.58 (t, J=11.3 Hz, 1H), 3.34 (t, J=8.1 Hz, 1H), 2.09 (dd, J=11.4, 4.4 Hz, 1H), 1.88 (td, J=12.5, 4.8 Hz, 1H), 1.78 (d, J=12.4 Hz, 1H), 1.46 (s, 3H). m/z (ESI-pos) M+1=376.2.

### Example 131

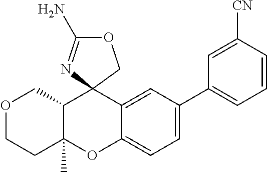

### 3-((4R,4a′S,10a′S)-2-amino-4a′-methyl-3′,4′,4a′,10a′-tetrahydro-1′H,5H-spiro[oxazole-4,10′-pyrano[4,3-b]chromen]-8′-yl)benzonitrile

The title compound was prepared from 8′-bromo-4a′-methyl-3′,4′,4a′,10a′-tetrahydro-1′H,5H-spiro[oxazole-4,10′-pyrano[4,3-b]chromen]-2-amine (synthesized as described in Example 126, Step H) according to the procedure for Example 126, Step I, substituting (3-cyano-phenyl)boronic acid for (5-chloro-3-pyridyl)boronic acid. Purification by silica gel provided a mixture of product diastereomers. The mixture was further purified by chiral SFC on a Chiralpak AD (2×15 cm) column eluting with 25% methanol (0.1% NH₄OH)/CO₂ at 100 bar at a flow rate of 70 mL/minute. The peaks isolated were analyzed on Chiralpak AD (50×0.46 cm) column eluting with 25% methanol (0.1% NH₄OH)/CO₂, at 120 bar (flow rate 5 mL/minute, 220 nm). From this purification 3-((4R,4a′a,10a′S)-2-amino-4a′-methyl-3′,4′,4a′,10a′-tetrahydro-1′H,5H-spiro[oxazole-4,10′-pyrano[4,3-b]chromene]-8′-yl)benzonitrile (peak-3, 8.0 mg, 8% yield, chemical purity >99%, ee>99%) was isolated. m/z (ESI-pos) M+1=376.2.

### Example 132

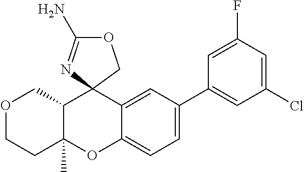

### (4R,4a′S,10a′S)-8′-(3-chloro-5-fluorophenyl)-4a′-methyl-3′,4′,4a′,10a′-tetrahydro-1′H,5H-spiro[oxazole-4,10′-pyrano[4,3-b]chromen]-2-amine

The title compound was prepared from 8′-bromo-4a′-methyl-3′,4′,4a′,10a′-tetrahydro-1′H,5H-spiro[oxazole-4,10′-pyrano[4,3-b]chromen]-2-amine (synthesized as described in Example 126, Step H) according to the procedure for Example 126, Step I, substituting (3-chloro-5-fluoro-phenyl)boronic acid for (5-chloro-3-pyridyl)boronic acid. Purification by silica gel provided a mixture of product diastereomers. The mixture was further purified by chiral SFC on a Chiralpak AD (2×15 cm) column eluting with 25% methanol (0.1% NH₄OH)/CO₂ at 100 bar at a flow rate of 70 mL/minute. The peaks isolated were analyzed on Chiralpak AD (50×0.46 cm) column eluting with 25% methanol (0.1% NH₄OH)/CO₂, at 120 bar (flow rate 5 mL/min, 220 nm). From the chiral SFC separation, (4R,4a′S,10a′S)-8′-(3-chloro-5-fluorophenyl)-4a′-methyl-3′,4′,4a′,10a′-tetrahydro-1′H,5H-spiro[oxazole-4,10′-pyrano[4,3-b]chromen]-2-amine (peak-4, 42.6 mg, 15% yield, chemical purity >99%, ee>99%) was isolated. ¹H NMR (400 MHz, (CD₃)₂SO) δ 7.51 (dd, J=6.9, 2.7 Hz, 1H), 7.42 (ddd, J=8.6, 4.2, 2.8 Hz, 1H), 7.34 (dd, J=13.0, 6.1 Hz, 2H), 7.26 (s, 1H), 6.83 (d, J=8.5 Hz, 1H), 6.13 (s, 2H), 4.44 (d, J=8.9 Hz, 1H), 4.06 (d, J=8.9 Hz, 1H), 3.92 (dd, J=11.8, 4.3 Hz, 1H), 3.71 (dd, J=11.2, 4.2 Hz, 1H), 3.54 (t, J=11.3 Hz, 2H), 2.21 (dd, J=11.3, 4.2 Hz, 1H), 1.96-1.83 (m, 1H), 1.80 (d, J=12.5 Hz, 1H), 1.32 (s, 3H). m/z (ESI-pos) M+1=403.0.

### Example 133

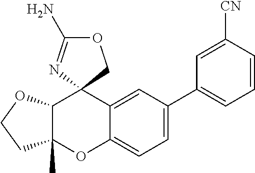

### 3-43aR,4′S,9aR)-2′-amino-3a-methyl-2,3,3a,9a-tetrahydro-5′H-spiro[furo[3,2-b]chromene-9,4′-oxazol]-7-yl)benzonitrile

Step A: To a solution of 6-bromo-2-(2-hydroxyethyl)-2-methylchroman-4-one (20 g, 70 mmol) in dichloromethane (200 mL) was added tert-butyldimethylsilyl chloride (12.7 g, 84 mmol) and imidazole (11.9 g, 176 mmol). The mixture was stirred at room temperature for 0.5 hours. The solvent was removed under reduced pressure, and the residue was purified by flash column chromatography on silica gel (eluting with 5% ethyl acetate in petroleum ether) to give crude 6-bromo-2-(2-(tert-butyldimethylsilyloxy)ethyl)-2-methylchroman-4-one (26.5 g) as an oil, which was used without further purification.

Step B: To a solution of crude 6-bromo-2-(2-(tert-butyldimethylsilyloxy)ethyl)-2-methylchroman-4-one (26.5 g) in acetonitrile (800 mL) was added Koser's reagent (38 g, 98 mmol). The mixture was stirred at reflux. After 3 hours, the solvent was removed under reduced pressure, and the resulting residue was purified by flash column chromatography on silica gel (eluting with 2.5% ethyl acetate in petroleum ether) to afford 7-bromo-3a-methyl-3,3a-dihydro-2H-furo[3,2-b]chromen-9(9aH)-one (8.8 g) as an oil. ¹H NMR (400 MHz, CDCl₃) δ 7.92 (d, J=2.8 Hz, 1H), 7.51-7.49 (dd, J=8.8, 2.8 Hz, 1H), 6.79 (d, J=8.8 Hz, 1H), 4.09-4.04 (m, 2H), 3.83 (s, 1H), 2.45-2.39 (m, 1H), 2.16-2.08 (m, 1H), 1.45 (s, 3H).

Step C: To a solution of 7-bromo-3a-methyl-3,3a-dihydro-2H-furo[3,2-b]chromen-9(9aH)-one (9.0 g, 31.8 mmol) in tetrahydrofuran (100 mL) was slowly added Tebbe reagent (58.3 mL, 35.0 mmol, 0.60 M). The reaction mixture was stirred at room temperature for 1 hour. The mixture was then quenched with 2N aqueous sodium hydroxide. To this mixture was added sodium sulfate, and the resulting mixture was filtered. The filtrate was concentrated, and the resulting residue was purified by flash column chromatography (2.5% ethyl acetate in hexanes) to afford 7-bromo-3a-methyl-9-methylene-3,3a,9,9a-tetrahydro-2H-furo[3,2-b]chromene cis-ring juction (4.2 g, 47.0%) as an oil. ¹H NMR (400 MHz, CDCl₃) δ 7.64 (d, J=2.4 Hz, 1H), 7.61-7.23 (dd, J=8.8, 2.4 Hz, 1H), 6.71 (d, J=8.4 Hz, 1H), 5.72 (s, 1H), 5.29 (s, 1H), 4.11 (s, 1H), 4.04-3.97 (m, 2H), 2.39-2.33 (m, 1H), 2.17-2.09 (m, 1H), 1.45 (s, 3H).

Step D: A solution of iodine (4.2 g, 16.5 mmol) in ethyl acetate (60 mL) was added dropwise to an ice-cooled suspension of AgOCN (3.5 g, 22.5 mmol) and 7-bromo-3a-methyl-9-methylene-3,3a,9,9a-tetrahydro-2H-furo[3,2-b]chromene (4.2 g, 15.0 mmol) in 1:1 acetonitrile/ethyl acetate (60 mL). After 3 hours, the reaction mixture was filtered through Celite®, and the solids were rinsed with ethyl acetate. The filtrate was concentrated in vacuo, and the resulting residue was dissolved in tetrahydrofuran (120 mL) Ammonium hydroxide (30 mL, 25% w/w) was added to the solution. The reaction mixture was stirred at room temperature overnight. The reaction mixture was partitioned between ethyl acetate and saturated aqueous sodium bicarbonate solution (200 mL). The aqueous layer was extracted with ethyl acetate (3×150 mL). The combined organic layers were dried over anhydrous sodium sulfate, filtered, and concentrated. Purification by flash column chromatography on silica gel (1→5% methanol in dichloromethane) provided 7-bromo-3a-methyl-2,3,3a,9a-tetrahydro-5′H-spiro[furo[3,2-b]chromene-9,4′-oxazol]-2′-amine (2.4 g, 48%) as a solid. ¹H NMR (400 MHz, CDCl₃) δ 7.31 (dd, J=2.4, 8.8 Hz, 1H), 7.21 (d, J=2.4 Hz, 1H), 6.76 (d, J=8.8 Hz, 1H), 4.31 (d, J=8.4 Hz, 1H), 3.95 (d, J=8.4 Hz, 1H), 3.61 (m, 2H), 2.09 (m, 2H), 1.75 (s, 3H).

Step E: A solution of 7-bromo-3a-methyl-2,3,3a,9a-tetrahydro-5′H-spiro[furo[3,2-b]chromene-9,4′-oxazol]-2′-amine (300 mg, 0.88 mmol), 3-cyanophenylboronic acid (259 mg, 1.76 mmol), Pd(PPh₃)₂Cl₂ (124 mg, 0.176 mmol) and Na₂CO₃ (466 mg, 4.4 mmol) in 5:1 dioxane/H₂O (6 mL) was heated at 80° C. After 16 hours, water was added, and the resulting mixture was extracted with ethyl acetate (2×). The collected organic extracts were concentrated. Purified by preparative HPLC provided 3-(2′-amino-3a-methyl-2,3,3a,9a-tetrahydro-5′H-spiro[furo[3,2-b]chromene-9,4′-oxazole]-7-yl)benzonitrile (100 mg).

Step F: Racemic 3-(2′-amino-3a-methyl-2,3,3a,9a-tetrahydro-5′H-spiro[furo[3,2-b]chromene-9,4′-oxazole]-7-yl)benzonitrile (100 mg) was purified by supercritical fluid chromatography (SFC-80 Separation Conditions: Column: Chiralcel OD 250×30 mm I.D., 5 um; Mobile phase: Supercritical CO₂/methanol (0.2% diethylamine)=60/40 Flow rate: 50 mL/minute Wavelength: 220 nm) to give 1st eluting peak 3-((3aR,4′S,9aR)-2′-amino-3a-methyl-2,3,3a,9a-tetrahydro-5′H-spiro[furo[3,2-b]chromene-9,4′-oxazole]-7-yl)benzonitrile (13 mg, 4.0% yield) and 2nd eluting peak 3-((3aS,4′R,9aS)-2′-amino-3a-methyl-2,3,3a,9a-tetrahydro-5′H-spiro[furo[3,2-b]chromene-9,4′-oxazole]-7-yl)benzonitrile (13 mg, 4.0% yield).

3-((3aR,4′S,9aR)-2′-amino-3a-methyl-2,3,3a,9a-tetrahydro-5′H-spiro[furo[3,2-b]chromene-9,4′-oxazole]-7-yl)benzonitrile: ¹H NMR (CD₃OD, 400 MHz) δ 7.75 (d, J=1.2 Hz, 1H), 7.71 (dd, J=1.2, 2.4 Hz, 1H), 7.49-7.42 (m, 2H), 7.40-7.32 (m, 1H), 7.30 (d, J=2.4 Hz, 1H), 6.88 (d, J=8.4 Hz, 1H), 4.39 (d, J=8.0 Hz, 1H), 4.02 (d, J=8.4 Hz, 1H), 3.96 (s, 1H), 3.72-3.68 (m, 2H), 2.21-2.14 (m, 1H), 2.10-2.03 (m, 1H), 1.54 (s, 3H). LCMS (ESI) m/z: 361.9 (M+H+).

### Example 134

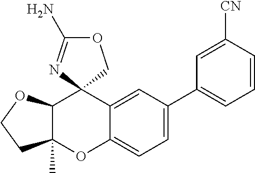

### 3-((3aS,4′R,9aS)-2′-amino-3a-methyl-2,3,3a,9a-tetrahydro-5′H-spiro[furo[3,2-b]chromene-9,4′-oxazol]-7-yl)benzonitrile

3-((3aS,4′R,9aS)-2′-Amino-3a-methyl-2,3,3a,9a-tetrahydro-5′H-spiro[furo[3,2-b]chromene-9,4′-oxazol]-7-yl)benzonitrile was prepared in Example 133. ¹H NMR (CD₃OD, 400 MHz) δ 7.75 (d, J=1.2 Hz, 1H), 7.71 (dd, J=1.2, 2.4 Hz, 1H), 7.49-7.42 (m, 2H), 7.40-7.32 (m, 1H), 7.30 (d, J=2.4 Hz, 1H), 6.88 (d, J=8.4 Hz, 1H), 4.39 (d, J=8.4 Hz, 1H), 4.02 (d, J=8.4 Hz, 1H), 3.96 (s, 1H), 3.73-3.69 (m, 2H), 2.21-2.14 (m, 1H), 2.10-2.03 (m, 1H), 1.54 (s, 3H). LCMS (ESI) m/z: 362.0 (M+H+).

### Example 135

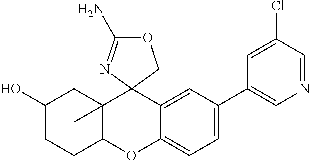

### 2-amino-7′-(5-chloropyridin-3-yl)-9a′-methyl-1′,2′,3′,4′,4a′,9a′-hexahydro-5H-spiro[oxazole-4,9′-xanthen]-2′-ol

Step A: To a solution of 7′-bromo-1′,4′,4a′,9a′-tetrahydrospiro[[1,3]dioxolane-2,2′-xanthen]-9′(3′H)-one (20.0 g, 59 mmol) in tetrahydrofuran (200 mL) was added potassium tert-butoxide (13.2 g, 118 mmol) in portions at −70° C. After 2 hours, iodomethane (33.6 g, 236 mmol) was added dropwise at −70° C. After 5 hours, saturated aqueous ammonium chloride solution (400 mL) was added at −70° C. The resulting mixture was warmed to room temperature and extracted with ethyl acetate (3×200 mL). The combined organic extracts were dried over anhydrous sodium sulfate, filtered, and concentrated. Purification by flash column chromatography (5% ethyl acetate in hexanes) afforded 7′-bromo-9a′-methyl-1′,4′,4a′,9a′-tetrahydrospiro[[1,3]dioxolane-2,2′-xanthen]-9′(3′H)-one (15.0 g, two diastereomers=71: 25, 72.3% yield) as an oil.

Step B: To an ice-cooled solution of 7′-bromo-9a′-methyl-1′,4′,4a′,9a′-tetrahydrospiro[[1,3]dioxolane-2,2′-xanthen]-9′(31-1)-one (15.0 g, 43 mmol) in tetrahydrofuran (100 mL) was added methyl magnesium bromide (42.6 mL, 128 mmol, 3M in diethyl ether), and the resulting reaction mixture was warmed to room temperature. After 3 hours, the reaction mixture was cooled to 0° C., and saturated aqueous ammonium chloride was added slowly. The resulting mixture was extracted with ethyl acetate (3×100 mL). The combined organic extracts were dried over anhydrous sodium sulfate, filtered, and concentrated to give 7′-bromo-9′,9a′-dimethyl-1′,3′,4′,4a′,9′,9a′-hexahydrospiro[[1,3]dioxolane-2,2′-xanthen]-9′-ol (15 g, crude) as an oil, which was used without further purification.

Step C: To an ice-cooled solution of 7′-bromo-9′,9a′-dimethyl-1′,3′,4′,4a′,9′,9a′-hexahydrospiro[[1,3]dioxolane-2,2′-xanthen]-9′-ol (15 g, crude) in toluene (120 mL) was added Burgess reagent (50.8 g, 213 mmol). The mixture was warmed to room temperature. After 16 hours, water was added to the reaction mixture, and the resulting mixture was extracted with ethyl acetate (3×100 mL). The combined organic extracts were dried over anhydrous sodium sulfate, filtered, and concentrated. Purification by flash column chromatography (5% ethyl acetate in hexanes) provided 7′-bromo-9a′-methyl-9′-methylene-1′,3′,4′,4a′,9′,9a′-hexahydrospiro[[1,3]dioxolane-2,2′-xanthene] (3.5 g, cis ring junction), 7′-bromo-9a′-methyl-9′-methylene-1′,3′,4′,4a′,9′,9a′-hexahydrospiro[[1,3]dioxolane-2,2′-xanthene] (900 mg, trans ring junction) and a mixture (5.9 g trans: cis=1:3).

7′-bromo-9a′-methyl-9′-methylene-1′,3′,4′,4a′,9′,9a′-hexahydrospiro[[1,3]dioxolane-2,2′-xanthene]cis ring junction: ¹H NMR (400 MHz, CDCl₃) δ 7.63 (d, J=2.4 Hz, 1H), 7.24-7.21 (dd, J=8.8, 2.4 Hz, 1H), 6.73 (d, J=8.8 Hz, 1H), 5.55 (s, 1H), 5.14 (s, 1H), 3.99-3.83 (m, 5H), 2.07-1.95 (m, 3H), 1.87 (d, J=14.4 Hz, 1H), 1.65-1.61 (m, 1H), 1.44 (d, J=1.2 Hz, 1H), 1.35 (s, 3H).

7′-bromo-9a′-methyl-9′-methylene-1′,3′,4′,4a′,9′,9a′-hexahydrospiro[[1,3]dioxolane-2,2′-xanthene]trans ring junction: ¹H NMR (400 MHz, CDCl₃) δ 7.65 (d, J=2.4 Hz, 1H), 7.25-7.22 (dd, J=8.8, 2.4 Hz, 1H), 6.72 (d, J=8.8 Hz, 1H), 5.40 (s, 1H), 4.88 (s, 1H), 4.05-3.92 (m, 4H), 3.77-3.73 (m, 1H), 2.12-1.83 (m, 3H), 1.73-1.55 (m, 3H), 1.13 (s, 3H).

Step D: To an ice-cooled solution of 7′-bromo-9a′-methyl-9′-methylene-1′,3′,4′,4a′,9′,9a′-hexahydrospiro[[1,3]dioxolane-2,2′-xanthene]trans ring junction (780 mg, 2.2 mmol) and AgOCN (499 mg, 3.3 mmol) in acetonitrile (15 mL) and ethyl acetate (15 mL) was added a solution of iodine (676 mg, 2.7 mmol) in EtOAc (30 mL). After 3 hours, the reaction mixture was filtered through Celite®, and the solids were rinsed with ethyl acetate. The filtrate was concentrated, and the resulting residue was dissolved in tetrahydrofuran (40 mL) and ammonium hydroxide (10 mL, 25% w/w). The reaction mixture was stirred at room temperature overnight. The reaction mixture was partitioned between ethyl acetate and saturated aqueous sodium bicarbonate solution (100 mL). The aqueous layer was extracted with EtOAc (3×100 mL). The combined organic layers were dried over anhydrous sodium sulfate, filtered, and concentrated to afford crude product which was triturated with hexanes to give the ethan-1,2-diol ketal of 2-amino-7′-bromo-9a′-methyl-1′,4′,4a′,9a′-tetrahydro-5H-spiro[oxazole-4,9′-xanthen]-2′(3′H)-one trans ring junction (830 mg, 91.3%). ¹H NMR (CDCl₃, 400 MHz) δ 7.26 (d, J=2.4 Hz, 1H), 7.16 (dd, J=2.4, 8.4 Hz, 1H), 6.62 (d, J=8.8 Hz, 1H), 4.44 (d, J=9.2 Hz, 1H), 4.29 (d, J=9.2 Hz, 1H), 4.22-4.18 (m, 1H), 3.94-3.91 (m, 2H), 3.86-3.82 (m, 2H), 1.92-1.86 (m, 3H), 1.72 (q, J=2.8 Hz, 2H).

Step E: A solution of the ethan-1,2-diol ketal of 2-amino-7′-bromo-9a′-methyl-1′,4′,4a′,9a′-tetrahydro-5H-spiro[oxazole-4,9′-xanthen]-2′(3′H)-one trans ring junction (730 mg, 1.8 mmol) in 2N hydrochloric acid (10 mL) and tetrahydrofuran (30 mL) was heated at 60° C. for 16 hours. The mixture was carefully quenched with Na₂CO₃ until pH greater than 10. The resulting mixture was extracted with ethyl acetate (3×30 mL). The combined organic extracts were dried over anhydrous sodium sulfate, filtered, and concentrated to give 2-amino-7′-bromo-9a′-methyl-1′,4′,4a′,9a′-tetrahydro-5H-spiro[oxazole-4,9′-xanthen]-2′(3′H)-one trans ring junction (600 mg, crude) as a solid, which was used without further purification.

Step F: To an ice-cooled solution of 2-amino-7′-bromo-9a′-methyl-1′,4′,4a′,9a′-tetrahydro-5H-spiro[oxazole-4,9′-xanthen]-2′(3′H)-one trans ring junction (670 mg, crude) in tetrahydrofuran (10 mL) and methanol (10 mL) was added sodium borohydride. The reaction mixture was warmed to room temperature. After 1 hour, the mixture was partitioned between ethyl acetate and saturated aqueous sodium bicarbonate. The organic was separated, and the aqueous was extracted with ethyl acetate (3×50 mL). The combined organic layers were dried over anhydrous sodium sulfate, filtered, and concentrated to afford 2-amino-7′-bromo-9a′-methyl-1′,2′,3′,4′,4a′,9a′-hexahydro-5H-spiro[oxazole-4,9′-xanthen]-2′-ol trans ring junction (630 mg) as a solid, which was used without further purification.

Step G: To a solution of 2-amino-7′-bromo-9a′-methyl-1′,2′,3′,4′,4a′,9a′-hexahydro-5H-spiro[oxazole-4,9′-xanthen]-2′-ol trans ring junction (600 mg, 1.63 mmol), 5-chloropyridin-3-ylboronic acid (385 mg, 2.45 mmol) in dioxane (8 mL) and water (2 mL) was added Pd(PPh₃)₂Cl₂ (172 mg, 0.25 mmol) and Na₂CO₃ (520 mg, 4.90 mmol). The reaction mixture was heated at 80° C. under nitrogen. After 2 hours, the reaction mixture was dilute with water (10 mL), and the resulting solution was extracted with ethyl acetate (3×30 mL). The combined organic extracts were concentrated. Purification by preparative HPLC provided racemic 2-amino-7′-(5-chloropyridin-3-yl)-9a′-methyl-1′,2′,3′,4′,4a′,9a′-hexahydro-5H-spiro[oxazole-4,9′-xanthen]-2′-ol trans ring junction diastereomer 1 (130 mg) and racemic 2-amino-7′-(5-chloropyridin-3-yl)-9a′-methyl-1′,2′,3′,4′,4a′,9a′-hexahydro-5H-spiro[oxazole-4,9′-xanthen]-2′-ol trans ring junction diastereomer 2 (130 mg).

Step H: Racemic 2-amino-7′-(5-chloropyridin-3-yl)-9a′-methyl-1′,2′,3′,4′,4a′,9a′-hexahydro-5H-spiro[oxazole-4,9′-xanthen]-2′-ol trans ring junction diastereomer 1 (130 mg) was purification by SFC separation (SFC-80 Separation Conditions:Column: Chiralcel AD 250×30 mm I.D., 20 um; Mobile phase: Supercritical CO₂/EtOH (0.2% ammonium hydroxide)=60/40 flow rate: 80 mL/min wavelength: 220 nm) to give 2-amino-7′-(5-chloropyridin-3-yl)-9a′-methyl-1′,2′,3′,4′,4a′,9a′-hexahydro-5H-spiro[oxazole-4,9′-xanthen]-2′-ol trans ring junction stereoisomer 1 (50.8 mg, 7.8% yield) and 2-amino-7′-(5-chloropyridin-3-yl)-9a′-methyl-1′,2′,3′,4′,4a′,9a′-hexahydro-5H-spiro[oxazole-4,9′-xanthen]-2′-ol trans ring junction stereoisomer 2 (50.6 mg, 7.7% yield).

2-Amino-7′-(5-chloropyridin-3-yl)-9a′-methyl-1′,2′,3′,4′,4a′,9a′-hexahydro-5H-spiro[oxazole-4,9′-xanthen]-2′-ol trans ring junction stereoisomer 1: ¹H NMR (CD₃OD, 400 MHz) δ 8.64 (d, J=2.0 Hz, 1H), 8.46 (d, J=2.0 Hz, 1H), 8.03-8.01 (m, 1H), 7.51-7.45 (m, 2H), 6.90 (d, J=8.4 Hz, 1H), 4.68 (d, J=9.6 Hz, 1H), 4.52 (d, J=9.6 Hz, 1H), 4.28-4.23 (m, 1H), 4.17-4.15 (m, 1H), 2.17-2.06 (m, 1H), 1.94-1.92 (m, 1H), 1.90-1.75 (m, 2H), 1.70-1.60 (m, 2H), 1.08 (s, 3H); LCMS (ESI) m/z: 400.1 (M+H+).

2-Amino-7′-(5-chloropyridin-3-yl)-9a′-methyl-1′,2′,3′,4′,4a′,9a′-hexahydro-5H-spiro[oxazole-4,9′-xanthen]-2′-ol trans ring junction stereoisomer 2: ¹H NMR (CD₃OD, 400 MHz) δ 8.64 (d, J=2.0 Hz, 1H), 8.46 (d, J=2.4 Hz, 1H), 8.03-8.02 (m, 1H), 7.51-7.45 (m, 2H), 6.90 (d, J=8.4 Hz, 1H), 4.69 (d, J=9.2 Hz, 1H), 4.52 (d, J=9.2 Hz, 1H), 4.27-4.23 (m, 1H), 4.17-4.15 (m, 1H), 2.17-2.06 (m, 1H), 1.94-1.89 (m, 1H), 1.83-1.70 (m, 2H), 1.69-1.60 (m, 2H), 1.07 (s, 3H); LCMS (ESI) m/z: 400.1 (M+H+).

Step I: Racemic 2-amino-7′-(5-chloropyridin-3-yl)-9a′-methyl-1′,2′,3′,4′,4a′,9a′-hexahydro-5H-spiro[oxazole-4,9′-xanthen]-2′-ol trans ring junction diastereomer 2 (60 mg) was purification by SFC separation (SFC-80 Separation Conditions:Column: Chiralcel AD 250×30 mm I.D., 20 um; Mobile phase: Supercritical CO₂/EtOH (0.2% ammonium hydroxide)=60/40; flow rate: 80 mL/minute Wavelength: 220 nm) to give 2-amino-7′-(5-chloropyridin-3-yl)-9a′-methyl-1′,2′,3′,4′,4a′,9a′-hexahydro-5H-spiro[oxazole-4,9′-xanthen]-2′-ol trans ring junction stereoisomer 3 (41.1 mg, 6.3% yield) and 2-amino-7′-(5-chloropyridin-3-yl)-9a′-methyl-1′,2′,3′,4′,4a′,9a′-hexahydro-5H-spiro[oxazole-4,9′-xanthen]-2′-ol trans ring junction stereoisomer 4 (47.3 mg, 7.2% yield).

2-Amino-7′-(5-chloropyridin-3-yl)-9a′-methyl-1′,2′,3′,4′,4a′,9a′-hexahydro-5H-spiro[oxazole-4,9′-xanthen]-2′-ol trans ring junction stereoisomer 3: ¹H NMR (CD₃OD, 400 MHz) δ 8.64 (d, J=2.0 Hz, 1H), 8.46 (d, J=2.4 Hz, 1H), 8.03 (m, 1H), 7.52 (d, J=2.0 Hz, 1H), 7.48-7.45 (m, 1H), 6.90 (d, J=8.8 Hz, 1H), 4.71 (d, J=9.6 Hz, 1H), 4.56 (d, J=9.6 Hz, 1H), 4.25-4.21 (m, 1H), 3.89-3.82 (m, 1H), 2.04-2.00 (m, 2H), 1.85-1.70 (m, 2H), 1.51-1.50 (m, 1H), 1.39-1.32 (m, 1H), 0.89 (s, 3H); LCMS (ESI) m/z: 400.1 (M+H+).

2-Amino-7′-(5-chloropyridin-3-yl)-9a′-methyl-1′,2′,3′,4′,4a′,9a′-hexahydro-5H-spiro[oxazole-4,9′-xanthen]-2′-ol trans ring junction stereoisomer 4: ¹H NMR (CD₃OD, 400 MHz) δ 8.64 (d, J=1.6 Hz, 1H), 8.46 (d, J=2.0 Hz, 1H), 8.03 (m, 1H), 7.53-7.46 (m, 2H), 6.90 (d, J=8.4 Hz, 1H), 4.72 (d, J=9.6 Hz, 1H), 4.57 (d, J=9.6 Hz, 1H), 4.24-4.21 (m, 1H), 3.89-3.81 (m, 1H), 2.04-2.00 (m, 2H), 1.85-1.73 (m, 2H), 1.58-1.52 (m, 1H), 1.36-1.28 (m, 1H), 0.88 (s, 3H); LCMS (ESI) m/z: 400.1 (M+H+).

### Example 136

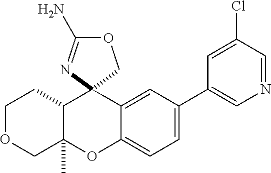

### (4R,4a′R,10a′R)-7′-(5-chloropyridin-3-yl)-10a′-methyl-3′,4′,4a′,10a′-tetrahydro-1′H,5H-spiro[oxazole-4,5′-pyrano[3,4-b]chromen]-2-amine

Step A: A round-bottomed flask was charged with anhydrous CH₂Cl₂ (200 mL) and titanium(IV) chloride (200 mL, 205 mmol, 1M in CH₂Cl₂) and fitted with a dropping funnel with septum. After purging with nitrogen gas and cooling to −78° C. in an ether/dry ice bath, the dropping funnel was filled with a solution of 2-methyl-2-vinyl-oxirane (13.4 mL, 137 mmol) in anhydrous CH₂Cl₂ (75 mL). This solution was added into the stirring reaction mixture dropwise over a period of 2.5 hours. After an additional hour of stirring, the reaction was quenched with 1M aqueous HCl (180 mL). The layers were partitioned and the organics were washed again with 1M aqueous HCl, dried (MgSO₄), and filtered through a short pad of silica gel. To the resulting solution was added imidazole (10.3 g, 150 mmol), 4-dimethylaminopyridine (1.76 g, 13.7 mmol), and tert-butyldimethylsilyl chloride (21.2 g, 137 mmol). The vessel was sealed with a septum and stirred for 1 hour, at which time the reaction was complete as determined by TLC analysis. The reaction mixture was filtered, rinsing with CH₂Cl₂, and the solids were discarded. The solvent was removed by rotary evaporation at 300 mbar vacuum at 30° C., and the residue was purified by flash column chromatography (100:0-95:5 pet. ether/EtOAc) to afford (E)-tert-butyl(4-chloro-2-methylbut-2-enyloxy)dimethylsilane (13% (Z) by NMR) as a colorless liquid (8.72 g, 27% over 2 steps); ¹H NMR (400 MHz, CDCl₃) δ 5.73 (t, J=8.0 Hz, 1H), 4.14 (d, J=8.0 Hz, 2H), 4.06 (s, 2H), 1.69 (s, 3H), 0.90 (s, 9H), 0.08 (s, J=3.2 Hz, 6H).

Step B: In an open flask, sodium hydride (1.22 g, 48.3 mmol, 95% by wt.) was added to a stirring solution of 4-bromophenol (7.14 g, 40.9 mmol) in anhydrous toluene (100 mL). The flask was capped with a septum and purged with nitrogen, stirring over a period of 35 min. A solution of (E)-tert-butyl(4-chloro-2-methylbut-2-enyloxy)dimethylsilane (8.72 g, 37.1 mmol) in anhydrous toluene (85 mL) was then added and the reaction mixture was stirred at 35° C. for 19.5 hours. The reaction mixture was washed with water and then the water was extracted again with EtOAc. The combined organics were then washed with brine, dried (MgSO₄), and concentrated to dryness. The residue was dissolved in anhydrous DMF (185 mL), charged with potassium carbonate (51.3 g, 371 mmol), purged under nitrogen gas, and cooled to 0° C. To this reaction mixture was added chloromethyl methyl ether (14.8 mL, 186 mmol) over a period of 1 hour, monitoring the disappearance of starting material by HPLC. The reaction mixture was then quenched with saturated aqueous NaHCO₃, warmed to room temperature and concentrated to dryness. The residue was partitioned between CH₂Cl₂ and water and the aqueous layer was extracted an additional time with CH₂Cl₂. The combined organic layers were washed with brine, dried (MgSO₄) and concentrated. Flash chromatographic purification of the residue thus obtained (100:0-60:40 heptanes/EtOAc) afforded (E)-(4-(5-bromo-2-(methoxymethoxy)phenyl)-2-methylbut-2-enyloxy)(tert-butyl)dimethylsilane (5.66 g, 37% over 2 steps), followed by (E)-4-(5-bromo-2-(methoxymethoxy)phenyl)-2-methylbut-2-en-1-01 (1.22 g, 11% over 2 steps), both colorless liquids. ¹H NMR (400 MHz, CDCl₃) δ 7.23 (m, 2H), 6.93 (d, J=9.1 Hz, 1H), 5.53 (t, J=7.6 Hz, 1H), 5.16 (s, 2H), 4.05 (s, 2H), 3.46 (s, 3H), 3.35 (d, J=7.6 Hz, 2H), 1.69 (s, 3H), 0.92 (s, 9H), 0.07 (s, 6H).

Step C: To a stirring solution of (E)-(4-(5-bromo-2-(methoxymethoxy)phenyl)-2-methylbut-2-enyloxy)(tert-butyl)dimethylsilane (4.89 g, 11.8 mmol) in anhydrous THF (24 mL) was added tetrabutylammonium fluoride (17.7 mL, 17.6 mmol, 1M in THF). The reaction was capped with a septum and stirring continued for 45 minutes before being quenched by the addition of saturated aqueous NH₄Cl. The mixture was extracted twice with CH₂Cl₂ and dried over MgSO₄. Concentration and purification by flash chromatography (100:0-60:40 heptanes/EtOAc) afforded (E)-4-(5-bromo-2-(methoxymethoxy)phenyl)-2-methylbut-2-en-1-ol as a liquid (2.41 g, 68%). ¹H NMR (400 MHz, CDCl₃) δ 7.24 (m, 2H), 6.89 (m, 1H), 5.56 (t, J=7.9 Hz, 1H), 5.17 (s, 2H), 4.23 (d, J=5.8 Hz, 1H), 4.05 (d, J=5.8 Hz, 2H), 3.47 (s, 3H), 3.36 (d, J=7.9 Hz, 2H), 1.77 (s, 3H).

Step D: In an open flask and at 0° C., sodium hydride (337 mg, 8.00 mmol, 60% wt. oil dispersion) was added to a stirring solution of (E)-4-(5-bromo-2-(methoxymethoxy)phenyl)-2-methylbut-2-en-1-01 (2.41 g, 8.00 mmol) and propargyl bromide (1.07 mL, 9.6 mmol, 80% wt. in toluene) in anhydrous DMF (27 mL). The flask was capped with a septum and purged with nitrogen, removed from the ice bath, and stirred over a period of 3.5 hours. The reaction mixture was concentrated to dryness, diluted with water, and extracted with CH₂Cl₂. The combined organics were then washed with brine, dried (MgSO₄), and concentrated to dryness. Flash chromatographic purification of the residue thus obtained (100:0-90:10 heptanes/EtOAc) afforded (E)-4-bromo-1-(methoxymethoxy)-2-(3-methyl-4-(prop-2-ynyloxy)but-2-enyl)benzene as a liquid (1.97 g, 73%). ¹H NMR (400 MHz, CDCl₃) δ 7.31-7.20 (m, 2H), 6.98-6.90 (m, 1H), 5.55 (t, J=7.5 Hz, 1H), 5.17 (s, 2H), 4.10 (s, 2H), 3.98 (s, 2H), 3.46 (s, 3H), 3.39 (d, J=7.5 Hz, 2H), 2.42 (s, 1H), 1.78 (s, 3H).

Step E: Under nitrogen atmosphere and at 0° C., HCl (14.3 mL, 56.9 mmol, 4 M in dioxane) was added to a stirring solution of (E)-4-bromo-1-(methoxymethoxy)-2-(3-methyl-4-(prop-2-ynyloxy)but-2-enyl)benzene (1.93 g, 5.69 mmol) in anhydrous i-PrOH (30 mL). After 2 hours, the ice bath was removed and stirring was continued for another 3 hours. The reaction mixture was concentrated to dryness and then diluted with CH₂Cl₂ and washed with brine, dried (MgSO₄) and concentrated to dryness to afford (E)-4-bromo-2-(3-methyl-4-(prop-2-ynyloxy)but-2-enyl)phenol (1.73 g, >99%). ¹H NMR (400 MHz, CDCl₃) δ 7.24-7.14 (m, 2H), 6.74-6.62 (m, 1H), 5.60 (t, J=7.8 Hz, 1H), 5.13 (br s, 1H), 4.12 (d, J=2.3 Hz, 2H), 4.00 (s, 2H), 3.37 (d, J=7.8 Hz, 2H), 2.42 (t, J=2.3 Hz, 1H), 1.76 (s, 3H).

Step F: A solution of (E)-4-bromo-2-(3-methyl-4-(prop-2-ynyloxy)but-2-enyl)phenol (3.25 g, 11.0 mmol) in anhydrous toluene (110 mL) under nitrogen atmosphere was cooled to −10° C. and charged with [Bis(trifluoromethanesulfonyl)imidate](triphenylphosphine)gold(I) (2:1) toluene adduct (346 mg, 2 mol %). Stirring was continued as the acetone/water ice bath expired over time (room temperature). After 68 hours had passed, the mixture was concentrated and chromatographed over silica gel (100:0-90:10 heptanes/EtOAc) to afford racemic (4aS,10aR)-7-bromo-10a-methyl-4-methylene-1,3,4,4a,5,10a-hexahydropyrano[3,4-b]chromene as a solid (1.37 g, 42%). ¹H NMR (400 MHz, CDCl₃) δ 7.24-7.18 (m, 2H), 6.69 (d, J=8.6 Hz, 1H), 5.04 (s, 1H), 4.86 (s, 1H), 4.26 (d, J=12.4 Hz, 1H), 4.01 (d, J=12.4 Hz, 1H), 3.91 (d, J=10.6 Hz, 1H), 3.54 (d, J=10.6 Hz, 1H), 2.76 (m, 2H), 2.54 (m, 1H), 1.15 (s, 3H).

Step G: A solution of racemic (4aS,10aR)-7-bromo-10a-methyl-4-methylene-1,3,4,4a,5,10a-hexahydropyrano[3,4-b]chromene (591 mg, 2.00 mmol) in anhydrous CH₂Cl₂ (40 mL) and at −78° C. was subjected to ozone gas until it turned blue, indicating completion of the reaction (30 minutes). After bubbling through nitrogen gas to remove excess ozone, the reaction vessel was charged with dimethylsulfide (0.30 mL, 4.0 mmol) and stirred for 45 minutes before removing the dry ice/acetone bath and stirring for another 45 minutes. The mixture was concentrated and chromatographed over silica gel (100:0-85:15 heptanes/EtOAc) to afford racemic (4aR,10aR)-7-bromo-10a-methyl-1,4a,5,10a-tetrahydropyrano[3,4-b]chromen-4(3H)-one as a solid (414 mg, 70%) ¹H NMR (400 MHz, CDCl₃) δ 7.27 (d, J=2.4 Hz, 1H), 7.23 (dd, J=8.7, 2.4 Hz, 1H), 6.72 (d, J=8.7 Hz, 1H), 4.07 (s, 2H), 4.02 (d, J=11.1 Hz, 1H), 3.93 (d, J=11.1 Hz, 1H), 2.97-2.86 (m, 3H), 1.22 (s, 3H).

Step H: To a suspension of racemic (4aR,10aR)-7-bromo-10a-methyl-1,4a,5,10a-tetrahydropyrano[3,4-b]chromen-4(3H)-one (377 mg, 1.27 mmol) in anhydrous MeOH (16.5 mL) was added toluene-4-sulfonyl hydrazine (244 mg, 1.27 mmol) and toluene-4-sulfonic acid (11 mg, 0.064 mmol). The mixture was stirred in a closed vial for 22 hr and then diluted with heptanes (17 mL) and stirred vigourously for 20 minutes. The precipitate was isolated by filtration, rinsing with heptanes to afford racemic (Z)—N′44aS,10aR)-7-bromo-10a-methyl-1,4a,5,10a-tetrahydropyrano[3,4-b]chromen-4(3H)-ylidene)-4-methylbenzenesulfonohydrazide as a solid (522 mg, 88%). ¹H NMR (400 MHz, CDCl₃) δ 7.83 (t, J=9.6 Hz, 2H), 7.35 (d, J=9.6 Hz, 2H), 7.20 (d, J=8.7 Hz, 1H), 7.14 (s, 1H), 6.67 (d, J=8.7 Hz, 1H), 4.54 (d, J=11.6 Hz, 1H), 3.81 (m, 2H), 3.66 (d, J=11.2 Hz, 1H), 2.87-2.68 (m, 3H), 2.44 (s, 3H), 1.00 (s, 3H).

Step I: A solution of racemic (Z)—N′-((4aS,10aR)-7-bromo-10a-methyl-1,4a,5,10a-tetrahydropyrano[3,4-b]chromen-4(3H)-ylidene)-4-methylbenzenesulfonohydrazide (376 mg, 0.808 mmol) in anhydrous CHCl₃ (16 mL) under nitrogen atmosphere was cooled to −10° C. and charged with catecholborane (0.444 mL, 4.04 mmol). Stirring was maintained for 20 minutes and then allowed to warm to room temperature. Then, sodium acetate trihydrate (1.65 g, 12.1 mmol) was added and the mixture was stirred at 70° C. under nitrogen for 3.5 hours. After cooling to room temperature, the mixture was diluted with CH₂Cl₂ and washed with sat. aq. NaHCO₃ and the aqueous layer was extracted again with CH₂Cl₂. The combined organics were dried with MgSO₄ and concentrated to dryness. The residue was purified by flash column chromatography (100:0-90:10 heptanes/EtOAc) to afford racemic (4aS,10aR)-7-bromo-10a-methyl-1,3,4,4a,5,10a-hexahydropyrano[3,4-b]chromene as a solid (172 mg, 75%) ¹H NMR (400 MHz, CDCl₃) δ 7.22-7.13 (m, 2H), 6.66 (d, J=8.4 Hz, 1H), 4.03 (dd, J=11.5, 4.8 Hz, 1H), 3.83 (d, J=10.5 Hz, 1H), 3.48 (ddd, J=11.5, 11.5, 2.7 Hz, 1H), 3.32 (d, J=10.5 Hz, 1H), 2.55 (m, 2H), 2.03-1.89 (m, 1H), 1.75-1.63 (m, 1H), 1.61-1.53 (m, 1H), 1.25 (s, 3H).

Step J: Into a vial was weighed racemic (4aS,10aR)-7-bromo-10a-methyl-1,3,4,4a,5,10a-hexahydropyrano[3,4-b]chromene (123 mg, 0.434 mmol), dirhodium caprolactamate (1.4 mg, 0.5 mol %), sodium bicarbonate (18.2 mg, 0.217 mmol). The mixture was dissolved in DCE (1.7 mL) and tert-butylhydroperoxide (0.40 mL, 2.17 mmol, 5.5 M in decane) was added slowly. The vial was sealed and stirred at 40° C. for 3.5 hours. An additional aliquot of dirhodium caprolactamate (1.4 mg, 0.5 mol %) and tert-butylhydroperoxide (0.40 mL, 2.17 mmol, 5.5 M in decane) was added again and stirred at 40° C. for 3 hours. A third aliquot of dirhodium caprolactamate (1.4 mg, 0.5 mol %) and tert-butylhydroperoxide (0.40 mL, 2.17 mmol, 5.5 M in decane) was added again and stirred at 40° C. for 17 hours. A fourth aliquot of dirhodium caprolactamate (1.4 mg, 0.5 mol %) and tert-butylhydroperoxide (0.40 mL, 2.17 mmol, 5.5 M in decane) was added again and stirred at 40° C. for 7 hours. This type of portion-wise addition is necessary for the reaction to proceed. The mixture was diluted with saturated aqueous NaHCO₃ and extracted twice with CH₂Cl₂ and dried (MgSO₄). The mixture was concentrated and chromatographed over silica gel (100:0-90:10 heptanes/EtOAc). First to elute was recovered starting material (46.4 mg, 38%), followed by racemic (4aS,10aR)-7-bromo-10a-methyl-1,4,4a,10a-tetrahydropyrano[3,4-b]chromen-5(3H)-one as a solid (60.2 mg, 47%). ¹H NMR (400 MHz, CDCl₃) 7.94 (d, J=2.5 Hz, 1H), 7.55 (dd, J=8.9, 2.5 Hz, 1H), 6.83 (d, J=8.9 Hz, 1H), 4.14 (dd, J=11.6, 4.8 Hz, 1H), 3.84 (d, J=10.5 Hz, 1H), 3.57 (d, J=10.5 Hz, 1H), 3.47 (ddd, J=11.6, 11.6, 2.5 Hz, 1H), 2.94 (dd, J=12.2, 4.0 Hz, 1H), 2.08 (m, 1H), 1.81 (m, 1H), 1.37 (s, 3H).

Step K: To a solution of racemic (4aS,10aR)-7-bromo-10a-methyl-1,4,4a,10a-tetrahydropyrano[3,4-b]chromen-5(3H)-one (73.5 mg, 0.247 mmol) in anhydrous THF (4.9 mL) at 0° C. and under nitrogen was added Tebbe reagent (2.47 mL, 1.24 mmol, 0.5 M in toluene). The ice bath was removed and stirring was continued for 30 minutes at which point another aliquot of Tebbe reagent (1.0 mL, 0.49 mmol) was added. After another 35 minutes had passed, the reaction mixture was cooled to 0° C. and 1 M aqueous NaOH was added slowly to quench. After vigorous gas evolution had ceased, the reaction mixture was allowed to warm to room temperature and filtered through Celite®, rinsing with CH₂Cl₂. The mixture was washed with brine, dried over MgSO₄, and concentrated to dryness. The residue thus obtained was purified by flash column chromatography (100:0-95:5 heptanes/EtOAc) to afford racemic (4aR,10aR)-7-bromo-10a-methyl-5-methylene-1,3,4,4a,5,10a-hexahydropyrano[3,4-b]chromene as a solid (30.3 mg, 42%). ¹H NMR (400 MHz, CDCl₃) 7.63 (d, J=2.4 Hz, 1H), 7.26 (m, 1H), 6.69 (d, J=8.7 Hz, 1H), 5.57 (d, J=2.0 Hz, 1H), 4.89 (d, J=2.0 Hz, 1H), 4.13 (dd, J=11.5, 4.8 Hz, 1H), 3.87 (d, J=10.4 Hz, 1H), 3.53 (ddd, J=12.0, 12.0, 2.4 Hz, 1H), 3.41 (d, J=10.4 Hz, 1H), 2.59-2.53 (m, 1H), 1.91 (m, 1H), 1.68 (td, J=12.0, 4.8 Hz, 1H), 1.18 (s, 3H).

Step L: To a suspension of racemic (4aR,10aR)-7-bromo-10a-methyl-5-methylene-1,3,4,4a,5,10a-hexahydropyrano[3,4-b]chromene (30.3 mg, 0.103 mmol) and silver(I) cyanate (23.3 mg, 0.154 mmol) in anhydrous MeCN (1.0 mL) at 0° C. and under nitrogen was added dropwise a solution of iodine (28.7 mg, 0.113 mmol) in anhydrous EtOAc (3.1 mL) over a period of 8 min. After stirring for 40 minutes, the mixture was allowed to warm to room temperature. A further 30 minutes was allowed to pass, at which point the mixture was filtered through a short pad of Celite®, rinsing with anhydrous THF (3.1 mL). After the addition of ammonium hydroxide (2.1 mL, 16 mmol, 28% in water) to the mother liquor, the mixture was stirring vigourously under air for 30 minutes before being diluted with brine and extracted twice with CH₂Cl₂. The combined organics were dried (MgSO₄) and concentrated to dryness. On top of this residue was weighed tetrakis(triphenylphosphine)palladium(0) (12.0 mg, 10 mol %), potassium carbonate (71 mg, 0.51 mmol), and 5-chloro-3-pyridineboronic acid (33.0 mg, 0.205 mmol). The reaction vessel was purged under nitrogen, charged with anhydrous 1,4-dioxane (1.5 mL) and degassed water (1.0 mL), and stirred at 80° C. for 18 hours. After cooling to room temperature, the mixture was filtered through Celite® and concentrated to dryness. The residue thus obtained was purified by flash column chromatography (100:0-0:100 CH₂Cl₂/[90:10:0.6:0.6 CH₂Cl₂/MeOH/NH₄OH/H₂O]) several times, pooling pure fractions of each of the desired diastereomers. First to elute was racemic (4S,4a′R,10a′R)-7′-(5-chloropyridin-3-yl)-10a′-methyl-3′,4′,4a′,10a′-tetrahydro-1′H,5H-spiro[oxazole-4,5′-pyrano[3,4-b]chromen]-2-amine (2.9 mg, 7% over two steps), followed by racemic (4R,4a′R,10a′R)-7′-(5-chloropyridin-3-yl)-10a′-methyl-3′,4′,4a′,10a′-tetrahydro-1′H,5H-spiro[oxazole-4,5′-pyrano[3,4-b]chromen]-2-amine (4.6 mg, 12%), both as solids. Data for first solid: ¹H NMR (400 MHz, CDCl₃) δ 8.65 (d, J=2.3 Hz, 1H), 8.50 (d, J=2.3 Hz, 1H), 7.78 (d, J=4.5 Hz, 1H), 7.39 (s, 1H), 7.36 (d, J=8.4 Hz, 1H), 6.90 (d, J=8.4 Hz, 1H), 4.46 (d, J=8.7 Hz, 1H), 4.28 (d, J=8.7 Hz, 1H), 4.19-4.12 (m, 2H), 3.84 (d, J=10.8 Hz, 1H), 3.54 (dd, J=10.7, 10.7 Hz, 1H), 3.37 (d, J=10.8 Hz, 1H), 2.04-1.98 (m, 1H), 1.82 (m, 1H), 1.51 (s, 3H); LRMS m/z calculated for C₂₀H₂₀N₃O₃Cl [M+1]+: 386.1. Found 386.1.

### Example 137

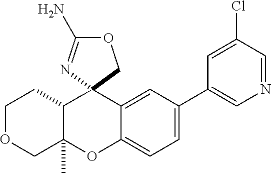

### (4S,4a′R,10aR)-7′-(5-chloropyridin-3-yl)-10a′-methyl-3′,4′,4a′,10a′-tetrahydro-1′H,5H-spiro[oxazole-4,5′-pyrano[3,4-b]chromen]-2-amine

(4S,4a′R,10a′R)-7′-(5-Chloropyridin-3-yl)-10a′-methyl-3′,4′,4a′,10a′-tetrahydro-1′H,5H-spiro[oxazole-4,5′-pyrano[3,4-b]chromen]-2-amine was prepared in Example 136. Data for second solid: ¹H NMR (400 MHz, CDCl₃) δ 8.66 (s, 1H), 8.49 (s, 1H), 7.80 (s, 1H), 7.40 (s, 1H), 7.36 (d, J=8.3 Hz, 1H), 6.87 (d, J=8.3 Hz, 1H), 4.65 (d, J=8.8 Hz, 1H), 4.23 (d, J=8.8 Hz, 1H), 4.13 (m, 1H), 3.83 (d, J=10.6 Hz, 1H), 3.54 (dd, J=10.4, 10.4 Hz, 1H), 3.42 (d, J=10.6 Hz, 1H), 2.37 (dd, J=12.4, 3.7 Hz, 1H), 1.73-1.63 (m, 2H), 1.31 (s, 3H); LRMS m/z calculated for C₂₀H₂₀N₃O₃Cl [M+1]+: 386.1. Found 386.1.

### Example 138

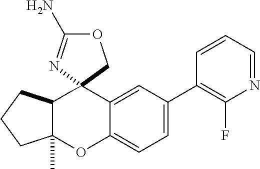

### racemic (3aS,4′S,9aS)-7-(2-fluoropyridin-3-yl)-3a-methyl-2,3,3a,9a-tetrahydro-1H,5′H-spiro[cyclopenta[b]chromene-9,4′-oxazol]-2′-amine

Step A: To a stirring solution of 2-methylcyclohexanone (27.01 g, 240.8 mmol) in CH₂Cl₂ (600 mL, 0.4 M) at 0° C. was added meta-chloroperbenzoic acid (100.0 g, 433.4 mmol, ˜70 wt. %) over 10 minutes. After 5 minutes, the ice bath was removed and stirring was continued for 5 hour, at which time the reaction mixture was filtered, and the solid was rinsed with CH₂Cl₂ and discarded. The mother liquor was carefully diluted with saturated aqueous Na₂SO₃ (exotherm), and the organic layer was washed twice more with saturated aqueous NaHCO₃ and dried (MgSO₄). After concentrating to dryness, additional solid was filtered off and discarded, rinsing with heptanes. The mother liquor was concentrated to dryness to obtain 7-methyloxepan-2-one as a colorless liquid (28.30 g, 92%). ¹H NMR (400 MHz, CDCl₃) δ 4.44 (dq, J=12.8, 6.4 Hz, 1H), 2.74-2.54 (m, 2H), 2.00-1.82 (m, 3H), 1.73-1.51 (m, 3H), 1.36 (d, J=6.4 Hz, 3H).

Step B: To a stirring solution of 2-bromo-4-chlorophenol (30.00 g, 144.6 mmol) in anhydrous diethylether (290 mL, 0.5 M) under nitrogen and at −78° C., was added n-butyllithium (120 mL, 289 mmol, 2.5 M in hexanes, untitrated) over a period of 15 minutes. The cooling bath was replaced with a 0° C. ice/water bath for 2 hours, then cooled back down to −78° C., followed by the addition of 7-methyloxepan-2-one (20.39 g, 159.1 mmol). After 3 hours had passed, the mixture was diluted with water and extracted with diethylether. The organics were washed with brine, dried (MgSO₄), and concentrated to dryness. The crude lactol was re-dissolved in HPLC-grade CH₂Cl₂ (580 mL, 0.25 M). Pyridinium dichromate (83.27 g, 216.9 mmol) was added, and the reaction mixture was stirred for 65 hours. Filtration through Florisil, rinsing with CH₂Cl₂, afforded a solution, which upon concentration contained the crude dione. The residue was re-dissolved in anhydrous methanol (100 mL, 1.5 M) and pyrrolidine (0.97 mL, 8 mol %) was added. The reaction mixture was stirred at 50° C. for 19.5 hours and then concentrated to dryness and partitioned between saturated aqueous NH₄Cl and CH₂Cl₂. The organic layer was dried over MgSO₄, concentrated to dryness, and the residue was subjected to flash column chromatography (100:0-90:10 heptanes/EtOAc) to afford, after being freed of volatiles, racemic cis-7-chloro-3a-methyl-1,2,3,3a-tetrahydrocyclopenta[b]chromen-9(9aH)-one as a solid (1.90 g, 6% over 3 steps). ¹H NMR (400 MHz, CDCl₃) δ 7.83 (d, J=2.7 Hz, 1H), 7.40 (dd, J=8.8, 2.7 Hz, 1H), 6.87 (d, J=8.8 Hz, 1H), 2.68-2.52 (m, 1H), 2.28-2.07 (m, 2H), 2.03-1.78 (m, 4H), 1.42 (s, 3H).

Step C: To a solution of racemic cis-7-chloro-3a-methyl-1,2,3,3a-tetrahydrocyclopenta[b]chromen-9(9aH)-one (829 mg, 3.50 mmol) and methyltriphenylphosphonium bromide (3.19 g, 8.75 mmol) in anhydrous toluene (10.5 mL) and under nitrogen atmosphere was added potassium bis(trimethylsilyl)amide (20 mL, 8.05 mmol, 0.5 M in toluene). The mixture was stirred at 80° C. for a period of 1.5 hours and then diluted with saturated aqueous NaHCO₃ and extracted with EtOAc. The organic layer was dried with MgSO₄ and concentrated to dryness. Purification of the reaction residue via flash column chromatography (100:0-95:5 heptanes/EtOAc) delivered racemic (3aS,9aS)-7-chloro-3a-methyl-9-methylene-1,2,3,3a,9,9a-hexahydrocyclopenta[b]chromene as a liquid (604 mg, 74%). ¹H NMR (400 MHz, CDCl₃) δ 7.52 (d, J=2.5 Hz, 1H), 7.09 (dd, J=8.7, 2.5 Hz, 1H), 6.73 (d, J=8.7 Hz, 1H), 5.52 (s, 1H), 4.98 (s, 1H), 2.51 (dd, J=11.5, 8.1 Hz, 1H), 2.15-2.05 (m, 1H), 1.89-1.53 (m, 5H), 1.23 (s, 3H).

Step D: To a solution of racemic (3aS,9aS)-7-chloro-3a-methyl-9-methylene-1,2,3,3a,9,9a-hexahydrocyclopenta[b]chromene (103 mg, 0.438 mmol) in anhydrous MeCN (1.75 mL) and anhydrous EtOAc (1.5 mL) under nitrogen atmosphere was added silver(I) cyanate (80.4 mg, 0.526 mmol). The suspension was cooled to 0° C. and a pre-cooled (0° C.) solution of iodine (122 mg, 0.482 mmol) in anhydrous EtOAc (4.5 mL) was added dropwise, rinsing with anhydrous MeCN (2 mL). The resultant mixture was stirred at 0° C. for 2.5 hours before being filtered through Celite®, rinsing with CH₂Cl₂ and concentrated to dryness. The residue was dissolved in THF (6 mL) and ammonium hydroxide (2.5 mL, 19 mmol, 28% in water) was added and the mixture was stirred vigorously for 16.5 hours. The mixture was partitioned between CH₂Cl₂ and brine and the organic layer was dried (MgSO₄) and concentrated. The residue thereby obtained was taken up in 1.4 mL DMSO, 2 drops of ammonium hydroxide (28% in water) were added, and after heating to a homogeneous solution, purified by preparative RPLC (Xbridge 3×10 cm, 10 μm packing, 70 mL/minute, 30:70-70:30 MeCN/0.1% aqueous NH₄OH), detecting by mass (single quadrupole, ESI+ ionization) to obtain racemic (3aS,4′S,9aS)-7-chloro-3a-methyl-2,3,3a,9a-tetrahydro-1H,5′H-spiro[cyclopenta[b]chromene-9,4′-oxazol]-2′-amine as a solid (38.5 mg, 30%). ¹H NMR (400 MHz, CDCl₃) δ 7.23 (d, J=2.5 Hz, 1H), 7.08 (dd, J=8.7, 2.5 Hz, 1H), 6.73 (d, J=8.7 Hz, 1H), 4.41 (d, J=8.7 Hz, 1H), 4.31 (d, J=8.6 Hz, 1H), 4.26 (br s, 2H), 2.13 (dd, J=11.2, 8.3 Hz, 1H), 2.06-1.97 (m, 1H), 1.91-1.60 (m, 4H), 1.48 (s, 3H), 1.40-1.27 (m, 1H).

Step E: In a 0.5-2.0 mL Biotage microwave vial was weighed racemic (3aS,4′S,9aS)-7-chloro-3a-methyl-2,3,3a,9a-tetrahydro-1H,5′H-spiro[cyclopenta[b]chromene-9,4′-oxazol]-2′-amine (34.4 mg, 0.117 mmol), tris(dibenzylideneacetone)dipalladium(0) (55.5 mg, 0.0587 mmol), tricyclohexylphosphonium tetrafluoroborate (31.3 mg, 0.117 mmol), potassium phosphate tribasic (153 mg, 0.705 mmol, dried under vacuum at 150° C. and stored in a desiccator) and 2-fluoropyridine-3-boronic acid (50.7 mg, 0.352 mmol). After purging with nitrogen, anhydrous, degassed 1,4-dioxane (1.65 mL) and degassed water (0.80 mL) were injected and the reaction mixture was subjected to microwave heating at 160° C. (Biotage Initiator, 40 min, normal absorbance). After cooling, the top layer of the biphasic mixture was decanted off and concentrated. The reaction mixture was then taken up in MeOH (1 mL), and purified by preparative RPLC (Xbridge 3×10 cm, 10 μm packing, 70 mL/minute, 30:70-70:30 MeCN/0.1% aqueous NH₄OH), detecting by mass (single quadrupole, ESI+ ionization) to obtain racemic (3aS,4′S,9aS)-7-(2-fluoropyridin-3-yl)-3a-methyl-2,3,3a,9a-tetrahydro-1H,5′H-spiro[cyclopenta[b]chromene-9,4′-oxazol]-2′-amine as a solid (8.0 mg, 19%). ¹H NMR (400 MHz, CDCl₃) δ 8.13 (d, J=4.8 Hz, 1H), 7.81 (ddd, J=9.6, 7.4, 1.8 Hz, 1H), 7.50-7.44 (m, 1H), 7.39-7.31 (m, 1H), 7.25-7.17 (m, 1H), 6.89 (d, J=8.5 Hz, 1H), 4.53 (d, J=8.7 Hz, 1H), 4.36 (d, J=8.7 Hz, 1H), 4.14 (br s, 2H), 2.19 (dd, J=11.4, 8.3 Hz, 1H), 2.15-2.05 (m, 1H), 1.94-1.65 (m, 5H), 1.55 (s, 3H), 1.46-1.36 (m, 1H); LRMS m/z calculated for C₂₀H₂₀N₃O₂F [M+1]+: 354.2. Found 354.2.

### Example 139

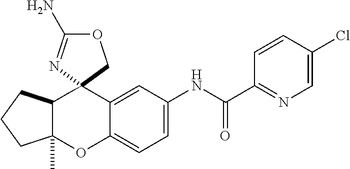

### racemic N-((3aS,4′S,9aS)-7′-amino-3a-methyl-2,3,3a,9a-tetrahydro-1H,5′H-spiro[cyclopenta[b]chromene-9,4′-oxazol]-7-yl)-5-chloropicolinamide

In a 0.5-2.0 mL Biotage microwave vial was weighed racemic (3aS,4′S,9aS)-7-chloro-3a-methyl-2,3,3a,9a-tetrahydro-1H,5′H-spiro[cyclopenta[b]chromene-9,4′-oxazol]-2′-amine (72.3 mg, 0.247 mmol), copper(I) iodide (94.1 mg, 0.494 mmol), potassium carbonate (172 mg, 1.23 mmol) and 5-chloropyridine-2-carboxamide (116 mg, 0.741 mmol). After purging with nitrogen, anhydrous, degassed 1,4-dioxane (1.65 mL) and trans-1,2-bis(methylamino)cyclohexane (169 μL, 1.04 mmol) were injected. and the reaction was mixture was subjected to microwave heating at 160° C. (Biotage Initiator, 80 minutes, normal absorbance). The reaction mixture was then filtered through Celite® and rinsed with CH₂Cl₂. The mother liquor was washed with saturated aqueous NH₄Cl, dried (MgSO₄) and concentrated to dryness. The residue was taken up in 1 mL DMSO, and purified by preparative RPLC (Xbridge 3×10 cm, 10 μm packing, 70 mL/minute 30:70-70:30 MeCN/0.1% aqueous NH₄OH), detecting by mass (single quadrupole, ESI+ ionization) to obtain racemic N-((3aS,4′S,9aS)-2′-amino-3a-methyl-2,3,3a,9a-tetrahydro-1H,5′H-spiro[cyclopenta[b]chromene-9,4′-oxazole]-7-yl)-5-chloropicolinamide as a solid (3.1 mg, 3%). ¹H NMR (400 MHz, CDCl₃) δ 8.48 (br s, 1H), 8.12 (d, J=8.5 Hz, 1H), 7.87 (br d, 1H), 7.70 (br s, 1H), 7.31 (s, 1H), 7.16 (dd, J=8.7, 2.4 Hz, 1H), 6.78 (d, J=8.7 Hz, 1H), 5.51 (br s, 1H), 4.59 (d, J=8.3 Hz, 1H), 4.35 (d, J=8.3 Hz, 1H), 2.42-2.29 (m, 1H), 2.08-1.62 (m, 6H), 1.42 (s, 3H); LRMS m/z calculated for C₂₁H₂₁N₄O₃Cl [M+1]+: 413.1. Found 413.1.

### Example 140

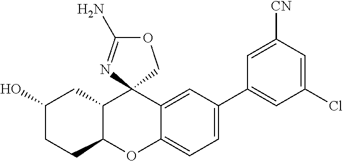

### 3-((2′S,4R,4a′S,9a′R)-2-amino-2′-hydroxy-1′,2′,3′,4′,4a′,9a′-hexahydro-5H-spiro[oxazole-4,9′-xanthen]-7′-yl)-5-chlorobenzonitrile

Step A: (4a′S*,9a′R*)-7′-Bromo-3′,4′,4a′,9a′-tetrahydro-1′H,2″H-dispiro[1,3-dioxolane-2,2′-xanthene-9′,3″-[1,4]oxazole]-5″-amine (from Example 23, Step F) was purified by chiral SFC to afford 4 single enantiomers:

First separation: MG III preparative SFC, ChiralPak AD-H, 250×50 mm I.D., SC—CO₂/40% (Ethanol+0.2% DEA), 150 mL/minute. Sample preparation: dissolved in methanol and DCM (3:1), about 50 mg/mL, 4 mL per injection: clean peak 1, mixture of peak 2+3, and peak 4.

Second separation (peak 2+3): MG III preparative SFC, ChiralCel OJ-H, 250×30 mm I.D., SC—CO₂/40% (Methanol+0.2% DEA), 60 mL/minute. Sample preparation: dissolved in methanol and DCM (3:1), about 30 mg/mL, 4 mL per injection: clean peak 2 and peak 3.

Peak 1: (4R,4a′R,9a′S)-7′-bromo-3′,4′,4a′,9a′-tetrahydro-1′H,2″H-dispiro[1,3-dioxolane-2,2′-xanthene-9′,3″-[1,4]oxazole]-5″-amine

Peak 2: (4S,4a′S,9a′R)-7′-bromo-3′,4′,4a′,9a′-tetrahydro-1′H,2″H-dispiro[1,3-dioxolane-2,2′-xanthene-9′,3″-[1,4]oxazole]-5″-amine

Peak 3: (4S,4a′R,9a′S)-7′-bromo-3′,4′,4a′,9a′-tetrahydro-1′H,2″H-dispiro[1,3-dioxolane-2,2′-xanthene-9′,3″-[1,4]oxazole]-5″-amine

Peak 4: (4R,4a′S,9a′R)-7′-bromo-3′,4′,4a′,9a′-tetrahydro-1′H,2″H-dispiro[1,3-dioxolane-2,2′-xanthene-9′,3″-[1,4]oxazole]-5″-amine

Step B: (4R,4a′S,9a′R)-2-Amino-7′-bromo-1′,4′,4a′,9a′-tetrahydro-5H-spiro[oxazole-4,9′-xanthen]-2′(3′H)-one was prepared according to the procedure in Example 23, Step G, using enantiopure Peak 4 from Step A.

Step C: (4R,4a′S,9a′R)-2-Amino-7′-(4,4,5,5-tetramethyl-1,3,2-dioxaborolan-2-yl)-1′,4′,4a′,9a′-tetrahydro-5H-spiro[oxazole-4,9′-xanthen]-2′(3′H)-one was prepared according to the procedure in Example 75, Step A, substituting (4R,4a′S,9a′R)-2-amino-7′-bromo-1′,4′,4a′,9a′-tetrahydro-5H-spiro[oxazole-4,9′-xanthen]-2′(3′H)-one: (206 mg, 0.52 mmol, 61%).

Step D: 3-((4R,4a′S,9a′R)-2-Amino-2′-oxo-1′,2′,3′,4′,4a′,9a′-hexahydro-5H-spiro[oxazole-4,9′-xanthene]-7′-yl)-5-chlorobenzonitrile was prepared according to the procedure on Example 75, Step B, substituting (4R,4a′S,9a′R)-2-amino-7′-(4,4,5,5-tetramethyl-1,3,2-dioxaborolan-2-yl)-1′,4′,4a′,9a′-tetrahydro-5H-spiro[oxazole-4,9′-xanthen]-2′(3′H)-one and 3-bromo-5-chlorobenzonitrile (60 mg, 0.15 mmol, 28%).

Step E: 3-((2′S,4R,4a′S,9a′R)-2-Amino-2′-hydroxy-1′,2′,3′,4′,4a′,9a′-hexahydro-5H-spiro[oxazole-4,9′-xanthene]-7′-yl)-5-chlorobenzonitrile was prepared according to the procedure from Example 63, Step B (19 mg, 0.046 mmol, 32%). m/z (APCI-pos) M+1=410.1 (100%). ¹H NMR (CD₃OD) δ 7.87 (m, 2H), 7.69 (t, J=1.6 Hz, 1H), 7.58 (d, J=2.3 Hz, 1H), 7.46 (dd, J=8.2, 2.3 Hz, 1H), 6.86 (d, J=8.6 Hz, 1H), 4.60 (d, J=9.0 Hz, 1H), 4.03 (d, J=9.0 Hz, 1H), 3.91 (td, J=11, 5.0 Hz, 1H), 3.68 (m, 1H), 2.26 (m, 1H), 2.06 (m, 2H), 1.87 (m, 1H), 1.62 (m, 1H), 1.45 (m, 1H), 1.21 (m, 1H).

### Example 141

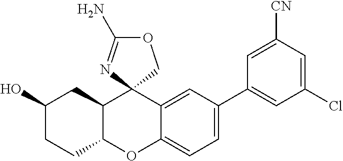

### 3-((2′R,4R,4a′R,9a′S)-2-amino-2′-hydroxy-1′,2′,3′,4′,4a′,9a′-hexahydro-5H-spiro[oxazole-4,9′-xanthen]-7′-yl)-5-chlorobenzonitrile

Step A: (4R,4a′R,9a′S)-2-Amino-7′-bromo-1′,4′,4a′,9a′-tetrahydro-5H-spiro[oxazole-4,9′-xanthen]-2′(3′H)-one was prepared according to the procedure in Example 23, Step G, using enantiopure Peak 1 from Example 140, Step A.

Step B: (4R,4a′R,9a′S)-2-Amino-7′-(4,4,5,5-tetramethyl-1,3,2-dioxaborolan-2-yl)-1′,4′,4a′,9a′-tetrahydro-5H-spiro[oxazole-4,9′-xanthen]-2′(3′H)-one was prepared according to the procedure in Example 75, Step A, substituting (4R,4a′R,9a′S)-2-amino-7′-bromo-1′,4′,4a′,9a′-tetrahydro-5H-spiro[oxazole-4,9′-xanthen]-2′(3′H)-one (254 mg, 0.64 mmol, 75%).

Step C: 3-((4R,4a′R,9a′S)-2-Amino-2′-oxo-1′,2′,3′,4′,4a′,9a′-hexahydro-5H-spiro[oxazole-4,9′-xanthene]-7′-yl)-5-chlorobenzonitrile was prepared according to the procedure on Example 75, Step B, substituting (4R,4a′R,9a′S)-2-amino-7′-(4,4,5,5-tetramethyl-1,3,2-dioxaborolan-2-yl)-1′,4′,4a′,9a′-tetrahydro-5H-spiro[oxazole-4,9′-xanthen]-2′(31-1)-one and 3-bromo-5-chlorobenzonitrile (178 mg, 0.44 mmol, 68%).

Step D: 3-((2′R,4R,4a′R,9a′S)-2-Amino-2′-hydroxy-1′,2′,3′,4′,4a′,9a′-hexahydro-5H-spiro[oxazole-4,9′-xanthene]-7′-yl)-5-chlorobenzonitrile was prepared according to the procedure from Example 63, Step B (45 mg, 0.11 mmol, 28%). m/z (APCI-pos) M+1=410.1 (100%). ¹H NMR (CD₃OD) δ 7.86 (d, J=2.0 Hz, 2H), 7.70 (t, J=1.6 Hz, 1H), 7.50 (d, J=2.0 Hz, 1H), 7.46 (dd, J=8.2, 2.3 Hz, 1H), 6.89 (d, J=8.6 Hz, 1H), 4.67 (d, J=9.0 Hz, 1H), 4.42 (d, J=9.0 Hz, 1H), 3.98 (td, J=11, 4.7 Hz, 1H), 3.74 (m, 1H), 2.25 (m, 1H), 2.01 (m, 2H), 1.81 (m, 1H), 1.61 (m, 1H), 1.38 (m, 2H).

### Example 142

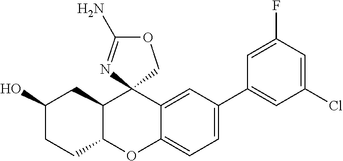

### (2′R,4R,4a′R,9a′S)-2-amino-7′-(3-chloro-5-fluorophenyl)-1′,2′,3′,4′,4a′,9a′-hexahydro-5H-spiro[oxazole-4,9′-xanthen]-2′-ol

Step A: (4R,4a′R,9a′S)-2-Amino-7′-(3-chloro-5-fluorophenyl)-1′,4′,4a′,9a′-tetrahydro-5H-spiro[oxazole-4,9′-xanthen]-2′(3′H)-one was prepared according to the procedure from Example 63, Step A, substituting 3-chloro-5-fluorophenylboronic acid and (4R,4a′R,9a′S)-2-amino-7′-bromo-1′,4′,4a′,9a′-tetrahydro-5H-spiro[oxazole-4,9′-xanthen]-2′(3′H)-one from Example 39, Step A: (1.43 g, 3.58 mmol, 99%).

Step B: (2′R,4R,4a′R,9a′S)-2-Amino-7′-(3-chloro-5-fluorophenyl)-1′,2′,3′,4′,4a′,9a′-hexahydro-5H-spiro[oxazole-4,9′-xanthen]-2′-ol was prepared according to the procedure from Example 63, Step B: (1.00 g, 2.48 mmol, 69%). m/z (APCI-pos) M+1=403.0 (100%). ¹H NMR (CD₃OD) δ 7.46 (m, 2H), 7.36 (s, 1H), 7.15 (d, J=9.8 Hz, 1H), 7.06 (d, J=7.8 Hz, 1H), 6.94 (d, J=8.0 Hz, 1H), 4.88 (d, J=9.4 Hz, 1H), 4.68 (d, J=9.4 Hz, 1H), 3.96 (td, J=11, 4.7 Hz, 1H), 3.76 (m, 1H), 2.33 (m, 1H), 2.08 (m, 2H), 1.92 (m, 1H), 1.64 (m, 1H), 1.45 (m, 1H), 1.35 (m, 1H).

### Example 143

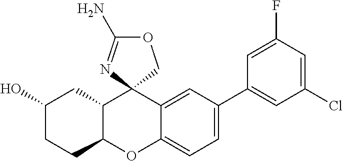

### (2′S,4R,4a′S,9a′R)-2-amino-7′-(3-chloro-5-fluorophenyl)-1′,2′,3′,4′,4a′,9a′-hexahydro-5H-spiro[oxazole-4,9′-xanthen]-7′-ol

Step A: (4R,4a′S,9a′R)-2-Amino-7′-(3-chloro-5-fluorophenyl)-1′,4′,4a′,9a′-tetrahydro-5H-spiro[oxazole-4,9′-xanthen]-2′(3′H)-one was prepared according to the procedure from Example 63, Step A, substituting 3-chloro-5-fluorophenylboronic acid and (4R,4a′S,9a′R)-2-amino-7′-bromo-1′,4′,4a′,9a′-tetrahydro-5H-spiro[oxazole-4,9′-xanthen]-2′(3′H)-one from Example 39, Step A (1.57 g, 3.92 mmol, 99%).

Step B: (2′S,4R,4a′S,9a′R)-2-Amino-7′-(3-chloro-5-fluorophenyl)-1′,2′,3′,4′,4a′,9a′-hexahydro-5H-spiro[oxazole-4,9′-xanthen]-2′-ol was prepared according to the procedure from Example 63, Step B (1.20 g, 2.98 mmol, 76%). m/z (APCI-pos) M+1=403.1 (100%). ¹H NMR (CD₃OD) δ 7.56 (m, 1H), 7.45 (dt, J=8.6, 2.0 Hz, 1H), 7.34 (s, 1H), 7.18 (m, 1H), 7.06 (m, 1H), 6.92 (m, 1H), 4.88 (d, J=9.4 Hz, 1H), 4.42 (d, J=9.4 Hz, 1H), 3.88 (td, J=11, 4.7 Hz, 1H), 3.75 (m, 1H), 2.33 (m, 1H), 2.10 (m, 3H), 1.69 (m, 1H), 1.46 (m, 1H), 1.26 (m, 1H).

### Example 144

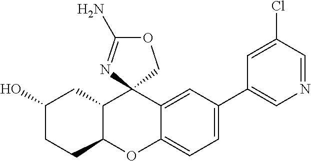

### (2′S,4R,4a′S,9a′R)-2-amino-7′-(5-chloropyridin-3-yl)-1′,2′,3′,4′,4a′,9a′-hexahydro-5H-spiro[oxazole-4,9′-xanthen]-2′-ol

The racemic mixture of diastereomers of (2′S,4a′S,9a′R)-2-amino-7′-(5-chloropyridin-3-yl)-1′,2′,3′,4′,4a′,9a′-hexahydro-5H-spiro[oxazole-4,9′-xanthen]-2′-ol from Example 23 was purified by SFC using two conditions. Purification 1: Chiralpak AD (3 cm×25 cm, 20 um), SC—CO₂/70% (EtOH+0.2% NH₄OH) at 300 mL/minute @ 100 bar, 40° C., UV 230 nm, Sample preparation: 5.07 g in 50 mL MeOH, 10 mL Chloroform, 2 mL formic acid, 1 mL water, and 20 mL acetonitrile, 1.5 mL injections (108 mg) every 7.25 minutes.

Peaks 1+2: Example 297 and Example 296.

Peak 3: Example 145.

Peak 4: Example 144.

Purification 2: Chiralpak AD (3 cm×25 cm, 20 um), SC—CO₂/80% (EtOH+0.1% NH₄OH) at 200 mL/min @ 100 bar, 40° C., UV 230 nm, Sample preparation: 1.7 g in 20 mL MeOH, 0.5 mL injections (42 mg) every 4.1 minutes.

Peak 1: Example 297.

Peak 2: Example 296.

Peak 4: (2′S,4R,4a′S,9aR)-2-Amino-7′-(5-chloropyridin-3-yl)-1′,2′,3′,4′,4a′,9a′-hexahydro-5H-spiro[oxazole-4,9′-xanthen]-2′-ol. m/z (APCI-pos) M+1=386.1 (100%). ¹H NMR (CD₃OD) δ 8.68 (br s, 1H), 8.46 (br s, 1H), 8.07 (br s, 1H), 7.64 (br s, 1H), 7.49 (d, J=8.2 Hz, 1H), 6.88 (d, J=8.2 Hz, 1H), 4.69 (d, J=9.2 Hz, 1H), 4.12 (d, J=9.2 Hz, 1H), 3.93 (td, J=11, 5.0 Hz, 1H), 3.68 (m, 1H), 2.27 (m, 1H), 2.06 (m, 2H), 1.91 (m, 1H), 1.63 (m, 1H), 1.45 (m, 1H), 1.23 (m, 1H).

### Example 145

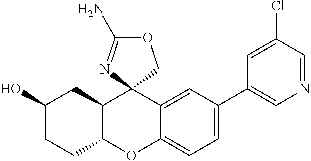

### (2′R,4R,4a′R,9a′S)-2-amino-7′45-chloropyridin-3-yl)-1′,2′,3′,4′,4a′,9a′-hexahydro-5H-spiro[oxazole-4,9′-xanthen]-2′-ol

From Example 144, Purification 1, Peak 3: (2′R,4R,4a′R,9a′S)-2-amino-7′-(5-chloropyridin-3-yl)-1′,2′,3′,4′,4a′,9a′-hexahydro-5H-spiro[oxazole-4,9′-xanthen]-2′-ol. m/z (APCI-pos) M+1=386.1 (100%). ¹H NMR (CD₃OD) δ 8.66 (d, J=2.0 Hz, 1H), 8.47 (d, J=2.0 Hz, 1H), 8.06 (t, J=2.0 Hz, 1H), 7.56 (d, J=2.0 Hz, 1H), 7.49 (dd, J=8.6, 2.3 Hz, 1H), 6.92 (d, J=8.2 Hz, 1H), 4.75 (d, J=9.0 Hz, 1H), 4.50 (d, J=9.4 Hz, 1H), 3.98 (td, J=11, 5.1 Hz, 1H), 3.75 (m, 1H), 2.26 (m, 1H), 2.03 (m, 2H), 1.87 (m, 1H), 1.62 (m, 1H), 1.37 (m, 2H).

### Example 146

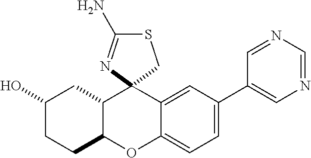

### (2′S,4R,4a′S,9a′R)-2-amino-7′-(pyrimidin-5-yl)-1′,2′,3′,4′,4a′,9a′-hexahydro-5H-spiro[thiazole-4,9′-xanthen]-2′-ol

Step A: (4a′S,9a′R)-2-Amino-7′-(pyrimidin-5-yl)-1′,4′,4a′,9a′-tetrahydro-5H-spiro[thiazole-4,9′-xanthen]-2′(3′H)-one was prepared from 2-amino-7′-bromo-1′,4′,4a′,9a′-tetrahydro-5H-spiro[thiazole-4,9′-xanthen]-2′(3′H)-one (Example 30, Step B) using the procedure from Example 63, Step A, substituting pyrimidin-5-ylboronic acid (170 mg, 0.46 mmol, 97%).

Step B: (2′S,4R,4a′S,9a′R)-2-Amino-7′-(pyrimidin-5-yl)-1′,2′,3′,4′,4a′,9a′-hexahydro-5H-spiro[thiazole-4,9′-xanthen]-2′-ol was prepared according to the procedure from Example 63, Step B (27 mg, 0.07 mmol, 15%). m/z (APCI-pos) M+1=369.1 (100%). ¹H NMR (CDCl₃) δ 9.11 (br s, 1H), 8.90 (br s, 2H), 7.78 (br s, 1H), 7.41 (br d, J=8.6 Hz, 1H), 6.97 (br d, J=8.6 Hz, 1H), 4.04 (m, 1H), 3.75 (m, 3H), 2.29 (m, 1H), 2.09 (m, 2H), 1.84 (m, 1H), 1.62 (m, 1H), 1.37 (m, 2H).

### Example 147

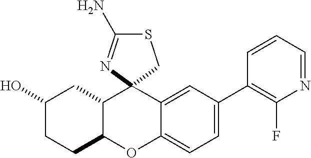

### (2′S,4R,4a′S,9a′R)-2-amino-7′-(2-fluoropyridin-3-yl)-1′,2′,3′,4′,4a′,9a′-hexahydro-5H-spiro[thiazole-4,9′-xanthen]-2′-ol

Step A: (4a′S,9a′R)-2-Amino-7′-(2-fluoropyridin-3-yl)-1′,4′,4a′,9a′-tetrahydro-5H-spiro[thiazole-4,9′-xanthen]-2′(3′H)-one was prepared from 2-amino-7′-bromo-1′,4′,4a′,9a′-tetrahydro-5H-spiro[thiazole-4,9′-xanthen]-2′(3′H)-one (Example 30, Step B) using the procedure from Example 63, Step A, substituting 2-fluoropyridin-3-ylboronic acid (23 mg, 0.06 mmol, 85%).

Step B: (2′S,4R,4a′S,9aR)-2-Amino-7′-(2-fluoropyridin-3-yl)-1′,2′,3′,4′,4a′,9a′-hexahydro-5H-spiro[thiazole-4,9′-xanthen]-2′-ol trifluoroacetic acid salt was prepared according to the procedure from Example 63, Step B (20 mg, 0.05 mmol, 74%): m/z (APCI-pos) M+1=386.1 (100%). ¹H NMR (CD₃OD) δ 8.16 (d, J=4.3 Hz, 1H), 8.05 (ddd, J=11, 7.4, 2.0 Hz, 1H), 7.84 (s, 2H), 7.54 (d, J=8.6 Hz, 1H), 7.41 (ddd, J=8.2, 4.7, 1.6 Hz, 1H), 7.01 (d, J=8.6 Hz, 1H), 4.12 (d, J=12 Hz, 1H), 3.98 (d, J=12 Hz, 1H), 3.93 (td, J=11, 4.7 Hz, 1H), 3.82 (m, 1H), 2.33 (m, 2H), 2.21 (td, J=12, 3.5 Hz, 1H), 2.09 (m, 1H), 1.69 (m, 1H), 1.42 (m, 1H), 1.29 (q, J=11 Hz, 1H).

### Example 148

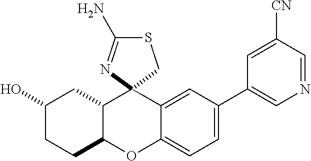

### 5-((2′S,4R,4a′S,9a′R)-2-amino-2′-hydroxy-1′,2′,3′,4′,4a′,9a′-hexahydro-5H-spiro[thiazole-4,9′-xanthen]-7′-yl)nicotinonitrile

Step A: 5-((4R,4a′S,9a′R)-2-Amino-2′-oxo-1′,2′,3′,4′,4a′,9a′-hexahydro-5H-spiro[thiazole-4,9′-xanthen]-7′-yl)nicotinonitrile was prepared from 2-amino-7′-bromo-1′,4′,4a′,9a′-tetrahydro-5H-spiro[thiazole-4,9′-xanthen]-2′(3′H)-one (Example 30, Step B) using the procedure from Example 63, Step A, substituting 5-(4,4,5,5-tetramethyl-1,3,2-dioxaborolan-2-yl)nicotinonitrile (20 mg, 0.05 mmol, 72%).

Step B: 5-((2′S,4R,4a′S,9a′R)-2-Amino-2′-hydroxy-1′,2′,3′,4′,4a′,9a′-hexahydro-5H-spiro[thiazole-4,9′-xanthen]-7′-yl)nicotinonitrile trifluoroacetic acid salt was prepared according to the procedure from Example 63, Step B (4 mg, 0.01 mmol, 15%): m/z (APCI-pos) M+1=393.1 (100%). ¹H NMR (CD₃OD) δ 9.06 (s, 1H), 8.86 (s, 1H), 8.49 (s, 1H), 7.93 (d, J=2.0 Hz, 1H), 7.68 (dd, J=8.2, 2.0 Hz, 1H), 7.05 (d, J=8.6 Hz, 1H), 4.26 (d, J=13 Hz, 1H), 3.96 (d, J=13 Hz, 1H), 3.92 (td, J=11, 4.7 Hz, 1H), 3.82 (m, 1H), 2.41 (m, 1H), 2.31 (m, 1H), 2.22 (m, 1H), 2.09 (m, 1H), 1.69 (m, 1H), 1.41 (m, 1H), 1.29 (q, J=11 Hz, 1H).

### Example 149

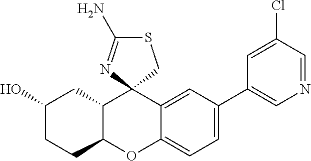

### (2′S,4R,4a′S,9a′R)-2-amino-7′-(5-chloropyridin-3-yl)-1′,2′,3′,4′,4a′,9a′-hexahydro-5H-spiro[thiazole-4,9′-xanthen]-2′-ol

(4R,4a′S,9a′R)-2-Amino-7′-(5-chloropyridin-3-yl)-1′,2′,3′,4′,4a′,9a′-hexahydro-5H-spiro[thiazole-4,9′-xanthen]-2′-ol from Example 30, Step E was purified by chiral SFC: Chiralcel OJ (3 cm×25 cm, 20 um), SC—CO₂/20% (methanol+0.1% NH₄OH), 300 mL/minute @ 100 Bar, 40° C., UV 230 nm. Sample preparation: dissolved 3.44 grams in 14 mL of MeOH and 3 mL of formic acid, 0.5 mL injections (˜81.9 mg) every 2.3 minutes.

Peak 1: (2′S,4R,4a′S,9a′R)-2-amino-7′-(5-chloropyridin-3-yl)-1′,2′,3′,4′,4a′,9a′-hexahydro-5H-spiro[thiazole-4,9′-xanthen]-2′-ol. m/z (APCI-pos) M+1=402.0 (100%). ¹H NMR (CD₃OD) δ 8.64 (d, J=2.0 Hz, 1H), 8.46 (d, J=2.0 Hz, 1H), 8.02 (t, J=2.0 Hz, 1H), 7.82 (d, J=2.0 Hz, 1H), 7.47 (dd, J=8.6, 2.0 Hz, 1H), 6.91 (d, J=8.6 Hz, 1H), 4.02 (td, J=11, 4.7 Hz, 1H), 3.84 (d, J=12 Hz, 1H), 3.74 (m, 1H), 3.72 (d, J=12 Hz, 1H), 2.24 (m, 1H), 2.11 (m, 1H), 2.04 (m, 1H), 1.87 (m, 1H), 1.61 (m, 1H), 1.35 (m, 2H).

Peak 2: Example 302, (2′R,4S,4a′R,9a′S)-2-Amino-7′-(5-chloropyridin-3-yl)-1′,2′,3′,4′,4a′,9a′-hexahydro-5H-spiro[thiazole-4,9′-xanthen]-2′-ol.

### Example 150

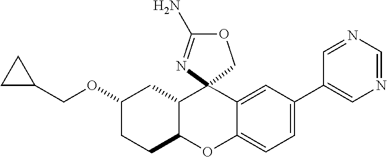

### (2′S,4R,4a′S,9a′R)-2′-(cyclopropylmethoxy)-7′-(pyrimidin-5-yl)-1′,2′,3′,4′,4a′,9a′-hexahydro-5H-spiro[oxazole-4,9′-xanthen]-2-amine

Step A: (4a′S,9a′R)-2-Amino-7′-(pyrimidin-5-yl)-1′,4′,4a′,9a′-tetrahydro-5H-spiro[oxazole-4,9′-xanthen]-2′(3′H)-one was prepared according to the procedure from Example 63, Step A, substituting pyrimidin-5-ylboronic acid (300 mg, 0.86 mmol, 93%).

Step B: (4a′S,9a′R)-2′-(Cyclopropylmethoxy)-7′-(pyrimidin-5-yl)-1′,2′,3′,4′,4a′,9a′-hexahydro-5H-spiro[oxazole-4,9′-xanthen]-2-amine trifluoroacetic acid salt was prepared using the procedure from Example 28, Step B, substituting (4a′S,9a′R)-2-amino-7′-(pyrimidin-5-yl)-1′,4′,4a′,9a′-tetrahydro-5H-spiro[oxazole-4,9′-xanthen]-2′(3′H)-one (11 mg, 0.03 mmol, 45% after C18 purification).

From C18 semi-preparative chromatography: Peak 1: (2′S,4R,4a′S,9a′R)-2′-(cyclopropylmethoxy)-7′-(pyrimidin-5-yl)-1′,2′,3′,4′,4′,9a′-hexahydro-5H-spiro[oxazole-4,9′-xanthen]-2-amine trifluoroacetic acid salt. m/z (APCI-pos) M+1=407.2 (100%). ¹H NMR (CD₃OD) δ 9.11 (s, 1H), 9.07 (s, 2H), 7.95 (s, 1H), 7.67 (d, J=8.6 Hz, 1H), 7.02 (d, J=8.6 Hz, 1H), 5.23 (d, J=10 Hz, 1H), 4.68 (d, J=10 Hz, 1H), 4.05 (td, J=10, 6.0 Hz, 1H), 3.51 (m, 1H), 3.39 (m, 2H), 2.34 (m, 1H), 2.22 (m, 1H), 2.13 (m, 2H), 1.67 (m, 1H), 1.41 (m, 2H), 1.05 (m, 1H), 0.53 (m, 2H), 0.22 (m, 2H).

Peak 2, Example 151.

Peak 3: Example 168, (2′R,4R,4a′S,9a′R)-2′-(cyclopropylmethoxy)-7′-(pyrimidin-5-yl)-1′,2′,3′,4′,4a′,9a′-hexahydro-5H-spiro[oxazole-4,9′-xanthen]-2-amine trifluoroacetic acid salt.

Peak 4, Example 172, (2′R,4S,4a′S,9aR)-2′-(cyclopropylmethoxy)-7′-(pyrimidin-5-yl)-1′,2′,3′,4′,4a′,9a′-hexahydro-5H-spiro[oxazole-4,9′-xanthen]-2-amine trifluoroacetic acid salt.

### Example 151

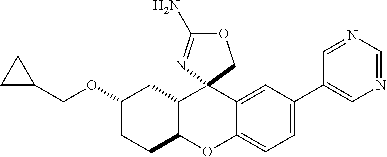

### (2′S,4S,4a′S,9a′R)-2′-(cyclopropylmethoxy)-7′-(pyrimidin-5-yl)-1′,2′,3′,4′,4a′,9a′-hexahydro-5H-spiro[oxazole-4,9′-xanthen]-2-amine

(2′S,4S,4a′S,9a′R)-2′-(Cyclopropylmethoxy)-7′-(pyrimidin-5-yl)-1′,2′,3′,4′,4a′,9a′-hexahydro-5H-spiro[oxazole-4,9′-xanthen]-2-amine trifluoroacetic acid salt was prepared in Example 150. m/z (APCI-pos) M+1=407.2 (100%). ¹H NMR (CD₃OD) δ 9.11 (s, 1H), 9.07 (s, 2H), 7.91 (d, J=2.0 Hz, 1H), 7.69 (dd, J=8.6, 2.0 Hz, 1H), 7.06 (d, J=8.6 Hz, 1H), 5.22 (d, J=10 Hz, 1H), 5.03 (d, J=10 Hz, 1H), 3.92 (td, J=11, 4.7 Hz, 1H), 3.59 (m, 1H), 3.41 (t, J=6.3 Hz, 2H), 2.25 (m, 4H), 1.67 (m, 1H), 1.35 (m, 1H), 1.22 (m, 1H), 1.06 (m, 1H), 0.54 (m, 2H), 0.23 (m, 2H).

### Example 152

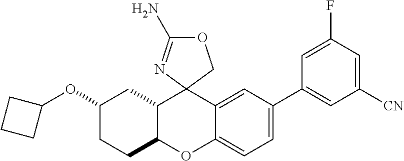

### 3-((2′S,4a′S,9a′R)-2-amino-2′-cyclobutoxy-1′,2′,3′,4′,4a′,9a′-hexahydro-5H-spiro[oxazole-4,9′-xanthen]-7′-yl)-5-fluorobenzonitrile

Step A: To a solution of cyclobutanol (0.513 g, 7.12 mmol) and 2,6-lutidine (0.829 mL, 7.12 mmol) in DCM (14.2 mL) at 0° C. was added TMSOTf (2.58 mL, 14.2 mmol). The reaction mixture was stirred at 0° C. for 2 hours. To this mixture was added (4a′S,9a′R)-2-amino-7′-bromo-1′,4′,4a′,9a′-tetrahydro-5H-spiro[oxazole-4,9′-xanthen]-2′(3′H)-one (0.50 g, 1.42 mmol, from Example 23, Step G) and triethylsilane (1.14 mL, 7.12 mmol), and the resulting mixture was stirred at room temperature for 1 day. The reaction mixture was concentrated, and the residue was purified by flash chromatography eluting with DCM/MeOH, 1% NH₄OH to afford (4a′S,9a′R)-7′-bromo-2′-cyclobutoxy-1′,2′,3′,4′,4a′,9a′-hexahydro-5H-spiro[oxazole-4,9′-xanthen]-2-amine (0.50 g, 1.23 mmol, 86%).

Step B: 3-((2′S,4a′S,9a′R)-2-Amino-2′-cyclobutoxy-1′,2′,3′,4′,4a′,9a′-hexahydro-5H-spiro[oxazole-4,9′-xanthen]-7′-yl)-5-fluorobenzonitrile trifluoroacetic acid salt was prepared using the procedure from Example 63, Step A, substituting 3-cyano-5-fluorophenylboronic acid (54 mg, 0.12 mmol, 12%). m/z (APCI-pos) M+1=447.8 (100%). Two diastereomers: ¹H NMR (CD₃OD) 7.89 (m, 4H), 7.76 (d, J=9.8 Hz, 2H), 7.64 (m, 2H), 7.49 (m, 2H), 7.00 (d, J=8.6 Hz, 1H), 6.96 (d, J=8.6 Hz, 1H), 5.22 (d, J=9.8 Hz, 1H), 5.20 (d, J=9.8 Hz, 1H), 5.01 (d, J=9.8 Hz, 1H), 4.66 (d, J=9.8 Hz, 1H), 4.10 (m, 2H), 4.02 (td, J=11, 4.3 Hz, 1H), 3.89 (td, J=11, 4.3 Hz, 1H), 3.57 (m, 1H), 3.49 (m, 1H), 2.31 (m, 2H), 2.23 (m, 5H), 2.13 (m, 4H), 2.04 (m, 1H), 1.94 (m, 4H), 1.67 (m, 4H), 1.54 (m, 2H), 1.36 (m, 3H), 1.21 (m, 1H).

### Example 153

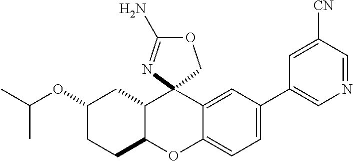

### 5-((2′S,4R,4a′S,9a′R)-2-amino-2′-isopropoxy-1′,2′,3′,4′,4a′,9a′-hexahydro-5H-spiro[oxazole-4,9′-xanthen]-7′-yl)nicotinonitrile

Step A: (4a′S,9a′R)-7′-Bromo-2′-isopropoxy-1′,2′,3′,4′,4a′,9a′-hexahydro-5H-spiro[oxazole-4,9′-xanthen]-2-amine was prepared according to the procedure in Example 152, Step A, substituting isopropanol (1.10 g, 2.78 mmol, 83%).

Step B: 5-((2′S,4R,4a′S,9a′R)-2-Amino-2′-isopropoxy-1′,2′,3′,4′,4a′,9a′-hexahydro-5H-spiro[oxazole-4,9′-xanthen]-7′-yl)nicotinonitrile trifluoroacetic acid salt was prepared using the procedure from Example 63, Step A (9.0 mg, 0.022 mmol, 15%). m/z (APCI-pos) M+1=419.2 (100%); ¹H NMR (CD₃OD) δ 9.07 (br s, 1H), 8.85 (br s, 1H), 8.50 (br s, 1H), 7.95 (m, 1H), 7.69 (m, 1H), 7.02 (m, 1H), 5.23 (br d, 1H), 4.67 (br d, 1H), 4.05 (br s, 1H), 3.88 (m, 1H), 3.58 (m, 1H), 2.33 (m, 1H), 2.17 (m, 2H), 2.02 (m, 1H), 1.68 (m, 1H), 1.41 (m, 2H), 1.17 (br s, 6H).

### Example 154

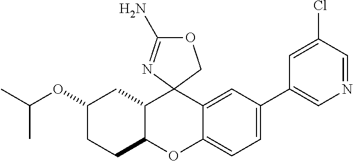

### (2′S,4a′S,9a′R)-7′-(5-chloropyridin-3-yl)-2′-isopropoxy-1′,2′,3′,4′,4a′,9a′-hexahydro-5H-spiro[oxazole-4,9′-xanthen]-2-amine

(2′S,4a′S,9aR)-7′-(5-Chloropyridin-3-yl)-2′-isopropoxy-1′,2′,3′,4′,4a′,9a′-hexahydro-5H-spiro[oxazole-4,9′-xanthen]-2-amine was prepared using the procedure from Example 63, Step A, substituting 5-chloropyridin-3-ylboronic acid (12 mg, 0.03 mmol, 18%). m/z (APCI-pos) M+1=428.1 (100%). Major diastereomer: ¹H NMR (CD₃OD) δ 8.77 (br s, 1H), 8.53 (br s, 1H), 8.20 (s, 1H), 7.91 (m, 1H), 7.64 (m, 1H), 6.99 (m, 1H), 5.21 (br d, 1H), 4.67 (br d, 1H), 4.04 (br s, 1H), 3.82 (m, 1H), 3.57 (m, 1H), 2.33 (m, 1H), 2.16 (m, 2H), 2.01 (m, 1H), 1.68 (m, 1H), 1.40 (m, 2H), 1.17 (br s, 6H).

### Example 155

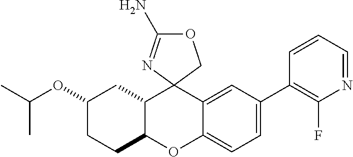

### (2′S,4a′S,9aR)-7′-(2-fluoropyridin-3-yl)-2′-isopropoxy-1′,2′,3′,4′,4a′,9a′-hexahydro-5H-spiro[oxazole-4,9′-xanthen]-2-amine

(2′S,4a′S,9a′R)-7′-(2-Fluoropyridin-3-yl)-2′-isopropoxy-1′,2′,3′,4′,4a′,9a′-hexahydro-5H-spiro[oxazole-4,9′-xanthen]-2-amine was prepared using the procedure from Example 63, Step A, substituting 2-fluoropyridin-3-ylboronic acid (35 mg, 0.09 mmol, 14%). m/z (APCI-pos) M+1=411.8 (100%). Major diastereomer: ¹H NMR (CD₃OD) δ 8.16 (m, 1H), 8.06 (m, 1H), 7.76 (br s, 1H), 7.53 (br d, 1H), 7.40 (m, 1H), 6.97 (d, J=8.6 Hz, 1H), 5.21 (d, J=10 Hz, 1H), 4.64 (d, J=10 Hz, 1H), 4.03 (td, J=11, 5.0 Hz, 1H), 3.81 (m, 1H), 3.57 (m, 1H), 2.32 (m, 1H), 2.16 (m, 2H), 2.01 (m, 1H), 1.68 (m, 1H), 1.41 (m, 2H), 1.16 (br s, 6H).

### Example 156

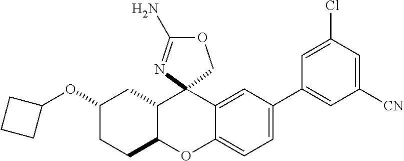

### 3-((2′S,4R,4a′S,9a′R)-2-amino-2′-cyclobutoxy-1′,2′,3′,4′,4a′,9a′-hexahydro-5H-spiro[oxazole-4,9′-xanthen]-7′-yl)-5-chlorobenzonitrile

Step A: A 100 mL 3 neck flask was fitted with stir bar, septum and condenser. The apparatus was purged with nitrogen and the flask was charged with (1,5-cyclooctadiene)(methoxy)iridium(I) dimer (0.36 g, 0.55 mmol), 4,4′-di-tert-butyl-[2,2′]bipyridinyl (0.29 g, 1.09 mmol) and 4,4,4′,4′,5,5,5′,5′-octamethyl-2,2′-bi(1,3,2-dioxaborolane) (9.23 g, 36.3 mmol). The apparatus was purged for an additional 1 minute. Hexanes (110 mL) and 3-chlorobenzonitrile (10.0 g, 72.7 mmol) were charged to the flask, and the mixture was stirred at 60° C. for 8 hours. The reaction mixture was concentrated, and the residue was purified by flash chromatography eluting with DCM/MeOH 0-3%. The oil was left under vacuum to afford 3-chloro-5-(4,4,5,5-tetramethyl-1,3,2-dioxaborolan-2-yl)benzonitrile as a low melting, colorless solid (12.5 g, 47.4 mmol, 65%).

Step B: 3-((2′S,4R,4a′S,9a′R)-2-Amino-2′-cyclobutoxy-1′,2′,3′,4′,4a′,9a′-hexahydro-5H-spiro[oxazole-4,9′-xanthen]-7′-yl)-5-chlorobenzonitrile trifluoroacetic acid salt was prepared using the procedure from Example 63, Step A, substituting (4a′S,9a′R)-7′-bromo-2′-cyclobutoxy-1′,2′,3′,4′,4a′,9a′-hexahydro-5H-spiro[oxazole-4,9′-xanthen]-2-amine and 3-chloro-5-(4,4,5,5-tetramethyl-1,3,2-dioxaborolan-2-yl)benzonitrile (5.2 mg, 0.011 mmol, 10%).

Peak 1 from C18 semi-preparative chromatography, 3-((2′S,4R,4a′S,9a′R)-2-amino-2′-cyclobutoxy-1′,2′,3′,4′,4a′,9a′-hexahydro-5H-spiro[oxazole-4,9′-xanthen]-7′-yl)-5-chlorobenzonitrile trifluoroacetic acid salt: m/z (APCI-pos) M+1=463.8 (100%); ¹H NMR (CD₃OD) δ 7.99 (m, 2H), 7.92 (br s, 1H), 7.74 (br s, 1H), 7.63 (m, 1H), 6.96 (d, J=8.6 Hz, 1H), 5.21 (d, J=9.6 Hz, 1H), 4.67 (d, J=9.6 Hz, 1H), 4.12 (m, 1H), 4.03 (m, 1H), 3.49 (m, 1H), 2.31 (m, 1H), 2.23 (m, 3H), 2.13 (m, 3H), 2.04 (m, 1H), 1.94 (m, 2H), 1.67 (m, 1H), 1.54 (m, 1H), 1.39 (m, 1H).

### Example 157

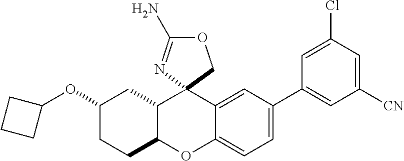

### 3-((2′S,4S,4a′S,9a′R)-2-amino-2′-cyclobutoxy-1′,2′,3′,4′,4a′,9a′-hexahydro-5H-spiro[oxazole-4,9′-xanthen]-7′-yl)-5-chlorobenzonitrile

Peak 2 from C18 semi-preparative chromatography in Example 156, Step B: 3-((2′S,4S,4a′S,9a′R)-2-amino-2′-cyclobutoxy-1′,2′,3′,4′,4a′,9a′-hexahydro-5H-spiro[oxazole-4,9′-xanthen]-7′-yl)-5-chlorobenzonitrile trifluoroacetic acid salt. m/z (APCI-pos) M+1=463.8 (100%); ¹H NMR (CD₃OD) δ 7.99 (br s, 2H), 7.87 (d, J=2.0 Hz, 1H), 7.75 (br s, 1H), 7.65 (dd, J=8.6, 2.0 Hz, 1H), 7.01 (d, J=8.6 Hz, 1H), 5.22 (d, J=10 Hz, 1H), 5.01 (d, J=10 Hz, 1H), 4.14 (m, 1H), 3.88 (td, J=11, 4.7 Hz, 1H), 3.57 (m, 1H), 2.31 (m, 1H), 2.24 (m, 2H), 2.15 (m, 3H), 1.94 (m, 2H), 1.69 (m, 2H), 1.54 (m, 1H), 1.35 (m, 1H), 1.20 (m, 1H).

### Example 158

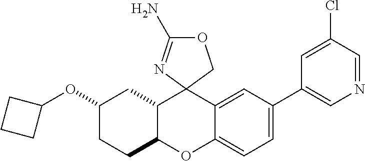

### (2′S,4a′S,9a′R)-7′-(5-chloropyridin-3-yl)-2′-cyclobutoxy-1′,2′,3′,4′,4a′,9a′-hexahydro-5H-spiro[oxazole-4,9′-xanthen]-2-amine

(2′S,4a′S,9a′R)-7′-(5-Chloropyridin-3-yl)-2′-cyclobutoxy-1′,2′,3′,4′,4a′,9a′-hexahydro-5H-spiro[oxazole-4,9′-xanthen]-2-amine trifluoroacetic acid salt (1:1 mixture of diastereomers at oxazoline, racemic) was prepared using the procedure from Example 63, Step A, substituting (4a′S,9a′R)-7′-bromo-7′-cyclobutoxy-1′,2′,3′,4′,4a′,9a′-hexahydro-5H-spiro[oxazole-4,9′-xanthen]-2-amine and 5-chloropyridin-3-ylboronic acid (26 mg, 0.059 mmol, 48%). m/z (APCI-pos) M+1=439.8 (100%). Two diastereomers: ¹H NMR (CD₃OD) δ 8.78 (br s, 2H), 8.55 (br s, 2H), 8.23 (d, J=2.0 Hz, 2H), 7.92 (d, J=2.0 Hz, 1H), 7.88 (d, J=2.0 Hz, 1H), 7.65 (m, 2H), 7.03 (d, J=8.6 Hz, 1H), 6.98 (d, J=8.6 Hz, 1H), 5.22 (d, J=10 Hz, 1H), 5.21 (d, J=10 Hz, 1H), 5.02 (d, J=10 Hz, 1H), 4.67 (d, J=10 Hz, 1H), 4.13 (m, 2H), 4.03 (td, J=11, 4.7 Hz, 1H), 3.88 (td, J=11, 4.7 Hz, 1H), 3.57 (m, 1H), 3.49 (m, 2.32 (m, 2H), 2.23 (m, 5H), 2.15 (m, 4H), 2.05 (m, 1H), 1.94 (m, 5H), 1.67 (m, 4H), 1.54 (m, 2H), 1.36 (m, 2H), 1.21 (m, 1H).

### Example 159

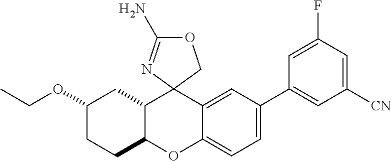

### 3-((2′S,4a′S,9a′R)-2-amino-2′-ethoxy-1′,2′,3′,4′,4a′,9a′-hexahydro-5H-spiro[oxazole-4,9′-xanthen]-7′-yl)-5-fluorobenzonitrile

Step A: To a solution of ethoxytrimethylsilane (1.68 g, 14.2 mmol) and (4a′S,9a′R)-2-amino-7′-bromo-1′,4′,4a′,9a′-tetrahydro-5H-spiro[oxazole-4,9′-xanthen]-2′(3′H)-one (1.00 g, 2.85 mmol) in DCM (14.2 mL) at 0° C. was added TMSOTf (2.58 mL, 14.2 mmol). The reaction mixture was stirred at 0° C. for 1 hour. To this mixture was added triethylsilane (2.274 mL, 14.24 mmol), and the resulting mixture was stirred at room temperature for 1 day.

Step B: 3-((2′S,4a′S,9a′R)-2-Amino-2′-ethoxy-1′,2′,3′,4′,4a′,9a′-hexahydro-5H-spiro[oxazole-4,9′-xanthen]-7′-yl)-5-fluorobenzonitrile trifluoroacetic acid salt was prepared using the procedure from Example 63, Step A, substituting (4a′S,9a′R)-7′-bromo-2′-ethoxy-1′,2′,3′,4′,4a′,9a′-hexahydro-5H-spiro[oxazole-4,9′-xanthen]-2-amine and 3-cyano-5-fluorophenylboronic acid (32 mg, 0.076 mmol, 55%). Two diastereomers: m/z (APCI-pos) M+1=421.8 (100%); ¹H NMR (CD₃OD) δ 7.89 (m, 4H), 7.77 (br s, 2H), 7.64 (br s, 2H), 7.49 (m, 2H), 7.01 (d, J=8.6 Hz, 1H), 6.96 (d, J=8.6 Hz, 1H), 5.21 (m, 2H), 5.02 (d, J=9.6 Hz, 1H), 4.67 (d, J=9.6 Hz, 1H), 4.04 (td, 1H), 3.91 (td, 1H), 3.59 (m, 5H), 3.48 (m, 1H), 2.33 (m, 2H), 2.23 (m, 2H), 2.12 (m, 3H), 1.67 (m, 2H), 1.38 (m, 3H), 1.20 (m, 8H).

### Example 160

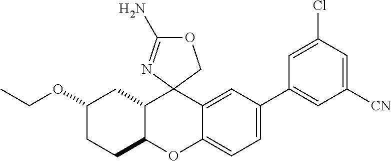

### 3-((2′S,4a′S,9a′R)-2-amino-2′-ethoxy-1′,2′,3′,4′,4a′,9a′-hexahydro-5H-spiro[oxazole-4,9′-xanthen]-7′-yl)-5-chlorobenzonitrile

3-((2′S,4a′S,9a′R)-2-Amino-2′-ethoxy-1′,2′,3′,4′,4a′,9a′-hexahydro-5H-spiro[oxazole-4,9′-xanthen]-7′-yl)-5-chlorobenzonitrile trifluoroacetic acid salt was prepared using the procedure from Example 63, Step A, substituting (4a′S,9a′R)-7′-bromo-2′-ethoxy-1′,2′,3′,4′,4a′,9a′-hexahydro-5H-spiro[oxazole-4,9′-xanthen]-2-amine and 3-chloro-5-(4,4,5,5-tetramethyl-1,3,2-dioxaborolan-2-yl)benzonitrile (34 mg, 0.078 mmol, 59%). Two diastereomers: m/z (APCI-pos) M+1=437.8 (100%); ¹H NMR (CD₃OD) δ 7.99 (br s, 4H), 7.92 (br d, 1H), 7.86 (br d, 1H), 7.73 (br s, 2H), 7.63 (m, 2H), 6.99 (m, 2H), 5.21 (m, 2H), 5.02 (d, J=9.6 Hz, 1H), 4.67 (d, J=9.6 Hz, 1H), 4.04 (td, J=10, 5.0 Hz, 1H), 3.91 (td, J=10, 5.0 Hz, 1H), 3.60 (m, 5H), 3.48 (m, 1H), 2.33 (m, 2H), 2.23 (m, 3H), 2.12 (m, 3H), 1.67 (m, 2H), 1.38 (m, 2H), 1.20 (m, 8H).

### Example 161

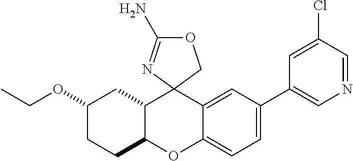

### (2′S,4a′S,9a′R)-7′-(5-chloropyridin-3-yl)-2′-ethoxy-1′,2′,3′,4′,4a′,9a′-hexahydro-5H-spiro[oxazole-4,9′-xanthen]-2-amine

(2′S,4a′S,9a′R)-7′-(5-chloropyridin-3-yl)-2′-ethoxy-1′,2′,3′,4′,4a′,9a′-hexahydro-5H-spiro[oxazole-4,9′-xanthen]-2-amine trifluoroacetic acid salt (racemic, mixture of diastereomers at oxazoline) was prepared using the procedure from Example 63, Step A, substituting (4a′S,9a′R)-7′-bromo-2′-ethoxy-1′,2′,3′,4′,4a′,9a′-hexahydro-5H-spiro[oxazole-4,9′-xanthen]-2-amine and 5-chloropyridin-3-ylboronic acid (17 mg, 0.041 mmol, 31%). Two diastereomers: m/z (APCI-pos) M+1=413.8 (100%); ¹H NMR (CD₃OD) δ 8.77 (s, 2H), 8.53 (s, 2H), 8.19 (s, 2H), 7.91 (m, 1H), 7.86 (m, 1H), 7.65 (m, 2H), 7.03 (d, J=8.6 Hz, 1H), 6.99 (d, J=8.6 Hz, 1H), 5.22 (m, 2H), 5.02 (d, J=9.6 Hz, 1H), 4.67 (d, J=9.6 Hz, 1H), 4.04 (td, 1H), 3.91 (td, 1H), 3.59 (m, 5H), 3.47 (m, 1H), 2.33 (m, 2H), 2.23 (m, 3H), 2.12 (m, 3H), 1.67 (m, 2H), 1.39 (m, 2H), 1.20 (m, 8H).

### Example 162

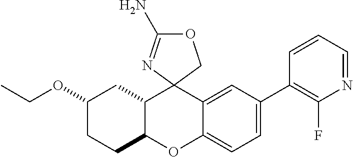

### (2′S,4a′S,9a′R)-2′-ethoxy-7′-(2-fluoropyridin-3-yl)-1′,2′,3′,4′,4a′,9a′-hexahydro-5H-spiro[oxazole-4,9′-xanthen]-2-amine

(2′S,4a′S,9a′R)-2′-ethoxy-7′-(2-fluoropyridin-3-yl)-1′,2′,3′,4′,4a′,9a′-hexahydro-5H-spiro[oxazole-4,9′-xanthen]-2-amine trifluoroacetic acid salt (racemic, mixture of diastereomers at oxazoline) was prepared using the procedure from Example 63, Step A, substituting (4a′S,9a′R)-7′-bromo-2′-ethoxy-1′,2′,3′,4′,4a′,9a′-hexahydro-5H-spiro[oxazole-4,9′-xanthen]-2-amine and 2-fluoropyridin-3-ylboronic acid (36 mg, 0.091 mmol, 51%). m/z (APCI-pos) M+1=397.8 (100%).

The following compounds in Table 2 were prepared according to the above procedures using appropriate intermediates.

TABLE 2

| Ex. | | | NMR/ |
|---|---|---|---|
| # | Structure | Name | MS |
| 163 | 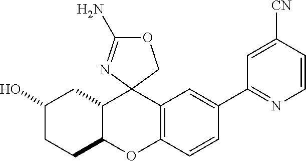 | 2-((2′S*,4a′S*,9a′R*)-2-amino-2′- hydroxy-1′,2′,3′,4′,4a′,9a′- hexahydro-5H-spiro[oxazole-4,9′- xanthen]-7′-yl)isonicotinonitrile bis(trifluoroacetic acid) salt | 377.1 |
| 164 | 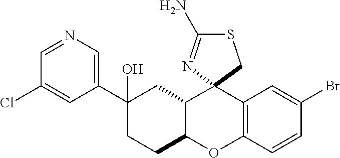 | (4S*,4a′S*,9a′R*)-2-amino-7′- bromo-2′-(5-chloropyridin-3-yl)- 1′,2′,3′,4′,4a′,9a′-hexahydro-5H- spiro[thiazole-4,9′-xanthen]-2′-ol | 480.0, 482.0 |
| 165 | 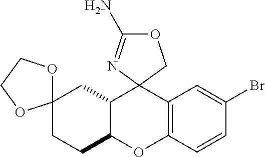 | (4′aS*,9′aR*)-7′-bromo- 3′,4′,4′a,9′a-tetrahydro-1′H,2″H- dispiro[1,3-dioxolane-2,2′- xanthene-9′,3″-[1,4]oxazole]-5″- amine | 395.0, 397.0 |
| 166 | 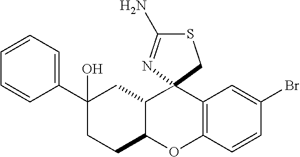 | (4S*,4a′S*,9a′R*)-2-amino-7′- bromo-2′-phenyl-1′,2′,3′,4′,4a′,9a′- hexahydro-5H-spiro[thiazole-4,9′- xanthen]-2′-ol | 445.0, 447.0 |
| 167 | 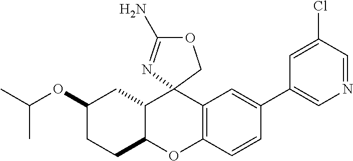 | (2′R,4S,4a′S,9a′R)-7′-(5- chloropyridin-3-yl)-2′-isopropoxy- 1′,2′,3′,4′,4a′,9a′-hexahydro-5H- spiro[oxazole-4,9′-xanthen]-2- amine TFA salt (axial ether, racemic, single diastereomer) | 428.1 |
| 168 | 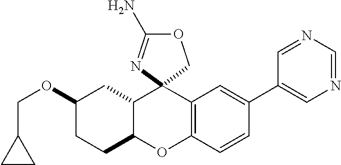 | (2′R,4R,4a′S,9a′R)-2′- (cyclopropylmethoxy)-7′- (pyrimidin-5-yl)-1′,2′,3′,4′,4a′,9a′- hexahydro-5H-spiro[oxazole-4,9′- xanthen]-2-amine TFA salt (single diastereomer, axial ether, racemic) | 407.1 |
| 169 | 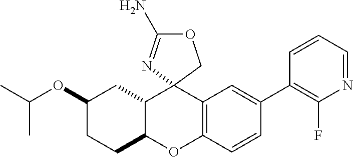 | (2′R,4S,4a′S,9a′R)-7′-(2- fluoropyridin-3-yl)-2′-isopropoxy- 1′,2′,3′,4′,4a′,9a′-hexahydro-5H- spiro[oxazole-4,9′-xanthen]-2- amine TFA salt (single diastereomer, axial ether, racemic) | 412.2 |
| 170 | 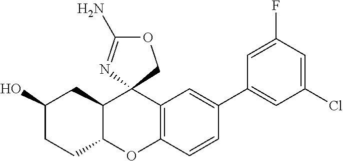 | (2′R,4S,4a′R,9a′S)-2-amino-7′-(3- chloro-5-fluorophenyl)- 1′,2′,3′,4′,4a′,9a′-hexahydro-5H- spiro[oxazole-4,9′-xanthen]-2′-ol (single enantiomer, axial hydroxyl) | 403.1 |
| 171 | 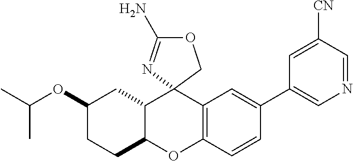 | 5-((2′R,4S,4a′S,9a′R)-2-amino-2′- isopropoxy-1′,2′,3′,4′,4a′,9a′- hexahydro-5H-spiro[oxazole-4,9′- xanthen]-7′-yl)nicotinonitrile TFA salt (axial ether, single diastereomer, racemic) | 419.2 |
| 172 | 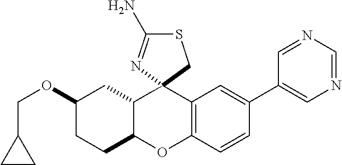 | (2′R,4S,4a′S,9a′R)-2′- (cyclopropylmethoxy)-7′- (pyrimidin-5-yl)-1′,2′,3′,4′,4a′,9a′- hexahydro-5H-spiro[thiazole-4,9′- xanthen]-2-amine TFA salt (axial ether, single diastereomer, racemic) | 423.1 |
| 173 | 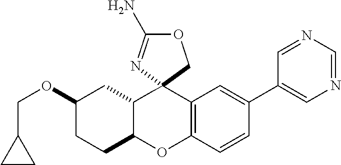 | (2′R,4S,4a′S,9a′R)-2′- (cyclopropylmethoxy)-7′- (pyrimidin-5-yl)-1′,2′,3′,4′,4a′,9a′- hexahydro-5H-spiro[oxazole-4,9′- xanthen]-2-amine TFA salt (axial ether, single diastereomer, racemic) | 407.1 |
| 174 | 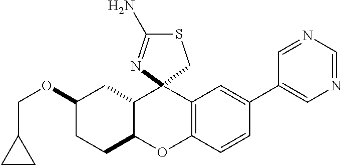 | (2′R,4R,4a′S,9a′R)-2′- (cyclopropylmethoxy)-7′- (pyrimidin-5-yl)-1′,2′,3′,4′,4a′,9a′- hexahydro-5H-spiro[thiazole-4,9′- xanthen]-2-amine TFA salt (axial ether, single diastereomer, racemic) | 423.1 |
| 175 | 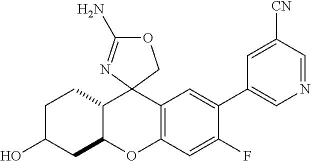 | (4a′S,9a′R)-2-amino-7′-(5- chloropyridin-3-yl)-6′-fluoro- 1′,2′,3′,4′,4a′,9a′-hexahydro-5H- spiro[oxazole-4,9′-xanthen]-3′-ol TFA salt (mixture of stereoisomers at alcohol and oxazole) | 404.0 |
| 176 | 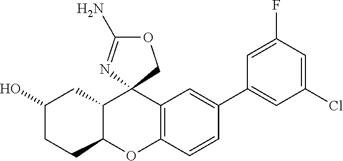 | (2′S,4S,4a′S,9a′R)-2-amino-7′-(3- chloro-5-fluorophenyl)- 1′,2′,3′,4′,4a′,9a′-hexahydro-5H- spiro[oxazole-4,9′-xanthen]-2′-ol (single enantiomer, equatorial hydroxyl) | 403.1 |
| 177 | 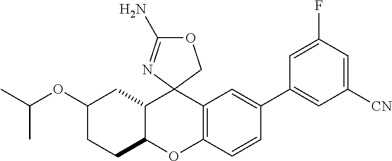 | 3-((4a′S,9a′R)-2-amino-2′- isopropoxy-1′,2′,3′,4′,4a′,9a′- hexahydro-5H-spiro[oxazole-4,9′- xanthen]-7′-yl)-5- fluorobenzonitrile (mixture of stereoisomers at ether and oxazole) | 436.1 |
| 178 | 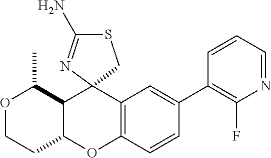 | (1R*,4aR*,4′R*,10aR*)-8-(2- fluoropyridin-3-yl)-1-methyl- 3,4,4a,10a-tetrahydro-1H,5′H- spiro[pyrano[4,3-b]chromene- 10,4′-thiazol]-2′-amine | 386 |
| 179 | 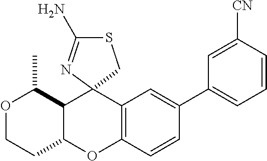 | 3-((1R*,4aR*,4′R*,10aR*)-2′- amino-1-methyl-3,4,4a,10a- tetrahydro-1H,5′H- spiro[pyrano[4,3-b]chromene- 10,4′-thiazol]-8-yl)benzonitrile 2,2,2-trifluoroacetate | 391 |
| 180 | 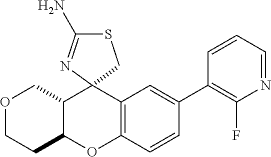 | (4aS*,4′R*,10aS*)-8-(2- fluoropyridin-3-yl)-3,4,4a,10a- tetrahydro-1H,5′H- spiro[pyrano[4,3-b]chromene- 10,4′-thiazol]-2′-amine | 372 |
| 181 | 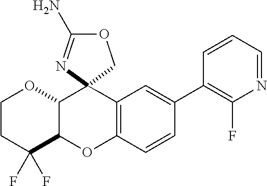 | (4R*,4a′R*,10a′R*)-4′,4′-difluoro- 8′-(2-fluoropyridin-3-yl)- 3′,4′,4a′,10a′-tetrahydro-2′H,5H- spiro[oxazole-4,10′-pyrano[3,2- b]chromen]-2-amine bis(2,2,2- trifluoroacetate | 392 |
| 182 | 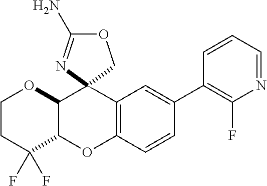 | (4R*,4a′S*,10a′S*)-4′,4′-difluoro- 8′-(2-fluoropyridin-3-yl)- 3′,4′,4a′,10a′-tetrahydro-2′H,5H- spiro[oxazole-4,10′-pyrano[3,2- b]chromen]-2-amine bis(2,2,2- trifluoroacetate | 392 |
| 183 | 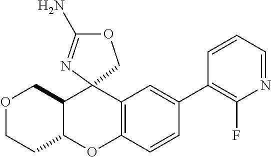 | (4R*,4a′R*,10a′R*)-8′-(2- fluoropyridin-3-yl)-3′,4′,4a′,10a′- tetrahydro-1′H,5H-spiro[oxazole- 4,10′-pyrano[4,3-b]chromen]-2- amine | 356 |
| 184 | 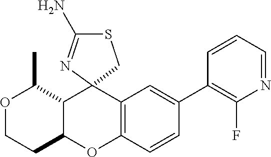 | (1S*,4aS*,4′R*,10aS*)-8-(2- fluoropyridin-3-yl)-1-methyl- 3,4,4a,10a-tetrahydro-1H,5′H- spiro[pyrano[4,3-b]chromene- 10,4′-thiazol]-2′-amine | 386 |
| 185 | 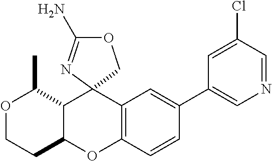 | (1′S*,4R*,4a′S*,10a′S*)-8′-(5- chloropyridin-3-yl)-1′-methyl- 3′,4′,4a′,10a′-tetrahydro-1′H,5H- spiro[oxazole-4,10′-pyrano[4,3- b]chromen]-2-amine | 386 |
| 186 | 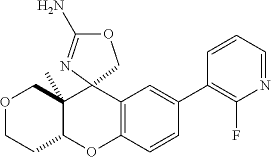 | (4R*,4a′R*,10a′R*)-8′-(2- fluoropyridin-3-yl)-10a′-methyl- 3′,4′,4a′,10a′-tetrahydro-1′H,5H- spiro[oxazole-4,10′-pyrano[4,3- b]chromen]-2-amine | 370 |
| 187 | 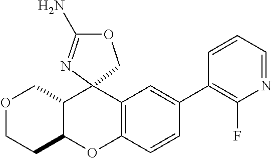 | (4R*,4a′S*,10a′S*)-8′-(2- fluoropyridin-3-yl)-3′,4′,4a′,10a′- tetrahydro-1′H,5H-spiro[oxazole- 4,10′-pyrano[4,3-b]chromen]-2- amine | 356 |
| 188 | 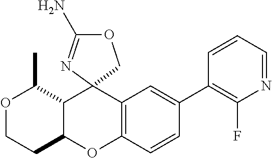 | (1′S*,4R*,4a′S*,10a′S*)-8′-(2- fluoropyridin-3-yl)-1′-methyl- 3′,4′,4a′,10a′-tetrahydro-1′H,5H- spiro[oxazole-4,10′-pyrano[4,3- b]chromen]-2-amine | 370 |
| 189 | 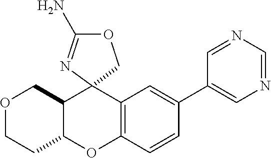 | (4R*,4a′R*,10a′R*)-8′-(pyrimidin- 5-yl)-3′,4′,4a′,10a′-tetrahydro- 1′H,5H-spiro[oxazole-4,10′- pyrano[4,3-b]chromen]-2-amine | 339 |
| 190 | 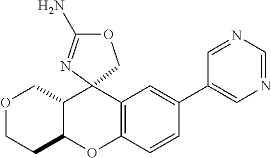 | (4R*,4a′S*,10a′S*)-8′-(pyrimidin- 5-yl)-3′,4′,4a′,10a′-tetrahydro- 1′H,5H-spiro[oxazole-4,10′- pyrano[4,3-b]chromen]-2-amine | 339 |
| 191 | 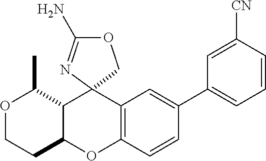 | 3-((1′S*,4R*,4a′S*,10a′S*)-2- amino-1′-methyl-3′,4′,4a′,10a′- tetrahydro-1′H,5H-spiro[oxazole- 4,10′-pyrano[4,3-b]chromen]-8′- yl)benzonitrile | 376 |
| 192 | 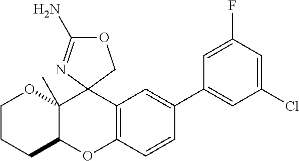 | (4a′S*,10a′R*)-8′-(3-chloro-5- fluorophenyl)-10a′-methyl- 3′,4′,4a′,10a′-tetrahydro-2′H,5H- spiro[oxazole-4,10′-pyrano[3,2- b]chromen]-2-amine | 403.1 |
| 193 | 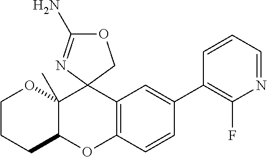 | (4a′S*,10a′R*)-8′-(2- fluoropyridin-3-yl)-10a′-methyl- 3′,4′,4a′-10a′-tetrahydro-2′H,5H- spiro[oxazole-4,10′-pyrano[3,2- b]chromen]-2-amine | 370.2 |
| 194 | 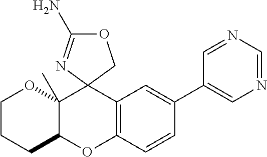 | (4a′S*,10a′R*)-10a′-methyl-8′- (pyrimidin-5-yl)-3′,4′,4a′,10a′- tetrahydro-2′H,5H-spiro[oxazole- 4,10′-pyrano[3,2-b]chromen]-2- amine | 353.2 |
| 195 | 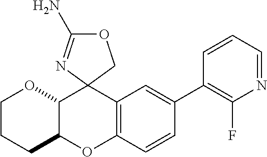 | (4a′S*,10a′R*)-8′-(2- fluoropyridin-3-yl)-3′,4′,4a′,10a′- tetrahydro-2′H,5H-spiro[oxazole- 4,10′-pyrano[3,2-b]chromen]-2- amine | 356.1 |
| 196 | 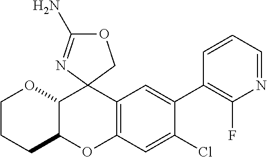 | (4a′S*,10a′R*)-7′-chloro-8′-(2- fluoropyridin-3-yl)-3′,4′,4a′,10a′- tetrahydro-2′H,5H-spiro[oxazole- 4,10′-pyrano[3,2-b]chromen]-2- amine | 390.1 |
| 197 | 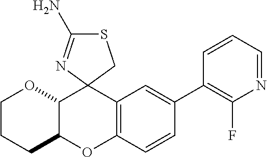 | (4aS*,10aR*)-8-(2-fluoropyridin- 3-yl)-3,4,4a,10a-tetrahydro- 2H,5′H-spiro[pyrano[3,2- b]chromene-10,4′-thiazol]-2′- amine | 372.1 |
| 198 | 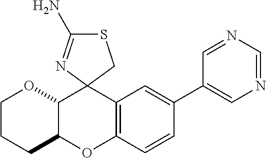 | (4aS*,10aR*)-8-(pyrimidin-5-yl)- 3,4,4a,10a-tetrahydro-2H,5′H- spiro[pyrano[3,2-b]chromene- 10,4′-thiazol]-2′-amine | 355.2 |
| 199 | 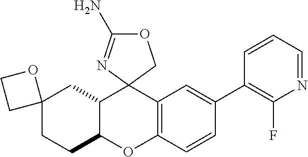 | | 396.1 |
| 200 | 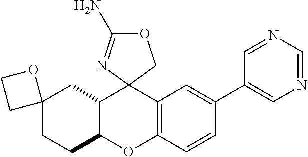 | | 379.1 |
| 201 | 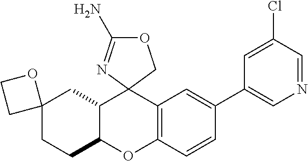 | | 412.1 |
| 202 | 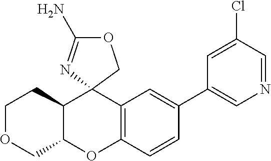 | (4S,4a′S,10a′S)-7′-(5- chloropyridin-3-yl)-3′,4′,4a′,10a′- tetrahydro-1′H,5H-spiro[oxazole- 4,5′-pyrano[3,4-b]chromen]-2- amine | 372.1 |
| 203 | 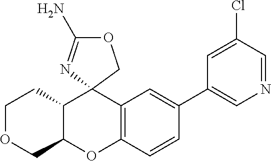 | (4S,4a′R,10a′R)-7′-(5- chloropyridin-3-yl)-3′,4′,4a′,10a′- tetrahydro-1′H,5H-spiro[oxazole- 4,5′-pyrano[3,4-b]chromen]-2- amine | 372.1 |
| 204 | 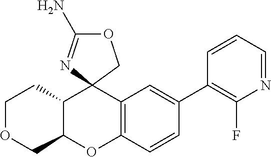 | (4R,4a′R,10a′R)-7′-(2- fluoropyridin-3-yl)-3′,4′,4a′,10a′- tetrahydro-1′H,5H-spiro[oxazole- 4,5′-pyrano[3,4-b]chromen]-2- amine | 356.0 |
| 205 | 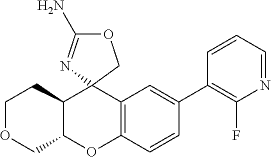 | (4S,4a′S,10a′S)-7′-(2- fluoropyridin-3-yl)-3′,4′,4a′,10a′- tetrahydro-1′H,5H-spiro[oxazole- 4,5′-pyrano[3,4-b]chromen]-2- amine | 356.0 |
| 206 | 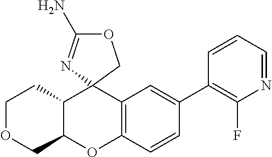 | (4S,4a′R,10a′R)-7′-(2- fluoropyridin-3-yl)-3′,4′,4a′,10a′- tetrahydro-1′H,5H-spiro[oxazole- 4,5′-pyrano[3,4-b]chromen]-2- amine | 356.0 |
| 207 | 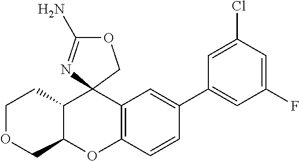 | (4R,4a′R,10a′R)-7′-(3-chloro-5- fluorophenyl)-3′,4′,4a′,10a′- tetrahydro-1′H,5H-spiro[oxazole- 4,5′-pyrano[3,4-b]chromen]-2- amine | 389.0 |
| 208 | 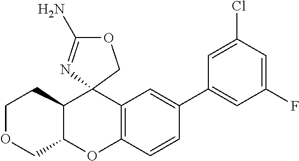 | (4S,4a′S,10a′S)-7′-(3-chloro-5- fluorophenyl)-3′,4′,4a′,10a′- tetrahydro-1′H,5H-spiro[oxazole- 4,5′-pyrano[3,4-b]chromen]-2- amine | 389.0 |
| 209 | 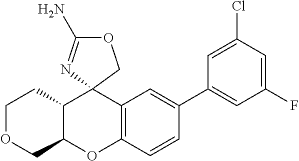 | (4S,4a′R,10a′R)-7′-(3-chloro-5- fluorophenyl)-3′,4′,4a′,10a′- tetrahydro-1′H,5H-spiro[oxazole- 4,5′-pyrano[3,4-b]chromen]-2- amine | 389.0 |
| 210 | 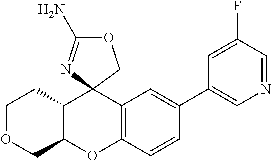 | (4R,4a′R,10a′R)-7′-(5- fluoropyridin-3-yl)-3′,4′,4a′,10a′- tetrahydro-1′H,5H-spiro[oxazole- 4,5′-pyrano[3,4-b]chromen]-2- amine | 356.1 |
| 211 | 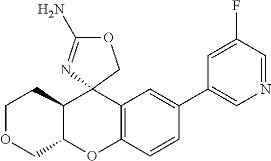 | (4S,4a′S,10a′S)-7′-(5- fluoropyridin-3-yl)-3′,4′,4a′,10a′- tetrahydro-1′H,5H-spiro[oxazole- 4,5′-pyrano[3,4-b]chromen]-2- amine | 356.1 |
| 212 | 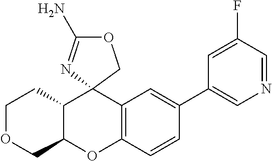 | (4S,4a′R,10a′R)-7′-(5- fluoropyridin-3-yl)-3′,4′,4a′,10a′- tetrahydro-1′H,5H-spiro[oxazole- 4,5′-pyrano[3,4-b]chromen]-2- amine | 356.0 |
| 213 | 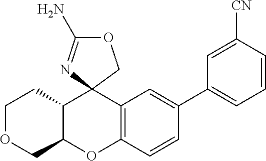 | 3-((4R,4a′R,10a′R)-2-amino- 3′,4′,4a′,10a′-tetrahydro-1′H,5H- spiro[oxazole-4,5′-pyrano[3,4- b]chromen]-7′-yl)benzonitrile | 362.0 |
| 214 | 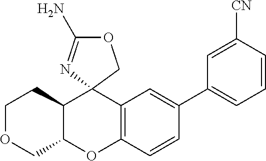 | 3-((4S,4a′S,10a′S)-2-amino- 3′,4′,4a′,10a′-tetrahydro-1′H,5H- spiro[oxazoIe-4,5′-pyrano[3,4- b]chromen]-7′-yl)benzonitrile | 362.0 |
| 215 | 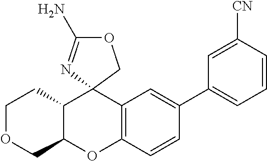 | 3-((4S,4a′R,10a′R)-2-amino- 3′,4′,4a′,10a′-tetrahydro-1′H,5H- spiro[oxazole-4,5′-pyrano[3,4- b]chromen]-7′-yl)benzonitrile | 362.0 |
| 216 | 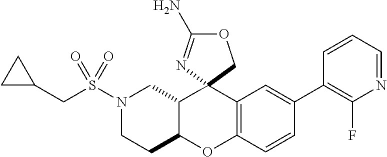 | (4aS*,4′S*,10aR*)-2- ((cyclopropylmethyl)sulfonyl)-8- (2-fluoropyridin-3-yl)- 1,2,3,4,4a,10a-hexahydro-5′H- spiro[chromeno[3,2-c]pyridine- 10,4′-oxazol]-2′-amine | 273 |
| 217 | 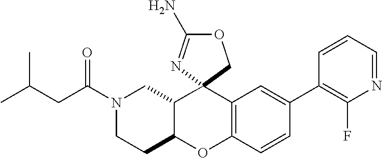 | 1-((4aS,4′S,10aR)-2′-amino-8-(2- fluoropyridin-3-yl)-1,4,4a,10a- tetrahydro-5′H- spiro[chromeno[3,2-c]pyridine- 10,4′-oxazol]-2(3H)-yl)-3- methylbutan-1-one | 439 |
| 218 | 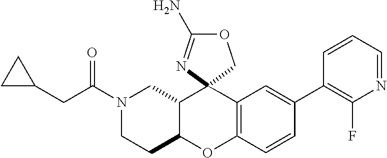 | 1-((4aS*,4′R*,10aR*)-2′-amino-8- (2-fluoropyridin-3-yl)-1,4,4a,10a- tetrahydro-5′H- spiro[chromeno[3,2-c]pyridine- 10,4′-oxazol]-2(3H)-yl)-2- cyclopropylethanone | 437 |
| 219 | 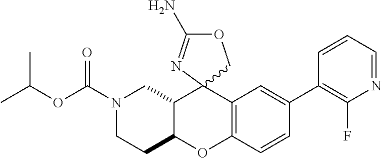 | (4aS,10aR)-isopropyl 2′-amino-8- (2-fluoropyridin-3-yl)-1,4,4a,10a- tetrahydro-5′H- spiro[chromeno[3,2-c]pyridine- 10,4′-oxazole]-2(3H)-carboxylate | 441 |
| 220 | 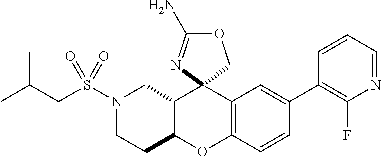 | (4aS,4′R,10aR)-8-(2- fluoropyridin-3-yl)-2- (isobutylsulfonyl)-1,2,3,4,4a,10a- hexahydro-5′H- spiro[chromeno[3,2-c]pyridine- 10,4′-oxazol]-2′-amine | 475 |
| 221 | 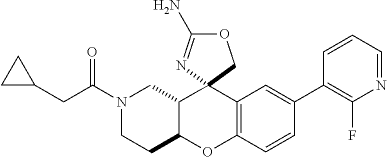 | 1-((4aS,4′S,10aR)-2′-amino-8-(2- fluoropyridin-3-yl)-1,4,4a,10a- tetrahydro-5′H- spiro[chromeno[3,2-c]pyridine- 10,4′-oxazol]-2(3H)-yl)-2- cyclopropylethanone | 437 |
| 222 | 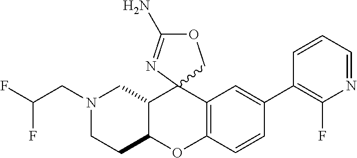 | (4aS,10aR)-2-(2,2-difluoroethyl)- 8-(2-fluoropyridin-3-yl)- 1,2,3,4,4a,10a-hexahydro-5′H- spiro[chromeno[3,2-c]pyridine- 10,4′-oxazol]-2′-amine | 419 |
| 223 | 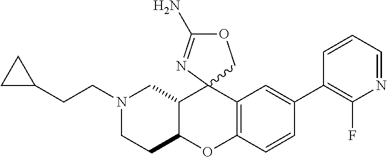 | (4aS,10aR)-2-(2- cyclopropylethyl)-8-(2- fluoropyridin-3-yl)-1,2,3,4,4a,10a- hexahydro-5′H- spiro[chromeno[3,2-c]pyridine- 10,4′-oxazol]-2′-amine | 423 |
| 224 | 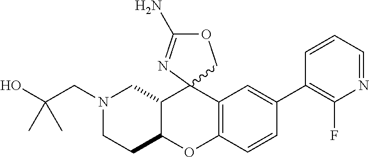 | 1-((4aS,10aR)-2′-amino-8-(2- fluoropyridin-3-yl)-1,4,4a,10a- tetrahydro-5′H- spiro[chromeno[3,2-c]pyridine- 10,4′-oxazol]-2(3H)-yl)-2- methylpropan-2-ol | 427 |
| 225 | 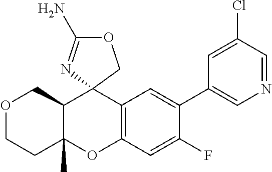 | (4S,4a′R,10a′R)-8′-(5- chloropyridin-3-yl)-7′-fluoro-4a′- methyl-3′,4′,4a′,10a′-tetrahydro- 1′H,5H-spiro[oxazole-4,10′- pyrano[4,3-b]chromen]-2-amine | 404.1 |
| 226 | 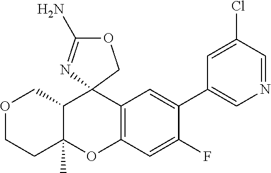 | (4S,4a′S,10a′S)-8′-(5- chloropyridin-3-yl)-7′-fluoro-4a′- methyl-3′,4′,4a′,10a′-tetrahydro- 1′H,5H-spiro[oxazole-4,10′- pyrano[4,3-b]chromen]-2-amine | 404.0 |
| 227 | 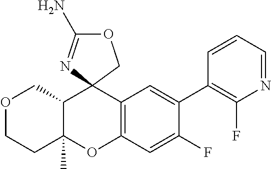 | (4R,4a′S,10a′S)-7′-fluoro-8′-(2- fluoropyridin-3-yl)-4a′-methyl- 3′,4′,4a′,10a′-tetrahydro-1′H,5H- spiro[oxazole-4,10′-pyrano[4,3- b]chromen]-2-amine | 388.1 |
| 228 | 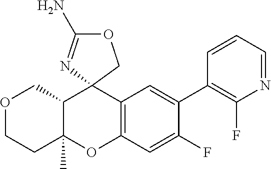 | (4S,4a′S,10a′S)-7′-fluoro-8′-(2- fluoropyridin-3-yl)-4a′-methyl- 3′,4′,4a′,10a′-tetrahydro-1′H,5H- spiro[oxazole-4,10′-pyrano[4,3- b]chromen]-2-amine | 388.1 |
| 229 | 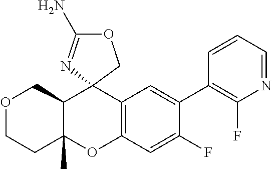 | (4S,4a′R,10a′R)-7′-fluoro-8′-(2- fluoropyridin-3-yl)-4a′-methyl- 3′,4′,4a′,10a′-tetrahydro-1′H,5H- spiro[oxazole-4,10′-pyrano[4,3- b]chromen]-2-amine | 388.1 |
| 230 | 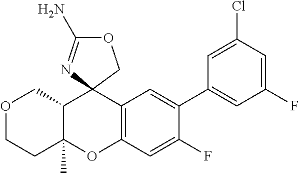 | (4R,4a′S,10a′S)-8′-(3-chloro-5- fluorophenyl)-7′-fluoro-4a′- methyl-3′,4′,4a′,10a′-tetrahydro- 1′H,5H-spiro[oxazole-4,10′- pyrano[4,3-b]chromen]-2-amine | 421.1 |
| 231 | 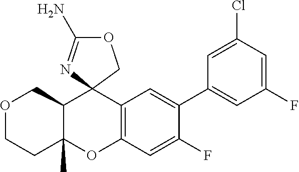 | (4R,4a′R,10a′R)-8′-(3-chloro-5- fluorophenyl)-7′-fluoro-4a′- methyl-3′,4′,4a′,10a′-tetrahydro- 1′H,5H-spiro[oxazole-4,10′- pyrano[4,3-b]chromen]-2-amine | 421.1 |
| 232 | 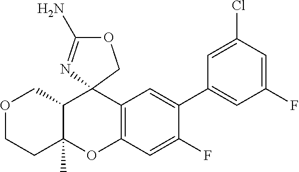 | (4S,4a′S,10a′S)-8′-(3-chloro-5- fluorophenyl)-7′-fluoro-4a′- methyl-3′,4′,4a′,10a′-tetrahydro- 1′H,5H-spiro[oxazole-4,10′- pyrano[4,3-b]chromen]-2-amine | 420.0 |
| 233 | 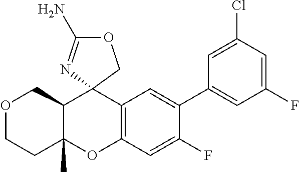 | (4S,4a′R,10a′R)-8′-(3-chloro-5- fluorophenyl)-7′-fluoro-4a′- methyl-3′,4′,4a′,10a′-tetrahydro- 1′H,5H-spiro[oxazole-4,10′- pyrano[4,3-b]chromen]-2-amine | 421.0 |
| 234 | 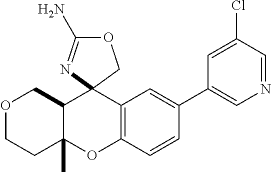 | (4R*,4a′R*,10a′R*)-8′-(5- chloropyridin-3-yl)-4a′-methyl- 3,′4′,4a′,10a′-tetrahydro-1′H,5H- spiro[oxazole-4,10′-pyrano[4,3- b]chromen]-2-amine | 386.1 |
| 235 | 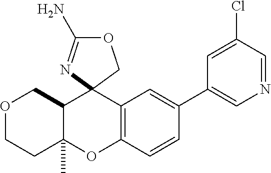 | (4R*,4a′S*,10a′R*)-8′-(5- chloropyridin-3-yl)-4a′-methyl- 3′,4′,4a′,10a′-tetrahydro-1′H,5H- spiro[oxazole-4,10′-pyrano[4,3- b]chromen]-2-amine | 386.1 |
| 236 | 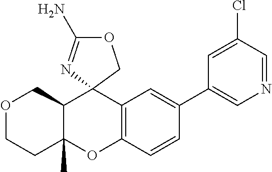 | (4S,4a′R,10a′R)-8′-(5- chloropyridin-3-yl)-4a′-methyl- 3′,4′,4a′,10a′-tetrahydro-1′H,5H- spiro[oxazole-4,10′-pyrano[4,3- b]chromen]-2-amine | 386.1 |
| 237 | 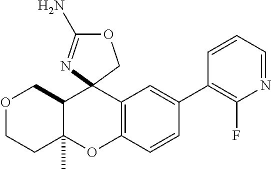 | (4R*,4a′S*,10a′R*)-8′-(2- fluoropyridin-3-yl)-4a′-methyl- 3′,4′,4a′,10a′-tetrahydro-1′H,5H- spiro[oxazole-4,10′-pyrano[4,3- b]chromen]-2-amine | 370.1 |
| 238 | 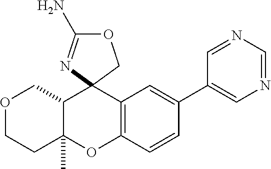 | (4R*,4a′S*,10a′S*)-4a′-methyl-8′- (pyrimidin-5-yl)-3′,4′,4a′,10a′- tetrahydro-1′H,5H-spiro[oxazole- 4,10′-pyrano[4,3-b]chromen]-2- amine | 353.1 |
| 239 | 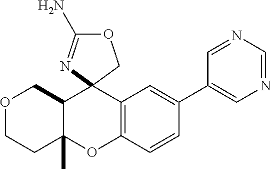 | (4R*,4a′R*,10a′R*)-4a′-methyl-8′- (pyrimidin-5-yl)-3′,4′,4a′,10a′- tetrahydro-1′H,5H-spiro[oxazole- 4,10′-pyrano[4,3-b]chromen]-2- amine | 353.1 |
| 240 | 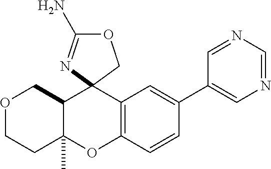 | (4R*,4a′S*,10a′R*)-methyl-8′- (pyrimidin-5-yl)-3′,4′,4a′,10a′- tetrahydro-1′H,5H-spiro[oxazole- 4,10′-pyrano[4,3-b]chromen]-2- amine | 353.1 |
| 241 | 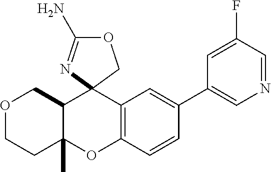 | (4R*,4a′R*,10a′R*)-8′-(5- fluoropyridin-3-yl)-4a′-methyl- 3′,4′,4a′,10a′-tetrahydro-1′H,5H- spiro[oxazole-4,10′-pyrano[4,3- b]chromen]-2-amine | 370.1 |
| 242 | 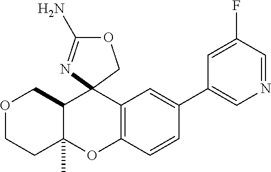 | (4R,4a′S,10a′R)-8′-(5- fluoropyridin-3-yl)-4a′-methyl- 3′,4′,4a′,10a′-tetrahydro-1′H,5H- spiro[oxazole-4,10′-pyrano[4,3- b]chromen]-2-amine | 370.1 |
| 243 | 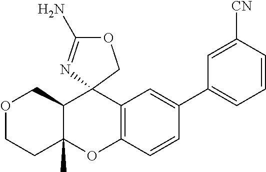 | 3-((4S,4a′R,10a′R)-2-amino-4a′- methyl-3′,4′,4a′,10a′-tetrahydro- 1′H,5H-spiro[oxazole-4,10′- pyrano[4,3-b]chromen]-8′- yl)benzonitrile | 376.2 |
| 244 | 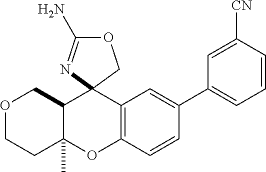 | 3-((4R*,4a′S*,10a′R*)-2-amino- 4a′-methyl-3′,4′,4a′,10a′- tetrahydro-1′H,5H-spiro[oxazole- 4,10′-pyrano[4,3-b]chromen]-8′- yl)benzonitrile | 376.2 |
| 255 | 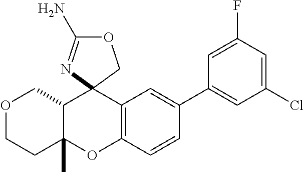 | (4R*,4a′R*,10a′S*)-8′-(3-chloro- 5-fluorophenyl)-4a′-methyl- 3′,4′,4a′,10a′-tetrahydro-1′H,5H- spiro[oxazole-4,10′-pyrano[4,3- b]chromen]-2-amine | 403.1 |
| 256 | 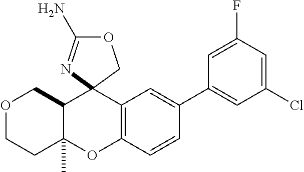 | (4R*,4a′S*,10a′R*)-8′-(3-chloro- 5-fluorophenyl)-4a′-methyl- 3′,4′,4a′,10a′-tetrahydro-1′H,5H- spiro[oxazole-4,10′-pyrano[4,3- b]chromen]-2-amine | 403.1 |
| 257 | 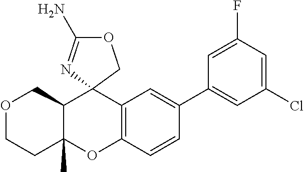 | (4S,4a′R,10a′R)-8′-(3-chloro-5- fluorophenyl)-4a′-methyl- 3′,4′,4a′,10a′-tetrahydro-1′H,5H- spiro[oxazole-4,10′-pyrano[4,3- b]chromen]-2-amine | 403.1 |
| 258 | 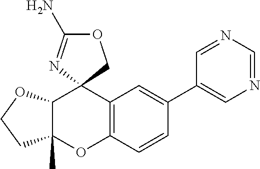 | (3aR,4′S,9aR)-3a-methyl-7- (pyrimidin-5-yl)-2,3,3a,9a- tetrahydro-5′H-spiro[furo[3,2- b]chromene-9,4′-oxazol]-2′-amine | 338.9 |
| 259 | 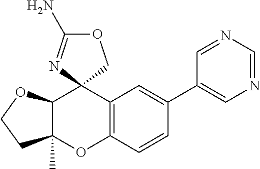 | (3aS,4′R,9aS)-3a-methyl-7- (pyrimidin-5-yl)-2,3,3a,9a- tetrahydro-5′H-spiro[furo[3,2- b]chromene-9,4′-oxazol]-2′-amine | 339.1 |
| 260 | 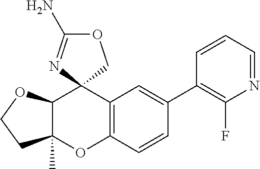 | (3aS,4′R,9aS)-7-(2-fluoropyridin- 3-yl)-3a-methyl-2,3,3a,9a- tetrahydro-5′H-spiro[furo[3,2- b]chromene-9,4′-oxazol]-2′-amine | 356.0 |
| 261 | 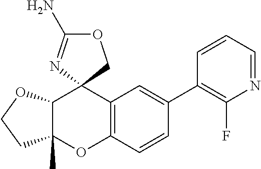 | (3aR,4′S,9aR)-7-(2-fluoropyridin- 3-yl)-3a-methyl-2,3,3a,9a- tetrahydro-5′H-spiro[furo[3,2- b]chromene-9,4′-oxazol]-2′-amine | 356.0 |
| 262 | 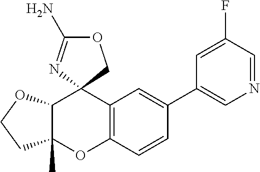 | (3aR,4′S,9aR)-7-(5-fluoropyridin- 3-yl)-3a-methyl-2,3,3a,9a- tetrahydro-5′H-spiro[furo[3,2- b]chromene-9,4′-oxazol]-2′-amine | 356.0 |
| 263 | 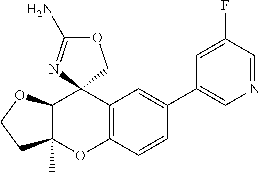 | (3aS,4′R,9aS)-7-(5-fluoropyridin- 3-yl)-3a-methyl-2,3,3a,9a- tetrahydro-5′H-spiro[furo[3,2- b]chromene-9,4′-oxazol]-2′-amine | 356.0 |
| 264 | 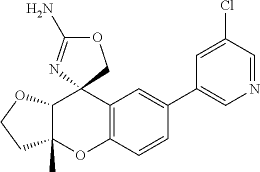 | (3aR,4′S,9aR)-7-(5-chloropyridin- 3-yl)-3a-methyl-2,3,3a,9a- tetrahydro-5′H-spiro[furo[3,2- b]chromene-9,4′-oxazol]-2′-amine | 372.0 |
| 265 | 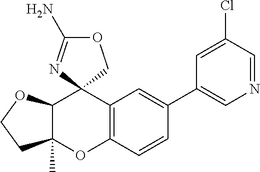 | (3aS,4′R,9aS)-7-(5-chloropyridin- 3-yl)-3a-methyl-2,3,3a,9a- tetrahydro-5′H-spiro[furo[3,2- b]chromene-9,4′-oxazol]-2′-amine | 372.0 |
| 266 | 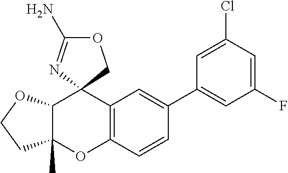 | (3aR,4′S,9aR)-7-(3-chloro-5- fluorophenyl)-3a-methyl- 2,3,3a,9a-tetrahydro-5′H- spiro[furo[3,2-b]chromene-9,4′- oxazol]-2′-amine | 389.0 |
| 267 | 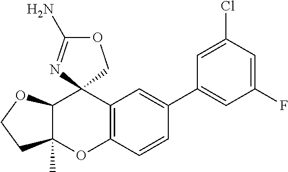 | (3aS,4′R,9aS)-7-(3-chloro-5- fluorophenyl)-3a-methyl- 2,3,3a,9a-tetrahydro-5′H- spiro[furo[3,2-b]chromene-9,4′- oxazol]-2′-amine | 289.0 |
| 268 | 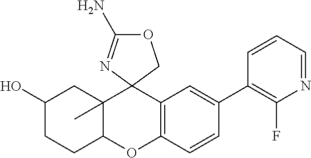 | 2-amino-7′-(2-fluoropyridin-3-yl)- 9a′-methyl-1′,2′,3′,4′,4a′,9a′- hexahydro-5H-spiro[oxazole-4,9′- xanthen]-2′-ol cis ring junction stereoisomer 1 | 384.1 |
| 269 | 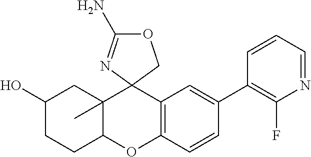 | 2-amino-7′-(2-fluoropyridin-3-yl)- 9a′-methyl-1′,2′,3′,4′,4a′,9a′- hexahydro-5H-spiro[oxazole-4,9′- xanthen]-2′-ol cis ring junction stereoisomer 2 | 384.1 |
| 270 | 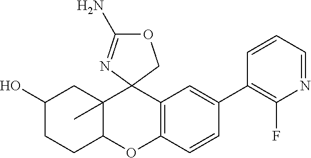 | 2-amino-7′-(2-fluoropyridin-3-yl)- 9a′-methyl-1′,2′,3′,4′,4a′,9a′- hexahydro-5H-spiro[oxazole-4,9′- xanthen]-2′-ol cis ring junction stereoisomer 3 | 384.1 |
| 271 | 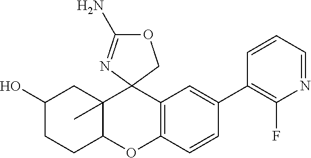 | 2-amino-7′-(2-fluoropyridin-3-yl)- 9a′-methyl-1′,2′,3′,4′,4a′,9a′- hexahydro-5H-spiro[oxazole-4,9′- xanthen]-2′-ol cis ring junction stereoisomer | 384.1 |
| 272 | 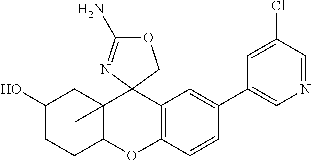 | 2-amino-7′-(5-chloropyridin-3-yl)- 9a′-methyl-1′,2′,3′,4′,4a′,9a′- hexahydro-5H-spiro[oxazole-4,9′- xanthen]-2′-ol cis ring junction stereoisomer 1 | 400.0 |
| 273 | 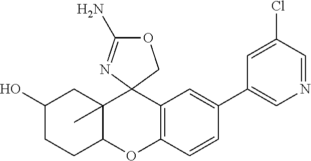 | 2-amino-7′-(5-chloropyridin-3-yl)- 9a′-methyl-1′,2′,3′,4′,4a′,9a′- hexahydro-5H-spiro[oxazole-4,9′- xanthen]-2′-ol cis ring junction stereoisomer 2 | 400.0 |
| 274 | 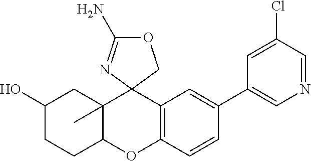 | 2-amino-7′-(5-chloropyridin-3-yl)- 9a′-methyl-1′,2′,3′,4′,4a′,9a′- hexahydro-5H-spiro[oxazole-4,9′- xanthen]-2′-ol cis ring junction stereoisomer 3 | 400.0 |
| 275 | 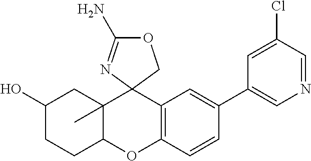 | 2-amino-7′-(5-chloropyridin-3-yl)- 9a′-methyl-1′,2′,3′,4′,4a′,9a′- hexahydro-5H-spiro[oxazole-4,9′- xanthen]-2′-ol cis ring junction stereoisomer 4 | 400.0 |
| 276 | 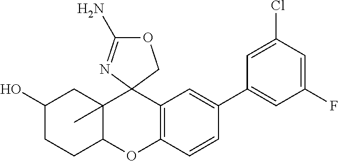 | 2-amino-7′-(3-chloro-5- fluorophenyl)-9a′-methyl- 1′,2′,3′,4′,4a′,9a′-hexahydro-5H- spiro[oxazole-4,9′-xanthen]-2′-ol cis ring junction stereoisomer 1 | 417.1 |
| 277 | 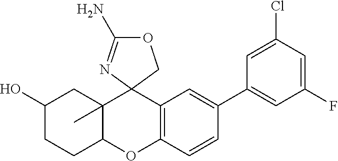 | 2-amino-7′-(3-chloro-5- fluorophenyl)-9a′-methyl- 1′,2′,3′,4′,4a′,9a′-hexahydro-5H- spiro[oxazole-4,9′-xanthen]-2′-ol cis ring junction stereoisomer 2 | 417.1 |
| 278 | 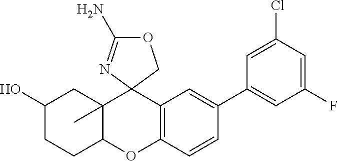 | 2-amino-7′-(3-chloro-5- fluorophenyl)-9a′-methyl- 1′,2′,3′,4′,4a′,9a′-hexahydro-5H- spiro[oxazole-4,9′-xanthen]-2′-ol cis ring junction stereoisomer 3 | 417.1 |
| 279 | 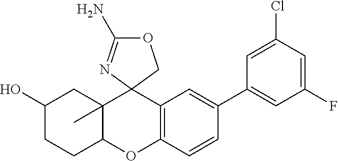 | 2-amino-7′-(3-chloro-5- fluorophenyl)-9a′-methyl- 1′,2′,3′,4′,4a′,9a′-hexahydro-5H- spiro[oxazole-4,9′-xanthen]-2′-ol cis ring junction stereoisomer 4 | 417.0 |
| 280 | 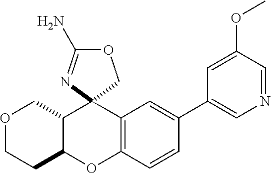 | (4R*,4a′S*,10a′S*)-8′-(5- methoxypyridin-3-yl)- 3′,4′,4a′,10a′-tetrahydro-1′H,5H- spiro[oxazoIe-4,10′-pyrano[4,3- b]chromen]-2-amine | 368.0 |
| 281 | 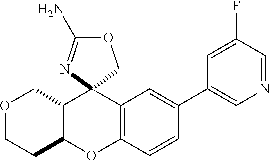 | (4R*,4a′S*,10a′S*)-8′-(5- fluoropyridin-3-yl)-3′,4′,4a′,10a′- tetrahydro-1′H,5H-spiro[oxazole- 4,10′-pyrano[4,3-b]chromen]-2- amine | 354.9 |
| 282 | 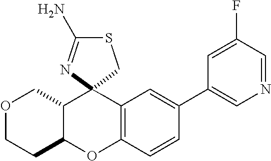 | (4aS*,4′R*,10aS*)-8-(5- fluoropyridin-3-yl)-3,4,4a,10a- tetrahydro-1H,5′H- spiro[pyrano[4,3-b]chromene- 10,4′-thiazol]-2′-amine | 371.9 |
| 283 | 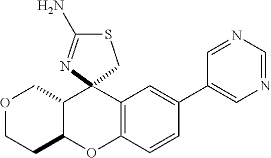 | (4aS*,4′R*,10aS*)-8-(pyrimidin- 5-yl)-3,4,4a,10a-tetrahydro- 1H,5′H-spiro[pyrano[4,3- b]chromene-10,4′-thiazol]-2′- amine | 355.0 |
| 284 | 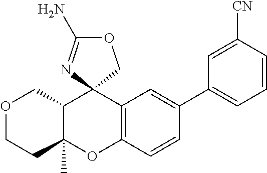 | 3-((4R,4a′S,10a′S)-2-amino-4a′- methyl-3′,4′,4a′,10a′-tetrahydro- 1′H,5H-spiro[oxazole-4,10′- pyrano[4,3-b]chromen]-8′- yl)benzonitrile | 376.0 |
| 285 | 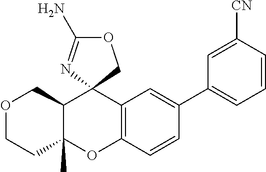 | 3-((4S,4a′R,10a′R)-2-amino-4a′- methyl-3′,4′,4a′,10a′-tetrahydro- 1′H,5H-spiro[oxazole-4,10′- pyrano[4,3-b]chromen]-8′- yl)benzonitrile | 376.1 |
| 286 | 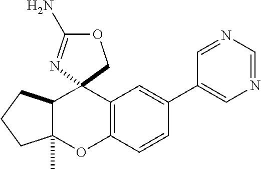 | racemic (3aS,4′S,9aS)-3a-methyl- 7-(pyrimidin-5-yl)-2,3,3a,9a- tetrahydro-1H,5′H- spiro[cyclopenta[b]chromene-9,4′- oxazol]-2′-amine | |
| 287 | 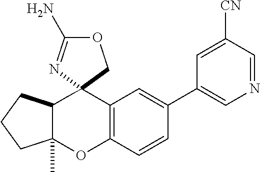 | racemic 5-((3aS,4′S,9aS)-2′- amino-3a-methyl-2,3,3a,9a- tetrahydro-1H,5′H- spiro[cyclopenta[b]chromene-9,4′- oxazol]-7-yl)nicotinonitrile | |
| 288 | 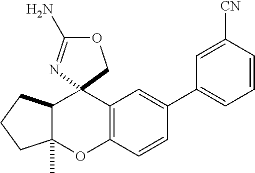 | racemic 3-((3aS,4′S,9aS)-2′- amino-3a-methyl-2,3,3a,9a- tetrahydro-1H,5′H- spiro[cyclopenta[b]chromene-9,4′- oxazol]-7-yl)benzonitrile | |
| 289 | 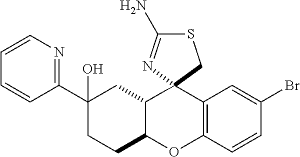 | (4S*,4a′S*,9a′R*)-2-amino-7′- bromo-2′-(pyridin-2-yl)- 1′,2′,3′,4′,4a′,9a′-hexahydro-5H- spiro[thiazole-4,9′-xanthen]-2′-ol | 446.0, 448.0 |
| 290 | 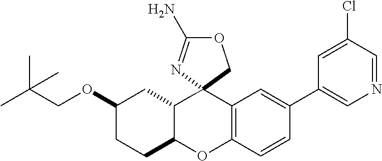 | (2′R,4S,4a′S,9a′R)-7′-(5- chloropyridin-3-yl)-2′- (neopentyloxy)-1′,2′,3′,4′,4a′,9a′- hexahydro-5H-spiro[oxazole-4,9′- xanthen]-2-amine trifluoroacetic acid salt (axial ether, racemic) | 456.1 |
| 291 | 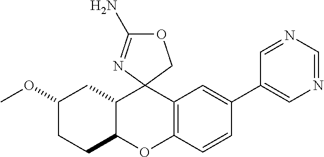 | (2′S,4a′S,9a′R)-2′-methoxy-7′- (pyrimidin-5-yl)-1′,2′,3′,4′,4a′,9a′- hexahydro-5H-spiro[oxazole-4,9′- xanthen]-2-amine trifluoroacetic acid salt (racemic, equatorial ether) | 367.1 |
| 292 | 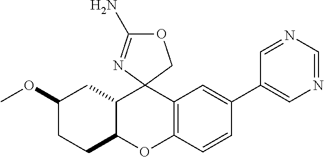 | (2′R,4a′S,9a′R)-2′-methoxy-7′- (pyrimidin-5-yl)-1′,2′,3′,4′,4a′,9a′- hexahydro-5H-spiro[oxazole-4,9′- xanthen]-2-amine trifluoroacetic acid salt (axial ether, mixture at warhead, racemic) | 367.1 |
| 293 | 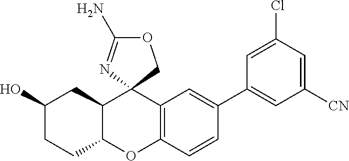 | 3-((2′R,4S,4a′R,9a′S)-2-amino-2′- hydroxy-1′,2′,3′,4′,4a′,9a′- hexahydro-5H-spiro[oxazole-4,9′- xanthen]-7′-yl)-5- chlorobenzonitrile | 410.1 |
| 294 | 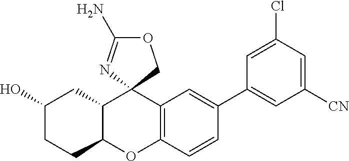 | 3-((2′S,4S,4a′S,9a′R)-2-amino-2′- hydroxy-1′,2′,3′,4′,4a′,9a′- hexahydro-5H-spiro[oxazole-4,9′- xanthen]-7′-yl)-5- chlorobenzonitrile | 410.1 |
| 295 | 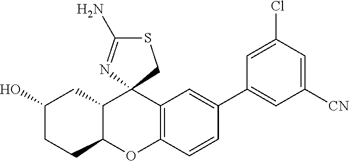 | 3-((2′S,4S,4a′S,9a′R)-2-amino-2′- hydroxy-1′,2′,3′,4′,4a′,9a′- hexahydro-5H-spiro[thiazole-4,9′- xanthen]-7′-yl)-5- chlorobenzonitrile (mixture of single diastereomer, racemic product and starting bromide) | 426.1 |
| 296 | 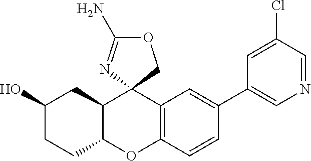 | (2′R,4S,4a′R,9a′S)-2-amino-7′-(5- chloropyridin-3-yl)- 1′,2′,3′,4′,4a′,9a′-hexahydro-5H- spiro[oxazole-4,9′-xanthen]-2′-ol | 386.1 |
| 297 | 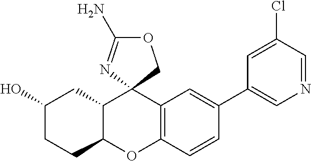 | (2′S,4S,4a′S,9a′R)-2-amino-7′-(5- chloropyridin-3-yl)- 1′,2′,3′,4′,4a′,9a′-hexahydro-5H- spiro[oxazole-4,9′-xanthen]-2′-ol | 386.1 |
| 298 | 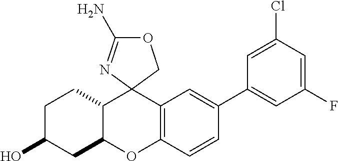 | (3′S,4a′S,9a′R)-2-amino-7′-(3- chloro-5-fluorophenyl)- 1′,2′,3′,4′,4a′,9a′-hexahydro-5H- spiro[oxazole-4,9′-xanthen]-3′-ol trifluoroacetic acid salt | 403.1 |
| 299 | 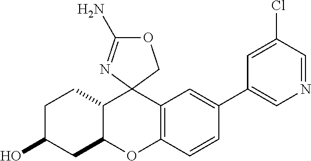 | (3′S,4a′S,9a′R)-2-amino-7′-(3- chloropyridin-3-yl)- 1′,2′,3′,4′,4a′,9a′-hexahydro-5H- spiro[oxazole-4,9′-xanthen]-3′-ol trifluoroacetic acid salt (racemic, mixture of diastereomers at oxazoline) | 386.1 |
| 300 | 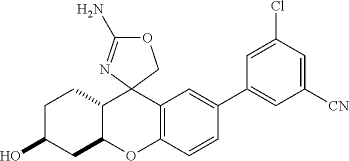 | 3-((3′S,4a′S,9a′R)-2-amino-3′- hydroxy-1′,2′,3′,4′,4a′,9a′- hexahydro-5H-spiro[thiazole-4,9′- xanthen]-7′-yl)-5- chlorobenzonitrile trifluoroacetic acid salt (racemic, mixture of diastereomers at oxazoline) | 410.1 |
| 301 | 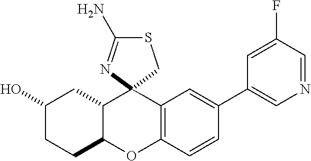 | (2′S,4R,4a′S,9a′R)-2-amino-7′-(5- fluoropyridin-3-yl)- 1′,2′,3′,4′,4a′,9a′-hexahydro-5H- spiro[thiazole-4,9′-xanthen]-2′-ol trifluoroacetic acid salt (racemic, single diastereomer) | 386.1 |
| 302 | 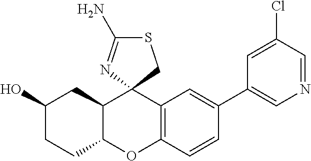 | (2′R,4S,4a′R,9a′S)-2-amino-7′-(5- chloropyridin-3-yl)- 1′,2′,3′,4′,4a′,9a′-hexahydro-5H- spiro[thiazole-4,9′-xanthen]-2′-ol (single enantiomer) | 402.0 |
| 303 | 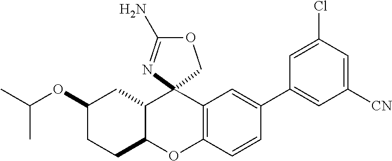 | 3-((2′R,4R,4a′S,9a′R)-2-amino-2′- isopropoxy-1′,2′,3′,4′,4a′,9a′- hexahydro-5H-spiro[oxazole-4,9′- xanthen]-7′-yl)-5- chlorobenzonitrile trifluoroacetic acid salt (single diastereomer, axial ether, racemic) | 452.1 |
| 304 | 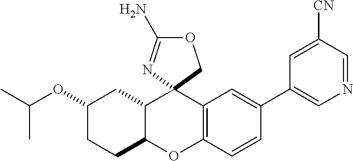 | 5-((2′S,4S,4a′S,9a′R)-2-amino-2′- isopropoxy-1′,2′,3′,4′,4a′,9a′- hexahydro-5H-spiro[oxazole-4,9′- xanthen]-7′-yl)nicotinonitrile trifluoroacetic acid salt (single diastereomer, racemic) | 419.2 |
| 305 | 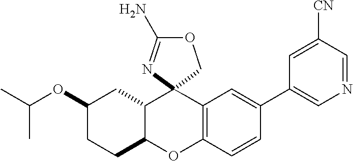 | 5-((2′R,4R,4a′S,9a′R)-2-amino-2′- isopropoxy-1′,2′,3′,4′,4a′,9a′- hexahydro-5H-spiro[oxazole-4,9′- xanthen]-7′-yl)nicotinonitrile trifluoroacetic acid salt (single ether, single diastereomer, racemic) | 428.1 |
| 306 | 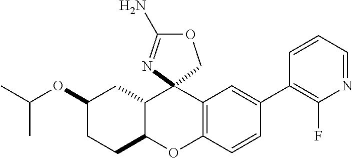 | (2′R,4R,4a′S,9a′R)-7′-(2- fluoropyridin-3-yl)-2′-isopropoxy- 1′,2′,3′,4′,4a′,9a′-hexahydro-5H- spiro[oxazole-4,9′-xanthen]-2- amine trifluoroacetic acid salt (axial ether, single diastereomer, racemic) | 412.1 |
| 307 | 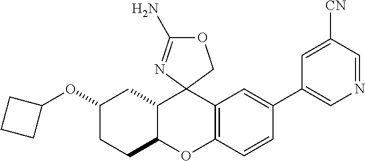 | 5-((2′S,4a′S,9a′R)-2-amino-2′- cyclobutoxy-1′,2′,3′,4′,4a′,9a′- hexahydro-5H-spiro[oxazole-4,9′- xanthen]-7′-yl)nicotinonitrile trifluoroacetic acid salt (mixture of diastereomers at oxazoline, racemic) | 430.9 |
| 308 | 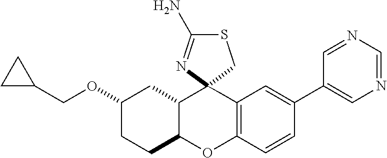 | (2′S,4R,4a′S,9a′R)-2′- (cyclopropylmethoxy)-7′- (pyrimidin-5-yl)-1′,2′,3′,4′,4a′,9a′- hexahydro-5H-spiro[thiazole-4,9′- xanthen]-2-amine (racemic, single diastereomer) | 423.1 |
| 309 | 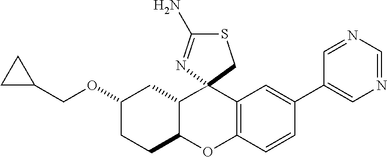 | (2′S,4S,4a′S,9a′R)-2′- (cyclopropylmethoxy)-7′- (pyrimidin-5-yl)-1′,2′,3′,4′,4a′,9a′- hexahydro-5H-spiro[thiazole-4,9′- xanthen]-2-amine | 423.1 |
| 310 | 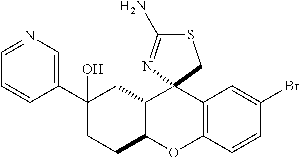 | (4S*,4a′S*,9a′R*)-2-amino-7′- bromo-2′-(pyridin-3-yl)- 1′,2′,3′,4′,4a′,9a′-hexahydro-5H- spiro[thiazole-4,9′-xanthen]-2′-ol | 446.0, 448.0 |

It will be understood that the enumerated embodiments are not intended to limit the invention to those embodiments. On the contrary, the invention is intended to cover all alternatives, modifications and equivalents, which may be included within the scope of the present invention as defined by the claims. Thus, the foregoing description is considered as illustrative only of the principles of the invention.

1. A compound selected from Formula I′: 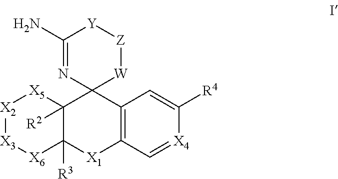
and stereoisomers, diastereomers, enantiomers, tautomers and pharmaceutically acceptable salts thereof, wherein:
W is CR¹²R¹³;
Y is O, S or NR¹;
Z is a bond, CH₂ or C(═O), provided when Z is C(═O) then Y is NR¹;
X₁ is selected from O, S, S(O), SO₂, NR¹⁰ and CHR¹⁰;
X₂ is selected from CR⁵R⁶, NR⁷ and O;
X₃ is selected from a bond, CR⁸R⁹ and O, provided when X₃ is O then X₂ is CR⁵R⁶;
X₄ is selected from CR¹¹ and N;
X₅ is selected from CR¹⁴R¹⁵ and O, provided when X₅ is O, then X₂ is CR⁵R⁶ and X₃ is CR⁸R⁹ or a bond;
X₆ is CR¹⁶R¹⁷;
R¹ is selected from hydrogen, alkyl, aralkyl, heteroaryl and heteroaralkyl;
R² and R³ are hydrogen or alkyl;
R⁴ is selected from hydrogen, hydroxy, halogen, amino, cyano, nitro, alkyl, alkoxy, acyl, acyloxy, acylamino, alkoxycarbonyl, sulfonyl, sulfinyl, sulfanyl, aryloxy, a carbocycle and a heterocycle wherein said amino, alkyl, alkoxy, acyl, acyloxy, alkoxycarbonyl, sulfonyl, sulfinyl, sulfanyl, aryloxy, carbocycle and heterocycle are optionally substituted with SF₅, hydroxy, halogen, amino, cyano, nitro, oxo, optionally substituted alkyl, optionally substituted alkoxy, sulfanyl, acyl, alkoxycarbonyl, haloalkyl, optionally substituted carbocycle or optionally substituted heterocycle;
R⁵ and R⁶ are independently selected from hydrogen, hydroxy, halogen, amino, cyano, nitro, alkyl, alkoxy, acyl, acyloxy, alkoxycarbonyl, sulfonyl, sulfinyl, sulfanyl, aryloxy, a carbocycle and a heterocycle, wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl or optionally substituted carbocycle, or
R⁵ and R⁶ taken together form an oxo group, or
R⁵ and R⁶ together with the atom to which they are attached form a carbocycle or heterocycle;
R⁷ is selected from hydrogen, alkyl, alkoxy, acyl, acyloxy, alkoxycarbonyl, sulfonyl, sulfinyl, sulfanyl, aryloxy, a carbocycle and a heterocycle, wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl or optionally substituted carbocycle;
R⁸ and R⁹ are independently selected from hydrogen, hydroxy, halogen, amino, cyano, nitro, alkyl, alkoxy, acyl, acyloxy, alkoxycarbonyl, sulfonyl, sulfinyl, sulfanyl, aryloxy, a carbocycle and a heterocycle, wherein said alkyl, alkoxy, acyl, acyloxy, alkoxycarbonyl, sulfonyl, sulfinyl, sulfanyl, aryloxy, carbocycle and heterocycle are optionally substituted with hydroxy, halogen, amino, cyano, nitro, oxo, optionally substituted alkyl, optionally substituted alkoxy, sulfanyl, acyl, alkoxycarbonyl, haloalkyl or optionally substituted carbocycle, or
R⁸ and R⁹ taken together form an oxo, or
R⁸ and R⁹ together with the atom to which they are attached form a carbocycle or heterocycle;
R¹⁰ is selected from hydrogen, halogen and alkyl;
R¹¹ is selected from hydrogen, halogen and alkyl;
R¹² and R¹³ are independently selected from hydrogen and alkyl, or
R¹² and R¹³ together with the atom to which they are attached form a 3 to 6 membered carbocycle or heterocycle;
R¹⁴ and R¹⁵ are independently selected from hydrogen and C₁-C₃ alkyl; and
R¹⁶ and R¹⁷ are independently selected from hydrogen and halogen.

2. A compound of claim 1, wherein:
W is CR¹²R¹³;
Y is O, S or NR¹;
Z is a bond, CH₂ or C(═O), provided when Z is C(═O) then Y is NR¹;
X₁ is selected from O, S, S(O), SO₂, NR¹⁰ and CHR¹⁰;
X₂ is selected from CR⁵R⁶, NR⁷ and O;
X₃ is selected from a bond, CR⁸R⁹ and O, provided when X₃ is O then X₂ is CR⁵R⁶;
X₄ is selected from CR¹¹ and N;
X₅ is selected from CR¹⁴R¹⁵ and O, provided when X₅ is O, then X₂ is CR⁵R⁶ and X₃ is CR⁸R⁹ or a bond;
X₆ is CR¹⁶R¹⁷;
R¹ is C₁-C₃ alkyl;
R² and R³ are independently selected from hydrogen and C₁-C₆ alkyl;
R⁴ is selected from hydrogen, halogen, C₁-C₆ alkyl, C₁-C₆ alkenyl, C₁-C₆ alkynyl, C₁-C₆ alkoxy, NHC(═O)R^{f}, C(═O)NHR^{f}, a 3 to 6 membered carbocycle, a 3 to 6 membered heterocycle, phenyl and a 5 to 6 membered heteroaryl, wherein the alkyl, alkenyl, alkynyl, alkoxy, carbocycle, heterocycle, phenyl and heteroaryl are optionally substituted with one or more R^{g} groups;
R⁵ and R⁶ are independently selected from hydrogen, halogen, OR^{a}, NR^{b}R^{c}, CN, C₁-C₆ alkyl, C₁-C₆ alkoxy, phenyl, a 3 to 6 membered heterocycle and a 5 to 6 membered heteroaryl, wherein the alkyl, phenyl, heterocycle and heteroaryl are optionally substituted with halogen or a 3 to 6 membered carbocycle, or
R⁵ and R⁶ taken together form an oxo group, or
R⁵ and R⁶ together with the atom to which they are attached form a 3 to 6 membered carbocycle or heterocycle;
R⁷ is selected from hydrogen, C₁-C₆ alkyl, C₁-C₆ alkoxycarbonyl, —C(═O)NR^{h}Rⁱ, —SO₂(C₁-C₆ alkyl), a 3 to 6 membered carbocycle, a 3 to 6 membered heterocycle, phenyl, and a 5 to 6 membered heteroaryl, wherein the alkyls, alkoxycarbonyl, carbocycle, heterocycle, phenyl and heteroaryl are optionally substituted with one or more R^{d} groups;
R⁸ and R⁹ are independently selected from hydrogen, halogen, CN, C₁-C₆ alkyl, C₁-C₆ alkenyl, C₁-C₆ alkynyl, phenyl, a 5 to 6 membered heteroaryl and OR^{e}, wherein the alkyl, alkenyl, alkynyl, alkoxy, phenyl and heteroaryl are optionally substituted with halogen, or
R⁸ and R⁹ taken together form an oxo group, or
R⁸ and R⁹ together with the atom to which they are attached form a 3 to 6 membered carbocycle or heterocycle;
R¹⁰ is selected from hydrogen, halogen and C₁-C₆ alkyl;
R¹¹ is selected from hydrogen, halogen and C₁-C₆ alkyl;
R¹² and R¹³ are independently selected from hydrogen and C₁-C₃ alkyl, or
R¹² and R¹³ together with the atom to which they are attached form a 3 to 6 membered carbocycle or heterocycle;
R¹⁴ and R¹⁵ are independently selected from hydrogen and C₁-C₃ alkyl;
R¹⁶ and R¹⁷ are independently selected from hydrogen and halogen;
R^{a} is selected from hydrogen, C₁-C₆ alkyl and (CH₂)₀₋₃ (3 to 6 membered carbocyclic);
R^{b} and R^{c} are independently selected from hydrogen and C₁-C₆ alkyl, or
R^{b} and R^{c} together with the nitrogen atom to which they are attached form a 3 to 6 membered heterocyclic;
each R^{d} is selected from halogen, hydroxy, oxo, C₃-C₆ cycloalkyl and phenyl, wherein the phenyl is optionally substituted with halogen, C₁-C₃ alkyl or C₁-C₃ alkoxy;
R^{e} is selected from hydrogen and C₁-C₆ alkyl;
R^{f} is C₁-C₆ alkyl, phenyl, a 5 to 6 membered heteroaryl, wherein the alkyl, phenyl and heteroaryl are optionally substituted with halogen or C₁-C₃ alkyl;
each R^{g} is independently selected from halogen, CN, SF₅, C₁-C₆ alkyl, C₁-C₆ alkoxy, a 3 to 6 membered carbocycle, a 3 to 6 membered heterocycle, phenyl, and a 5 to 6 membered heteroaryl, wherein the alkyl, alkoxy, carbocycle, heterocycle, phenyl and heteroaryl are optionally substituted with halogen; and
R^{h} and Rⁱ are independently selected from hydrogen and C₁-C₆ alkyl, wherein the alkyl is optionally substituted with halogen, CN or C₁-C₆ alkoxy.

3. A compound of claim 1, wherein:
W is CR¹²R¹³;
Y is O, S or NR¹;
Z is a bond, CH₂ or C(═O), provided when Z is C(═O) then Y is NR¹;
X₁ is O;
X₂ is selected from CR⁵R⁶, NR⁷ and O;
X₃ is selected from CR⁸R⁹ and O, provided when X₃ is O then X₂ is CR⁵R⁶;
X₄ is CR¹¹;
X₅ is selected from CR¹⁴R¹⁵ and O, provided when X₅ is O, then X₂ is CR⁵R⁶ and X₃ is CR⁸R⁹ or a bond;
X₆ is CR¹⁶R¹⁷;
R¹ is CH₃;
R² and R³ are independently selected from hydrogen and C₁-C₆ alkyl;
R⁴ is selected from halogen, C₁-C₆ alkoxy, NHC(═O)R^{f}, phenyl and a 5 to 6 membered heteroaryl, wherein the alkoxy, phenyl and heteroaryl are optionally substituted with one or more R^{g} groups;
R⁵ and R⁶ are independently selected from hydrogen, halogen, OR^{a}, NR^{b}R^{c}, C₁-C₆ alkyl, C₁-C₆ alkoxy, phenyl, a 3 to 6 membered heterocycle and a 5 to 6 membered heteroaryl, wherein the phenyl, heterocycle and heteroaryl are optionally substituted with halogen, or
R⁵ and R⁶ together with the atom to which they are attached form a 3 to 6 membered heterocycle;
R⁷ is selected from hydrogen, C₁-C₆ alkyl, C₁-C₆ alkoxycarbonyl, —SO₂(C₁-C₆ alkyl), and a 3 to 6 membered heterocyclic, wherein the alkyls and alkoxycarbonyl are optionally substituted with one or more R^{d} groups;
R⁸ and R⁹ are independently selected from hydrogen and OR^{e}, or
R⁸ and R⁹ taken together form an oxo group, or
R⁸ and R⁹ together with the atom to which they are attached form a 3 to 6 membered heterocycle;
R¹¹ is hydrogen or halogen;
R¹² and R¹³ are hydrogen;
R¹⁴ and R¹⁵ are independently selected from hydrogen and C₁-C₃ alkyl;
R¹⁶ and R¹⁷ are independently selected from hydrogen and halogen;
R^{a} is selected from hydrogen, C₁-C₆ alkyl and (CH₂)₀₋₃ (3 to 6 membered carbocyclic);
R^{b} and R^{c} are independently selected from hydrogen and C₁-C₆ alkyl, or
R^{b} and R^{c} together with the nitrogen atom to which they are attached form a 3 to 6 membered heterocyclic;
each R^{d} is selected from halogen, hydroxy, oxo, C₃-C₆ cycloalkyl and phenyl, wherein the phenyl is optionally substituted with C₁-C₃ alkoxy;
R^{e} is hydrogen;
R^{f} is a 5 to 6 membered heteroaryl optionally substituted with halogen or C₁-C₃ alkyl; and
each R^{g} is independently selected from halogen, CN, C₁-C₆ alkyl and C₁-C₆ alkoxy, wherein the alkyl and alkoxy are optionally substituted with halogen.

4. A compound of claim 1, having the Formula I′a:

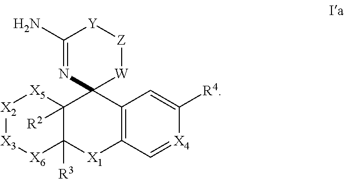

5. A compound of claim 1, having the Formula I′b:

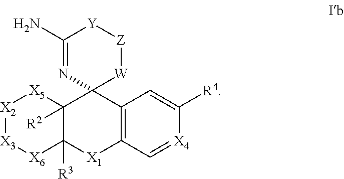

6. A compound of claim 1, having the Formula I′c:

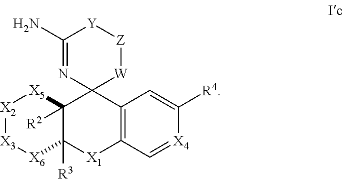

7. A compound of claim 1, having the Formula I′d:

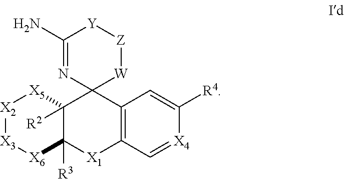

8. A compound of claim 1, having the

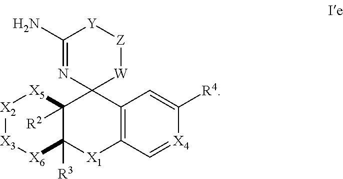

9. A compound of claim 1, having the Formula I′f:

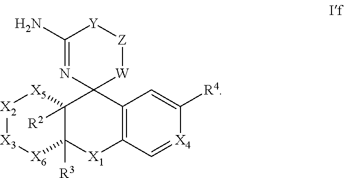

10. A compound of claim 1, having the Formula I′g:

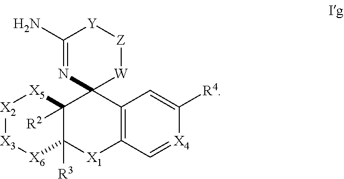

11. A compound of claim 1, having the Formula I′h:

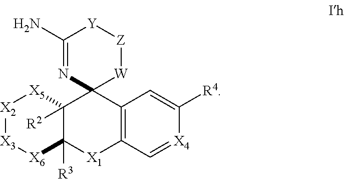

12. A compound of claim 1, having the Formula I′i:

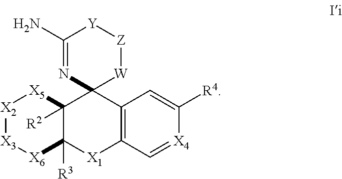

13. A compound of claim 1, having the Formula I′j:

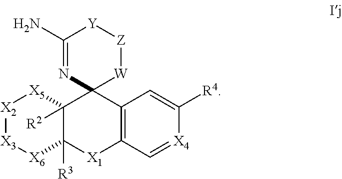

14. A compound of claim 1, having the Formula I′k:

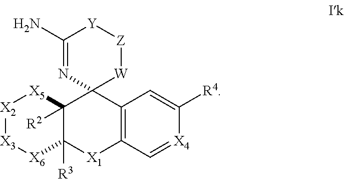

15. A compound of claim 1, having the Formula I′l:

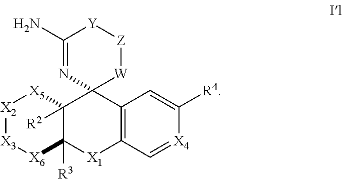

16. A compound of claim 1, having the Formula I′m:

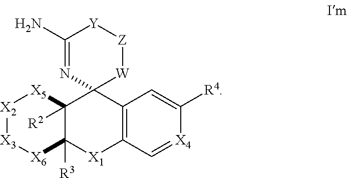

17. A compound of claim 1, having the Formula I′n:

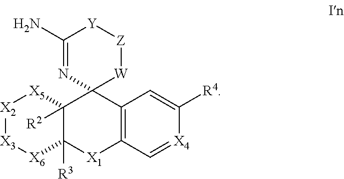

18. A compound of claim 1, having the Formula I′o:

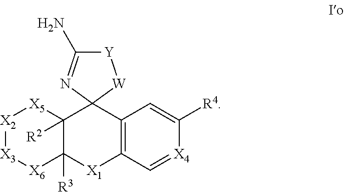

19. A compound of claim 1, having the Formula I′p:

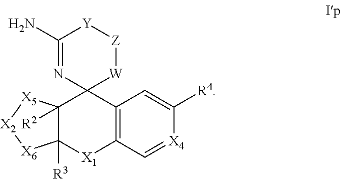

20. A compound of claim 1, wherein R¹² and R¹³ are hydrogen.

21. A compound of claim 20, wherein Y is O and Z is a bond.

22. A compound of claim 20, wherein Y is S and Z is a bond.

23. A compound of claim 1, wherein Y is NR¹ and Z is C(═O).

24. A compound of claim 1, wherein Y is S and Z is CH₂.

25. A compound of claim 1, wherein X₁ is O.

26. A compound of claim 1, wherein X₄ is CR¹¹.

27. A compound of claim 26, wherein X₄ is CH, CF or CCl.

28. A compound of claim 1, wherein R² and R³ are independently selected from hydrogen or methyl.

29. A compound of claim 1, wherein X₂ is CR⁵R⁶.

30. A compound of claim 1, wherein X₂ is NR⁷.

31. A compound of claim 1, wherein X₂ is O.

32. A compound of claim 1, wherein R⁵ and R⁶ are independently selected from hydrogen, F, hydroxy, methyl, ethyl, methoxy, neopentyloxy, cyclopropylmethoxy, cyclobutoxy, isopropoxy, methoxymethyl, ethoxymethyl, dimethylamine, phenyl, pyrrolidin-1-yl, pyridin-2-yl and 5-chloropyridin-3-yl.

33. A compound of claim 1, wherein R⁷ is selected from hydrogen, isopropyl, isobutyl, 2,2,2-trifluoroethyl, cyclopropylethyl, (4-methoxyphenyl)methyl, 2,2-difluoroethyl, CH₂CH(CH₃)₂OH, C(═O)CH₂CH(CH₃)₂, C(═O)CH₂(cyclopropyl), C(═O)O(benzyl), C(═O)OCH(CH₃)₂, S(O₂)CH₂CH(CH₃)₂, S(O₂)CH₂(cyclopropyl) and tetrahydropyran-4-yl.

34. A compound of claim 1, wherein; X₃ is CR⁸R⁹.

35. A compound of claim 1, wherein R⁸ and R⁹ are independently selected from hydrogen and OH.

36. A compound of claim 1, wherein R⁸ and R⁹ taken together form an oxo group.

37. A compound of claim 1, wherein R⁸ and R⁹ together with the atom to which they are attached form 1,3-dioxolan-2-yl.

38. A compound of claim 1, wherein X₃ is O.

39. A compound of claim 1, wherein X₅ is CR¹⁴R¹⁵.

40. A compound of claim 39, wherein R¹⁴ and R¹⁵ are independently selected from hydrogen and methyl.

41. A compound of claim 1, wherein X₅ is O.

42. A compound of claim 1, wherein R¹⁶ and R¹⁷ are independently selected from hydrogen and F.

43. A compound of claim 1, wherein R¹ is methyl.

44. A compound of claim 1, wherein R⁴ is selected from Br, methoxy, 5-chloropyridin-3-yl, 2-fluoropyridin-3-yl, pyrimidin-5-yl, 5-cyanopyridin-3-yl, 5-fluoropyridin-3-yl, 5-(trifluoromethyl)pyridin-3-yl, 4-(trifluoromethyl)pyridin-2-yl, 5-chloro-2-fluoropyridin-3-yl, 4-cyanopyridin-2-yl, 5-methoxypridin3-yl, 3-chloro-5-fluorophenyl, 3-chlorophenyl, 3-cyanophenyl, 3-(difluoromethoxy)phenyl, 3,5-difluorophenyl, 3-chloro-2-fluorophenyl, 3-cyano-6-fluorophenyl, 5-chloro-2-fluorophenyl, 3-chloro-4-fluorophenyl, 3-cyano-5-fluorophenyl, 3-cyano-5-bromophenyl, 3-cyano-2-fluorophenyl, 3,5-dichlorophenyl, 3-chloro-5-(trifluoromethyl)phenyl, 3-cyano-5-chlorophenyl, NHC(═O)(2-methyloxazol-4-yl), NHC(═O)(5-chloropyridin-2-yl) and NHC(═O)(5-bromochloropyridin-2-yl).

45. (canceled)

46. A compound of Formula I as defined in claim 1 and named in any one of Example 1 to 310 herein, or a stereoisomer, diastereomer, enantiomer, tautomer or pharmaceutically acceptable salt thereof.

47. (canceled)

48. A method of inhibiting cleavage of APP by β-secretase in a mammal comprising administering to said mammal an effective amount of a compound of claim 1.

49. A method for treating a disease or condition mediated by the cleavage of APP by β-secretase in a mammal, comprising administering to said mammal an effective amount of a compound of claim 1.

50. The method of claim 49, wherein the disease is Alzheimer's disease.

51. A pharmaceutical compositions comprising a compound of claim 1 and a pharmaceutically acceptable carrier, diluent or excipient.

52. (canceled)

53. (canceled)

54. (canceled)

55. (canceled)
